(19) United States
(12) Patent Application Publication
(10) Pub. No.: US 2020/0017917 A1
(43) Pub. Date: Jan. 16, 2020

(54) Mapping a Functional Cancer Genome Atlas of Tumor Suppressors Using AAV-CRISPR Mediated Direct In Vivo Screening

(71) Applicant: YALE UNIVERSITY, New Haven, CT (US)

(72) Inventors: Sidi CHEN, Milford, CT (US); Ryan CHOW, San Jose, CA (US)

(73) Assignee: Yale University, New Haven, CT (US)

(21) Appl. No.: 16/489,612

(22) PCT Filed: Mar. 2, 2018
(86) PCT No.: PCT/US2018/020712

§ 371 Date: Aug. 28, 2019

Related U.S. Application Data
(60) Provisional application No. 62/600,802, filed on Mar. 3, 2017.

(51) Int. Cl. C12Q 1/6886; C12N 15/86; C12N 15/90; C12N 9/22; C12N 9/96; C12Q 1/6809; C12N 15/11

(52) U.S. Cl. CPC C12Q 1/6886; C12N 15/86; C12N 15/90; C12N 9/22; C12N 9/96; C12Q 1/6809; C12Q 2600/154; A01K 2217/072; A01K 2227/105; A01K 2267/0393; C07K 2319/85; C12N 2310/20; C12N 2320/12; C12N 15/111

(57) ABSTRACT

The present invention includes compositions and methods for identifying cancer driver mutations through use of an AAV-CRISPR library and molecular inversion sequencing probes (MIPs).

### CROSS-REFERENCE TO RELATED APPLICATION

The present application is entitled to priority under 35 U.S.C. § 119(e) to U.S. Provisional Patent Application No. 62/600,802 filed Mar. 3, 2017, which is hereby incorporated by reference in its entirety herein.

### STATEMENT REGARDING FEDERALLY SPONSORED RESEARCH OR DEVELOPMENT

This invention was made with government support under CA209992, CA121974, CA196530, GM007205 awarded by National Institutes of Health. The government has certain rights in the invention.

### BACKGROUND OF THE INVENTION

Large-scale molecular profiling of patient samples has tremendously improved the understanding of human cancers. The multidimensional landscapes produced by international consortia such as The Cancer Genome Atlas (TCGA) and Catalog of Somatic Mutations In Cancer (COSMIC), encompassing key datasets such as somatic mutations, copy number variants, epigenetic marks, mRNA and microRNA transcriptomes, as well as protein levels, have illuminated the molecular underpinnings of cancer at an unprecedented resolution and scale. Consequently, there is now an extensive catalog of significantly mutated genes (SMGs) that are recurrently mutated across different patients, both within and across histological subtypes. While some SMGs are well-known tumor suppressors or oncogenes, other SMGs have not been previously implicated in cancer. Though the identification of SMGs is an important first step towards the development of new therapeutic avenues, functional evidence is required to definitively determine which genomic alterations are essential for the growth of an individual cancer. A number of statistical algorithms have been developed that aim to distinguish SMGs that are “drivers” of cancer growth from those that are mere “passengers” mutations. However, the functional role of many of these SMGs remains to be explicitly tested in controlled experimental settings. In order to pinpoint the most relevant targets for clinical intervention, it is essential to systematically assess the contribution of each SMG, and combinations of SMGs, to cancer progression.

Genetically engineered mouse models (GEMMS) have been instrumental for studying the mechanisms of oncogenes and tumor suppressors in vivo. Conditional or germline knockout alleles enable in vivo modeling of diverse diseases, including a wide variety of cancer types. However, GEMMS are time-consuming to produce, involving a complex multi-step process that requires embryonic stem cell modification, the generation of chimeras, germline transmission, and mouse colony expansion. Owing to the technical difficulties of this process, and the complexity of breeding with large numbers of genetic modifications, GEMMS have largely been limited to the study of only a handful of genes at a time. Thus, a systematic characterization of the hundreds of SMGs identified through tumor sequencing studies is impractical using GEMMS.

There is a need in the art for compositions and methods to interrogate in vivo the functional roles of genes in cancer progression in a high-throughput manner. The present invention satisfies this need.

### SUMMARY OF THE INVENTION

The present invention relates to compositions and methods for determining cancer driver mutations.

One aspect of the invention includes a method of determining at least one cancer driver mutation in vivo in a cancer-affected subject. The method comprises administering to the subject a plurality of AAV-CRISPR vectors, wherein the AAV-CRISPR vectors comprise Cas9 and a plurality of short guide RNAs (sgRNAs) homologous to a plurality of tumor suppressor genes (TSGs). The plurality of nucleic acids isolated from the subject's cancer are sequenced and analysis of the sequencing data indicates whether any cancer driver mutation is present in the subject's cancer.

Another aspect of the invention includes a method of identifying a plurality of cancer driver mutations in a sample. The method comprises hybridizing a plurality of Molecular Inversion Probes (MIPs) to a plurality of nucleic acids from the sample and performing targeted capture sequencing on the plurality of nucleic acids. Analyzing the data from the targeted capture sequencing indicates the presence and/or nature of any plurality of cancer driver mutations in the sample.

Yet another aspect of the invention includes a composition comprising a set of Molecular Inversion Probes (MIPs) comprising at least one selected from the group consisting of SEQ ID NOs. 289-554. Still another aspect of the invention includes a composition comprising a set of Molecular Inversion Probes (MIPs) comprising SEQ ID NOs. 289-554.

Another aspect of the invention includes a kit comprising a set of Molecular Inversion Probes (MIPs) comprising at least one selected from the group consisting of SEQ ID NOs. 289-554, and instructional material for use thereof. Yet another aspect of the invention includes a kit comprising a composition comprising a set of Molecular Inversion Probes (MIPs) comprising SEQ ID NOs. 289-554, and instructional material for use thereof. Still another aspect of the invention includes a kit for determining at least one cancer driver mutation in a sample comprising a set of Molecular Inversion Probes (MIPs) comprising at least one selected from the group consisting of SEQ ID NOs. 289-554, reagents for measuring the at least one cancer driver mutation, and instructional material for use thereof. Another aspect of the invention includes a kit for determining at least one cancer driver mutation in a sample comprising a set of Molecular Inversion Probes (MIPs) comprising SEQ ID NOs. 289-554, reagents for measuring the at least one cancer driver mutation, and instructional material for use thereof.

Still another aspect of the invention includes a method of determining at least one cancer driver mutation in a sample. The method comprises contacting a plurality of Adeno-Associated Virus-Clustered Regularly Interspaced Short Palidromic Repeats (AAV-CRISPR) vectors with the sample. The vectors comprise Cas9 and a plurality of nucleotide sequences homologous to a plurality of tumor suppressor genes (TSGs). A reaction mixture is generated. A plurality of nucleic acids isolated from the reaction mixture are sequenced and the sequencing data are analyzed as to identify any cancer driver mutation therein.

Another aspect of the invention includes a method of determining treatment for a subject suffering from cancer. The method comprises contacting a plurality of AAV-CRISPR vectors with a sample from the subject. The vectors comprise Cas9 and a plurality of nucleotide sequences homologous to a plurality of tumor suppressor genes (TSGs). A reaction mixture is generated. A plurality of nucleic acids isolated from the reaction mixture are sequenced and the data from the sequencing are analyzed as to identify any mutation in the plurality of nucleic acids. Treatment for the subject suffering from cancer is determined based on the presence and/or nature of any mutation in the plurality of nucleic acids.

Yet another aspect of the invention includes an AAV-CRISPR mTSG library comprising a plurality of AAV vectors comprising Cas9 and a plurality of nucleic acids homologous to a plurality of Tumor Suppressor Gene (TSGs).

Still another aspect of the invention includes a vector comprising an adeno-associated virus (AAV) genome, a U6 promoter gene, an sgRNA sequence, an EFS promoter gene, and a Cre recombinase gene.

Another aspect of the invention includes a vector comprising an adeno-associated virus (AAV) genome, a U6 promoter gene, an sgRNA sequence, a TBG promoter gene, and a Cre recombinase gene. Yet another aspect of the invention includes a vector comprising the nucleic acid sequence of SEQ ID NO: 555. Still another aspect of the invention includes a vector comprising the nucleic acid sequence of SEQ ID NO: 556.

Yet another aspect of the invention includes a kit comprising a vector comprising the nucleic acid sequence of SEQ ID NO: 555, and instructional material for use thereof. Another aspect of the invention includes a kit comprising a vector comprising the nucleic acid sequence of SEQ ID NO: 556, and instructional material for use thereof. Still another aspect of the invention includes a kit comprising an adeno-associated virus (AAV) genome, a U6 promoter gene, an sgRNA sequence, an EFS promoter gene, a Cre recombinase gene, and instructional material for use thereof. Yet another aspect of the invention includes a kit comprising an adeno-associated virus (AAV) genome, a U6 promoter gene, an sgRNA sequence, an TBG promoter gene, a Cre recombinase gene, and instructional material for use thereof.

In various embodiments of the above aspects or any other aspect of the invention delineated herein, the sgRNA sequences comprise at least one selected from the group consisting of SEQ ID NOs. 1-280. In one embodiment, the sgRNA sequences comprise SEQ ID NOs. 1-280.

In one embodiment, the sequencing comprises targeted capture sequencing. In another embodiment, the targeted capture sequencing is performed using a plurality of Molecular Inversion Probes (MIPs). In yet another embodiment, the plurality of MIPs comprises at least one selected from the group consisting of SEQ ID NOs. 289-554. In still another embodiment, the plurality of MIPs comprises SEQ ID NOs. 289-554.

In one embodiment, the mutation is a nucleotide insertion. In another embodiment, the insertion comprises more than one nucleotide base. In yet another embodiment, the mutation is a nucleotide deletion. In still another embodiment, the deletion comprises more than one nucleotide base.

In one embodiment, the subject is a mammal. In another embodiment, the animal is a mouse or a human.

In one embodiment, the MIPs comprise at least one selected from the group consisting of SEQ ID NOs. 289-554. In another embodiment, the plurality of MIPs comprises at least one selected from the group consisting of SEQ ID NOs. 289-554.

In one embodiment, the plurality of nucleotide sequences homologous to a plurality of TSGs comprises at least one selected from the group consisting of SEQ ID NOs. 1-280. In another embodiment, the plurality of nucleotide sequences homologous to a plurality of TSGs comprises SEQ ID NOs. 1-280.

In one embodiment, the sample is a plurality of cancer cells from the subject. In another embodiment, the sample is a tumor from the subject.

In one embodiment, the TBG promoter gene comprises the nucleic acid sequence of SEQ ID NO: 557.

### BRIEF DESCRIPTION OF THE DRAWINGS

The following detailed description of specific embodiments of the invention will be better understood when read in conjunction with the appended drawings. For the purpose of illustrating the invention, there are shown in the drawings exemplary embodiments. It should be understood, however, that the invention is not limited to the precise arrangements and instrumentalities of the embodiments shown in the drawings.

FIGS. 1A-1I are a series of plots and images illustrating that the AAV-CRISPR mTSG library rapidly induces liver tumor growth in LSL-Cas9 mice. FIG. 1A is a schematic describing the AAV-CRISPR mTSG library design and experimental outline. First, the top significantly mutated genes were identified from pan-cancer TCGA datasets. After removing known oncogenes and genes without mouse orthologs, a set of 49 most significantly mutated putative tumor suppressor genes were chosen (mTSG). Seven additional genes with housekeeping functions were spiked-in, leading to a final set of 56 genes. SgRNAs targeting these genes were then identified computationally and 5 were chosen for each gene. 280 sgRNAs plus 8 non-targeting control (NTC) sgRNAs were synthesized, and then the sgRNA library (mTSG, 288 sgRNAs) was cloned into an expression vector that also contained Cre recombinase and a Trp53 sgRNA. AAVs carrying the mTSG library were produced, and the pooled AAVs injected into the tail veins of LSL-Cas9 mice. After a specified time period, the mice were subjected to MRI, histology, and MIPs capture sequencing analysis. FIG. 1B shows magnetic resonance imaging of abdomens of mice treated with PBS, vector, or mTSG library. Detectable tumors are circled with dashed lines. PBS treated mice (n=3) did not have any detectable tumors, while vector treated mice (n=3) occasionally had small nodules. In contrast, mTSG-treated mice (n=4) often had multiple detectable tumors. FIG. 1C shows Kaplan-Meier survival curves for PBS (n=10), vector (teal, n=11), and mTSG (orange, n=27) treated mice. No mTSG-treated mice survived longer than four months post treatment, while all PBS and vector treated animals survived the duration of the experiment. Statistical significance was assessed by log-rank test (p=1.8*10⁻¹¹). FIG. 1D shows brightfield images with GFP fluorescence overlay of livers from representative PBS, vector, and mTSG-treated mice, 4 months post-treatment. Large GFP+ tumors are marked with arrowheads. In contrast to PBS or vector-treated mice, mTSG-treated mice had numerous detectable GFP+ liver nodules. FIG. 1E shows hematoxylin and eosin staining of liver sections from mice treated with PBS (n=7), vector (n=5), or mTSG library (n=13). Tumor-normal boundaries are demarcated with dashed lines. No tumors were found in PBS samples, while small nodules were found, although rare, in vector samples. On the other hand, mTSG-treated livers were replete with tumors (statistics in FIGS. 1F-1G). FIG. 1F is a dot plot of the total tumor area per mouse (mm²) in liver sections from mice treated with PBS (n=7), vector (n=5), or mTSG library (n=13). mTSG-treated mice had a significantly higher total tumor burden than PBS (one-sided Welch's t-test, p=0.027) or vector-treated mice (p=0.034). FIG. 1G is a dot plot of the individual tumor area (mm²) in liver sections from mice treated with PBS (n=7), vector (n=9), or mTSG library (n=49). mTSG-treated mice had a significantly larger tumors than PBS (one-sided Welch's t-test, p<0.0001) or vector-treated mice (p=0.0003). FIG. 1H is a plot of median log₂ sequencing coverage across all sequenced samples in amplicons targeted by the 266 MIPs (black dots). MIPs were designed to amplify the genomic regions flanking the predicted cut sites of each sgRNA. 95% confidence intervals for the median are depicted with blue lines. Median read depth across all MIPs approximated a lognormal distribution, indicating relatively even capture of the target loci. FIG. 1I illustrates representative IHC staining of a liver hepatocellular carcinoma (LIHC or HCC) marker, pan-cytokeratin (AE1/AE3) from mice treated with PBS, vector, or mTSG library. The tumors from mTSG-treated samples shown revealed positive staining for AE1/AE3, consistent with LIHC pathology. Certain mTSG tumors were partially positive for cytokeratin, revealing tumor heterogeneity. The tumors from vector-treated samples were relatively small and almost always negative or slightly positive for cytokeratin. Scale bar is 0.5 mm.

FIGS. 2A-2C are a series of plots and images illustrating MIPs capture sequencing enables direct, high-throughput assessment of AAV-CRISPR library induced mutagenesis and mutational variant level landscape of mouse AAV-mTSG induced LIHC. FIG. 2A shows unique variants observed at the genomic region targeted by Setd2 sg1 in representative PBS, vector, and mTSG-treated liver samples. The percentage of total reads that correspond to each genotype is indicated on the right in the boxes. No indels were found in the PBS or vector-treated samples, while several unique variants were identified in the mTSG-treated sample (mTSG liver 042). FIG. 2B is a set of waterfall plots of two mTSG-treated liver samples (042, 066) detailing sum variant frequencies in significantly mutated sgRNA sites (SMSs). Individual mice presented with distinct mutational signatures, suggesting that a wide variety of mutations induced by the mTSG library had undergone positive selection. FIG. 2C is a global heatmap detailing the square-root of sum variant frequency across all sequenced samples (n=133) from mTSG (n=98 samples), vector (n=21 samples), or PBS-treated mice (n=14 samples) in terms of sgRNAs. Each row represents one sgRNA, while each column represents one sample. Treatment conditions and tissue type are annotated at the top of the heatmap: big abdominal tumor, detectable tumor outside liver, liver, and other organs. Bar plots of the mean average variant frequencies for each sgRNA (right panel) and each sample (bottom panel) are also shown. mTSG-treated organs without visible tumors (0.11±0.01 SEM) had significantly lower mean square-root variant frequencies compared to mTSG-treated tumors and livers: BATs (0.52±0.27, p<0.0001 by unpaired t-test), non-liver tumors (0.33±0.04, p<0.0001), and livers (0.50±0.04, p<0.0001). Livers and other organs from vector-treated animals (0.22±0.06 and 0.08±0.004, respectively) and PBS-treated animals (0.12±0.03 and 0.08±0.01, respectively) all had significantly lower variant frequencies than mTSG-treated livers (p<0.0001 for all comparisons).

FIG. 3 is a heatmap illustrating the mouse gene-level mutational landscape of liver hepatocellular carcinoma (LIHC aka HCC). Each row in the figure corresponds to one gene in the mTSG library, while each column corresponds to one mTSG-treated liver sample. Top: Bar plots of the total number of significantly mutated genes (SMGs) identified in each mTSG-treated liver sample (n=37). Samples originating from the same mouse are grouped together, and denoted with a gray bar underneath. Center: Tile chart depicting the mutational landscape of primary liver samples infected with the mTSG library. Genes are grouped and colored according to their functional classifications (DNA repair/replication, epigenetic modifier, cell death/cycle, repressor, immune regulator, ubiquitination, transcription factor, cadherin, ribosome related and RNA synthesis/splicing), as noted in the legend in the top-right corner. Colored boxes indicate that the gene was significantly mutated in a given sample, while a gray box indicates no significant mutation. Right: Bar plots of the percentage of liver samples that had a mutation in each of the genes in the mTSG library. Trp53, Setd2, Pik3r1, Cic, B2m, Vh1, Notch1, Cdh1, Rp122 and Polr2a were the top mutated genes in each of the 10 functional classifications, respectively. Bottom: Stacked bar plots describing the type of indels observed in each sample, color-coded according to the legend in the bottom-right corner. Frameshift insertions or deletions comprised the majority of variant reads (median=59.2% across all samples). Left: Heatmap of the number of significantly mutated sgRNA sites (0-5 SMSs) for each gene. Multiple significantly mutated sgRNA sites for a given gene are indicative of a strong selective force for loss-of-function mutations in that gene.

FIGS. 4A-4M are a series of plots and images illustrating co-mutation analysis of liver samples from mTSG-treated mice reveals potential synergistic combinations of driver mutations. FIG. 4A, upper-left triangle of the heatmap, shows co-occurrence rates for each gene pair. To calculate co-occurrence rates, the “intersection” is defined as the number of double-mutant samples, and the “union” as the number of samples with a mutation in either of the two genes. The co-occurrence rate was then calculated as the intersection divided by the union. FIG. 4A, lower-right triangle of the heatmap, illustrates −log₁₀ p-values by hypergeometric test to evaluate whether specific pairs of genes are statistically significantly co-mutated. FIG. 4B is a scatterplot of the co-occurrence rates for each gene pair, plotted against −log₁₀ Benjamini-Hochberg adjusted q- values by hypergeometric test. The top co-occurring pair was Setd2+Trp53, with 75% co-occurrence rate (18 double mutated samples out of 24 samples with either samples mutated, co-occurrence rate=18/24) (hypergeometric test, Benjamini-Hochberg adjusted q=0.0117). Other labeled top co-mutated pairs were Cdkn2a+Pten (co-occurrence rate=7/10=70%, q=0.0203), Cdkn2a+Rasa1 (6/9=67%, q=0.0352), and Arid2+Cdkn1b (11/17=65%, q=0.0352). FIG. 4C is a set of Venn diagrams showing the strong co-occurrence of mutations in Setd2+Trp53 (top left), Cdkn2a+Pten (top right), Cdkn2a+Rasa1 (bottom left), and Arid2+Cdkn1b (bottom right). Numbers shown correspond to the number of mTSG-treated liver samples with a given mutation profile. FIG. 4D, upper-left triangle of the heatmap, illustrates the pairwise Pearson correlation of sum % variant frequency for each gene, averaged across sgRNAs. FIG. 4D, lower-right triangle of the heatmap, illustrates −log₁₀ p-values by t-distribution to evaluate the statistical significance of the pairwise correlations. FIG. 4E is a scatterplot of pairwise Pearson correlations plotted against −log₁₀ Benjamini-Hochberg adjusted q-values. The top four correlated gene pairs were Casp8+Kdm6a (corr.=0.933, q=6.16*10⁻¹⁴), Map2k4+Nf1 (corr.=0.928, q=9.86*10⁻¹⁴), Arid1a+Casp8 (corr.=0.927, q=9.96*10⁻¹⁴), and Fbxw7+Pcna (corr.=0.911, q=2.05*10⁻¹²).

FIG. 4F is a scatterplot comparing sum level % variant frequency for Map2k4 vs. Nf1 across all mTSG-treated liver samples. The Pearson correlation coefficient is noted on the plot (corr. (R)=0.928, q=9.86*10⁻¹⁴). FIG. 4G is a heatmap of the p-values associated with the top 10 mutation pairs that were found to be statistically significant in both co-occurrence (left) and correlation (right) analyses. 5 of the 10 mutation pairs included Cdkn2a, suggesting that loss-of-function in Cdkn2a amplifies the oncogenic effects of mutations in other tumor suppressors. FIG. 4H is a scatterplot of the cooccurrence rates for each gene pair, plotted against −log₁₀ p-values by hypergeometric test. Highly co-occurring pairs include Cdkn2a+Pten (co-occurrence rate=7/10=70%; hypergeometric test, p=2.63*10⁻⁵), Cdkn2a+Rasa1 (6/9=67%; p=7.96*10⁻⁵), Arid2+Cdkn1b (11/17=65%; p=9.13*10⁻⁵) and Kansl1+B2m (11/18=61%; p=3.6*10⁻⁴). FIG. 4I is a series of Venn diagrams showing the strong co-occurrence of mutations in B2m+Kansl1 (top left), Cdkn2a+Pten (top right), Cdkn2a+Rasa1 (bottom left), and Arid2+Cdkn1b (bottom right). Numbers shown correspond to the number of mTSG-treated liver samples with a given mutation profile. FIG. 4J, upper-left triangle, is a heat map of the pairwise Spearman correlation of sum % variant frequency for each gene, summed across sgRNAs. Lower-right triangle: heat map of −log₁₀ p-values by t-distribution to evaluate the statistical significance of the pairwise correlations. FIG. 4K is a scatterplot of pairwise Spearman correlations plotted against −log₁₀ p-values. The top four correlated pairs were Cdkn2a+Pten (Spearman R=0.817, p=6.97*10⁻¹⁰), Nf1+Rasa1 (R=0.791, p=5.86*10⁻⁹), Arid2+Cdkn1b (R=0.788, p=7.16*10⁻⁹), and Cdkn2a+Rasa1 (R=0.761, p=4.45*10⁻⁸). FIG. 4L is a scatterplot comparing sum level % variant frequency for Arid2 vs. Cdkn1b across all mTSG-treated liver samples. Spearman and Pearson correlation coefficients are noted on the plot (Spearman R=0.788; Pearson R=0.746). FIG. 4M is a heat map of the p-values associated with the top mutation pairs that were found to be statistically significant (Benjamini-Hochberg adjusted p<0.05) in both cooccurrence (left) and correlation (right) analyses.

FIGS. 5A-5E are a series of plots and images illustrating systematic dissection of variant compositions across individual liver lobes within a single mTSG-treated mouse reveals substantial clonal mixture between lobes. FIG. 5A is a schematic of the experimental workflow for analysis of multiple liver lobes (n=5) from a single mTSG-treated mouse. FIG. 5B is a heatmap of Spearman's rank correlation coefficients among 5 liver samples from a single mTSG-treated mouse, calculated on the basis of variant frequency for all unique variants present within the 5 samples. Notably, lobes 1-4 are all significantly correlated with lobe 5, with lobe 3 having the strongest correlation to lobe 5. FIG. 5C is a heatmap of variant frequencies for each unique variant identified across the 5 individual liver lobes after square-root transformation. Rows correspond to different liver lobes, while columns denote unique variants. Eight clusters were identified based on binary mutation calls, and are indicated on the bottom of the heatmap. FIG. 5D is a series of pie charts depicting the proportional contribution of each cluster to the 5 liver lobes. In order for a cluster to be considered, at least half of the variants within the cluster must be present in that particular sample. For each lobe, variant frequencies within a cluster were averaged and converted to relative proportions, as shown in the pie charts. The pie charts accurately recapture the correlation analysis in FIG. 5B, while additionally providing quantitative insight into the shared variants between the 5 liver lobes. FIG. 5E is an image wherein each box corresponds to one cluster, color-coded as in FIG. 5C-5D, showing the top four variants in each cluster. On the basis of whether a variant cluster was present in multiple liver lobes, each box is also classified as either a private or a shared variant cluster. Clusters 1, 2, 3, 5 and 6 are largely unique to individual lobes (“private” variant clusters), while clusters 4, 7 and 8 are present in multiple lobes (“shared” variant clusters). Cluster #8 was found in 4 out of 5 lobes, and is characterized by mutations in Ml13, Setd2 and Trp53.

FIGS. 6A-6E are a series of images and plots illustrating Setd2 and Trp53 mutations drive liver tumorigenesis in mice, and define a subset of liver hepatocellular carcinoma (LIHC or HCC) patients with poor prognosis. FIG. 6A is a schematic of the experimental strategy to functionally test individual and gene pairs as drivers of liver tumorigenesis. Plasmids contained one sgRNA targeting Trp53, and either a non-targeting sgRNA (NTC+Trp53) or an sgRNA targeting Setd2 (Setd2+Trp53). The plasmids also contained a liver-specific TBG promoter driving the expression of firefly luciferase (FLuc) and Cre recombinase. AAVs were generated with these plasmids and injected via i.v. into LSL-Cas9 mice. FIG. 6B shows bioluminescence imaging of mice injected with NTC+Trp53 or Setd2+Trp53 AAVs, one month post treatment. No tumors were found in NTC+Trp53 AAV treated mice (n=4), while all Setd2+Trp53 AAV treated mice developed tumors (n=5) (one tailed Chi-square test, p=0.0013). Luminescence intensities are shown in units of photons/sec/cm²/sr. FIG. 6C shows Kaplan-Meier survival analysis of human LIHC patients from TCGA. Patients were classified in terms of SETD2 status, based on somatic mutations, copy number variation, and expression profiles. SETD2⁻ patients (n=26) had significantly worse prognosis than SETD2+ patients (n=346) (log-rank test, p=0.042). FIG. 6D shows Kaplan-Meier survival analysis of human LIHC patients from TCGA. Patients were classified in terms of TP53 status, based on somatic mutations, copy number variation, and expression profiles. TP53⁻ patients (n=126) had significantly worse prognosis than TP53+ patients (n=246) (log-rank test, p=0.0043). FIG. 6E shows Kaplan-Meier survival analysis of human LIHC patients from TCGA. Patients were classified in terms of both SETD2 and TP53 status, based on somatic mutations, copy number variation, and expression profiles. SETD2⁻TP53⁻ patients (n=11) had significantly worse prognosis than all other patients (log-rank test, p=0.0011 comparing all 4 survival curves. Pairwise comparisons for SETD2TP53″ group: p <0.0001 vs. SETD2+TP53+(n=231), p=0.039 vs. SETD2+TP53⁻ (n=115), p=0.039 vs. SETD2TP53+(n=15)).

FIGS. 7A-7C are a series of images and plots illustrating representative full-spectrum MRI series of livers from PBS, vector, and mTSG-treated mice. FIG. 7A shows full-spectrum MRI slices from representative PBS, vector, and mTSG-treated mice. FIG. 7B is a dot plot of the sum tumor volume per mouse (in mm³) in mice treated with PBS (n=3), vector (n=3), or mTSG library (n=4). mTSG-treated mice had significantly higher tumor burden than PBS (one-sided Mann-Whitney test, p=0.0286) or vector-treated animals (p=0.0286). FIG. 7C is a dot plot of individual tumor volume (in mm³) in mice treated with PBS (n=3), vector (n=3), or mTSG library (n=6). mTSG-treated mice had significantly larger tumors than PBS (one-sided Mann-Whitney test, p=0.0119) or vector-treated animals (one-sided Mann-Whitney test, p=0.0357).

FIG. 8 is a series of images showing representative full slide scanning images of mouse liver sections in PBS, vector and mTSG treatment groups. Full slide scans of liver sections from PBS, vector, and mTSG-treated mice. Two representative mice from each group are shown. Slide scan data from additional mice (PBS (n=7), vector (n=5), and mTSG (n=13)) were also analyzed. Some brain sections are also present in the same scanned field, noted with asterisks. PBS samples did not have any detectable nodules, while vector-treated samples occasionally had developed small nodules. In contrast, mTSG-treated samples were replete with tumors.

FIGS. 9A-9Q are a series of plots illustrating significantly mutated sgRNA sites across all liver samples from mice treated with AAV-CRISPR mTSG library. Waterfall plots of significantly mutated sgRNA sites across all mTSG-treated liver samples, sorted by sum variant frequency. Four samples (mTSG liver 17, mTSG liver 54, mTSG liver 96, and mTSG liver 115) are not shown, as these samples were not found to have any significantly mutated sgRNA sites per our stringent variant calling strategy. The extensive mutational heterogeneity amongst the liver samples is suggestive of strong positive selective forces acting on diverse loss-of-function mutations induced by the mTSG library.

FIG. 10 is a metaplot of indel size distribution in livers from mice treated with AAV-CRISPR mTSG library. Heatmap detailing indel size distribution and abundance across all significantly mutated sgRNA sites from mTSG-treated liver samples. Positive indel sizes denote insertions, while negative indel sizes indicate deletions. Depicted values are in terms of total log 2 normalized reads per million (rpm) for each sample. Most variant reads are deletions (80.8%) compared to insertions (19.2%).

FIG. 11 illustrates the mutational frequencies in mice that correlate with human hepatocellular carcinomas. Scatterplot of gene population-wide mutant frequencies for the genes represented in the mTSG library, comparing mTSG treated mouse samples to human samples (TCGA LIHC dataset). Pearson correlation coefficient is shown on the plot, revealing mouse and human mutation frequencies were significantly correlated (R=0.461, t-test for correlation, p=4.′78*10⁻⁴).

FIG. 12 is a heatmap of all unique variants across all mTSG liver samples. Variant frequencies for all unique variants identified across mTSG liver samples after square-root transformation are depicted. Rows denote unique variants, while columns denote different liver samples. Data was clustered using Euclidean distance and average linkage. 70.25% (418/595) of the variants were sample-specific, while 29.75% (177/595) variants were found across multiple samples.

FIGS. 13A-13C are a series of images illustrating direct in vivo validation of multiple strong drivers in combination with Trp53. Representative bioluminescence imaging of LSL-Cas9 mice injected with liver-specific AAV-CRISPR vectors containing dual-sgRNAs. All images are taken one month post-treatment. Luminescence intensities are shown in units of photons/sec/cm²/sr. FIG. 13A depicts Arid2 and Trp53 (one tailed Chi-square test, p=0.0023), B2m and Trp53 (p=0.0192), Cic and Trp53 (p=0.0023), and Kdm5c and Trp53 (p=0.0023). FIG. 13B depicts Pik3r1 and Trp53 (p=0.0008), Pten and Trp53 (p=0.0142), Stk11 and Trp53 (p=0.0023), and Vh1 and Trp53 (p=0.0142). FIG. 13C depicts Zc3h13 and Trp5 (p=0.0023). All tested gene pairs led to efficient, rapid tumor growth, validating the findings of the high-throughput screen.

FIG. 14 is a table showing tumor volume data as measured by MRI.

FIG. 15 is a table showing tumor area data as measured by tissue histology.

FIG. 16 is a table showing data from Spearman rank correlation matrix for 5 individual liver lobes within a single mouse.

FIGS. 17A-17H are a series of tables showing sequences (SEQ ID NOs 289-554) of the Molecular Inversion Probes (MIPs) illustrated herein.

FIGS. 18A-18B are a series of images illustrating additional brightfield images of mTSG-treated livers with GFP overlay. Brightfield images with GFP fluorescence overlay of livers from 15 mTSG-treated mice at the time of sacrifice are shown.

FIGS. 19A-19C show representative histology and immunohistochemistry images of mouse liver sections in PBS, vector, and mTSG groups. FIG. 19A shows representative liver sections from PBS, vector, and mTSG-treated mice with hematoxylin and eosin staining. The vector sample and mTSG replicate 4 pictured here are from the same mice shown in FIG. 1I. Scale bar is 1 mm for low magnification images, 200 μm for high magnification images. FIG. 19B shows representative liver sections from PBS, vector, and mTSG-treated mice with Ki67 staining. Sections correspond to the same mice shown in Fig. S4A. Scale bar is 1 mm for low magnification images, 200 μm for high magnification images. FIG. 19C) Representative liver sections from PBS, vector, and mTSG-treated mice with pan-cytokeratin AE1/AE3 staining. Sections correspond to the same mice shown in fig. S4A. Scale bar is 1 mm for low magnification images, 200 μm for high magnification images.

FIG. 20 is a plot of median log 2 sequencing coverage across all sequenced samples in amplicons targeted by the 266 MIPs (black dots). MIPs were designed to amplify the genomic regions flanking the predicted cut sites of each sgRNA. 95% confidence intervals for the median are depicted with grey lines. Median read depth across all MIPs approximated a lognormal distribution, indicating relatively even capture of the target loci.

FIG. 21 is a heat map of gene level sum variant frequency across all mTSG liver samples. Heat map depicts sum variant frequencies for the 56 genes represented in the library, across all mTSG liver samples. Genes are ordered according to average sum variant frequency (top to bottom row).

FIGS. 22A-22B are a set of plots showing additional co-mutation analysis. FIG. 22A is a scatterplot of the cooccurrence rates for each gene pair, excluding all pairs involving Trp53, plotted against −log₁₀ p-values by hypergeometric test. FIG. 22D is a scatterplot of the Spearman correlations for each gene pair, excluding all pairs involving Trp53, plotted against −log₁₀ p-values.

FIGS. 23A-23D are a series of plots and images illustrating investigation and comparison of single or combinatorial knockout of screened TSGs in liver tumorigenesis. FIG. 23A shows schematics of the design and cloning of liver-specific AAV-CRISPR vectors to functionally study target genes for their potential roles as independent and synergistic drivers of liver tumor in immunocompetent mice. The AAV-CRISPR plasmids contain two U6 promoter-driving sgRNA expression cassettes, with the 1st sgRNA targeting Trp53, and another one either as a non-targeting sgRNA (NTC+Trp53) or a geneX-targeting sgRNA (GeneX+Trp53). The plasmids also contain a liver-specific TBG promoter driving a co-cistronic expression cassette of firefly luciferase (FLuc) and Cre recombinase. AAVs were generated with these plasmids and injected intravenously into LSL-Cas9 mice. FIG. 23B shows representative bioluminescence images of LSL-Cas9 mice injected with AAV9 that contains liver-specific TBG promoter-driving Cre and CRISPR dual-sgRNAs expression cassettes. Undetectable or weak luciferase activity was detected in NTC+Trp53 AAV treated mice (n=8) at 121 days post-injection, whereas persistent and robust luciferase activity was detected in the mice that were injected with the top scoring genes (GeneX+Trp53) or the highly co-mutated gene pairs from the screen. FIG. 23C shows quantification of bioluminescence intensities of AAV-CRISPR injected LSL-Cas9 mice at 121 days post-injection in units of photons/sec/cm2/sr (Data represented as mean±SEM). The mice that were injected with AAVs targeting the top screened genes or the highly correlated gene pairs had robust luciferase activity after 121 days of injection, indicating the role of these TSGs in accelerating development of tumors compared to NTC controls (two-sided unpaired t test, N.S. p>0.05, * p<0.05, ** p<0.01, *** p<0.001). In comparison to NTC (n=7), Cic (n=4, p=0.018), Pik3r1 (n=7, p=0.015), Pten (n=4, p=0.011), Stk11 (n=8, p=0.03), Arid2 (n=3, p=0.001) and Kdm5c (n=3, p=0.0005) knockout had significantly higher bioluminescence intensities. Double knockout of Pik3r1+Pten (n=3) had significantly stronger luciferase activity compared to NTC (two-sided unpaired t test, p<0.0001), but was not significantly different from knocking out Pik3r1 or Pten alone (two-sided unpaired t test, N.S.). Double knockout of Pik3r1+Stk11 (n=2) had significantly stronger luciferase activity compared to NTC (two-sided unpaired t test, p=0.01), but was not significantly different from knocking out Pik3r1 or Stk11 alone (two-sided unpaired t test, N.S.). In contrast, double knockout of B2m+Kansl1 led to significantly higher luminescence intensities compared to NTC (two-sided unpaired t test, p=0.005), B2m alone (p=0.001) and Kansl1 alone (p=0.02). FIG. 23D shows longitudinal IVIS live imaging of single or combinatorial AAV-CRISPR knockout of TSGs in driving liver tumorigenesis. The bioluminescence intensities of LSL-Cas9 mice injected with liver-specific AAVs containing either NTCs or sgRNAs targeting single gene or combinations of two genes. Left to right, B2m+Kansl1, Pik3r1+Pten, Pik3r1+Stk11, and Arid2+Kdm5c.

FIGS. 24A-24C are a series of plots illustrating mutant clonality and clustering analysis. Gaussian kernel density estimate of variant frequencies within each mTSG liver sample are shown. The number of peaks in the kernel density estimate is an approximation for the clonality of each sample. From this analysis, most (24/30) samples appeared to be composed of multiple clones, with six monoclonal samples.

### DETAILED DESCRIPTION OF THE INVENTION

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

As used herein the term “amount” refers to the abundance or quantity of a constituent in a mixture.

As used herein, the term “bp” refers to base pair.

The term “complementary” refers to the degree of anti-parallel alignment between two nucleic acid strands. Complete complementarity requires that each nucleotide be across from its opposite. No complementarity requires that each nucleotide is not across from its opposite. The degree of complementarity determines the stability of the sequences to be together or anneal/hybridize. Furthermore various DNA repair functions as well as regulatory functions are based on base pair complementarity.

The term “CRISPR/Cas” or “clustered regularly interspaced short palindromic repeats” or “CRISPR” refers to DNA loci containing short repetitions of base sequences followed by short segments of spacer DNA from previous exposures to a virus or plasmid. Bacteria and archaea have evolved adaptive immune defenses termed CRISPR/CRISPR-associated (Cas) systems that use short RNA to direct degradation of foreign nucleic acids. In bacteria, the CRISPR system provides acquired immunity against invading foreign DNA via. RNA-guided DNA cleavage.

The “CRISPR/Cas9” system or “CRISPR/Cas9-mediated gene editing” refers to a type II CRISPR/Cas system that has been modified for genome editing/engineering. It is typically comprised of a “guide” RNA (gRNA) and a non-specific CRISPR-associated endonuclease (Cas9). “Guide RNA (gRNA)” is used interchangeably herein with “short guide RNA (sgRNA)” or “single guide RNA (sgRNA). The sgRNA is a short synthetic RNA composed of a “scaffold” sequence necessary for Cas9-binding and a user-defined ˜20 nucleotide “spacer” or “targeting” sequence which defines the genomic target to be modified. The genomic target of Cas9 can be changed by changing the targeting sequence present in the sgRNA.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

The term “expression” as used herein is defined as the transcription and/or translation of a particular nucleotide sequence driven by its promoter.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., Sendai viruses, lentiviruses, retroviruses, adenoviruses, and adeno-associated viruses) that incorporate the recombinant polynucleotide.

“Homologous” as used herein, refers to the subunit sequence identity between two polymeric molecules, e.g., between two nucleic acid molecules, such as, two DNA molecules or two RNA molecules, or between two polypeptide molecules. When a subunit position in both of the two molecules is occupied by the same monomeric subunit; e.g., if a position in each of two DNA molecules is occupied by adenine, then they are homologous at that position. The homology between two sequences is a direct function of the number of matching or homologous positions; e.g., if half (e.g., five positions in a polymer ten subunits in length) of the positions in two sequences are homologous, the two sequences are 50% homologous; if 90% of the positions (e.g., 9 of 10), are matched or homologous, the two sequences are 90% homologous.

“Identity” as used herein refers to the subunit sequence identity between two polymeric molecules particularly between two amino acid molecules, such as, between two polypeptide molecules. When two amino acid sequences have the same residues at the same positions; e.g., if a position in each of two polypeptide molecules is occupied by an Arginine, then they are identical at that position. The identity or extent to which two amino acid sequences have the same residues at the same positions in an alignment is often expressed as a percentage. The identity between two amino acid sequences is a direct function of the number of matching or identical positions; e.g., if half (e.g., five positions in a polymer ten amino acids in length) of the positions in two sequences are identical, the two sequences are 50% identical; if 90% of the positions (e.g., 9 of 10), are matched or identical, the two amino acids sequences are 90% identical.

As used herein, an “instructional material” includes a publication, a recording, a diagram, or any other medium of expression which can be used to communicate the usefulness of the compositions and methods of the invention. The instructional material of the kit of the invention may, for example, be affixed to a container which contains the nucleic acid, peptide, and/or composition of the invention or be shipped together with a container which contains the nucleic acid, peptide, and/or composition. Alternatively, the instructional material may be shipped separately from the container with the intention that the instructional material and the compound be used cooperatively by the recipient.

A “mutation” as used herein is a change in a DNA sequence resulting in an alteration from a given reference sequence (which may be, for example, an earlier collected DNA sample from the same subject). The mutation can comprise deletion and/or insertion and/or duplication and/or substitution of at least one deoxyribonucleic acid base such as a purine (adenine and/or thymine) and/or a pyrimidine (guanine and/or cytosine). Mutations may or may not produce discernible changes in the observable characteristics (phenotype) of an organism (subject).

By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine and uracil).

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

The term “oligonucleotide” typically refers to short polynucleotides, generally no greater than about 60 nucleotides. It will be understood that when a nucleotide sequence is represented by a DNA sequence (i.e., A, T, G, C), this also includes an RNA sequence (i.e., A, U, G, C) in which “U” replaces “T”.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “polynucleotide” includes DNA, cDNA, RNA, DNA/RNA hybrid, anti-sense RNA, siRNA, miRNA, snoRNA, genomic DNA, synthetic forms, and mixed polymers, both sense and antisense strands, and may be chemically or biochemically modified to contain non-natural or derivatized, synthetic, or semisynthetic nucleotide bases. Also, included within the scope of the invention are alterations of a wild type or synthetic gene, including but not limited to deletion, insertion, substitution of one or more nucleotides, or fusion to other polynucleotide sequences.

Conventional notation is used herein to describe polynucleotide sequences: the left-hand end of a single-stranded polynucleotide sequence is the 5′-end; the left-hand direction of a double-stranded polynucleotide sequence is referred to as the 5′-direction.

The term “promoter” as used herein is defined as a DNA sequence recognized by the synthetic machinery of the cell, or introduced synthetic machinery, required to initiate the specific transcription of a polynucleotide sequence.

A “sample” or “biological sample” as used herein means a biological material from a subject, including but is not limited to organ, tissue, exosome, blood, plasma, saliva, urine and other body fluid. A sample can be any source of material obtained from a subject.

The term “subject” is intended to include living organisms in which an immune response can be elicited (e.g., mammals). A “subject” or “patient,” as used therein, may be a human or non-human mammal. Non-human mammals include, for example, livestock and pets, such as ovine, bovine, porcine, canine, feline and murine mammals. Preferably, the subject is human.

A “target site” or “target sequence” refers to a genornic nucleic acid sequence that defines a portion of a nucleic acid to which a binding molecule may specifically bind under conditions sufficient for binding to occur.

The term “therapeutic” as used herein means a treatment and/or prophylaxis. A therapeutic effect is obtained by suppression, remission, or eradication of a disease state.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny. In certain embodiments, “transfected” means an exogenous nucleic acid is transferred transiently into a cell, often a mammalian cell; while “transduced” means an exogenous nucleic acid is transferred permanently into a cell, often a mammalian cell, for example by viruses or viral vectors; “transformed” means an exogenous nucleic acid is transferred into a cell, often bacterial or yeast cells.

To “treat” a disease as the term is used herein, means to reduce the frequency or severity of at least one sign or symptom of a disease or disorder experienced by a subject.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, Sendai viral vectors, adenoviral vectors, adeno-associated virus vectors, retroviral vectors, lentiviral vectors, and the like.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Description

Herein, a Functional Cancer Genome Atlas (FCGA) of tumor suppressors in the autochthonous mouse liver was mapped using massively parallel CRISPR/Cas9 genome editing. A direct in vivo CRISPR screen was performed by intravenously injecting adeno-associated virus (AAV) pools carrying a library of 280 sgRNAs targeting 56 cancer genes into Rosa-LSL-Cas9-EGFP knock-in mice (LSL-Cas9 mice) to generate highly complex autochthonous liver tumors, and subsequently readout the Cas9-generated variants at predicted sgRNA cut sites using molecular inversion probe sequencing (MIPS). This combination of direct mutagenesis and pooled variant readout illuminated the mutational landscape of the tumors, demonstrating that the present approach can be used to quantitatively analyze numerous putative TSGs in a high-throughput manner. Mutagenesis of individual or combinations of genes represented by high frequency variants validated certain functional drivers of liver tumorigenesis in fully immunocompetent mice.

### Methods

The present invention includes methods for identifying cancer driver mutations in vivo. One aspect of the method comprises selecting nucleotide sequences in silica from a plurality of tumor suppressor genes (TSGs) and designing a plurality of short guide RNA (sgRNA) sequences in Silk® homologous to the plurality of TSGs. In certain embodiments, the plurality of sgRNA sequences are synthesized into oligonucleotides and introduced into a plurality of AAV-CRISPR vectors. In certain embodiments, the AAV-CRISPR vectors comprise Cas9. In certain embodiments, the AAV-CRISPR vectors containing the plurality of oligonucleotides are administered into an animal. In certain embodiments, a tumor is isolated from the animal. In certain embodiments, nucleic acids are isolated from the tumor and sequenced. In certain embodiments, the sequencing data are analyzed, thus identifying the cancer driver mutation(s).

Another aspect of the invention includes a method of determining at least one cancer driver mutation in vivo in a cancer-affected subject. In certain embodiments, the method comprises administering to the subject a plurality of AAV-CRISPR vectors, wherein the AAV-CRISPR vectors comprise Cas9 and a plurality of short guide RNAs (sgRNAs) homologous to a plurality of tumor suppressor genes (TSGs). In certain embodiments, a plurality of nucleic acids isolated from the subject's cancer is sequenced and analysis of the sequencing data indicates whether any cancer driver mutation is present in the subject's cancer.

In certain embodiments of the invention, the sgRNA sequences comprise at least one selected from the group consisting of SEQ ID NOs. 1-280.

In certain embodiments of the invention, the sgRNA sequences comprise SEQ ID NOs. 1-280.

In certain embodiments of the invention, the AAV-CRISPR vector is comprised of the components as described herein. In certain embodiments, the AAV-CRISPR can also include (1) constitutive EFS promoter or tissue-specific TBG promoter, for example polII promoters, (2) a constitutive U6 polIII promoter, (3) sgRNA spacer cloning site with double SapI type II restriction enzyme cutting site; (4) an sgRNA backbone derived from an 89 bp chimeric backbone from Streptococcus pyogenes Cas9 tracrRNA; and (5) a Cre recombinase.

In certain embodiments of the invention, the animal is a mouse. Other animals that can be used include but are not limited to rats, rabbits, dogs, cats, horses, pigs, cows and birds. In certain embodiments, the animal is a human. The AAV-CRISPR vectors can be administered to an animal by any means standard in the art. For example the vectors can be injected into the animal. The injections can be intravenous, subcutaneous, intraperitoneal, or directly into a tissue or organ.

Nucleotide sequencing or ‘sequencing’, as it is commonly known in the art, can be performed by standard methods commonly known to one of ordinary skill in the art. In certain embodiments of the invention, sequencing comprises targeted capture sequencing. Targeted capture sequencing can be performed as described herein, or by methods commonly performed by one of ordinary skill in the art. In certain embodiments, the targeted capture sequencing is performed using a plurality of Molecular Inversion Probes (MIPs). In certain embodiments, the plurality of MIPs comprises at least one selected from the group consisting of SEQ ID NOs. 289-554. In certain embodiments, the plurality of MIPs comprises SEQ ID NOs. 289-554.

Another aspect of the invention includes a method of identifying a plurality of cancer driver mutations in a sample comprising hybridizing a plurality of Molecular Inversion Probes (MIPs) to a plurality of nucleic acids from the sample. In certain embodiments, targeted capture sequencing is performed on the plurality of nucleic acids. In certain embodiments, data from the targeted capture sequencing is then analyzed, thus identifying the plurality of cancer driver mutations in the sample. In certain embodiments, the MIPs comprise at least one selected from the group consisting of SEQ ID NOs. 289-554. In certain embodiments, the MIPs comprise SEQ ID NOs. 289-554.

Yet another aspect of the invention includes a method of determining at least one cancer driver mutation in a sample comprising administering an AAV-CRISPR vectors to the sample, wherein the vectors comprise Cas9 and a plurality of nucleotide sequences homologous to a plurality of tumor suppressor genes (TSGs). In certain embodiments, the nucleic acids are isolated from the sample and sequenced. In certain embodiments, the sequencing data are analyzed, thus determining the at least one cancer driver mutation in the sample.

Another aspect of the invention includes a method of determining a treatment for cancer in a subject. The method comprises administering a plurality of AAV-CRISPR vectors to a sample from the subject. In certain embodiments, the vectors comprise Cas9 and a plurality of nucleotide sequences homologous to a plurality of tumor suppressor genes (TSGs). In certain embodiments, the nucleic acids are isolated from the sample and sequenced. In certain embodiments, the sequencing data are analyzed, thus identifying at least one cancer driver mutation in the sample. In certain embodiments, identifying the at least one cancer driver mutation determines the cancer treatment for the subject.

The mutations claimed herein can be any combination of insertions or deletions, including but not limited to a single base insertion, a single base deletion, a frameshift, a rearrangement, and an insertion or deletion of 2, 3, 4, 5, 6, 7, 8, 9, 10, 15, 20, 25, 30, 40, 50, 60, 70, 80, 90, 100, 150, 200, 250, 300, any and all numbers in between, bases. The mutation can occur in a gene or in a non-coding region. The location of the mutation can provide information as to the type of treatment needed. For example, if a mutation occurs in a specific gene rendering that gene non-functional, a drug that acts on that particular gene will not be considered for treatment. Likewise if a drug is known to act on a particular gene and that gene is not mutated, that drug will be considered for treatment.

In certain embodiments the plurality of nucleotide sequences homologous to a plurality of TSGs comprises at least one selected from the group consisting of SEQ ID NOs. 1-280.

In certain embodiments the plurality of nucleotide sequences homologous to a plurality of TSGs comprises SEQ ID NOs. 1-280.

The sample of the present invention can comprise a cancer cell or a plurality of cancer cells. The sample can also comprise a tumor. In some embodiments, multiple sections of the same tumor can make up multiple samples.

The compositions described herein may be administered to a patient transarterially, subcutaneously, intradermally, intratumorally, intranodally, intramedullary, intramuscularly, by intravenous (i.v.) injection, or intraperitoneally. In other instances, the composition of the invention are injected directly into a site of inflammation in the subject, a local disease site in the subject, a lymph node, an organ, a tumor, and the like.

### Compositions

One aspect of the invention provides a composition comprising a set of Molecular Inversion Probes (MIPs) comprised of at least one selected from the group consisting of SEQ ID NOs. 289-554. Another aspect includes a kit comprising a set of Molecular Inversion Probes (MIPs) comprised of at least one selected from the group consisting of SEQ ID NOs. 289-554, and instructional material for use thereof. Yet another aspect includes a kit for determining at least one cancer driver mutation in a sample comprising a set of Molecular Inversion Probes (MIPs) comprised of at least one selected from the group consisting of SEQ ID NOs. 289-554, reagents for measuring the at least one cancer driver mutation, and instructional material for use thereof.

Another aspect includes a composition comprising an AAV-CRISPR mTSG library comprised of a plurality of AAV vectors. The AVV vectors are comprised of Cas9 and a plurality of nucleic acids homologous to a plurality of Tumor Suppressor Gene (TSGs). In one embodiment, the plurality of nucleic acids comprises at least one selected from the group consisting of SEQ ID NOs. 1-280.

In one aspect, the invention includes a vector comprising an adeno-associated virus (AAV) genome, a U6 promoter gene, an sgRNA sequence, an EFS promoter gene, and a Cre recombinase gene. In another aspect, the invention includes a vector comprising an adeno-associated virus (AAV) genome, a U6 promoter gene, an sgRNA sequence, a TBG promoter gene, and a Cre recombinase gene. In yet another aspect, the invention includes a vector comprising the nucleic acid sequence of SEQ ID NO: 555. In still another aspect, the invention includes a vector comprising the nucleic acid sequence of SEQ ID NO: 556. In certain embodiments, the TBG promoter gene comprises the nucleic acid sequence of SEQ ID NO: 557. In certain embodiments, the AAV-CRISPR can also include (1) constitutive EFS promoter or tissue-specific TBG promoter, for example polII promoters, (2) a constitutive U6 polIII promoter, (3) sgRNA spacer cloning site with double SapI type II restriction enzyme cutting site; (4) an sgRNA backbone derived from an 89 bp chimeric backbone from Streptococcus pyogenes Cas9 tracrRNA; and (5) a Cre recombinase.

Another aspect of the invention includes a kit comprising an adeno-associated virus (AAV) genome, a U6 promoter gene, an sgRNA sequence, an EFS promoter gene, and a Cre recombinase gene, and instructional material for use thereof. Yet another aspect includes a kit comprising an adeno-associated virus (AAV) genome, a U6 promoter gene, an sgRNA sequence, an TBG promoter gene, and a Cre recombinase gene, and instructional material for use thereof.

### CRISPR/Cas9

The CRISPR/Cas9 system is a facile and efficient system for inducing targeted genetic alterations. Target recognition by the Cas9 protein requires a ‘seed’ sequence within the guide RNA (gRNA) and a conserved di-nucleotide containing protospacer adjacent motif (PAM) sequence upstream of the gRNA-binding region. The CRISPR/Cas9 system can thereby be engineered to cleave virtually any DNA sequence by redesigning the gRNA in cell lines (such as 2931 cells), primary cells, and CAR T cells. The CRISPR/Cas9 system can simultaneously target multiple genomic loci by co-expressing a single Cas9 protein with two or more gRNAs, making this system uniquely suited for multiple gene editing or synergistic activation of target genes.

The Cas9 protein and guide RNA form a complex that identifies and cleaves target sequences. Cas9 is comprised of six domains: REC I, REC II, Bridge Helix, PAM interacting, HNH, and RuvC. The Red domain binds the guide RNA, while the Bridge helix binds to target DNA. The HNH and RuvC domains are nuclease domains. Guide RNA is engineered to have a 5′ end that is complementary to the target DNA sequence. Upon binding of the guide RNA to the Cas9 protein, a conformational change occurs activating the protein. Once activated, Cas9 searches for target DNA by binding to sequences that match its protospacer adjacent motif (PAM) sequence. A PAM is a two or three nucleotide base sequence within one nucleotide downstream of the region complementary to the guide RNA. In one non-limiting example, the PANT sequence is 5′-NG-G-3′. When the Cas9 protein finds its target sequence with the appropriate PAM, it melts the bases upstream of the PAM and pairs them with the complementary region on the guide RNA. Then the RuvC and HNH nuclease domains cut the target DNA after the third nucleotide base upstream of the PAM.

One non-limiting example of a CRISPR/Cas system used to inhibit gene expression, CRISPRi, is described in U.S. Patent Appl. Publ. No. US20140068797. CRISPRi induces permanent gene disruption that utilizes the RNA-guided Cas9 endonuclease to introduce DNA double stranded breaks which trigger error-prone repair pathways to result in frame shift mutations. A catalytically dead Cas9 lacks endonuclease activity. When coexpressed with a guide RNA, a DNA recognition complex is generated that specifically interferes with transcriptional elongation, RNA polymerase binding, or transcription factor binding. This CRISPRi system efficiently represses expression of targeted genes.

CRISPR/Cas gene disruption occurs when a guide nucleic acid sequence specific for a target gene and a Cas endonuclease are introduced into a cell and form a complex that enables the Cas endonuclease to introduce a double strand break at the target gene. In certain embodiments, the CRISPR/Cas system comprises an expression vector, such as, but not limited to, an pAd5F35-CRISPR vector. In other embodiments, the Cas expression vector induces expression of Cas9 endonuclease. Other endonucleases may also be used, including but not limited to, T7, Cas3, Cas8a, Cas8b, Cas10d, Cse1, Csy1, Csn2, Cas4, Cas10, Csm2, Cmr5, Fok1, other nucleases known in the art, and any combination thereof.

In certain embodiments, inducing the Cas expression vector comprises exposing the cell to an agent that activates an inducible promoter in the Cas expression vector. In such embodiments, the Cas expression vector includes an inducible promoter, such as one that is inducible by exposure to an antibiotic (e.g., by tetracycline or a derivative of tetracycline, for example doxycycline). However, it should be appreciated that other inducible promoters can be used. The inducing agent can be a selective condition (e.g., exposure to an agent, for example an antibiotic) that results in induction of the inducible promoter. This results in expression of the Cas expression vector.

In certain embodiments, guide RNA(s) and Cas9 can be delivered to a cell as a ribonucleoprotein (RNP) complex. RNPs are comprised of purified Cas9 protein complexed with gRNA and are well known in the art to be efficiently delivered to multiple types of cells, including but not limited to stem cells and immune cells (Addgene, Cambridge, Mass., Mirus Bio LLC, Madison, Wis.).

The guide RNA is specific for a genomic region of interest and targets that region for Cas endonuclease-induced double strand breaks. The target sequence of the guide RNA sequence may be within a loci of a gene or within a non-coding region of the genome. In certain embodiments, the guide nucleic acid sequence is at least 10, 11, 12, 13, 14, 15, 16, 17, 18, 19, 20, 21, 22, 23, 24, 25, 26, 27, 30, 31, 32, 33, 34, 35, 36, 37, 38, 39, 40 or more nucleotides in length.

Guide RNA (gRNA), also referred to as “short guide RNA” or “sgRNA”, provides both targeting specificity and scaffolding/binding ability for the Cas9 nuclease. The gRNA can be a synthetic RNA composed of a targeting sequence and scaffold sequence derived from endogenous bacterial crRNA and tracrRNA. gRNA is used to target Cas9 to a specific genomic locus in genome engineering experiments. Guide RNAs can be designed using standard tools well known in the art.

In the context of formation of a CRISPR complex, “target sequence” refers to a sequence to which a guide sequence is designed to have some complementarity, where hybridization between a target sequence and a guide sequence promotes the formation of a CRISPR complex. Full complementarity is not necessarily required, provided there is sufficient complementarity to cause hybridization and promote formation of a CRISPR complex. A target sequence may comprise any polynucleotide, such as DNA or RNA polynucleotides. In certain embodiments, a target sequence is located in the nucleus or cytoplasm of a cell. In other embodiments, the target sequence may be within an organelle of a eukaryotic cell, for example, mitochondrion or nucleus. Typically, in the context of an endogenous CRISPR system, formation of a CRISPR complex (comprising a guide sequence hybridized to a target sequence and complexed with one or more Cas proteins) results in cleavage of one or both strands in or near (e.g., within about 1, 2, 3, 4, 5, 6, 7, 8, 9, 10, 20, 50 or more base pairs) the target sequence. As with the target sequence, it is believed that complete complementarity is not needed, provided this is sufficient to be functional.

In certain embodiments, one or more vectors driving expression of one or more elements of a CRISPR system are introduced into a host cell, such that expression of the elements of the CRISPR system direct formation of a CRISPR complex at one or more target sites. For example, a Cas enzyme, a guide sequence linked to a tracr-mate sequence, and a tracr sequence could each be operably linked to separate regulatory elements on separate vectors. Alternatively, two or more of the elements expressed from the same or different regulatory elements may be combined in a single vector, with one or more additional vectors providing any components of the CRISPR system not included in the first vector. CRISPR system elements that are combined in a single vector may be arranged in any suitable orientation, such as one element located 5′ with respect to (“upstream” of) or 3′ with respect to (“downstream” of) a second element. The coding sequence of one element may be located on the same or opposite strand of the coding sequence of a second element, and oriented in the same or opposite direction. In certain embodiments, a single promoter drives expression of a transcript encoding a CRISPR enzyme and one or more of the guide sequence, tracr mate sequence (optionally operably linked to the guide sequence), and a tracr sequence embedded within one or more intron sequences (e.g., each in a different intron, two or more in at least one intron, or all in a single intron).

In certain embodiments, the CRISPR enzyme is part of a fusion protein comprising one or more heterologous protein domains (e.g. about or more than about 1, 2, 3, 4, 5, 6, 7, 8, 9, 10, or more domains in addition to the CRISPR enzyme). A CRISPR enzyme fusion protein may comprise any additional protein sequence, and optionally a linker sequence between any two domains. Examples of protein domains that may be fused to a CRISPR enzyme include, without limitation, epitope tags, reporter gene sequences, and protein domains having one or more of the following activities: methylase activity, demethylase activity, transcription activation activity, transcription repression activity, transcription release factor activity, histone modification activity, RNA cleavage activity and nucleic acid binding activity. Additional domains that may form part of a fusion protein comprising a CRISPR enzyme are described in US20110059502, incorporated herein by reference. In certain embodiments, a tagged CRISPR enzyme is used to identify the location of a target sequence.

Conventional viral and non-viral based gene transfer methods can be used to introduce nucleic acids in mammalian and non-mammalian cells or target tissues. Such methods can be used to administer nucleic acids encoding components of a CRISPR system to cells in culture, or in a host organism. Non-viral vector delivery systems include DNA plasmids, RNA (e.g., a transcript of a vector described herein), naked nucleic acid, and nucleic acid complexed with a delivery vehicle, such as a liposome. Viral vector delivery systems include DNA and RNA viruses, which have either episomal or integrated genomes after delivery to the cell (Anderson, 1992, Science 256:808-813; and Yu, et al., 1994, Gene Therapy 1:13-26).

In certain embodiments, the CRISPR/Cas is derived from a type II CRISPR/Cas system. In other embodiments, the CRISPR/Cas system is derived from a Cas9 protein. The Cas9 protein can be from Streptococcus pyogenes, Streptococcus thermophilus, or other species.

In general, Cas proteins comprise at least one RNA recognition and/or RNA binding domain. RNA recognition and/or RNA binding domains interact with the guiding RNA. Cas proteins can also comprise nuclease domains (i.e., DNase or RNase domains), DNA binding domains, helicase domains, RNAse domains, protein-protein interaction domains, dimerization domains, as well as other domains. The Cas proteins can be modified to increase nucleic acid binding affinity and/or specificity, alter an enzymatic activity, and/or change another property of the protein. In certain embodiments, the Cas-like protein of the fusion protein can be derived from a wild type Cas9 protein or fragment thereof. In other embodiments, the Cas can be derived from modified Cas9 protein. For example, the amino acid sequence of the Cas9 protein can be modified to alter one or more properties (e.g., nuclease activity, affinity, stability, and so forth) of the protein. Alternatively, domains of the Cas9 protein not involved in RNA-guided cleavage can be eliminated from the protein such that the modified Cas9 protein is smaller than the wild type Cas9 protein. In general, a Cas9 protein comprises at least two nuclease (i.e., DNase) domains. For example, a Cas9 protein can comprise a RuvC-like nuclease domain and a HNH-like nuclease domain. The RuvC and HNH domains work together to cut single strands to make a double-stranded break in DNA. (Jinek, et al., 2012, Science, 337:816-821). In certain embodiments, the Cas9-derived protein can be modified to contain only one functional nuclease domain (either a RuvC-like or a HNH-like nuclease domain). For example, the Cas9-derived protein can be modified such that one of the nuclease domains is deleted or mutated such that it is no longer functional (i.e., the nuclease activity is absent). In some embodiments in which one of the nuclease domains is inactive, the Cas9-derived protein is able to introduce a nick into a double-stranded nucleic acid (such protein is termed a “nickase”), but not cleave the double-stranded DNA. In any of the above-described embodiments, any or all of the nuclease domains can be inactivated by one or more deletion mutations, insertion mutations, and/or substitution mutations using well-known methods, such as site-directed mutagenesis, PCR-mediated mutagenesis, and total gene synthesis, as well as other methods known in the art.

In one non-limiting embodiment, a vector drives the expression of the CRISPR system. The art is replete with suitable vectors that are useful in the present invention. The vectors to be used are suitable for replication and, optionally, integration in eukaryotic cells. Typical vectors contain transcription and translation terminators, initiation sequences, and promoters useful for regulation of the expression of the desired nucleic acid sequence. The vectors of the present invention may also be used for nucleic acid standard gene delivery protocols. Methods for gene delivery are known in the art (U.S. Pat. Nos. 5,399,346, 5,580,859 & 5,589,466, incorporated by reference herein in their entireties).

Further, the vector may be provided to a cell in the form of a viral vector. Viral vector technology is well known in the art and is described, for example, in Sambrook et al. (4^{th} Edition, Molecular Cloning: A Laboratory Manual, Cold Spring Harbor Laboratory, New York, 2012), and in other virology and molecular biology manuals. Viruses, which are useful as vectors include, but are not limited to, retroviruses, adenoviruses, adeno-associated viruses, herpes viruses, Sindbis virus, gammaretrovirus and lentiviruses. In general, a suitable vector contains an origin of replication functional in at least one organism, a promoter sequence, convenient restriction endonuclease sites, and one or more selectable markers (e.g., WO 01/96584; WO 01/29058; and U.S. Pat. No. 6,326,193).

### Introduction of Nucleic Acids

Methods of introducing nucleic acids into a cell include physical, biological and chemical methods. Physical methods for introducing a polynucleotide, such as DNA or RNA, into a cell include transfection, transformation, transduction, calcium phosphate precipitation, lipofection, particle bombardment, microinjection, electroporation, and the like. RNA and DNA can be introduced into cells using commercially available methods which include electroporation (Amaxa Nucleofector-II (Amaxa Biosystems, Cologne, Germany)), (ECM 830 (BTX) (Harvard Instruments, Boston, Mass.) or the Gene Pulser II (BioRad, Denver, Colo.), Multiporator (Eppendort, Hamburg Germany). RNA and DNA can also be introduced into cells using cationic liposome mediated transfection using lipofection, using polymer encapsulation, using peptide mediated transfection, or using biolistic particle delivery systems such as “gene guns” (see, for example, Nishikawa, et al. Hum Gene Ther., 12(8):861-70 (2001).

Biological methods for introducing a polynucleotide of interest into a cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362. Non-viral vector such as plasmids can also be used to introduce nucleic acids or polynucleotides into a cell. In certain embodiments plasmids containing guide RNAs are transfected into a cell.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

Regardless of the method used to introduce exogenous nucleic acids into a host cell, in order to confirm the presence of the nucleic acids in the host cell, a variety of assays may be performed. Such assays include, for example, “molecular biological” assays well known to those of skill in the art, such as gel electrophoresis, Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the invention.

It should be understood that the methods and compositions that would be useful in the present invention are not limited to the particular formulations set forth in the examples. The following examples are put forth so as to provide those of ordinary skill in the art with a complete disclosure and description, and are not intended to limit the scope of what the inventors regard as their invention.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, Molecular Cloning: A Laboratory Manual”, fourth edition (Sambrook et al. (2012) Molecular Cloning, Cold Spring Harbor Laboratory); “Oligonucleotide Synthesis” (Gait, M. J. (1984). Oligonucleotide synthesis. IRL press); “Culture of Animal Cells” (Freshney, R. (2010). Culture of animal cells. Cell Proliferation, 15(2.3), 1); “Methods in Enzymology” “Weir's Handbook of Experimental Immunology” (Wiley-Blackwell; 5 edition (Jan. 15, 1996); “Gene Transfer Vectors for Mammalian Cells” (Miller and Carlos, (1987) Cold Spring Harbor Laboratory, New York); “Short Protocols in Molecular Biology” (Ausubel et al., Current Protocols; 5 edition (Nov. 5, 2002)); “Polymerase Chain Reaction: Principles, Applications and Troubleshooting”, (Babar, M., VDM Verlag Dr. Miller (Aug. 17, 2011)); “Current Protocols in Immunology” (Coligan, John Wiley & Sons, Inc. Nov. 1, 2002).

Those skilled in the art will recognize, or be able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that wherever values and ranges are provided herein, all values and ranges encompassed by these values and ranges, are meant to be encompassed within the scope of the present invention. Moreover, all values that fall within these ranges, as well as the upper or lower limits of a range of values, are also contemplated by the present application.

The following examples further illustrate aspects of the present invention. However, they are in no way a limitation of the teachings or disclosure of the present invention as set forth herein.

### EXPERIMENTAL EXAMPLES

The invention is now described with reference to the following Examples. These Examples are provided for the purpose of illustration only, and the invention is not limited to these Examples, but rather encompasses all variations that are evident as a result of the teachings provided herein.

The materials and methods employed in these experiments are now described.

Design, Synthesis and Cloning of the mTSG Library:

Pan-cancer mutation data from 15 cancer types were retrieved from The Cancer Genome Atlas (TCGA portal) via cBioPortal (Gao et al., Sci. Signal. 6, pl 1 (2013); Cerami et al., Cancer Discov. 2, 401-404 (2012)) and Synapse (www dot synapse dot org). Recurrently mutated genes were calculated similarly to previously described methods (Kandoth et al., Nature 502, 333-339 (2013); Lawrence et al., Nature 499, 214-218 (2013); Davoli et al., Cell 155, 948-962 (2013)). Known oncogenes were excluded and only known or predicted tumor suppressor genes (TSGs) were included. The top 50 TSGs were chosen, and their mouse homologs (mTSG) were retrieved from mouse genome informatics (MGI) (www dot informatics dot jax dot org). A total of 49 mTSGs were found. A total of 7 known housekeeping genes were chosen as internal controls. sgRNAs against these 56 genes were designed using a previously described method (Shalem et al., Science 343, 84-87 (2014); Wang et al., Science 343, 80-84 (2014)) with custom scripts. Five sgRNAs were chosen for each gene, plus 8 non-targeting controls (NTCs), making a total 288 sgRNAs in the mTSG library (Table 1). NTCs do not target any predicted sites in the genome, thus were not included in subsequent MIPs analysis. Of note, two sgRNA pairs happened to be identical by design, namely Rp122_sg4/sg5, and Cdkn2a_sg2/sg5. These sgRNAs were treated as the same in subsequent analyses.

Design, Cloning of AAV-CRISPR Vectors and mTSG sgRNA Library Cloning:

AAV-CRISPR vectors were designed to express Cre recombinase for induction of Cas9 expression using constitutive or conditional promoters when delivered to LSL-Cas9 mice (Plasmids available at Addgene). Two sgRNA cassettes were built in these vectors, one encoding an sgRNA targeting Trp53, with the other being an open sgRNA cassette (double SapI sites for sgRNA cloning). The vector was generated by gBlock gene fragment synthesis (IDT) followed by Gibson assembly (NEB). The mTSG library were generated by oligo synthesis, pooled, and cloned into the double SapI sites of the AAV-CRISPR vectors. Library cloning was done at over 100× coverage to ensure proper representation. Representation of plasmid libraries was readout by barcoded Illumina sequencing (Chen et al., Cell 160, 1246-1260 (2015)) with customized primers.

| Vector pAAV-sgRNA-EFS-Cre: | |
|---|---|
| (SEQ ID NO: 555) | |
| 1 | cctgcaggca gctgcgcgct cgctcgctca ctgaggccgc ccgggcaaag cccgggcgtc |
| 61 | gggcgacctt tggtcgcccg gcctcagtga gcgagcgagc gcgcagagag ggagtggcca |
| 121 | actccatcac taggggttcc tgcggccgca cgcgtgaggg cctatttccc atgattcctt |
| 181 | catatttgca tatacgatac aaggctgtta gagagataat tggaattaat ttgactgtaa |
| 241 | acacaaagat attagtacaa aatacgtgac gtagaaagta ataatttctt gggtagtttg |
| 301 | cagttttaaa attatgtttt aaaatggact atcatatgct taccgtaact tgaaagtatt |
| 361 | tcgatttctt ggctttatat atcttGTGGA AAGGACGAAA CACCGTGTAA TAGCTCCTGC |
| 421 | ATGGgtttta gagctaGAAA tagcaagtta aaataaggct agtccgttat caacttgaaa |
| 481 | aagtggcacc gagtcggtgc TTTTTTtcta gaagagggcc tatttcccat gattccttca |
| 541 | tatttgcata tacgatacaa ggctgttaga gagataattg gaattaattt gactgtaaac |
| 601 | acaaagatat tagtacaaaa tacgtgacgt agaaagtaat aatttcttgg gtagtttgca |
| 661 | gttttaaaat tatgttttaa aatggactat catatgctta ccgtaacttg aaagtatttc |
| 721 | gatttcttgg ctttatatat cttGTGGAAA GGACGAAACA CCggaagagc gagctcttct |
| 781 | gttttagagc taGAAAtagc aagttaaaat aaggctagtc cgttatcaac ttgaaaaagt |
| 841 | ggcaccgagt cggtgcTTTT TTggtaccag gtcttgaaag gagtgggaat tggctccggt |
| 901 | gcccgtcagt gggcagagcg cacatcgccc acagtccccg agaagttggg gggaggggtc |
| 961 | ggcaattgaa ccggtgccta gagaaggtgg cgcggggtaa actgggaaag tgatgtcgtg |
| 1021 | tactggctcc gcctttttcc cgagggtggg ggagaaccgt atataagtgc agtagtcgcc |
| 1081 | gtgaacgttc tttttcgcaa cgggtttgcc gccagaacac aggcgtacgg ccaccatgga |
| 1141 | agacgccaaa aacataaaga aaggcccggc gccattctat ccgctggaag atggaaccgc |
| 1201 | tggagagcaa ctgcataagg ctatgaagag atacgccctg gttcctggaa caattgcttt |
| 1261 | tacagatgca catatcgagg tggacatcac ttacgctgag tacttcgaaa tgtccgttcg |
| 1321 | gttggcagaa gctatgaaac gatatgggct gaatacaaat cacagaatcg tcgtatgcag |
| 1381 | tgaaaactct cttcaattct ttatgccggt gttgggcgcg ttatttatcg gagttgcagt |
| 1441 | tgcgcccgcg aacgacattt ataatgaacg tgaattgctc aacagtatgg gcatttcgca |
| 1501 | gcctaccgtg gtgttcgttt ccaaaaaggg gttgcaaaaa attttgaacg tgcaaaaaaa |
| 1561 | gctcccaatc atccaaaaaa ttattatcat ggattctaaa acggattacc agggatttca |
| 1621 | gtcgatgtac acgttcgtca catctcatct acctcccggt tttaatgaat acgattttgt |
| 1681 | gccagagtcc ttcgataggg acaagacaat tgcactgatc atgaactcct ctggatctac |
| 1741 | tggtctgcct aaaggtgtcg ctctgcctca tagaactgcc tgcgtgagat tctcgcatgc |
| 1801 | cagagatcct atttttggca atcaaatcat tccggatact gcgattttaa gtgttgttcc |
| 1861 | attccatcac ggttttggaa tgtttactac actcggatat ttgatatgtg gatttcgagt |
| 1921 | cgtcttaatg tatagatttg aagaGgagct gtttctgagg agccttcagg attacaagat |
| 1981 | tcaaagtgcg ctgctggtgc caaccctatt ctccttcttc gccaaaagca ctctgattga |
| 2041 | caaatacgat ttatctaatt tacacgaaat tgcttctggt ggcgctcccc tctctaagga |
| 2101 | agtcggggaa gcggttgcca agaggttcca tctgccaggt atcaggcaag gatatgggct |
| 2161 | cactgagact acatcagcta ttctgattac acccgagggg gatgataaac cgggcgcggt |
| 2221 | cggtaaagtt gttccatttt ttgaagcgaa ggttgtggat ctggataccg ggaaaacgct |
| 2281 | gggcgttaat caaagaggcg aactgtgtgt gagaggtcct atgattatgt ccggttatgt |
| 2341 | aaacaatccg gaagcgacca acgccttgat tgacaaggat ggatggctac attctggaga |
| 2401 | catagcttac tgggacgaag acgaacactt cttcatcgtt gaccgcctga agtctctgat |
| 2461 | taagtacaaa ggctatcagg tggctcccgc tgaattggaa tccatcttgc tccaacaccc |
| 2521 | caacatcttc gacgcaggtg tcgcaggtct tcccgacgat gacgccggtg aacttcccgc |
| 2581 | cgccgttgtt gttttggagc acggaaagac gatgacggaa aaagagatcg tggattacgt |
| 2641 | cgccagtcaa gtaacaaccg cgaaaaagtt gcgcggagga gttgtgtttg tggacgaagt |
| 2701 | accgaaaggt cttaccggaa aactcgacgc aagaaaaatc agagagatcc tcataaaggc |
| 2761 | caagaagggc ggaaagatcg ccgtgGCTAG Cggaagcgga gccactaact tctccctgtt |
| 2821 | gaaacaagca ggggatgtcg aagagaatcc cgggccaccc aagaagaaga ggaaggtgtc |
| 2881 | caatctcctg actgttcacc agaacctccc tgcgctgcca gtagatgcca ctagcgatga |
| 2941 | ggtcaggaaa aatctcatgg atatgtttag ggatagacag gcgttttctg aacacacctg |
| 3001 | gaaaatgctg cttagcgtgt gccgatcctg ggcagcctgg tgtaagctga acaatcgcaa |
| 3061 | atggttcccc gccgagccgg aggacgtgcg cgattacctg ctgtatctcc aggcaagagg |
| 3121 | gctggctgtc aagactatcc agcagcactt gggccaactg aatatgctgc atcgacgcag |
| 3181 | cgggctcccc cggcctagcg attcaaacgc agtctccctt gttatgagga gaattagaaa |
| 3241 | ggaaaacgta gatgcgggtg agagggctaa gcaggctctc gcttttgagc ggactgattt |
| 3301 | cgaccaggtc agatccctga tggagaacag cgatcggtgc caggacatca ggaacctcgc |
| 3361 | atttctggga attgcatata acacacttct gcgcatagct gagatcgccc ggatcagagt |
| 3421 | gaaagacatc agtcgaacgg acggcggccg gatgcttatt catattggac gcacaaagac |
| 3481 | attggtcagc accgctggcg ttgaaaaggc cttgtccctg ggcgtaacga agctggtgga |
| 3541 | aagatggatc tcagtgtccg gcgtggctga cgaccctaat aattacttgt tctgtcgagt |
| 3601 | gagaaaaaac ggagtcgccg cgccctctgc caccagccaa ttgagtacac gggcccttga |
| 3661 | agggatcttt gaggcaaccc accgactcat atacggagcc aaggatgaca gtggccagag |
| 3721 | gtatctcgcc tggtcaggtc attctgctag ggtgggggcc gcacgagaca tggcgcgggc |
| 3781 | aggagtctcc ataccagaga ttatgcaagc tggaggttgg acaaatgtga acatcgttat |
| 3841 | gaactatatc cgcaatcttg actctgaaac cggggccatg gtgagactgc tcgaagatgg |
| 3901 | tgactaccca tacgatgttc cagattacgc tTAAGAATTC gatatcaagc ttAATAAAAG |
| 3961 | ATCTTTATTT TCATTAGATC TGTGTGTTGG TTTTTTGTGT ggtaaccacg tgcggaccga |
| 4021 | gcggccgcag gaacccctag tgatggagtt ggccactccc tctctgcgcg ctcgctcgct |
| 4081 | cactgaggcc gggcgaccaa aggtcgcccg acgcccgggc tttgcccggg cggcctcagt |
| 4141 | gagcgagcga gcgcgcagct gcctgcaggg gcgcctgatg cggtattttc tccttacgca |
| 4201 | tctgtgcggt atttcacacc gcatacgtca aagcaaccat agtacgcgcc ctgtagcggc |
| 4261 | gcattaagcg cggcgggtgt ggtggttacg cgcagcgtga ccgctacact tgccagcgcc |
| 4321 | ctagcgcccg ctcctttcgc tttcttccct tcctttctcg ccacgttcgc cggctttccc |
| 4381 | cgtcaagctc taaatcgggg gctcccttta gggttccgat ttagtgcttt acggcacctc |
| 4441 | gaccccaaaa aacttgattt gggtgatggt tcacgtagtg ggccatcgcc ctgatagacg |
| 4501 | gtttttcgcc ctttgacgtt ggagtccacg ttctttaata gtggactctt gttccaaact |
| 4561 | ggaacaacac tcaaccctat ctcgggctat tcttttgatt tataagggat tttgccgatt |
| 4621 | tcggcctatt ggttaaaaaa tgagctgatt taacaaaaat ttaacgcgaa ttttaacaaa |
| 4681 | atattaacgt ttacaatttt atggtgcact ctcagtacaa tctgctctga tgccgcatag |
| 4741 | ttaagccagc cccgacaccc gccaacaccc gctgacgcgc cctgacgggc ttgtctgctc |
| 4801 | ccggcatccg cttacagaca agctgtgacc gtctccggga gctgcatgtg tcagaggttt |
| 4861 | tcaccgtcat caccgaaacg cgcgagacga aagggcctcg tgatacgcct atttttatag |
| 4921 | gttaatgtca tgataataat ggtttcttag acgtcaggtg gcacttttcg gggaaatgtg |
| 4981 | cgcggaaccc ctatttgttt atttttctaa atacattcaa atatgtatcc gctcatgaga |
| 5041 | caataaccct gataaatgct tcaataatat tgaaaaagga agagtatgag tattcaacat |
| 5101 | ttccgtgtcg cccttattcc cttttttgcg gcattttgcc ttcctgtttt tgctcaccca |
| 5161 | gaaacgctgg tgaaagtaaa agatgctgaa gatcagttgg gtgcacgagt gggttacatc |
| 5221 | gaactggatc tcaacagcgg taagatcctt gagagttttc gccccgaaga acgttttcca |
| 5281 | atgatgagca cttttaaagt tctgctatgt ggcgcggtat tatcccgtat tgacgccggg |
| 5341 | caagagcaac tcggtcgccg catacactat tctcagaatg acttggttga gtactcacca |
| 5401 | gtcacagaaa agcatcttac ggatggcatg acagtaagag aattatgcag tgctgccata |
| 5461 | accatgagtg ataacactgc ggccaactta cttctgacaa cgatcggagg accgaaggag |
| 5521 | ctaaccgctt ttttgcacaa catgggggat catgtaactc gccttgatcg ttgggaaccg |
| 5581 | gagctgaatg aagccatacc aaacgacgag cgtgacacca cgatgcctgt agcaatggca |
| 5641 | acaacgttgc gcaaactatt aactggcgaa ctacttactc tagcttcccg gcaacaatta |
| 5701 | atagactgga tggaggcgga taaagttgca ggaccacttc tgcgctcggc ccttccggct |
| 5761 | ggctggttta ttgctgataa atctggagcc ggtgagcgtg ggtctcgcgg tatcattgca |
| 5821 | gcactggggc cagatggtaa gccctcccgt atcgtagtta tctacacgac ggggagtcag |
| 5881 | gcaactatgg atgaacgaaa tagacagatc gctgagatag gtgcctcact gattaagcat |
| 5941 | tggtaactgt cagaccaagt ttactcatat atactttaga ttgatttaaa acttcatttt |
| 6001 | taatttaaaa ggatctaggt gaagatcctt tttgataatc tcatgaccaa aatcccttaa |
| 6061 | cgtgagtttt cgttccactg agcgtcagac cccgtagaaa agatcaaagg atcttcttga |
| 6121 | gatccttttt ttctgcgcgt aatctgctgc ttgcaaacaa aaaaaccacc gctaccagcg |
| 6181 | gtggtttgtt tgccggatca agagctacca actctttttc cgaaggtaac tggcttcagc |
| 6241 | agagcgcaga taccaaatac tgtccttcta gtgtagccgt agttaggcca ccacttcaag |
| 6301 | aactctgtag caccgcctac atacctcgct ctgctaatcc tgttaccagt ggctgctgcc |
| 6361 | agtggcgata agtcgtgtct taccgggttg gactcaagac gatagttacc ggataaggcg |
| 6421 | cagcggtcgg gctgaacggg gggttcgtgc acacagccca gcttggagcg aacgacctac |
| 6481 | accgaactga gatacctaca gcgtgagcta tgagaaagcg ccacgcttcc cgaagggaga |
| 6541 | aaggcggaca ggtatccggt aagcggcagg gtcggaacag gagagcgcac gagggagctt |
| 6601 | ccagggggaa acgcctggta tctttatagt cctgtcgggt ttcgccacct ctgacttgag |
| 6661 | cgtcgatttt tgtgatgctc gtcagggggg cggagcctat ggaaaaacgc cagcaacgcg |
| 6721 | gcctttttac ggttcctggc cttttgctgg ccttttgctc acatgt |

Vector pAAV-sgRNA-TBG-Cre:
(SEQ ID NO: 556)

| | |
|---|---|
| 1 | cctgcaggca gctgcgcgct cgctcgctca ctgaggccgc ccgggcaaag cccgggcgtc |
| 61 | gggcgacctt tggtcgcccg gcctcagtga gcgagcgagc gcgcagagag ggagtggcca |
| 121 | actccatcac taggggttcc tgcggccgca cgcgtgaggg cctatttccc atgattcctt |
| 181 | catatttgca tatacgatac aaggctgtta gagagataat tggaattaat ttgactgtaa |
| 241 | acacaaagat attagtacaa aatacgtgac gtagaaagta ataatttctt gggtagtttg |
| 301 | cagttttaaa attatgtttt aaaatggact atcatatgct taccgtaact tgaaagtatt |
| 361 | tcgatttctt ggctttatat atcttGTGGA AAGGACGAAA CACCGTGTAA TAGCTCCTGC |
| 421 | ATGGgtttta gagctaGAAA tagcaagtta aaataaggct agtccgttat caacttgaaa |
| 481 | aagtggcacc gagtcggtgc TTTTTTtcta gaagagggcc tatttcccat gattccttca |
| 541 | tatttgcata tacgatacaa ggctgttaga gagataattg gaattaattt gactgtaaac |
| 601 | acaaagatat tagtacaaaa tacgtgacgt agaaagtaat aatttcttgg gtagtttgca |
| 661 | gttttaaaat tatgttttaa aatggactat catatgctta ccgtaacttg aaagtatttc |
| 721 | gatttcttgg ctttatatat cttGTGGAAA GGACGAAACA CCggaagagc gagctcttct |
| 781 | gttttagagc taGAAAtagc aagttaaaat aaggctagtc cgttatcaac ttgaaaaagt |
| 841 | ggcaccgagt cggtgcTTTT TTggtaccgc ggcctctaga ctcgaggggc tggaagctac |
| 901 | ctttgacatc atttcctctg cgaatgcatg tataatttct acagaaccta ttagaaagga |
| 961 | tcacccagcc tctgcttttg tacaactttc ccttaaaaaa ctgccaattc cactgctgtt |
| 1021 | tggcccaata gtgagaactt tttcctgctg cctcttggtg cttttgccta tggcccctat |
| 1081 | tctgcctgct gaagacactc ttgccagcat ggacttaaac ccctccagct ctgacaatcc |
| 1141 | tctttctctt ttgttttaca tgaagggtct ggcagccaaa gcaatcactc aaagttcaaa |
| 1201 | ccttatcatt ttttgctttg ttcctcttgg ccttggtttt gtacatcagc tttgaaaata |
| 1261 | ccatcccagg gttaatgctg gggttaattt ataactaaga gtgctctagt tttgcaatac |
| 1321 | aggacatgct ataaaaatgg aaagataccg gtgccaccat ggccccaaag GTTAACcgta |
| 1381 | cggccaccat ggaagacgcc aaaaacataa agaaaggccc ggcgccattc tatccgctgg |
| 1441 | aagatggaac cgctggagag caactgcata aggctatgaa gagatacgcc ctggttcctg |
| 1501 | gaacaattgc ttttacagat gcacatatcg aggtggacat cacttacgct gagtacttcg |
| 1561 | aaatgtccgt tcggttggca gaagctatga aacgatatgg gctgaataca aatcacagaa |
| 1621 | tcgtcgtatg cagtgaaaac tctcttcaat tctttatgcc ggtgttgggc gcgttattta |
| 1681 | tcggagttgc agttgcgccc gcgaacgaca tttataatga acgtgaattg ctcaacagta |
| 1741 | tgggcatttc gcagcctacc gtggtgttcg tttccaaaaa ggggttgcaa aaaattttga |
| 1801 | acgtgcaaaa aaagctccca atcatccaaa aaattattat catggattct aaaacggatt |
| 1861 | accagggatt tcagtcgatg tacacgttcg tcacatctca tctacctccc ggttttaatg |
| 1921 | aatacgattt tgtgccagag tccttcgata gggacaagac aattgcactg atcatgaact |
| 1981 | cctctggatc tactggtctg cctaaaggtg tcgctctgcc tcatagaact gcctgcgtga |
| 2041 | gattctcgca tgccagagat cctatttttg gcaatcaaat cattccggat actgcgattt |
| 2101 | taagtgttgt tccattccat cacggttttg gaatgtttac tacactcgga tatttgatat |
| 2161 | gtggatttcg agtcgtctta atgtatagat ttgaagaGga gctgtttctg aggagccttc |
| 2221 | aggattacaa gattcaaagt gcgctgctgg tgccaaccct attctccttc ttcgccaaaa |
| 2281 | gcactctgat tgacaaatac gatttatcta atttacacga aattgcttct ggtggcgctc |
| 2341 | ccctctctaa ggaagtcggg gaagcggttg ccaagaggtt ccatctgcca ggtatcaggc |
| 2401 | aaggatatgg gctcactgag actacatcag ctattctgat tacacccgag ggggatgata |
| 2461 | aaccgggcgc ggtcggtaaa gttgttccat tttttgaagc gaaggttgtg gatctggata |
| 2521 | ccgggaaaac gctgggcgtt aatcaaagag gcgaactgtg tgtgagaggt cctatgatta |
| 2581 | tgtccggtta tgtaaacaat ccggaagcga ccaacgcctt gattgacaag gatggatggc |
| 2641 | tacattctgg agacatagct tactgggacg aagacgaaca cttcttcatc gttgaccgcc |
| 2701 | tgaagtctct gattaagtac aaaggctatc aggtggctcc cgctgaattg gaatccatct |
| 2761 | tgctccaaca ccccaacatc ttcgacgcag gtgtcgcagg tcttcccgac gatgacgccg |
| 2821 | gtgaacttcc cgccgccgtt gttgttttgg agcacggaaa gacgatgacg gaaaaagaga |
| 2881 | tcgtggatta cgtcgccagt caagtaacaa ccgcgaaaaa gttgcgcgga ggagttgtgt |
| 2941 | ttgtggacga agtaccgaaa ggtcttaccg gaaaactcga cgcaagaaaa atcagagaga |
| 3001 | tcctcataaa ggccaagaag ggcggaaaga tcgccgtgGC TAGCggaagc ggagccacta |
| 3061 | acttctccct gttgaaacaa gcaggggatg tcgaagagaa tcccgggcca cccaagaaga |
| 3121 | agaggaaggt gtccaatctc ctgactgttc accagaacct ccctgcgctg ccagtagatg |
| 3181 | ccactagcga tgaggtcagg aaaaatctca tggatatgtt tagggataga caggcgtttt |
| 3241 | ctgaacacac ctggaaaatg ctgcttagcg tgtgccgatc ctgggcagcc tggtgtaagc |
| 3301 | tgaacaatcg caaatggttc cccgccgagc cggaggacgt gcgcgattac ctgctgtatc |
| 3361 | tccaggcaag agggctggct gtcaagacta tccagcagca cttgggccaa ctgaatatgc |
| 3421 | tgcatcgacg cagcgggctc ccccggccta gcgattcaaa cgcagtctcc cttgttatga |
| 3481 | ggagaattag aaaggaaaac gtagatgcgg gtgagagggc taagcaggct ctcgcttttg |
| 3541 | agcggactga tttcgaccag gtcagatccc tgatggagaa cagcgatcgg tgccaggaca |
| 3601 | tcaggaacct cgcatttctg ggaattgcat ataacacact tctgcgcata gctgagatcg |
| 3661 | cccggatcag agtgaaagac atcagtcgaa cggacggcgg ccggatgctt attcatattg |
| 3721 | gacgcacaaa gacattggtc agcaccgctg gcgttgaaaa ggccttgtcc ctgggcgtaa |
| 3781 | cgaagctggt ggaaagatgg atctcagtgt ccggcgtggc tgacgaccct aataattact |
| 3841 | tgttctgtcg agtgagaaaa aacggagtcg ccgcgccctc tgccaccagc caattgagta |
| 3901 | cacgggccct tgaagggatc tttgaggcaa cccaccgact catatacgga gccaaggatg |
| 3961 | acagtggcca gaggtatctc gcctggtcag gtcattctgc tagggtgggg gccgcacgag |
| 4021 | acatggcgcg ggcaggagtc tccataccag agattatgca agctggaggt tggacaaatg |
| 4081 | tgaacatcgt tatgaactat atccgcaatc ttgactctga aaccggggcc atggtgagac |
| 4141 | tgctcgaaga tggtgactac ccatacgatg ttccagatta cgctTAAGAA TTCgatatca |
| 4201 | agcttAATAA AAGATCTTTA TTTTCATTAG ATCTGTGTGT TGGTTTTTTG TGTggtaacc |
| 4261 | acgtgcggac cgagcggccg caggaacccc tagtgatgga gttggccact ccctctctgc |
| 4321 | gcgctcgctc gctcactgag gccgggcgac caaaggtcgc ccgacgcccg ggctttgccc |
| 4381 | gggcggcctc agtgagcgag cgagcgcgca gctgcctgca ggggcgcctg atgcggtatt |
| 4441 | ttctccttac gcatctgtgc ggtatttcac accgcatacg tcaaagcaac catagtacgc |
| 4501 | gccctgtagc ggcgcattaa gcgcggcggg tgtggtggtt acgcgcagcg tgaccgctac |
| 4561 | acttgccagc gccctagcgc ccgctccttt cgctttcttc ccttcctttc tcgccacgtt |
| 4621 | cgccggcttt ccccgtcaag ctctaaatcg ggggctccct ttagggttcc gatttagtgc |
| 4681 | tttacggcac ctcgacccca aaaaacttga tttgggtgat ggttcacgta gtgggccatc |
| 4741 | gccctgatag acggtttttc gccctttgac gttggagtcc acgttcttta atagtggact |
| 4801 | cttgttccaa actggaacaa cactcaaccc tatctcgggc tattcttttg atttataagg |
| 4861 | gattttgccg atttcggcct attggttaaa aaatgagctg atttaacaaa aatttaacgc |
| 4921 | gaattttaac aaaatattaa cgtttacaat tttatggtgc actctcagta caatctgctc |
| 4981 | tgatgccgca tagttaagcc agccccgaca cccgccaaca cccgctgacg cgccctgacg |
| 5041 | ggcttgtctg ctcccggcat ccgcttacag acaagctgtg accgtctccg ggagctgcat |
| 5101 | gtgtcagagg ttttcaccgt catcaccgaa acgcgcgaga cgaaagggcc tcgtgatacg |
| 5161 | cctattttta taggttaatg tcatgataat aatggtttct tagacgtcag gtggcacttt |
| 5221 | tcggggaaat gtgcgcggaa cccctatttg tttatttttc taaatacatt caaatatgta |
| 5281 | tccgctcatg agacaataac cctgataaat gcttcaataa tattgaaaaa ggaagagtat |
| 5341 | gagtattcaa catttccgtg tcgcccttat tccctttttt gcggcatttt gccttcctgt |
| 5401 | ttttgctcac ccagaaacgc tggtgaaagt aaaagatgct gaagatcagt tgggtgcacg |
| 5461 | agtgggttac atcgaactgg atctcaacag cggtaagatc cttgagagtt ttcgccccga |
| 5521 | agaacgtttt ccaatgatga gcacttttaa agttctgcta tgtggcgcgg tattatcccg |
| 5581 | tattgacgcc gggcaagagc aactcggtcg ccgcatacac tattctcaga atgacttggt |
| 5641 | tgagtactca ccagtcacag aaaagcatct tacggatggc atgacagtaa gagaattatg |
| 5701 | cagtgctgcc ataaccatga gtgataacac tgcggccaac ttacttctga caacgatcgg |
| 5761 | aggaccgaag gagctaaccg cttttttgca caacatgggg gatcatgtaa ctcgccttga |
| 5821 | tcgttgggaa ccggagctga atgaagccat accaaacgac gagcgtgaca ccacgatgcc |
| 5881 | tgtagcaatg gcaacaacgt tgcgcaaact attaactggc gaactactta ctctagcttc |
| 5941 | ccggcaacaa ttaatagact ggatggaggc ggataaagtt gcaggaccac ttctgcgctc |
| 6001 | ggcccttccg gctggctggt ttattgctga taaatctgga gccggtgagc gtgggtctcg |
| 6061 | cggtatcatt gcagcactgg ggccagatgg taagccctcc cgtatcgtag ttatctacac |
| 6121 | gacggggagt caggcaacta tggatgaacg aaatagacag atcgctgaga taggtgcctc |
| 6181 | actgattaag cattggtaac tgtcagacca agtttactca tatatacttt agattgattt |
| 6241 | aaaacttcat ttttaattta aaaggatcta ggtgaagatc ctttttgata atctcatgac |
| 6301 | caaaatccct taacgtgagt tttcgttcca ctgagcgtca gaccccgtag aaaagatcaa |
| 6361 | aggatcttct tgagatcctt tttttctgcg cgtaatctgc tgcttgcaaa caaaaaaacc |
| 6421 | accgctacca gcggtggttt gtttgccgga tcaagagcta ccaactcttt ttccgaaggt |
| 6481 | aactggcttc agcagagcgc agataccaaa tactgtcctt ctagtgtagc cgtagttagg |
| 6541 | ccaccacttc aagaactctg tagcaccgcc tacatacctc gctctgctaa tcctgttacc |
| 6601 | agtggctgct gccagtggcg ataagtcgtg tcttaccggg ttggactcaa gacgatagtt |
| 6661 | accggataag gcgcagcggt cgggctgaac ggggggttcg tgcacacagc ccagcttgga |
| 6721 | gcgaacgacc tacaccgaac tgagatacct acagcgtgag ctatgagaaa gcgccacgct |
| 6781 | tcccgaaggg agaaaggcgg acaggtatcc ggtaagcggc agggtcggaa caggagagcg |
| 6841 | cacgagggag cttccagggg gaaacgcctg gtatctttat agtcctgtcg ggtttcgcca |
| 6901 | cctctgactt gagcgtcgat ttttgtgatg ctcgtcaggg gggcggagcc tatggaaaaa |
| 6961 | cgccagcaac gcggcctttt tacggttcct ggccttttgc tggccttttg ctcacatgt |

TBG:
(SEQ ID NO: 557)
gcggcctctagactcgaggggctggaagctacctttgacatcatttcctctgcgaatgcatgtataatttct
acagaacctattagaaaggatcacccagcctctgcttttgtacaactttcccttaaaaaactgccaattcca
ctgctgtttggcccaatagtgagaactttttcctgctgcctcttggtgcttttgcctatggcccctattctg
cctgctgaagacactcttgccagcatggacttaaacccctccagctctgacaatcctattctcttttgtttt
acatgaagggtctggcagccaaagcaatcactcaaagttcaaaccttatcattttttgctttgttcctcttg
gccttggttttgtacatcagctttgaaaataccatcccagggttaatgctggggttaatttataactaagag
tgctctagttttgcaatacaggacatgctataaaaatggaaagataccggtgccaccatggccccaaag

AAV-mTSG Viral Library Production:

The AAV-CRISPR plasmid vector (AAV-vector) and library (AAV-mTSG) were subjected to AAV9 production and chemical purification. Briefly, HEK 293FT cells (ThermoFisher) were transiently transfected with AAV-vector or AAV-mTSG, AAV9 serotype plasmid and pDF6 using polyethyleneimine (PEI). Each replicate consist of five of 80% confluent HEK 293FT cells in 15-cm tissue culture dishes or T-175 flasks (Corning). Multiple replicates were pooled to enhance production yield. Approximately 72 hours post transfection, cells were dislodged and transferred to a conical tube in sterile PBS. 1/10 volume of pure chloroform was added and the mixture was incubated at 37° C. and vigorously shaken for 1 hour. NaCl was added to a final concentration of 1 M and the mixture was shaken until dissolved and then pelleted at 20 k g at 4° C. for 15 minutes. The aqueous layer was discarded while the chloroform layer was transferred to another tube. PEG8000 was added to 10% (w/v) and shaken until dissolved. The mixture was incubated at 4° C. for 1 hour and then spun at 20 k g at 4° C. for 15 minutes. The supernatant was discarded and the pellet was resuspended in DPBS plus MgCl₂ and treated with Benzonase (Sigma) and incubated at 37° C. for 30 minutes. Chloroform (1:1 volume) was then added, shaken, and spun down at 12 k g at 4C for 15 min. The aqueous layer was isolated and passed through a 100 kDa MWCO (Millipore). The concentrated solution was washed with PBS and the filtration process was repeated. Genomic copy number (GC) of AAV was titrated by real-time quantitative PCR (qPCR) using custom Taqman assays (ThermoFisher) targeted to Cre.

Intravenous (i.v.) Virus Injection for Liver Transduction:

Conditional LSL-Cas9 knock-in mice were bred in a mixed 129/C57BL/6 background. Mixed gender (randomized males and females) 8-14 week old mice were used in experiments. Mice were maintained and bred in standard individualized cages with maximum of 5 mice per cage, with regular room temperature (65-75° F., or 18-23° C.), 40-60% humidity, and a 12 h:12 h light cycle. To intravenously inject AAVs, mice were restrained in rodent restrainer (Braintree Scientific), their tails were dilated using a heat lamp or warm water, sterilized by 70% ethanol, and 200 microliters of concentrated AAV (˜1e10GC/μL, 2e12 GC per mouse) was injected into the tail vein of each mouse. 100% of the mice survived the procedure. Animals that failed injections (<70% of total volume injected into tail vein after multiple attempts) were excluded from the study. No specific methods were implemented to choose sample sizes.

MRI:

MRI imaging was performed using standard imaging protocol with MRI machines (Varian 7T/310/ASR—whole mouse MRI system, or Bruker 9.4T horizontal small animal systems). Briefly, animals were anesthetized using isoflurane, and positioned in the imaging bed with a nosecone providing constant isoflurane. A total of 20-30 frontal views were acquired for each mouse using a custom setting: echo time (TE)=20, repetition time (TR)=2000, slicing=1.0 mm. Raw image stacks were processed using Osirix or Slicer tools. Rendering and quantification were performed using Slicer (slicer dot org). Tumor size was calculated with the following formula: Volume (mm³)=⅙*3.14*length (mm)*height (mm)*depth (mm). Statistical significance was assessed by non-parametric Mann-Whitney test, as samples numbers and sample distributions varied across treatment conditions.

Survival Analysis:

LSL-Cas9 mice receiving AAV-mTSG i.v. injections rapidly deteriorated in their body condition scores (due to tumor development in most cases). Mice with body condition score (BSC)<2 were euthanized and the euthanasia date was recorded as the last survival date. Occasionally mice bearing tumors died unexpectedly early, and the date of death was recorded as the last survival date. Cohorts of mice intravenously injected with PBS, AAV-vector or AAV-mTSG virus were monitored for their survival. Survival analysis was analyzed by standard Kaplan-Meier method, using the survival and survminer R packages. Differences among the three treatment groups were assessed by log-rank test. Of note, several AAV-vector or PBS injected mice were sacrificed at time points earlier than the last day of survival analysis (at times when a certain AAV-mTSG mice were found dead or euthanized due to poor body conditions), to provide time-matched histology, even though those mice presented with good body condition (BSC≥4). Mice euthanized early in a healthy state were excluded from calculation of survival percentages.

Mouse Organ Dissection, Fluorescent Imaging, and Histology:

Mice were sacrificed by carbon dioxide asphyxiation or deep anesthesia with isoflurane followed by cervical dislocation. Mouse livers and other organs were manually dissected and examined under a fluorescent stereoscope (Zeiss, Olympus or Leica). Brightfield and/or GFP fluorescent images were taken for the dissected organs, and overlaid using ImageJ. Organs were then fixed in 4% formaldehyde or 10% formalin for 48 to 96 hours, embedded in paraffin, sectioned at 6 μm and stained with hematoxylin and eosin (H&E) for pathology. For tumor size quantification, H&E slides were scanned using an Aperio digital slidescanner (Leica). Tumors were manually outlined as region-of-interest (ROI), and subsequently quantified using ImageScope (Leica). Statistical significance was assessed by Welch's t-test, given the unequal sample numbers and variances for each treatment condition.

Mouse Tissue Collection for Molecular Biology:

Mouse livers and various other organs were dissected and collected manually. For molecular biology, tissues were flash frozen with liquid nitrogen, ground in 24 Well Polyethylene Vials with metal beads in a GenoGrinder machine (OPS diagnostics). Homogenized tissues were used for DNA/RNA/protein extractions using standard molecular biology protocols.

Genomic DNA Extraction from Cells and Mouse Tissues:

For genomic DNA extraction, 50-200 mg of frozen ground tissue were resuspended in 6 ml of Lysis Buffer (50 mM Tris, 50 mM EDTA, 1% SDS, pH 8) in a 15 ml conical tube, and 30 μl of 20 mg/ml Proteinase K (Qiagen) were added to the tissue/cell sample and incubated at 55° C. overnight. The next day, 30 IA of 10 mg/ml RNAse A (Qiagen) was added to the lysed sample, which was then inverted 25 times and incubated at 37° C. for 30 minutes. Samples were cooled on ice before addition of 2 ml of pre-chilled 7.5M ammonium acetate (Sigma) to precipitate proteins. The samples were vortexed at high speed for 20 seconds and then centrifuged at >4,000×g for 10 minutes. Then, a tight pellet was visible in each tube and the supernatant was carefully decanted into a new 15 ml conical tube. Then 6 ml 100% isopropanol was added to the tube, inverted 50 times and centrifuged at >4,000×g for 10 minutes. Genomic DNA was visible as a small white pellet in each tube. The supernatant was discarded, 6 ml of freshly prepared 70% ethanol was added, the tube was inverted 10 times, and then centrifuged at >4,000×g for 1 minute. The supernatant was discarded by pouring; the tube was briefly spun, and remaining ethanol was removed using a P200 pipette. After air-drying for 10-30 minutes, the DNA changed appearance from a milky white pellet to slightly translucent. Then, 500 μl of ddH₂O was added, the tube was incubated at 65° C. for 1 hour and at room temperature overnight to fully resuspend the DNA. The next day, the gDNA samples were vortexed briefly. The gDNA concentration was measured using a Nanodrop (Thermo Scientific).

Molecular Inversion Probe (MIP) Design and Synthesis:

MIPs were designed according to previously published protocols (Hardenbol, P. et al., Nat. Biotechnol. 21, 673-678 (2003); O'Roak, B. J. et al., Science 338, 1619-1622 (2012). Briefly, the 70 bp flanking the predicted cut site of each sgRNA of all 278 unique sgRNA were chosen as targeting regions, and the bed file with these coordinates was used as an input. Since Trp53 sg4 targets a similar region as the p53 sgRNA within the base vector, the same MIP was used to sequence both of these loci.

These coordinates contained overlapping regions which were subsequently merged into 173 unique regions. Each probe contains an extension probe sequence, a ligation probe sequence, and a 7 bp degenerate barcode (NN) for PCR duplicate removal. A total of 266 MIP probes were designed covering a total amplicon of 42,478 bp. MIP target size stats: min=155 bp, max=190 bp, mean=159.7 bp, median=156.0 bp. Each of the mTSG-MIPs were synthesized using standard oligo synthesis with IDT, normalized and pooled.

MIP Capture Sequencing:

150 ng of genomic DNA sample from each mouse organ was used as input. MIP capture sequencing was done according to previously published protocols (Hardenbol, P. et al., Nat. Biotechnol. 21, 673-678 (2003); O'Roak, B. J. et al., Science 338, 1619-1622 (2012) with some slight modifications. The multiplexed library was then quality controlled using qPCR, and subjected to high-throughput sequencing using the Hiseq-2500 or Hiseq-4000 platforms (Illumina) at Yale Center for Genome Analysis. 280/281 (99.6%) of targeted sgRNAs were captured for all samples from this experiment, with the missing one being Arid1a sg5.

Illumina Sequencing Data Pre-Processing:

FASTQ reads were mapped to the mm10 genome using the bwa mem function in BWA v0.7.13. Bam files were merged, sorted, and indexed using bamtools v2.4.0 and samtools v1.3.

Variant Calling:

For each sample, indel variants were called using samtools and VarScan v2.3.9. Specifically, samtools mpileup (-d 1000000000 -B -q 10) was used, and the output piped to VarScan pileup2indel ( - - - min-coverage 1 min-reads2 1 - - - min-var-freq 0.001 - - - p-value 0.05). To link each indel to the sgRNA that most likely caused the mutation, the center position of each indel was mapped to the closest sgRNA cut site.

Calling Mutated sgRNA Sites and Mutated Genes:

All detected indels were further filtered by requiring that each indel must overlap the ±3 basepair flank of the closest sgRNA cut site, as Cas9-induced double-strand breaks are expected to occur within a narrow window of the predicted cut site. To exclude any possible germline mutations, any sgRNAs with indels present in more than half of the control samples with greater than 5% variant frequency were removed. In particular, high variant frequencies were observed across all samples at the Rps19 sg5 cut site, suggesting these were germline variants; thus, Rps19 sg5 was excluded from all analyses.

To determine significantly mutated sgRNA sites in each liver sample, a false-discovery approach was used based on the PBS and vector control samples. For each sgRNA, the highest % variant read frequency across all control liver samples were first taken: in order for a mutation to be called in an mTSG sample, the % variant read frequency had to exceed the control sample cutoff. However, since the base vector contained a Trp53 sgRNA (p53 sg8) whose cut site was only 1 bp away from the target site of Trp53 sg4 (from mTSG library), PBS samples were considered only when calculating the false-discovery cutoff for Trp53 sg4. To identify the dominant clones in each sample, a 5% variant frequency cutoff was set on top of the false-discovery cutoff. These criteria yielded a binary table (i.e. not significantly mutated vs. significantly mutated) detailing each sgRNA and whether its target site was significantly mutated in each sample. To convert significantly mutated sgRNA sites into significantly mutated genes, the binary sgRNA scores were collapsed by gene, such that if any of the 5 sgRNAs for a gene were found to be significantly cutting, the entire gene would be called as significantly mutated.

Coding Frame Analysis:

For coding frame and exonic/intronic analysis, only indels that were associated with an sgRNA that had been considered significantly mutated in that particular sample were considered. This final set of significant indels was converted to .avinput format and subsequently annotated using ANNOVAR v. 2016 Feb. 1, using default settings.

Co-Occurrence and Correlation Analysis:

Co-occurrence analysis was performed by first generating a double-mutant count table for each pairwise combination of genes in the mTSG library. Statistical significance of the co-occurrence was assessed by two-sided hypergeometric test. To calculate co-occurrence rates, the “intersection” was defined as the number of double-mutant samples, and the “union” defined as the number of samples with a mutation in either (or both) of the two genes, and then divided the intersection by the union. For correlation analysis, the table of variant frequencies was first collapsed to the gene level (in other words, summing the variant frequencies for all 5 of the targeting sgRNAs for each gene). Using these summed variant frequency values, the Pearson correlation was calculated between all gene pairs, across each mTSG sample. Statistical significance of the correlation was determined by converting the correlation coefficient to a t-statistic, and then using the t-distribution to find the associated probability. For both co-occurrence and correlation analyses, p-values were adjusted for multiple hypothesis testing by the Benjamini-Hochberg method to obtain q-values.

Unique Variant Analysis:

Instead of first collapsing variant calls to the sgRNA level as above, unique variants and their associated mutant frequencies were compiled across all sequenced samples. To be considered present in a given sample, a particular variant must have a mutant frequency ≥1%. Hierarchically clustered heatmaps of the unique variant landscape were created in R using the NMF package, with average linkage and Euclidean distance.

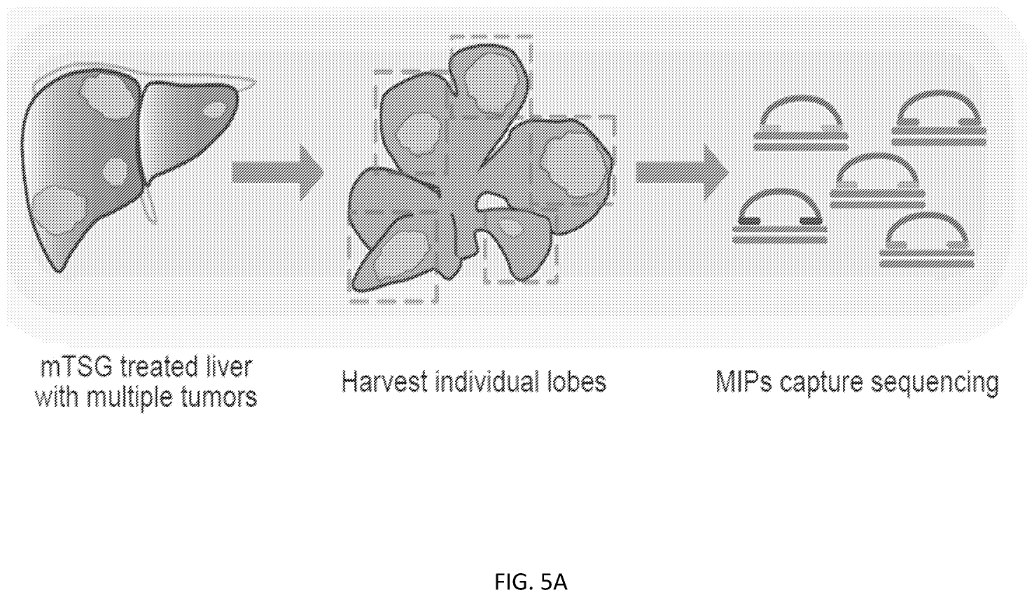

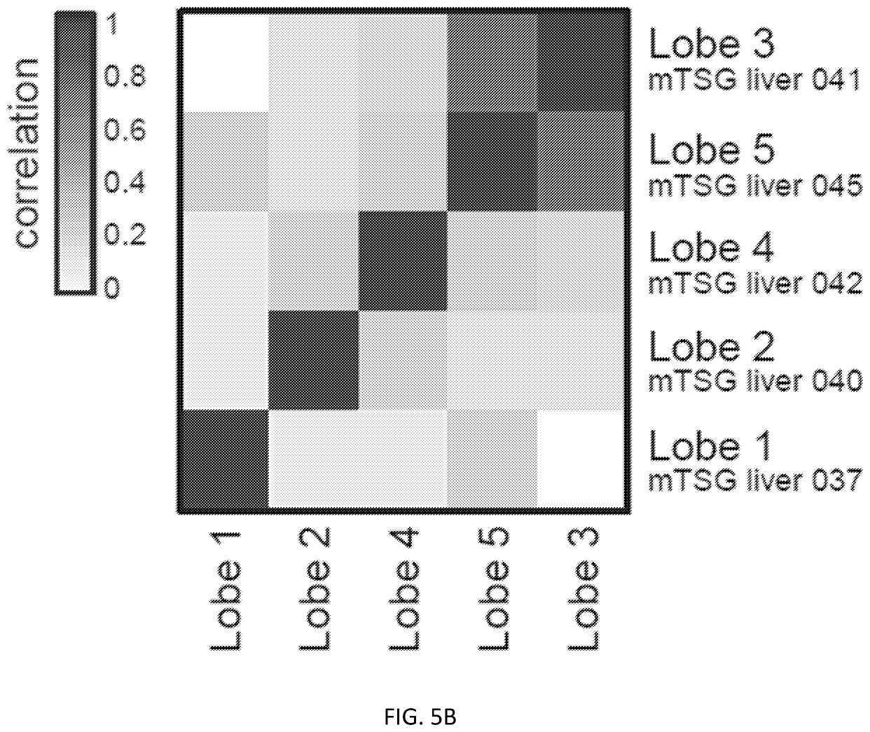

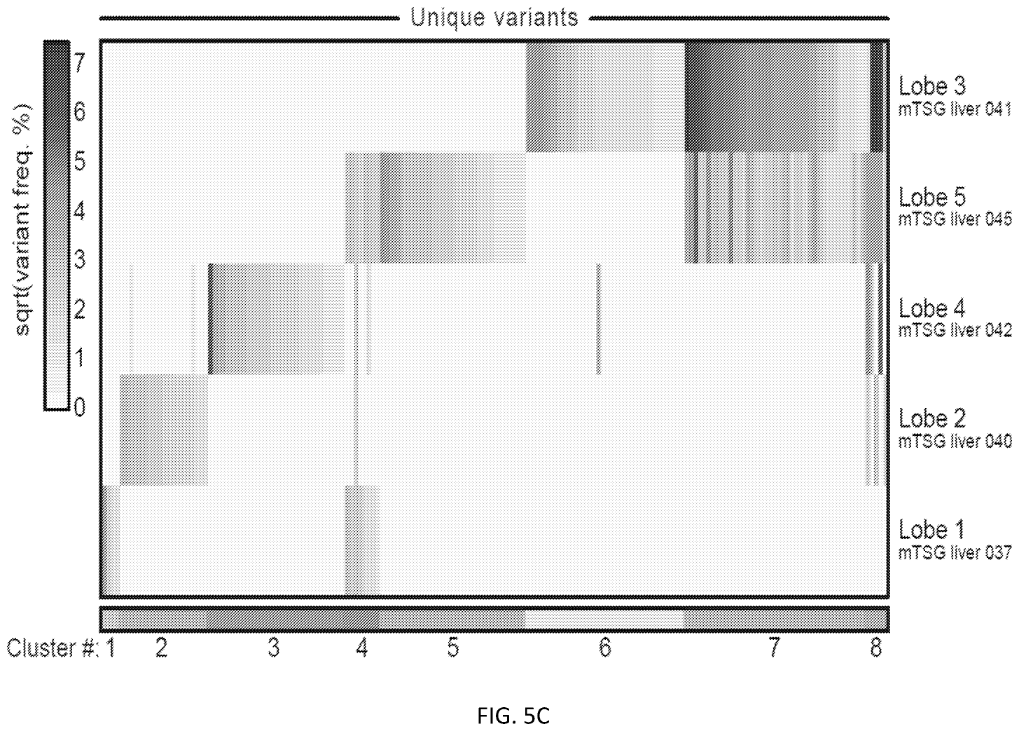

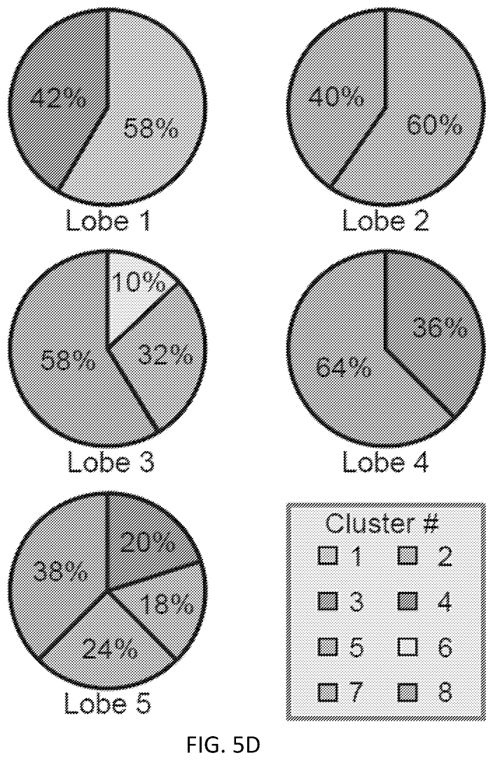

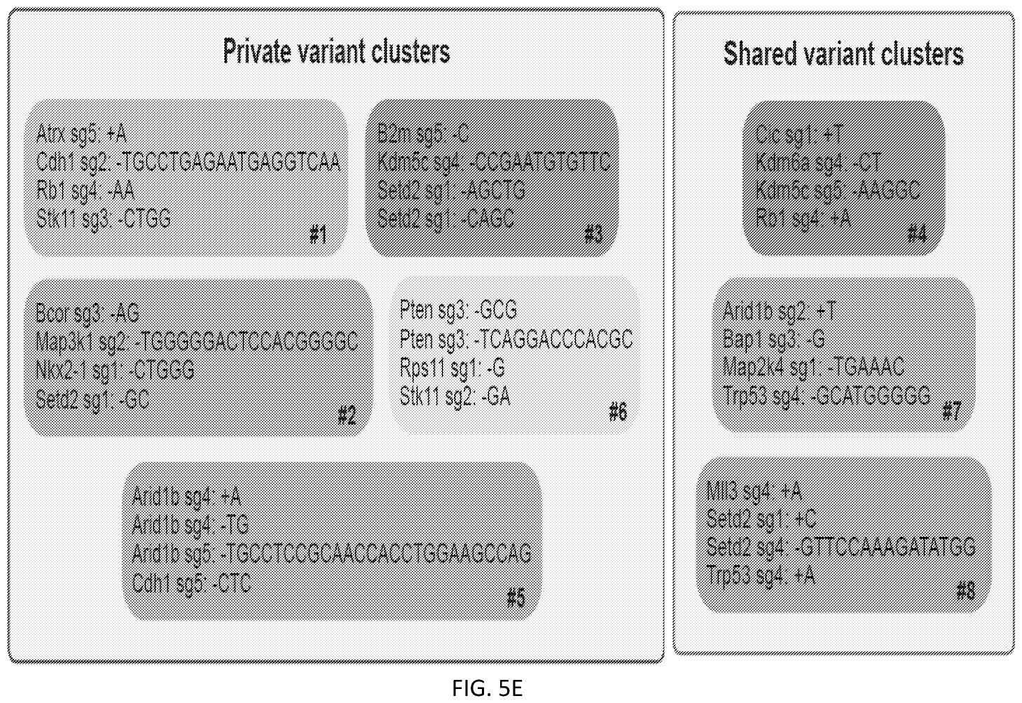

A focused analysis on the unique variant landscape within a single mouse was also performed, as presented in FIGS. 5A-5E. For the correlation heatmap in FIG. 5B, Spearman rank correlation was used to assess the pairwise correlation between different liver lobes. In FIG. 5C, clusters of variants were defined on the basis of binary mutation calls—i.e. whether a given variant is present or not within each sample. To determine the proportional contribution of each cluster, for each sample, only included were the clusters in which at least half of the variants in the cluster are present in that sample. The average mutant frequency was taken across the variants within each cluster, and these values were used to determine the relative contribution of each cluster to the overall sample. To identify the top four variants in each cluster, the variants were ranked by the average variant frequency across all lobes in which the variant cluster was considered present.

Clustering of Variant Frequencies to Infer Clonality of Tumors:

For each mTSG liver sample, the individual variants that comprised the MS calls in that sample were extracted, with a cutoff of 5% variant frequency to eliminate low-abundance variants. To identify clusters of variant frequencies in an unbiased manner, the variant frequency distribution was modeled with a Gaussian kernel density estimate, using the Sheather-Jones method to select the smoothing bandwidth. From the kernel density estimate, the number of local maxima (i.e. “peaks”) within the density function were then identified. The number of peaks thus represented the number of variant frequency clusters for an individual sample, which is an approximation for the clonality of the tumors.

Direct In Vivo Validation of Drivers or Combinations:

Liver-specific AAV-CRISPR vectors were designed to co-cistronically expresses firefly luciferase (FLuc) and Cre recombinase for induction of Cas9 expression under a TBG promoter when delivered to LSL-Cas9 mice (Plasmids available at Addgene). Two sgRNA cassettes were built in these vectors, one encoding an sgRNA targeting Trp53, with the other being an open sgRNA cassette (double SapI sites for GeneX targeting sgRNA cloning). The vector was generated by gBlock gene fragment synthesis (IDT) followed by Gibson assembly (NEB). Each specific sgRNA targeting a driver gene was cloned separately into this vector. AAV9 virus was produced and qPCR-titrated as described above. 1e11 total viral particles were introduced by intravenous injection into LSL-Cas9 mice. For combinations of two AAVs, 5e10 viral particles were used from each AAV to generate equal titer mixtures and injected. Four to six mice were injected per group. One month after injection, mice were imaged by IVIS each month. Briefly, mice were anesthetized by intraperitoneal injection of ketamine (100 mg/kg) and xylazine (10 mg/kg), and imaged for in vivo tumor growth using an IVIS machine (PerkinElmer) with 150 mg/kg body weight Firefly D-Luciferin potassium salt injected I.P. Relative tumor burden were quantified using LivingImage software (PerkinElmer).

LIHC comparative cancer genomics analysis and patient survival analysis using TCGA datasets: Somatic mutation calls, copy number variation calls, RNA-seq expression z-scores, and clinical data containing patient survival information were obtained through cBioPortal for liver hepatocellular carcinoma (LIHC data set) (Gao, et al., Sci. Signal. 6, pl 1 (2013); Cerami, et al., Cancer Discov. 2, 401-404 (2012)) on Nov. 15, 2016. Pearson correlation coefficients were calculated comparing mouse and human mutation frequencies; statistical significance was calculated by converting the correlation coefficient to a t-statistic, and then using the t-distribution to find the associated probability. All patients with sequencing data and survival data were considered (n=372). A tumor was defined as being “negative” for a given gene if it had one or more of the following: 1) a non-silent somatic mutation, 2) homozygous deletion, or 3) an expression z-score <−2. On the basis of these negative vs. positive classifications, Kaplan-Meier survival analysis was performed, using the log-rank test to determine statistical significance.

The results of the experiments are now described.

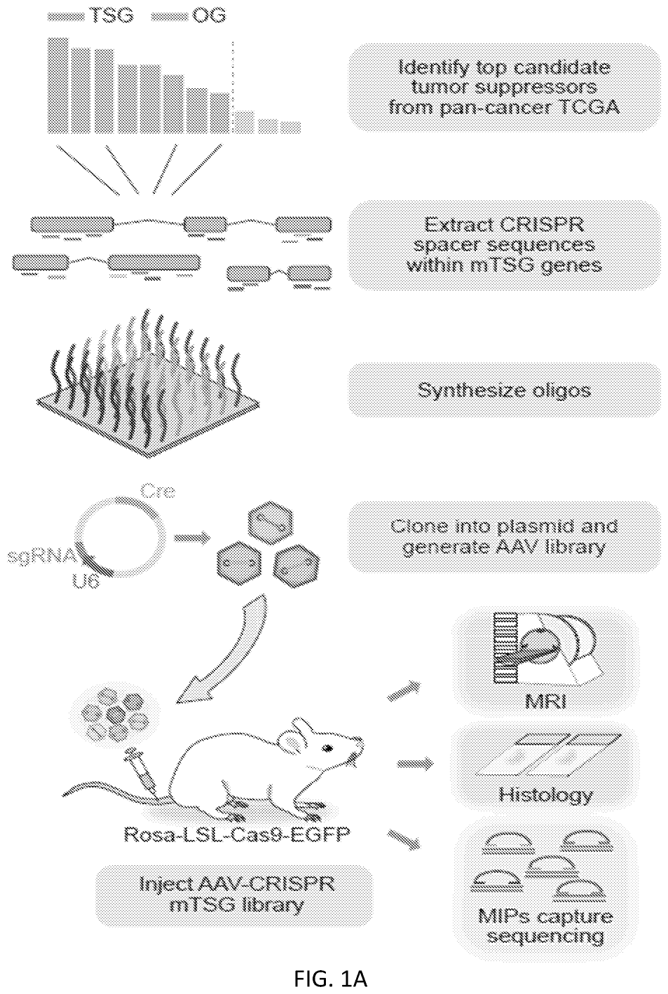

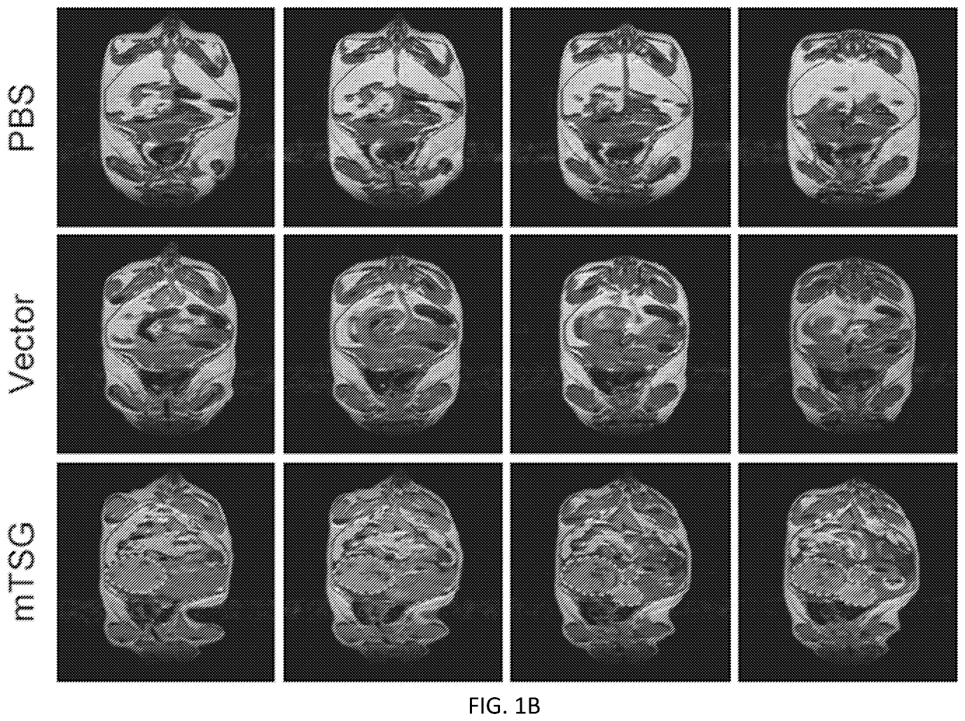

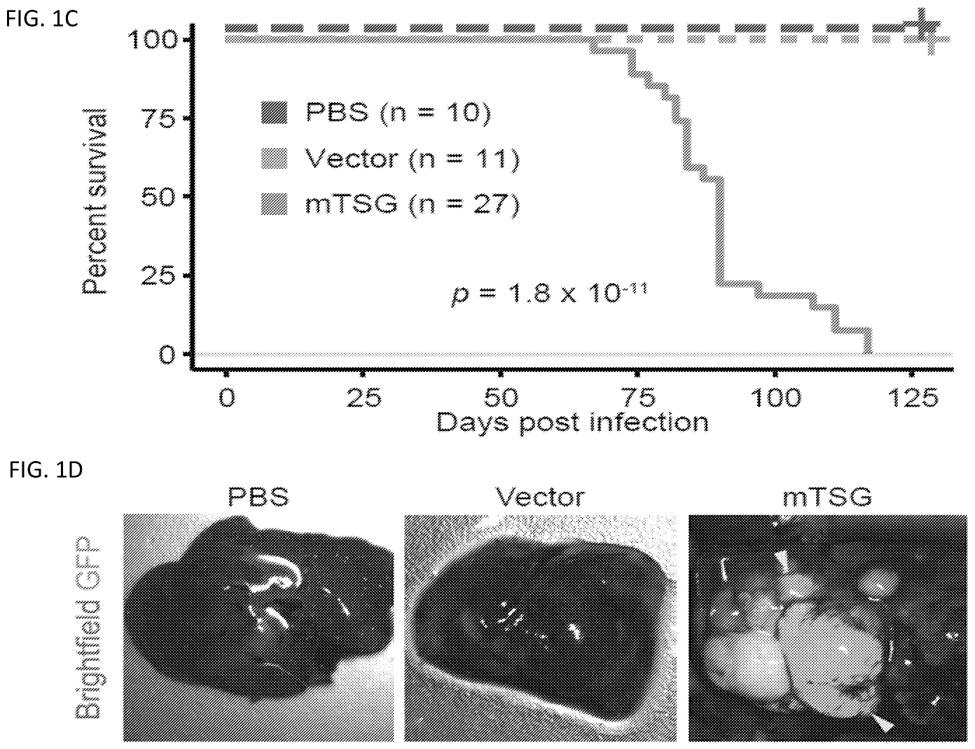

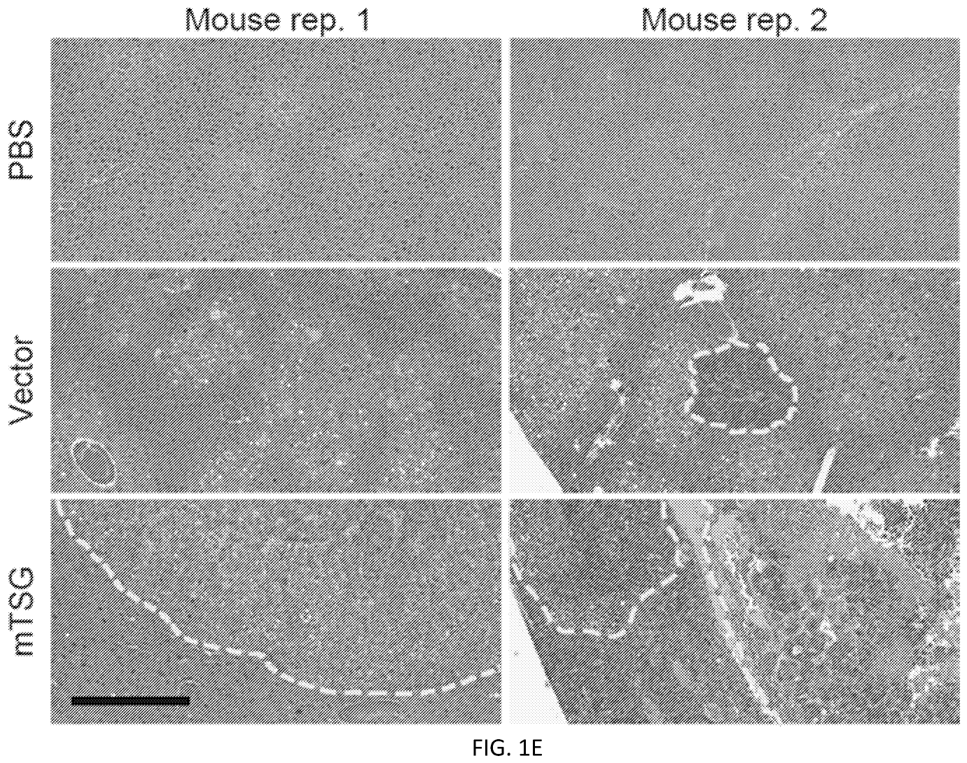

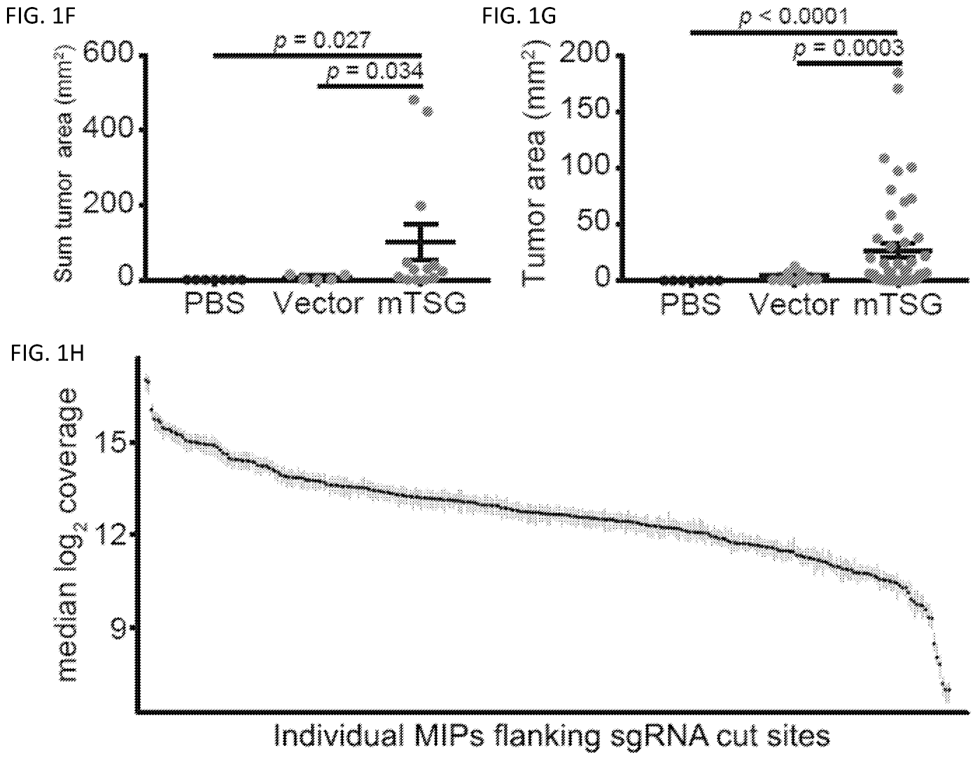

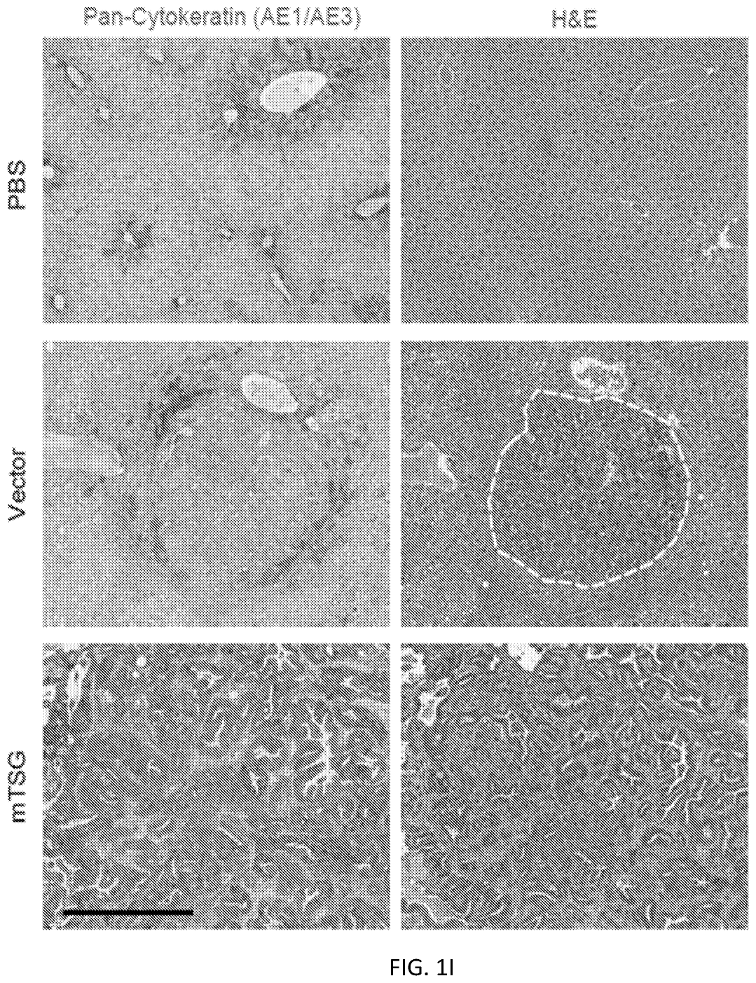

A list of the top SMGs in the pan-cancer TCGA datasets was compiled. The top 50 SMGs were identified after excluding known oncogenes (FIG. 1A). Of the top 50 putative TSGs, 49 genes had mouse orthologs (mouse TSGs, hereafter referred to as mTSG). Seven additional genes were selected from a set of housekeeping genes, to serve as controls. A library of sgRNAs was designed targeting these 56 different genes, with 5 sgRNAs for each gene, totaling 280 sgRNAs (hereafter referred to as the mTSG library) (FIG. 1A; Table 1). For Cdkn2a and Rpl22, only four unique sgRNAs were synthesized, with the fifth sgRNA being a duplicate. The duplicates were treated as identical in downstream analyses. After oligo synthesis, the mTSG library was cloned into a base vector expression cassette containing a U6 promoter driving the expression of the sgRNA cassette, as well as a Cre expression cassette (FIG. 1A). Because mutation of a single TSG rarely leads to rapid tumorigenesis in humans or autochthonous mouse models, an sgRNA targeting Trp53 in the base vector was included, with the initial hypothesis that concomitant Trp53 loss-of-function might facilitate tumorigenesis. Sequencing of the plasmid pool revealed a complete coverage of the 280 sgRNAs represented in the mTSG library (Table 2). After generating AAVs (serotype AAV9) containing the base vector or the mTSG library, PBS, vector AAVs, or mTSG AAVs were intravenously injected into fully immunocompetent LSL-Cas9 mice (FIG. 1A). Upon AAV infection, Cre is expressed and excises the stop codon, activating Cas9 and EGFP expression.

TABLE 1
mTSG library

| sgRNA_name | Spacer sequence | SEQ ID NO |
|---|---|---|
| Fat1_sg1 | GGGCAGTGTTTCAAAATCCA | SEQ ID NO: 1 |
| Fat1_sg2 | GGAACACGAGCCGTCAGCGG | SEQ ID NO: 2 |
| Fat1_sg3 | GGATTTCTGTTCTGCATCAA | SEQ ID NO: 3 |
| Fat1_sg4 | GGTCCCATCTGTTGCCTCCA | SEQ ID NO: 4 |
| Fat1_sg5 | GTTTGGAGATCCACTCGATA | SEQ ID NO: 5 |
| Arid1b_sg1 | GTACCCAGTGCAAGCTACAG | SEQ ID NO: 6 |
| Arid1b_sg2 | GGGTACCCCGCTATATGTTG | SEQ ID NO: 7 |
| Arid1b_sg3 | GCATCTGGCCCCCAGGAGAT | SEQ ID NO: 8 |
| Arid1b_sg4 | GGTCCGTACTGAGACATCTG | SEQ ID NO: 9 |
| Arid1b_sg5 | GATTCTGACTGGCTTCCAGG | SEQ ID NO: 10 |
| Trp53_sg1 | GTGAAATACTCTCCATCAAG | SEQ ID NO: 11 |
| Trp53_sg2 | GGGAGAGGCGCTTGTGCAGG | SEQ ID NO: 12 |
| Trp53_sg3 | GCAAAGAGAGGTACGCAGGC | SEQ ID NO: 13 |
| Trp53_sg4 | GCGGTTCATGCCCCCCATGC | SEQ ID NO: 14 |
| Trp53_sg5 | GGTATACTCAGAGCCGGCCT | SEQ ID NO: 15 |
| Grlf1_sg1 | GCCATGGCTGAAGGGGAGCC | SEQ ID NO: 16 |
| Grlf1_sg2 | GACTCTGTAGGTGACAGCAA | SEQ ID NO: 17 |
| Grlf1_sg3 | GCTCCTAAACCTACTTGTTC | SEQ ID NO: 18 |
| Grlf1_sg4 | GCATGCTGTATGGTACCAGG | SEQ ID NO: 19 |
| Grlf1_sg5 | GAAGGCATCTACCGGGTCAG | SEQ ID NO: 20 |
| Npm1_sg1 | GGACGATGATGAGGACGATG | SEQ ID NO: 21 |
| Npm1_sg2 | GTGTAATTTCAAAGCCCCCT | SEQ ID NO: 22 |
| Npm1_sg3 | GTGGACAGCATCTAGTAGGT | SEQ ID NO: 23 |
| Npm1_sg4 | GGGCGGTTCTCTTCCCAAAG | SEQ ID NO: 24 |
| Npm1_sg5 | GGAAGAGAACCGCCCTATTG | SEQ ID NO: 25 |
| Ep300_sg1 | GCATATGCTCGTAAAGTGGA | SEQ ID NO: 26 |
| Ep300_sg2 | GATGAGTGAAAATGCTGGTG | SEQ ID NO: 27 |
| Ep300_sg3 | GGCTGAGCTGCTGTTGGCAA | SEQ ID NO: 28 |
| Ep300_sg4 | GCCCTAGGTGCTAGTCCTAT | SEQ ID NO: 29 |
| Ep300_sg5 | GCTAGTCCTATGGGTGTAAA | SEQ ID NO: 30 |
| Mll2_sg1 | GCCGGCTATGTCGGGCCTGT | SEQ ID NO: 31 |
| Mll2_sg2 | GGGATTCAGTTCTGCTGAGC | SEQ ID NO: 32 |
| Mll2_sg3 | GTGTGTGAGACATGTGACAA | SEQ ID NO: 33 |
| Mll2_sg4 | GCAGGCAGGTCCTCCATAGG | SEQ ID NO: 34 |
| Mll2_sg5 | GCCTCCTCTGCCGGAGAAAG | SEQ ID NO: 35 |
| Setd2_sg1 | GACTGTGAATGGACAGCTGA | SEQ ID NO: 36 |
| Setd2_sg2 | GTCTTCTCAAAACATTTCAG | SEQ ID NO: 37 |
| Setd2_sg3 | GCACTGATGGAAAATGGTGA | SEQ ID NO: 38 |
| Setd2_sg4 | GACTACCAGTTCCAAAGATA | SEQ ID NO: 39 |
| Setd2_sg5 | GAAGCTTCTGGTTACTTTCC | SEQ ID NO: 40 |
| Cdkn2a_sg1 | GGGATTGGCCGCGAAGTTCC | SEQ ID NO: 41 |
| Cdkn2a_sg2 | GGGGTACGACCGAAAGAGTT | SEQ ID NO: 42 |
| Cdkn2a_sg3 | GGGTCGCCTGCCGCTCGACT | SEQ ID NO: 43 |
| Cdkn2a_sg4 | GGGAACGTCGCCCAGACCGA | SEQ ID NO: 44 |
| Cdkn2a_sg5 | GGGGTACGACCGAAAGAGTT | SEQ ID NO: 45 |
| Rpl7_sg1 | GTACCTGCACCAGGAAAACC | SEQ ID NO: 46 |
| Rpl7_sg2 | GTGGAGCCATACATTGCATG | SEQ ID NO: 47 |
| Rpl7_sg3 | GGGTGAGTTTTCTGTCTAGT | SEQ ID NO: 48 |
| Rpl7_sg4 | GCCTTTGTCATCAGAATTCG | SEQ ID NO: 49 |
| Rpl7_sg5 | GAAGGCAAAGCACTATCACA | SEQ ID NO: 50 |
| Pbrm1_sg1 | GCAATGGTCTTGAGATCTAT | SEQ ID NO: 51 |
| Pbrm1_sg2 | GACCATTGCTCAGAGGATAC | SEQ ID NO: 52 |
| Pbrm1_sg3 | GCCTGGGTCTCAAGTATTCA | SEQ ID NO: 53 |
| Pbrm1_sg4 | GCCAAAACATACAATGAGCC | SEQ ID NO: 54 |
| Pbrm1_sg5 | GTGCGAAGGACCTGTCAGCC | SEQ ID NO: 55 |
| Pik3r1_sg1 | GACTGCATGGGCAGAAGGGA | SEQ ID NO: 56 |
| Pik3r1_sg2 | GAGACGGCACTTTCCTTGTC | SEQ ID NO: 57 |
| Pik3r1_sg3 | GTTGGCTACAGTAGTGGGCT | SEQ ID NO: 58 |
| Pik3r1_sg4 | GGCAGTGCTGCAGGCAAAAG | SEQ ID NO: 59 |
| Pik3r1_sg5 | GGCTGACGCAGAAAGGTGTG | SEQ ID NO: 60 |
| Rps19_sg1 | GGCCGCAAGCTGACGCCTCA | SEQ ID NO: 61 |
| Rps19_sg2 | GCCTCAGGGACAGAGAGACC | SEQ ID NO: 62 |
| Rps19_sg3 | GTCCCTGAGGCGTCAGCTTG | SEQ ID NO: 63 |
| Rps19_sg4 | GGGCCGCAAGCTGACGCCTC | SEQ ID NO: 64 |
| Rps19_sg5 | GTTGAAACAGAGCGGGGGGG | SEQ ID NO: 65 |
| Bcor_sg1 | GTGGATGAAAGGCTCTTCAT | SEQ ID NO: 66 |
| Bcor_sg2 | GGTTTTGCACAGTCTCTTCC | SEQ ID NO: 67 |
| Bcor_sg3 | GACCTCAGGCTGAACAGCCT | SEQ ID NO: 68 |
| Bcor_sg4 | GGCCCAGGCTGTTCAGCCTG | SEQ ID NO: 69 |
| Bcor_sg5 | GTCCACCACCCCCTGGTCAC | SEQ ID NO: 70 |
| Mll3_sg1 | GTTGGCACTGATTTCATAAC | SEQ ID NO: 71 |
| Mll3_sg2 | GGGAGAAGATAGCAAGATGC | SEQ ID NO: 72 |
| Mll3_sg3 | GTGGCTACTGACCAAACCCA | SEQ ID NO: 73 |
| Mll3_sg4 | GAGAATTCCTAACAGCTATG | SEQ ID NO: 74 |
| Mll3_sg5 | GCTGCCGATACTCCAAACTT | SEQ ID NO: 75 |
| Kdm6a_sg1 | GCAACTATTTTACAACAATT | SEQ ID NO: 76 |
| Kdm6a_sg2 | GGTAAATTAAAACACTCACC | SEQ ID NO: 77 |
| Kdm6a_sg3 | GTAAATTAAAACACTCACCT | SEQ ID NO: 78 |
| Kdm6a_sg4 | GCAGCATTTTCAGTTAGCTT | SEQ ID NO: 79 |
| Kdm6a_sg5 | GGCTATTAAAGCATTTCAGG | SEQ ID NO: 80 |
| Atm_sg1 | GTGATTTTGATCTCGTGCCT | SEQ ID NO: 81 |
| Atm_sg2 | GCAAGGTACACTGTAATCAG | SEQ ID NO: 82 |
| Atm_sg3 | GTGCTTATGAATCCATGAAA | SEQ ID NO: 83 |
| Atm_sg4 | GTCCAAATATATAGTAAGGT | SEQ ID NO: 84 |
| Atm_sg5 | GAGACTTGAGGAAAATGTTA | SEQ ID NO: 85 |
| Rnf43_sg1 | GGGGCCAAGGGTATGCCAGA | SEQ ID NO: 86 |
| Rnf43_sg2 | GACTGTGGGATCCCAGTTTC | SEQ ID NO: 87 |
| Rnf43_sg3 | GTAGGTAGGAGGTGAACTCA | SEQ ID NO: 88 |
| Rnf43_sg4 | GCATGTTCAACATCGTAGGT | SEQ ID NO: 89 |
| Rnf43_sg5 | GGAGTCTTCTGCCTGGTTCC | SEQ ID NO: 90 |
| Vhl_sg1 | GGACTACCCAAGTGTGCGGA | SEQ ID NO: 91 |
| Vhl_sg2 | GCACCTTGAGAGTCAGCACC | SEQ ID NO: 92 |
| Vhl_sg3 | GGTTAACCAGAAGTCCATCA | SEQ ID NO: 93 |
| Vhl_sg4 | GTGCCATCCCTCAATGTCGA | SEQ ID NO: 94 |
| Vhl_sg5 | GTCCTGAGGAGATGGAGGCT | SEQ ID NO: 95 |
| Sf3b3_sg1 | GTCTCCTTCTTCTAGAGGCA | SEQ ID NO: 96 |
| Sf3b3_sg2 | GGCAAAACAGAATAGGAGAG | SEQ ID NO: 97 |
| Sf3b3_sg3 | GGCAATTTGATACAAGTAAC | SEQ ID NO: 98 |
| Sf3b3_sg4 | GCAATTTGATACAAGTAACT | SEQ ID NO: 99 |
| Sf3b3_sg5 | GCACAGTATCAAAATACTTG | SEQ ID NO: 100 |
| Map2k4_sg1 | GACAAAGTTGATGAAACTGG | SEQ ID NO: 101 |
| Map2k4_sg2 | GCCGATTTCCTTATCCAAAG | SEQ ID NO: 102 |
| Map2k4_sg3 | GACCCAAGTGCATCAAGACA | SEQ ID NO: 103 |
| Map2k4_sg4 | GCACTTGGGTCTATTCTTTC | SEQ ID NO: 104 |
| Map2k4_sg5 | GGGCGACTGTTGGATCTGTA | SEQ ID NO: 105 |
| Arid2_sg1 | GTCCAGTAAAAGCTGGAGGA | SEQ ID NO: 106 |
| Arid2_sg2 | GAGTGGTTCTGAAATCCACA | SEQ ID NO: 107 |
| Arid2_sg3 | GGAGAGCAATGTTAAGCTCT | SEQ ID NO: 108 |
| Arid2_sg4 | GACTGTGTGCAGAGAGCAAC | SEQ ID NO: 109 |
| Arid2_sg5 | GTCACTTCTCATTACAGTTT | SEQ ID NO: 110 |
| Tgfbr2_sg1 | GATGCCCTGCAGAGGAAAGG | SEQ ID NO: 111 |
| Tgfbr2_sg2 | GGCAGAGCGCTTCAGTGAGC | SEQ ID NO: 112 |
| Tgfbr2_sg3 | GACAGTGTGCTGAGAGACCG | SEQ ID NO: 113 |
| Tgfbr2_sg4 | GGCCGGAAATTCCCAGCTTC | SEQ ID NO: 114 |
| Tgfbr2_sg5 | GTGTTTCTTTTGGTCTTAGG | SEQ ID NO: 115 |
| Atrx_sg1 | GTGTTTCTCCCTTTAAGTCT | SEQ ID NO: 116 |
| Atrx_sg2 | GGCAGCCCCAATTCTGCTCA | SEQ ID NO: 117 |
| Atrx_sg3 | GATATTAGCCGTGACTCAGA | SEQ ID NO: 118 |
| Atrx_sg4 | GAAGACAAAGATGATTTTAA | SEQ ID NO: 119 |
| Atrx_sg5 | GGTTTCCTACAAAAGAGTTA | SEQ ID NO: 120 |
| Rpl22_sg1 | GCTCATTGGTTGGTTTCTGC | SEQ ID NO: 121 |
| Rpl22_sg2 | GCCTTTCTCCAAAAGGTATG | SEQ ID NO: 122 |
| Rpl22_sg3 | GGTTAGTATGGCTCCGCGTG | SEQ ID NO: 123 |
| Rpl22_sg4 | GCGTTACTTCCAGATTAACC | SEQ ID NO: 124 |
| Rpl22_sg5 | GCGTTACTTCCAGATTAACC | SEQ ID NO: 125 |
| Fubp1_sg1 | GATAAACCTCTTAGGATTAC | SEQ ID NO: 126 |
| Fubp1_sg2 | GGAACGGGCTGGTGTTAAAA | SEQ ID NO: 127 |
| Fubp1_sg3 | GTCTTCCCTTTTCAACAATC | SEQ ID NO: 128 |
| Fubp1_sg4 | GAAAAGGGAAGACCAGCCCC | SEQ ID NO: 129 |
| Fubp1_sg5 | GTTAGCATACAAGACCTTTC | SEQ ID NO: 130 |
| Pcna_sg1 | GGAGGCGGTGAGTAGTAAGG | SEQ ID NO: 131 |
| Pcna_sg2 | GAGGAGGCGGTGAGTAGTAA | SEQ ID NO: 132 |
| Pcna_sg3 | GAATTTTGGACATGCTAGGG | SEQ ID NO: 133 |
| Pcna_sg4 | GTGAGCCTGTTTTCTCCTCT | SEQ ID NO: 134 |
| Pcna_sg5 | GGTTACCTAGAGGAGAAAAC | SEQ ID NO: 135 |
| Notch1_sg1 | GTATACACCTTCATAACCTG | SEQ ID NO: 136 |
| Notch1_sg2 | GCAGTGGCCATTGTGCAGAC | SEQ ID NO: 137 |
| Notch1_sg3 | GGCACCTGGTGAAAGAGGCA | SEQ ID NO: 138 |
| Notch1_sg4 | GCCAACCCTTGTGAGCACGC | SEQ ID NO: 139 |
| Notch1_sg5 | GAGCACACTCATCCACGTCC | SEQ ID NO: 140 |
| Casp8_sg1 | GGTGACAAGGGTGTCGTCTA | SEQ ID NO: 141 |
| Casp8_sg2 | GTGTCGTCTATGGAACGGAT | SEQ ID NO: 142 |
| Casp8_sg3 | GGTAAACTTTGTCTGAAGTC | SEQ ID NO: 143 |
| Casp8_sg4 | GGAGTTGGGTTATGTCTTCC | SEQ ID NO: 144 |
| Casp8_sg5 | GACTCACTGTCTTGTTCTCT | SEQ ID NO: 145 |
| Stag2_sg1 | GAGTGTTTGTACATAGATAC | SEQ ID NO: 146 |
| Stag2_sg2 | GCAGAACGGAATAAAATGAT | SEQ ID NO: 147 |
| Stag2_sg3 | GATGACTGCTTTGGTAAATG | SEQ ID NO: 148 |
| Stag2_sg4 | GATTACCCACTTACCATGGC | SEQ ID NO: 149 |
| Stag2_sg5 | GAGGACCAGCCATGGTAAGT | SEQ ID NO: 150 |
| Kdm5c_sg1 | GCTCTGCAGAGTATATTCCC | SEQ ID NO: 151 |
| Kdm5c_sg2 | GCATGTAGGTGATGCAGGGC | SEQ ID NO: 152 |
| Kdm5c_sg3 | GTTTGTCATCTTCATCTCCT | SEQ ID NO: 153 |
| Kdm5c_sg4 | GTATGCCGAATGTGTTCCCG | SEQ ID NO: 154 |
| Kdm5c_sg5 | GACCTTCCTAGAAGGCAAGG | SEQ ID NO: 155 |
| Smad4_sg1 | GTCCATTTCAAAGTAAGCAA | SEQ ID NO: 156 |
| Smad4_sg2 | GCAATGGAGCACCAGTACTC | SEQ ID NO: 157 |
| Smad4_sg3 | GATGATTGGAAATGGGAGGC | SEQ ID NO: 158 |
| Smad4_sg4 | GTCACAACAGGGCAGCTTGA | SEQ ID NO: 159 |
| Smad4_sg5 | GATGGCTATGTGGATCCTTC | SEQ ID NO: 160 |
| Cdkn1a_sg1 | GGGCTCCCGTGGGCACTTCA | SEQ ID NO: 161 |
| Cdkn1a_sg2 | GAAAACCCTGAAGTGCCCAC | SEQ ID NO: 162 |
| Cdkn1a_sg3 | GAAGATTCCCCGGGTGGGCC | SEQ ID NO: 163 |
| Cdkn1a_sg4 | GGGTGGGCCCGGAACATCTC | SEQ ID NO: 164 |
| Cdkn1a_sg5 | GATTGCGATGCGCTCATGGC | SEQ ID NO: 165 |
| Runx1_sg1 | GCGCGCGGGGGGCATGTTGG | SEQ ID NO: 166 |
| Runx1_sg2 | GCCTCCTCCAGGCGCGCGGG | SEQ ID NO: 167 |
| Runx1_sg3 | GTCCTAGTGTAGGGACCGGG | SEQ ID NO: 168 |
| Runx1_sg4 | GAGGGTTGGGCGTGGGGGCT | SEQ ID NO: 169 |
| Runx1_sg5 | GTAGAGGTGCGTATCTGTCA | SEQ ID NO: 170 |
| Rb1_sg1 | GTTCGAGGTGAACCATTAAT | SEQ ID NO: 171 |
| Rb1_sg2 | GAGGTCAGAACAGGAGCGCT | SEQ ID NO: 172 |
| Rb1_sg3 | GGCTCTCTGAGTAGTGCAGG | SEQ ID NO: 173 |
| Rb1_sg4 | GAATCATGGAATCCCTTGCA | SEQ ID NO: 174 |
| Rb1_sg5 | GAACCTTTTTATTCCTAGGA | SEQ ID NO: 175 |
| Zc3h13_sg1 | GAGGCAGAACGTCGTAAAGA | SEQ ID NO: 176 |
| Zc3h13_sg2 | GTTCTCTTCCGGCGAGGAGA | SEQ ID NO: 177 |
| Zc3h13_sg3 | GGAGGTGGACTCGGAGTGCG | SEQ ID NO: 178 |
| Zc3h13_sg4 | GAGATGGGAAGGACAGAGGC | SEQ ID NO: 179 |
| Zc3h13_sg5 | GACTTTCTCAGAGAAGGTGA | SEQ ID NO: 180 |
| Bap1_sg1 | GAACCGACAAACAGTCCTGG | SEQ ID NO: 181 |
| Bap1_sg2 | GGTCAGGCACCACTGCCATC | SEQ ID NO: 182 |
| Bap1_sg3 | GTCCTCTCCCCAGGGCCCTA | SEQ ID NO: 183 |
| Bap1_sg4 | GTGGACAGATAAAGCTCGAA | SEQ ID NO: 184 |
| Bap1_sg5 | GCTATGTGCCTATCACAGGG | SEQ ID NO: 185 |
| Map3k1_sg1 | GGGATACCTACCTGAATCCA | SEQ ID NO: 186 |
| Map3k1_sg2 | GGGAGGTGGGGGACTCCACG | SEQ ID NO: 187 |
| Map3k1_sg3 | GTCCCCTTTGTAGATCTAAG | SEQ ID NO: 188 |
| Map3k1_sg4 | GGAGATCCCATGACTTCTAC | SEQ ID NO: 189 |
| Map3k1_sg5 | GGGGAGGGGACACCTACAGA | SEQ ID NO: 190 |
| Rasa1_sg1 | GAGATTATTCTCTGTATTTT | SEQ ID NO: 191 |
| Rasa1_sg2 | GTCTTAATGTCTTTCCTTTA | SEQ ID NO: 192 |
| Rasa1_sg3 | GATCTTCTTCTCGGCCCTAA | SEQ ID NO: 193 |
| Rasa1_sg4 | GTTCACAATGAGTTAGAAGA | SEQ ID NO: 194 |
| Rasa1_sg5 | GGACACTGAGATATATCTAT | SEQ ID NO: 195 |
| Nf1_sg1 | GTCCATGGTAGTTGATCTTA | SEQ ID NO: 196 |
| Nf1_sg2 | GCTGCAGCCAAGAGCTCTTG | SEQ ID NO: 197 |
| Nf1_sg3 | GATTATCCGAATTCTTAGCA | SEQ ID NO: 198 |
| Nf1_sg4 | GACAATCTGATGCTATATCT | SEQ ID NO: 199 |
| Nf1_sg5 | GGTATATTTTCCAAGTCTTG | SEQ ID NO: 200 |
| Kansl1_sg1 | GTGGAGAGCTGTCTCACCAG | SEQ ID NO: 201 |
| Kansl1_sg2 | GGGTGTGGAGGTGTCTGATG | SEQ ID NO: 202 |
| Kansl1_sg3 | GGTCATGCACAGGTGGCGGC | SEQ ID NO: 203 |
| Kansl1_sg4 | GATGGCACAGCTCTGAAGAG | SEQ ID NO: 204 |
| Kansl1_sg5 | GCTCTGGAAGTGCAGGCTTG | SEQ ID NO: 205 |
| Gata3_sg1 | GGTGGTGAGGTCCGAAGGAG | SEQ ID NO: 206 |
| Gata3_sg2 | GGAAGGGTGGTGAGGTCCGA | SEQ ID NO: 207 |
| Gata3_sg3 | GCCCACAGGCATTGCAGACC | SEQ ID NO: 208 |
| Gata3_sg4 | GAGGAACGCTAATGGGGACC | SEQ ID NO: 209 |
| Gata3_sg5 | GTACCATCTCGCCGCCACAG | SEQ ID NO: 210 |
| Pten_sg1 | GGTTCATTGTCACTAACATC | SEQ ID NO: 211 |
| Pten_sg2 | GAATGCTGATCTTCATCAAA | SEQ ID NO: 212 |
| Pten_sg3 | GAACTTGTCCTCCCGCCGCG | SEQ ID NO: 213 |
| Pten_sg4 | GTTCTTCATACCAGGACCAG | SEQ ID NO: 214 |
| Pten_sg5 | GGGAATTGTGACTCCCTGAT | SEQ ID NO: 215 |
| Rps18_sg1 | GCTGCAGAAGAAAAAGATAC | SEQ ID NO: 216 |
| Rps18_sg2 | GCGCCACTTTTGGGGGTAAG | SEQ ID NO: 217 |
| Rps18_sg3 | GAACCTAGATTTTGAGACAG | SEQ ID NO: 218 |
| Rps18_sg4 | GAATTTTCTTCAGCCTCTCC | SEQ ID NO: 219 |
| Rps18_sg5 | GAGGGCTGCGCCACTTTTGG | SEQ ID NO: 220 |
| Arid1a_sg1 | GGCTACCCAAATATGAATCA | SEQ ID NO: 221 |
| Arid1a_sg2 | GGACCCCCATATCCTATGGG | SEQ ID NO: 222 |
| Arid1a_sg3 | GCTGCCTAGGATAGCCTCCT | SEQ ID NO: 223 |
| Arid1a_sg4 | GACGCATGAGCCATTCTCCC | SEQ ID NO: 224 |
| Arid1a_sg5 | GAAGTGTACTGGGGCATCTG | SEQ ID NO: 225 |
| Apc_sg1 | GGAGAGAGTTTACTTCCGAG | SEQ ID NO: 226 |
| Apc_sg2 | GTCTTTGTCCTGAGGCCTTA | SEQ ID NO: 227 |
| Apc_sg3 | GTGGAGTGCTGCACTGGCCC | SEQ ID NO: 228 |
| Apc_sg4 | GCTGTGAGTGAATGATGTTG | SEQ ID NO: 229 |
| Apc_sg5 | GCCAGTGTTTTGAGTTCTAG | SEQ ID NO: 230 |
| Ctcf_sg1 | GTCTACAAGCATAATCACAC | SEQ ID NO: 231 |
| Ctcf_sg2 | GATTATGCTTGTAGACAGGT | SEQ ID NO: 232 |
| Ctcf_sg3 | GATGGCGTAGAGGGGGAAAA | SEQ ID NO: 233 |
| Ctcf_sg4 | GATAACTGTGCTGGTCCAGA | SEQ ID NO: 234 |
| Ctcf_sg5 | GCTATGACAGTGTCACAATG | SEQ ID NO: 235 |
| Cic_sg1 | GTACAGGCAGGAGGCAACTG | SEQ ID NO: 236 |
| Cic_sg2 | GCAGGAGGCAACTGGGGACT | SEQ ID NO: 237 |
| Cic_sg3 | GGGGTGCACAGTCTTGATGG | SEQ ID NO: 238 |
| Cic_sg4 | GTGTAGCCGTTCTGCTCCAC | SEQ ID NO: 239 |
| Cic_sg5 | GTACCTTGGCCACTAGTGGG | SEQ ID NO: 240 |
| Polr2a_sg1 | GTGGAACGGCACATGTGTGA | SEQ ID NO: 241 |
| Polr2a_sg2 | GGAACGGCACATGTGTGATG | SEQ ID NO: 242 |
| Polr2a_sg3 | GACTTCAGGAATTAGTACGC | SEQ ID NO: 243 |
| Polr2a_sg4 | GAAGGTCACTGGGCTTAGGA | SEQ ID NO: 244 |
| Polr2a_sg5 | GTCTGCAGATGAAGGTCACT | SEQ ID NO: 245 |
| Rps11_sg1 | GACTCCTTGTCTGACCCCAC | SEQ ID NO: 246 |
| Rps11_sg2 | GAGGACCATTGTCATCCGCC | SEQ ID NO: 247 |
| Rps11_sg3 | GGATGTAATGGAGATAGTCC | SEQ ID NO: 248 |
| Rps11_sg4 | GTCACCTGAAACAGGGGGAC | SEQ ID NO: 249 |
| Rps11_sg5 | GTCGGATCCTGTCTGGTGAG | SEQ ID NO: 250 |
| Stk11_sg1 | GGAGCCCGAGGAGGGGTTTG | SEQ ID NO: 251 |
| Stk11_sg2 | GGGCGCAGGCCTTCCTGGAG | SEQ ID NO: 252 |
| Stk11_sg3 | GAAGAAACACCCTCTGGCTG | SEQ ID NO: 253 |
| Stk11_sg4 | GTGTCTGGGCTTGGTGGGAT | SEQ ID NO: 254 |
| Stk11_sg5 | GTGCTGCCTAATCTGTCGGA | SEQ ID NO: 255 |
| Cdkn1b_sg1 | GCTCCACAGTGCCAGCGTTC | SEQ ID NO: 256 |
| Cdkn1b_sg2 | GCGAAGAAGAATCTAAGAGG | SEQ ID NO: 257 |
| Cdkn1b_sg3 | GGAGAAGCACTGCCGGGATA | SEQ ID NO: 258 |
| Cdkn1b_sg4 | GGTTAGCGGAGCAGTGTCCA | SEQ ID NO: 259 |
| Cdkn1b_sg5 | GGTGCTGGCGCAGGAGAGCC | SEQ ID NO: 260 |
| Cdh1_sgl | GAAAACAGCCAAGGTTTGTA | SEQ ID NO: 261 |
| Cdh1_sg2 | GGGTCAAGTGCCTGAGAATG | SEQ ID NO: 262 |
| Cdh1_sg3 | GAGTTACCCTACATACACTC | SEQ ID NO: 263 |
| Cdh1_sg4 | GTTCAGGCTGCTGACCTTCA | SEQ ID NO: 264 |
| Cdh1_sg5 | GGAGGTTCCTGTCAAAGGAG | SEQ ID NO: 265 |
| B2m_sgl | GGTCTTGGGCTCGGCCATAC | SEQ ID NO: 266 |
| B2m_sg2 | GGGTGAATTCAGTGTGAGCC | SEQ ID NO: 267 |
| B2m_sg3 | GAGCCCAAGACCGTCTACTG | SEQ ID NO: 268 |
| B2m_sg4 | GTATGTATCAGTCTCAGTGG | SEQ ID NO: 269 |
| B2m_sg5 | GGTCGCTTCAGTCGTCAGCA | SEQ ID NO: 270 |
| Fbxw7_sg1 | GCCGCTTGCAGCAGGTCTTT | SEQ ID NO: 271 |
| Fbxw7_sg2 | GCAGCAGGTCTTTGGGTTCC | SEQ ID NO: 272 |
| Fbxw7_sg3 | GAGTGTATACATACTTTATA | SEQ ID NO: 273 |
| Fbxw7_sg4 | GTATGCATCTCCATGAAAAA | SEQ ID NO: 274 |
| Fbxw7_sg5 | GATCTGTACACTTTTCTTAT | SEQ ID NO: 275 |
| Nkx2-1_sg1 | GCGGGGCGCACTGGGCAGCG | SEQ ID NO: 276 |
| Nkx2-1_sg2 | GCCACCGCTGCCCACTGAGA | SEQ ID NO: 277 |
| Nkx2-1_sg3 | GACGGCAAACCCTGCCAGGC | SEQ ID NO: 278 |
| Nkx2-1_sg4 | GCCATGCAGCAGCACGCCGT | SEQ ID NO: 279 |
| Nkx2-1_sg5 | GCCGTGGGGGGCTACTGCAA | SEQ ID NO: 280 |
| Control_sg1 | ACGGAGGCTAAGCGTCGCAA | SEQ ID NO: 281 |
| Control_sg2 | CGCTTCCGCGGCCCGTTCAA | SEQ ID NO: 282 |
| Control_sg3 | ATCGTTTCCGCTTAACGGCG | SEQ ID NO: 283 |
| Control_sg4 | GTAGGCGCGCCGCTCTCTAC | SEQ ID NO: 284 |
| Control_sg5 | CCATATCGGGGCGAGACATG | SEQ ID NO: 285 |
| Control_sg6 | TACTAACGCCGCTCCTACAG | SEQ ID NO: 286 |
| Control_sg7 | TGAGGATCATGTCGAGCGCC | SEQ ID NO: 287 |
| Control_sg8 | GGGCCCGCATAGGATATCGC | SEQ ID NO: 288 |

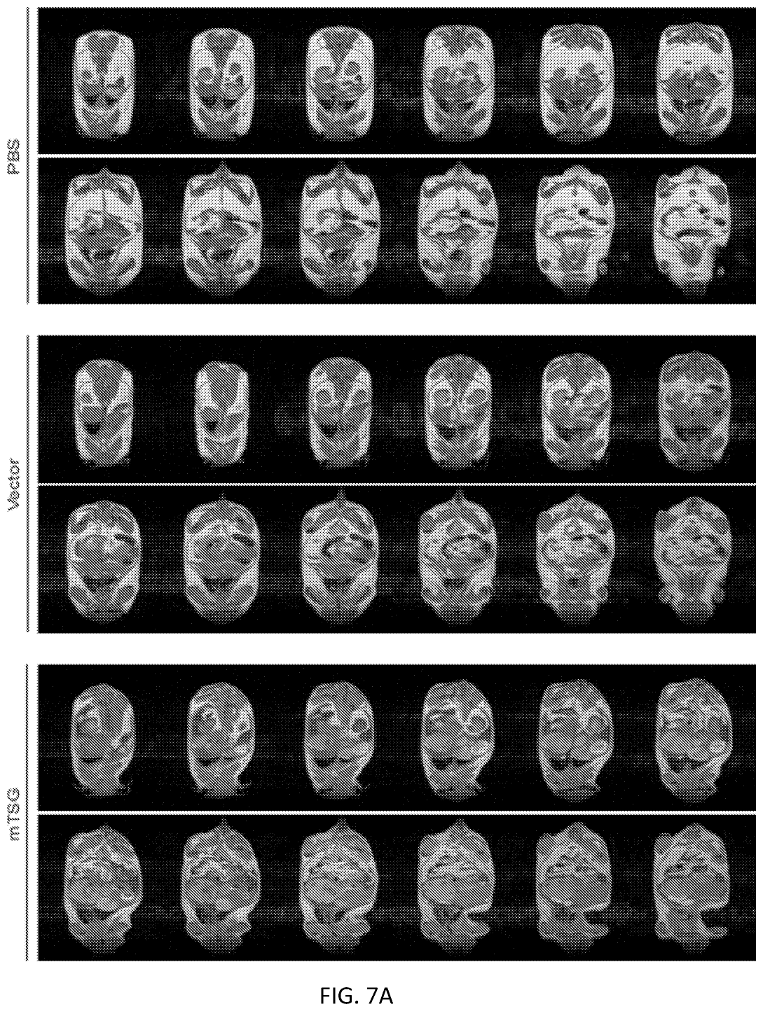

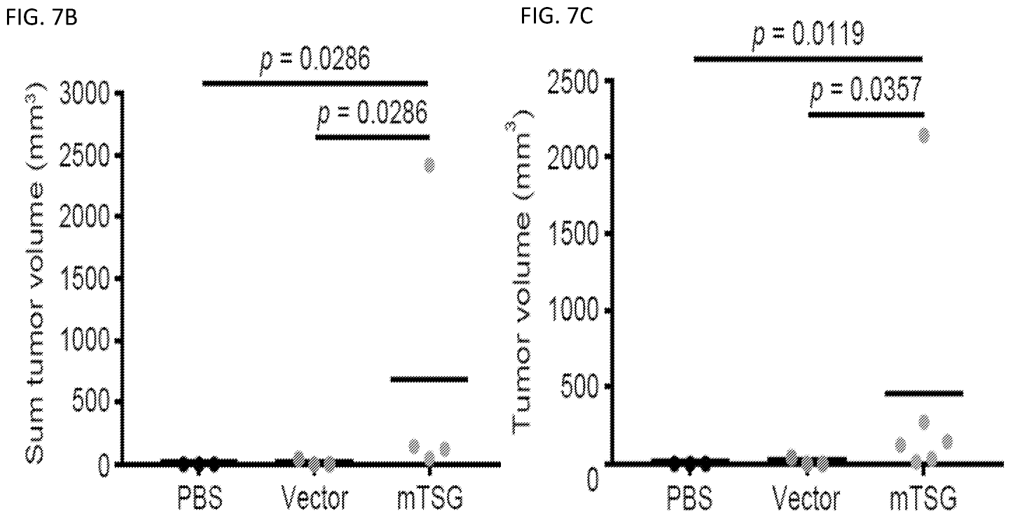

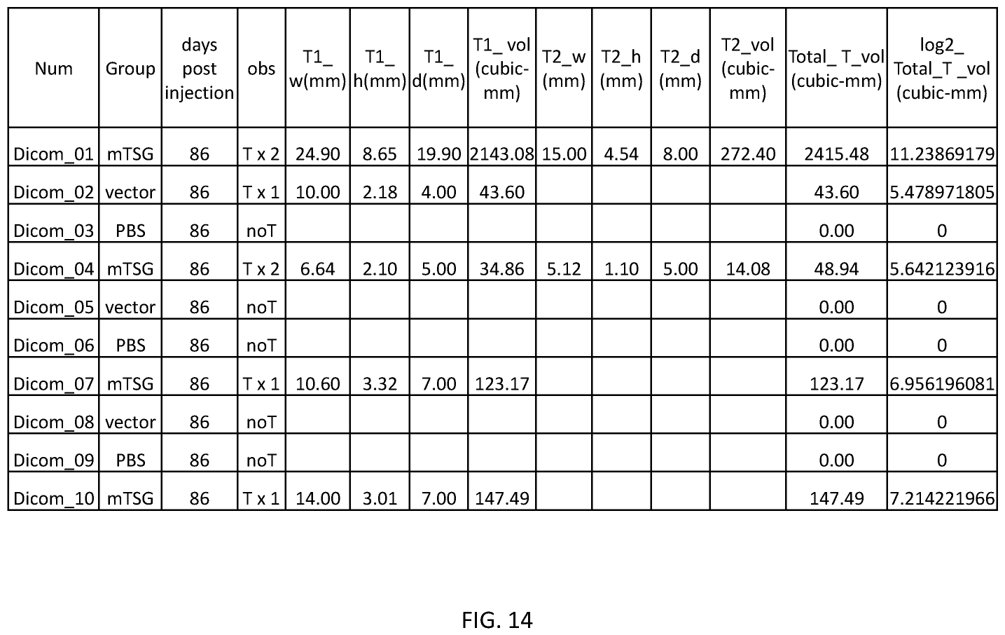

Live magnetic resonance imaging (MRI) of mice 3 months post-treatment revealed large nodules in mTSG-treated animals (n=4), while vector-treated animals (n=3) only occasionally had small nodules and PBS animals (n=3) were devoid of detectable nodules (FIG. 1B; FIGS. 7A-7C; FIG. 14). The total tumor volume in each mouse was significantly larger in mTSG samples compared to PBS and vector samples (one-sided Mann-Whitney test, p=0.0286 and p=0.0286) (FIG. 7B). mTSG-treated mice had multiple tumors. The volumes of individual tumors were compared and mTSG samples had significantly larger individual tumors compared to PBS (p=0.0119) and vector samples (p=0.0357) (FIG. 7C). These data demonstrated that the AAV-CRISPR mTSG library was sufficient to induce rapid tumorigenesis in the livers of LSL-Cas9 transgenic mice.

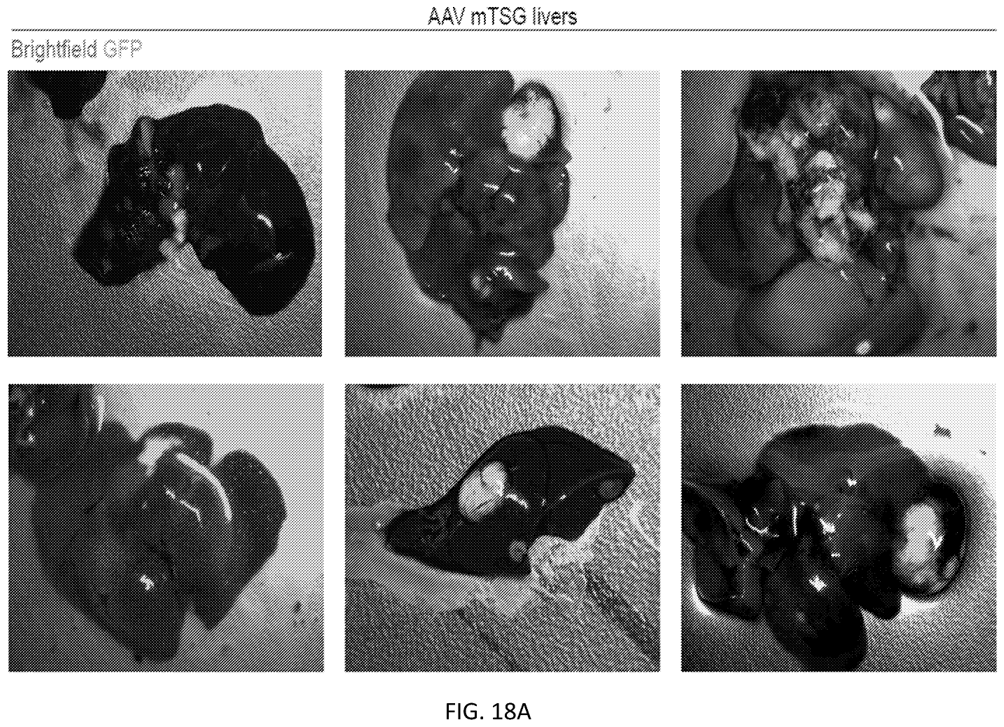

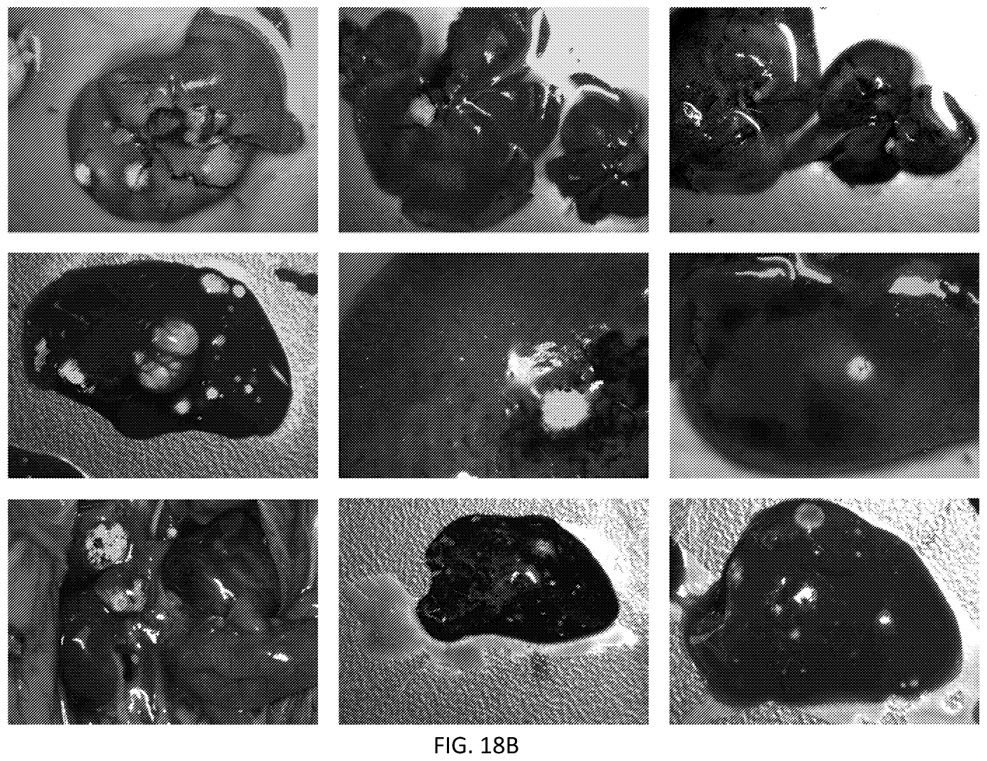

Mice that received the AAV-CRISPR mTSG library (n=27) did not survive more than four months (median survival=90 days, 95% confidence interval CI=84-90 days), while mice that were treated with PBS (n=10) or vector control (n=11) all survived the duration of the experiment (log-rank test, p=1.8*10⁻¹¹) (FIG. 1C; Table 3). By gross examination under a fluorescent dissecting scope, detectable GFP+ nodules were observed in mTSG-treated livers, but not in PBS or vector samples (FIG. 1D and FIGS. 18A-18B). In mTSG-treated mice, tumors were occasionally observed that were not primarily located in the liver. Chief among these were several big abdominal tumors (BATs, n=6), as well as a few sarcomas (n=4) and ear tumors (n=2), although BATs were later found to be of liver origin on the basis of histological analysis.

TABLE 3
Survival data for PBS, vector, or mTSG-treated animals.

| Group | ID | last_day_post_injection_survived |
|---|---|---|
| vector | GvIV pilot m1 | NA |
| vector | GvIV pilot m2 | NA |
| vector | GvIV pilot m3 | NA |
| vector | GvIV pilot m4 | NA |
| vector | GvIV pilot m5 | NA |
| vector | GvIV pilot m6 | NA |
| vector | GvIV m1 | NA |
| vector | GvIV m2 | NA |
| vector | GvIV m3 | NA |
| vector | GvIV m4 | NA |
| vector | GvIV m5 | NA |
| PBS | PBS M1 | NA |
| PBS | PBS M2 | NA |
| PBS | PBS M3 | NA |
| PBS | PBS M4 | NA |
| PBS | PBS M5 | NA |
| PBS | PBS M6 | NA |
| PBS | PBS M7 | NA |
| PBS | PBS M8 | NA |
| PBS | PBS M9 | NA |
| PBS | PBS M10 | NA |
| mTSG | mTSG pilot | 97 |
| mTSG | mTSG | 107 |
| mTSG | mTSG | 111 |
| mTSG | mTSG | 111 |
| mTSG | mTSG | 117 |
| mTSG | mTSG | 117 |
| mTSG | mTSG | 67 |
| mTSG | mTSG | 74 |
| mTSG | mTSG | 77 |
| mTSG | mTSG | 84 |
| mTSG | mTSG | 74 |
| mTSG | mTSG | 82 |
| mTSG | mTSG | 84 |
| mTSG | mTSG | 84 |
| mTSG | mTSG | 84 |
| mTSG | mTSG | 80 |
| mTSG | mTSG | 82 |
| mTSG | mTSG | 87 |
| mTSG | mTSG | 90 |
| mTSG | mTSG | 90 |
| mTSG | mTSG | 90 |
| mTSG | mTSG | 90 |
| mTSG | mTSG | 90 |
| mTSG | mTSG | 90 |
| mTSG | mTSG | 90 |
| mTSG | mTSG | 90 |
| mTSG | mTSG | 90 |

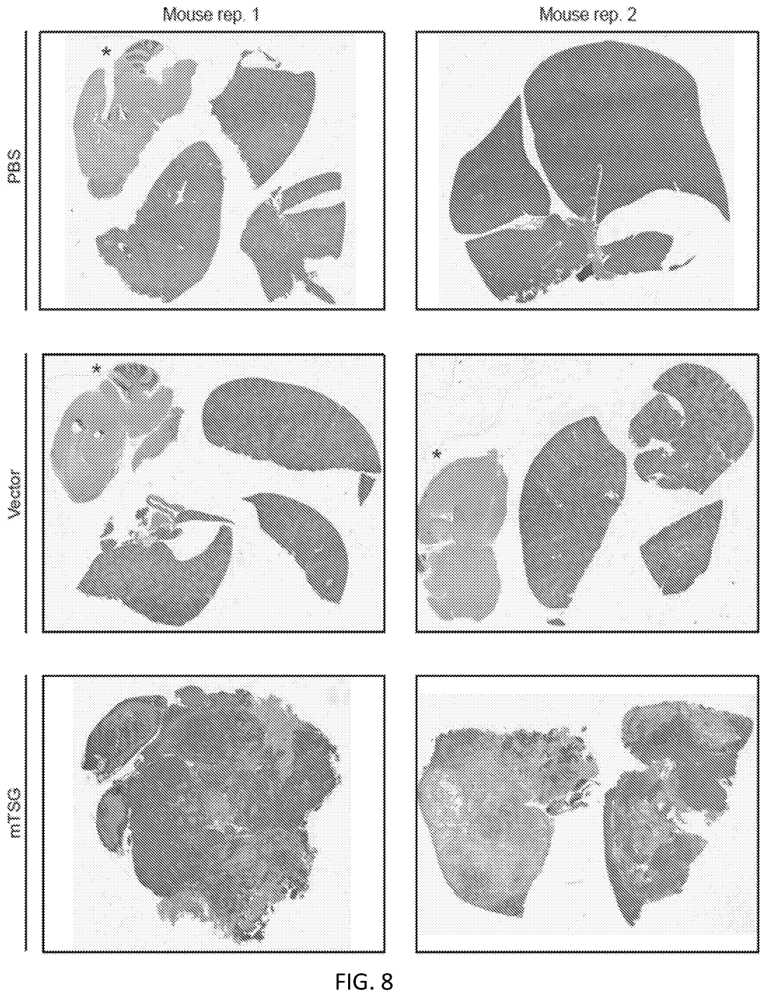

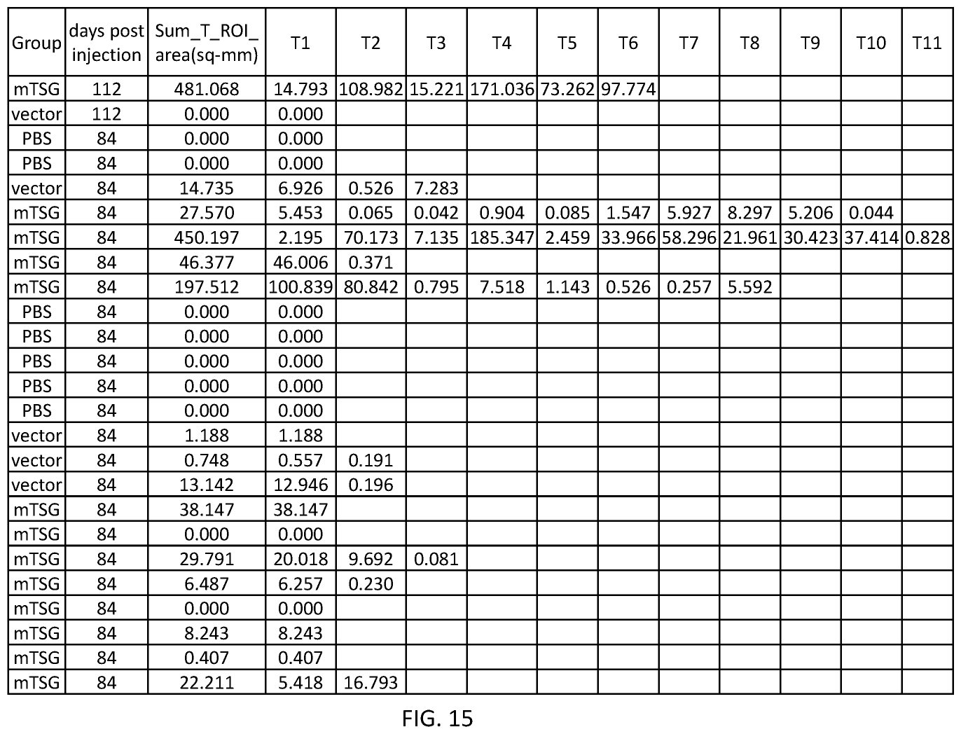

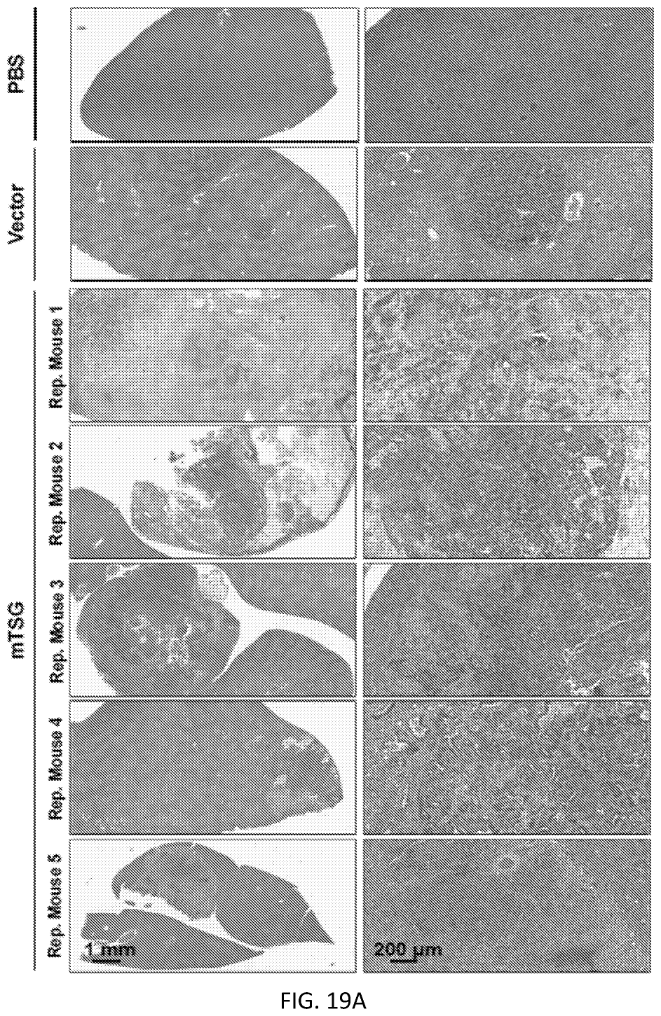

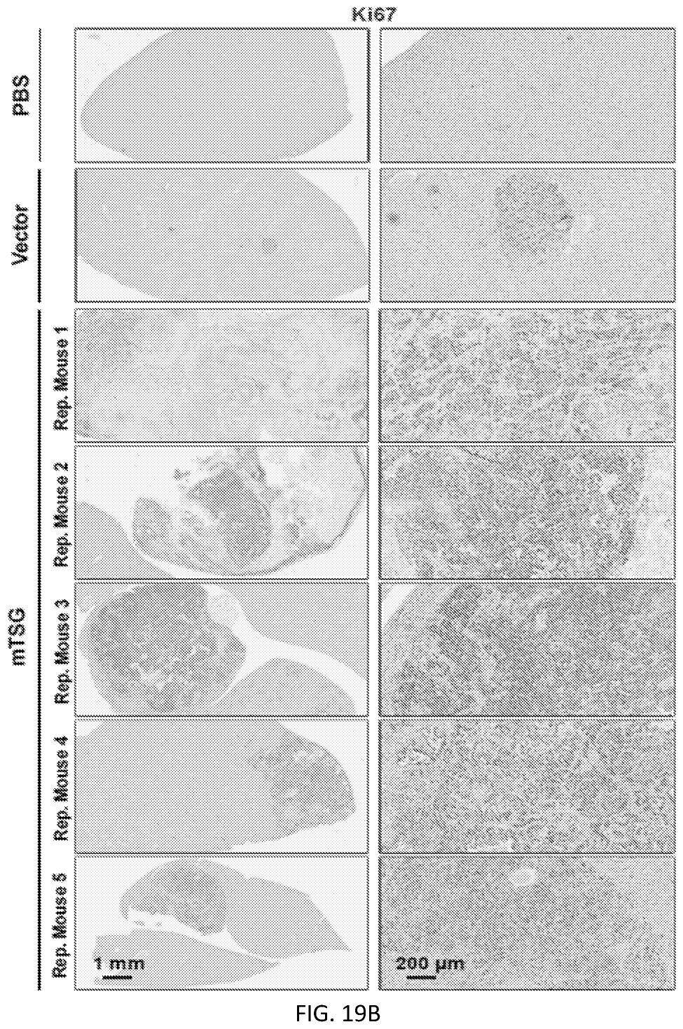

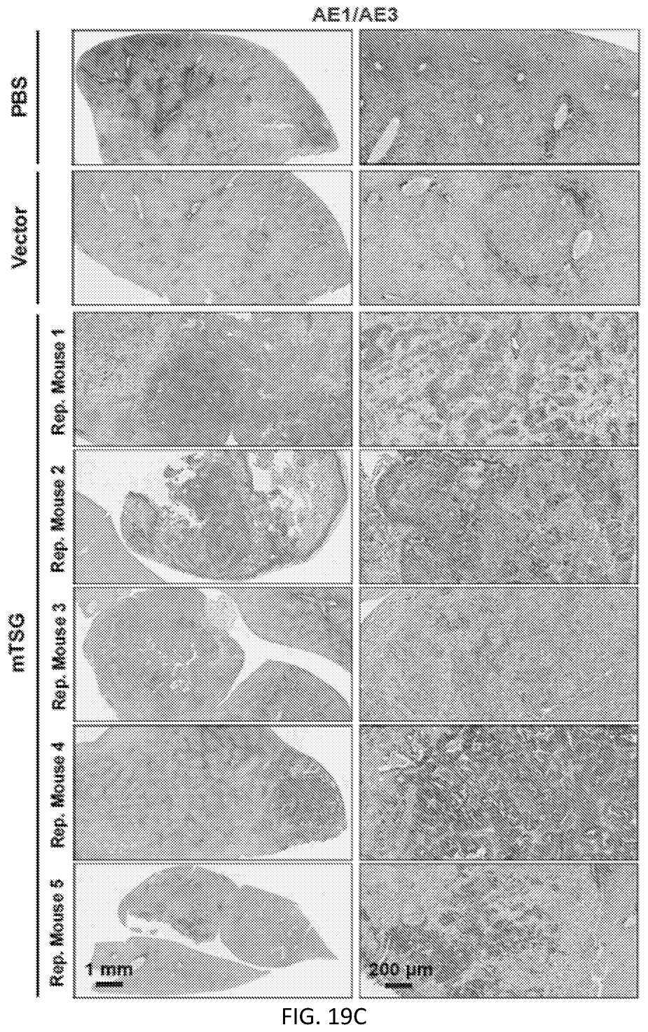

Endpoint histological sections were analyzed from PBS (n=7), vector (n=5), and mTSG-treated mice (n=13), sacrificed 3-4 months post-treatment for controls (FIG. 1E, FIG. 8, and FIGS. 19A-19C). No tumors were found in PBS-treated mice, while rare small tumors were found in vector-treated mice (total tumor area=5.96±3.27 mm²) (FIG. 1F). Consistent with the MRI results, mice that received the mTSG library had significantly larger liver tumors, with the pathology of LIHC (total tumor area=100.6±47.19 mm²; one-sided Welch's t-test, p=0.027 compared to PBS, p=0.034 compared to vector) (FIGS. 1E-1F; FIG. 15). Some mice were found to have multiple liver tumors, so the size of each individual tumor was compared across the 3 treatment groups (FIG. 1G). The mTSG-treated mice collectively had tumors that were significantly larger (26.69±6.18 mm²) than the tumors found in PBS treated (0±0 mm²; one-sided Welch's t-test, p<0.0001) or vector-treated animals (3.31±1.55 mm²; p=0.0003), though the latter were too small to be detected by gross examination under a GFP dissecting scope. The proliferation of liver samples from PBS, vector, and mTSG-treated mice by Ki67 expression were assessed, and it was discovered that rapid proliferation was restricted to tumor cells (FIG. 19B). Additionally, the tumors in mTSG treated mice, but not vector treated mice, were largely positive for AE1/AE3 (pan-cytokeratin), which is a marker of LIHC (FIG. 1I and FIG. 19C). These data collectively indicated that the AAV-CRISPR mTSG library directly promotes aggressive liver tumorigenesis in otherwise wildtype LSL-Cas9 mice.

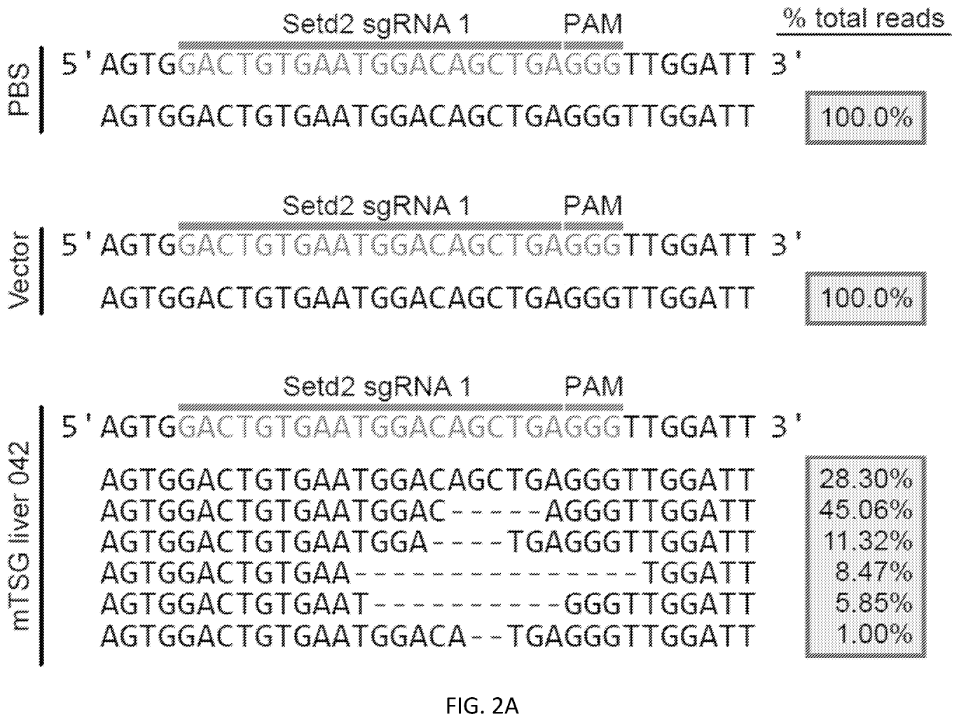

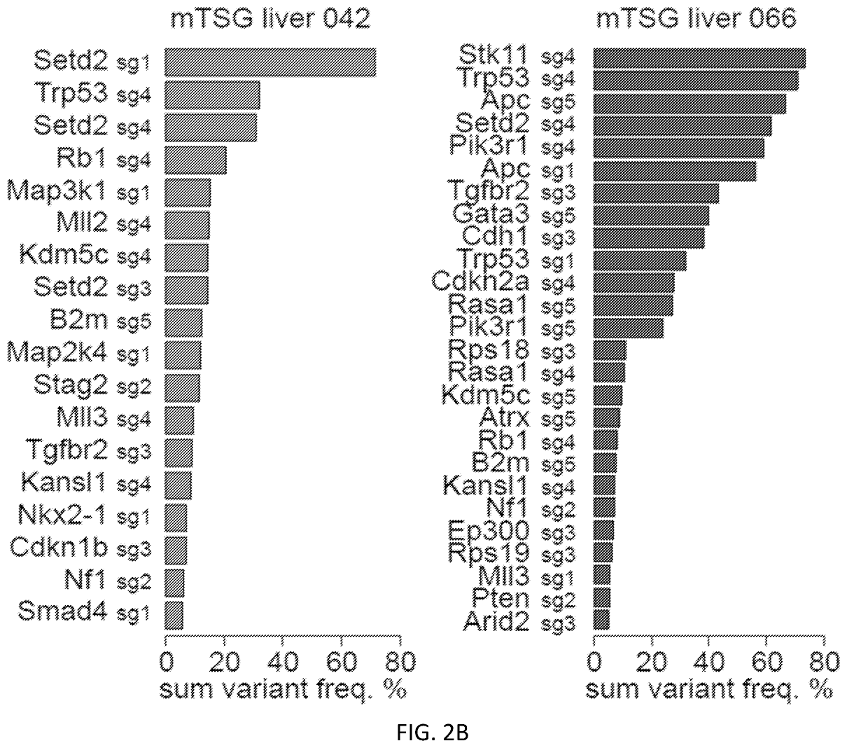

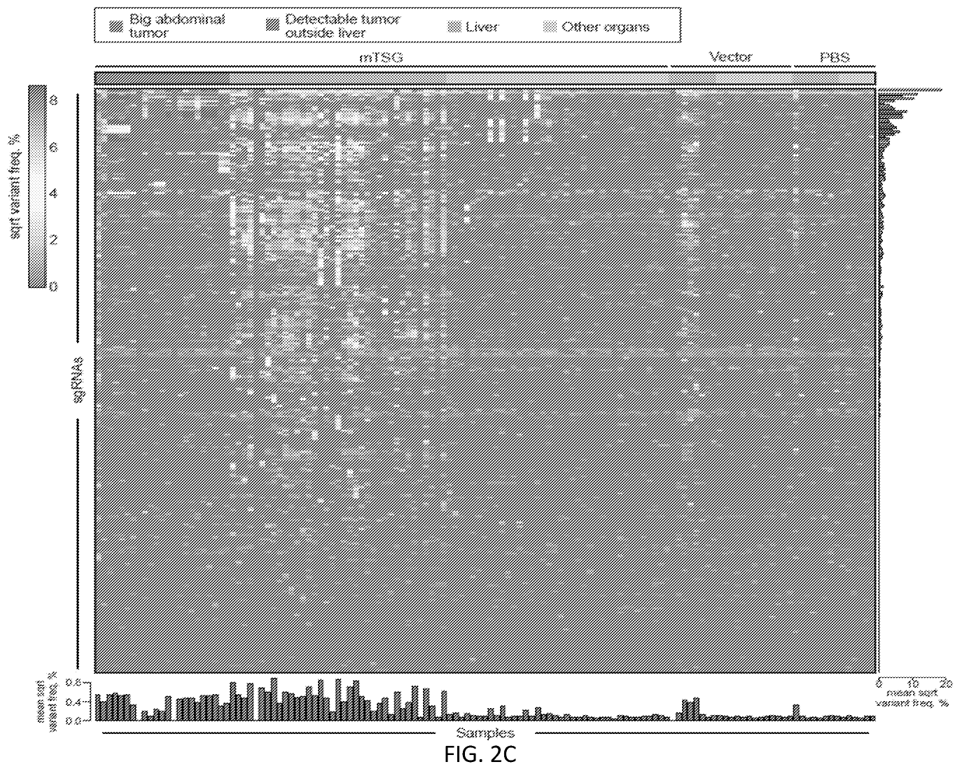

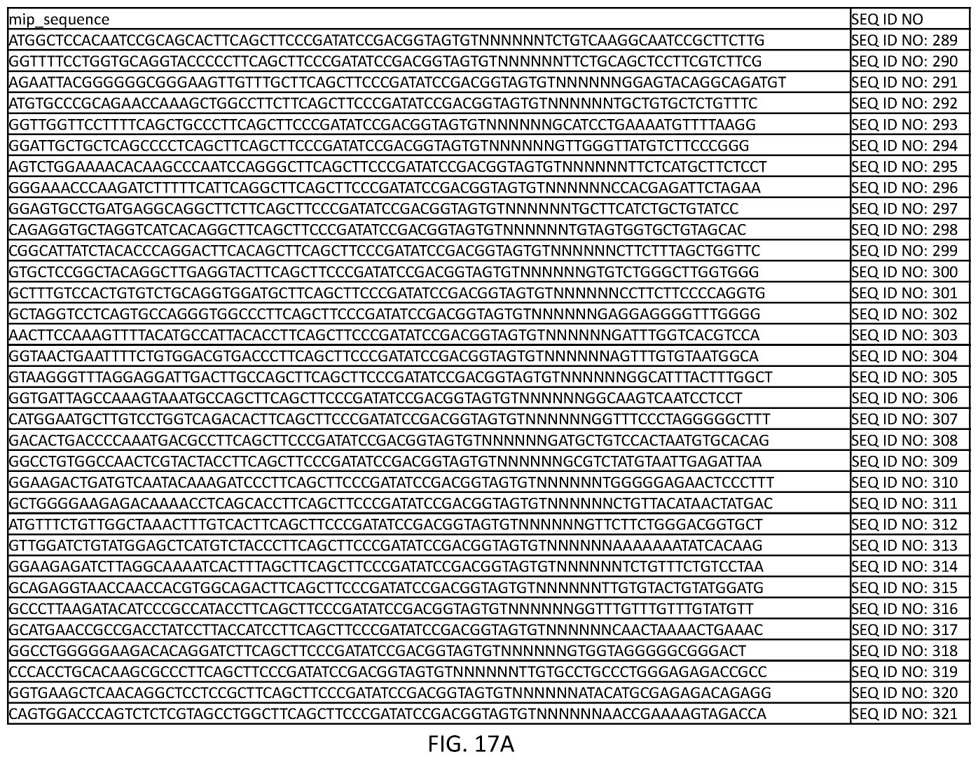

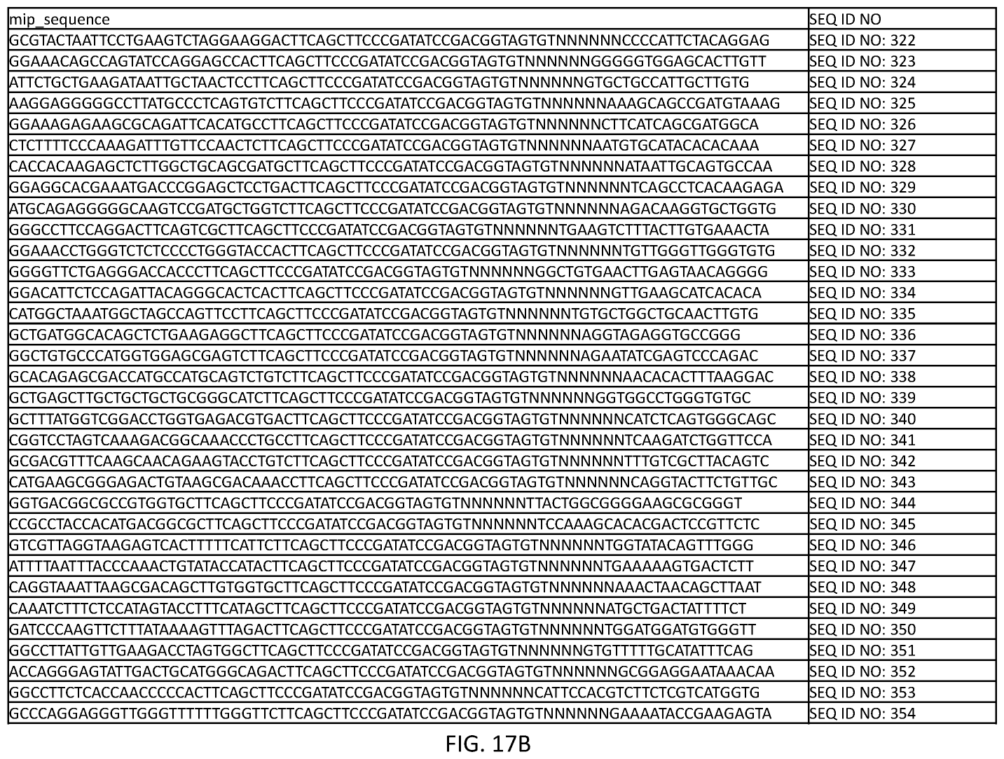

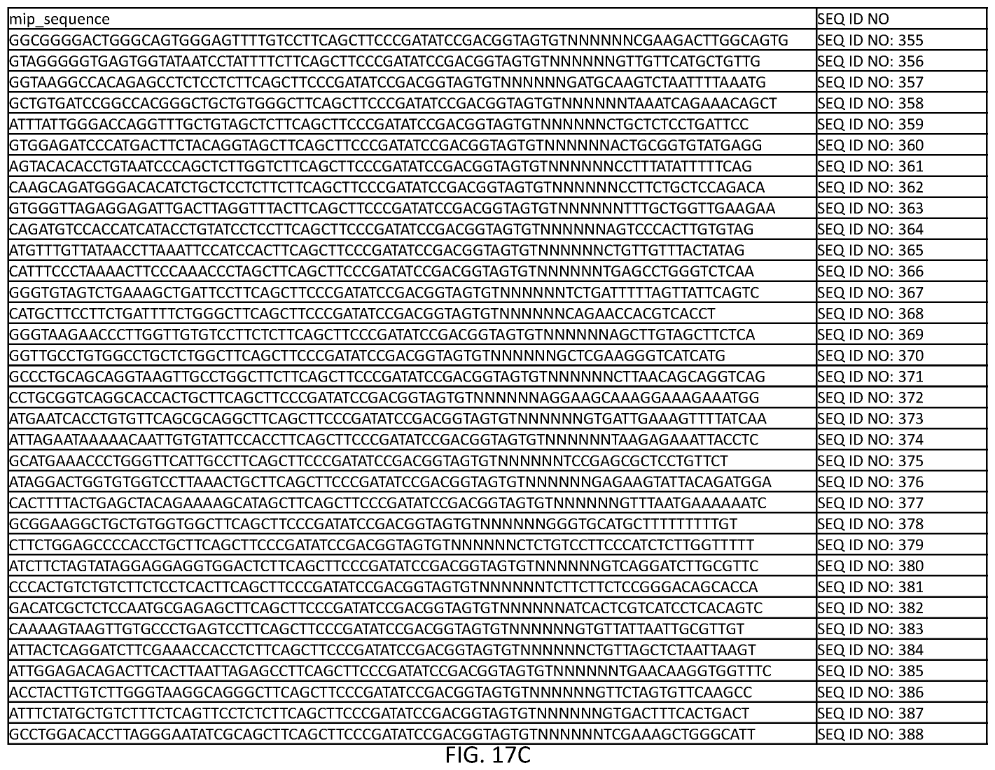

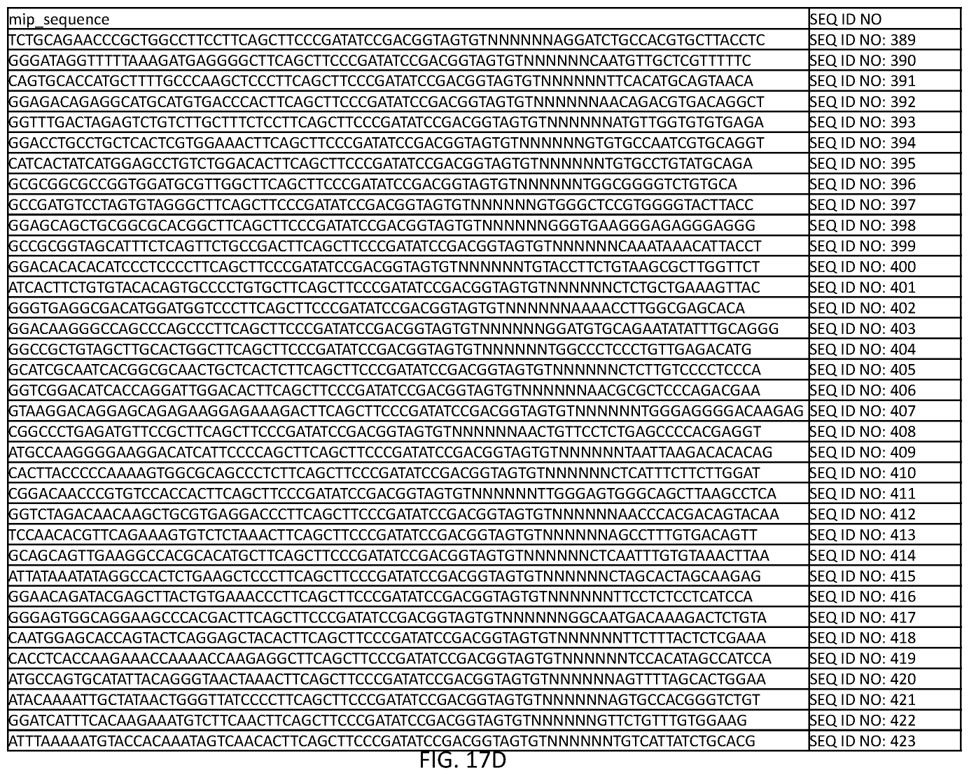

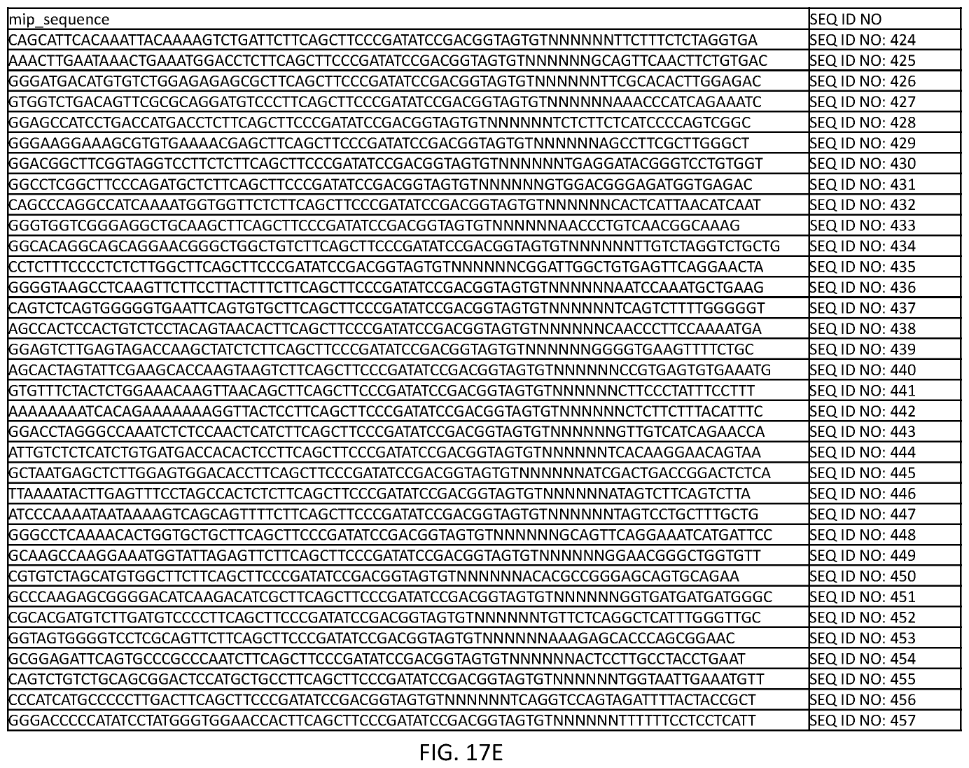

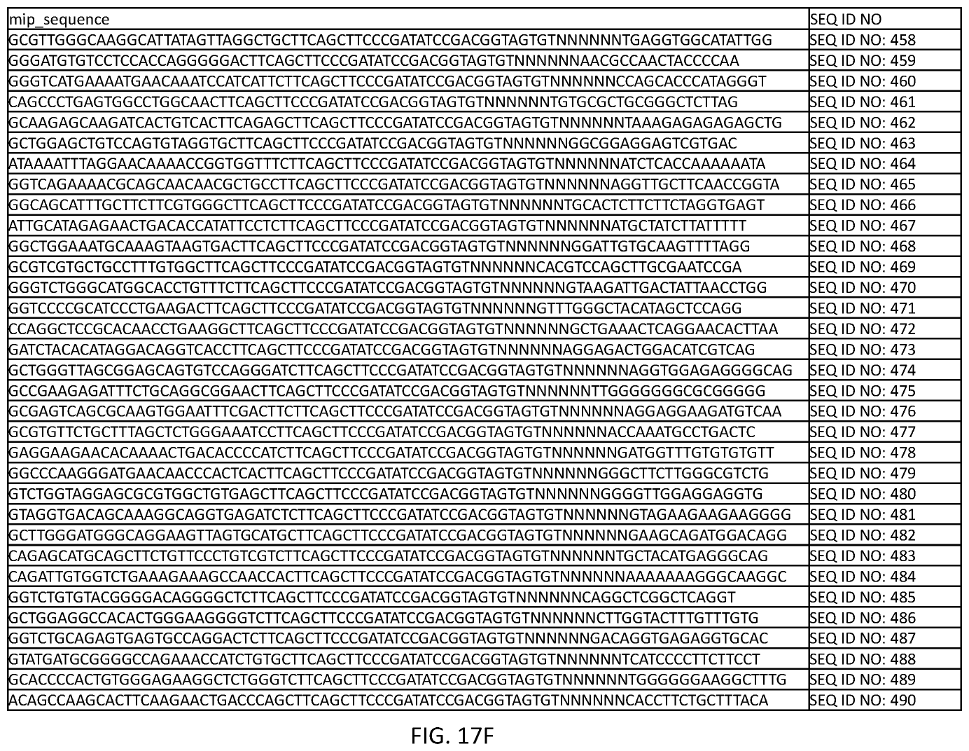

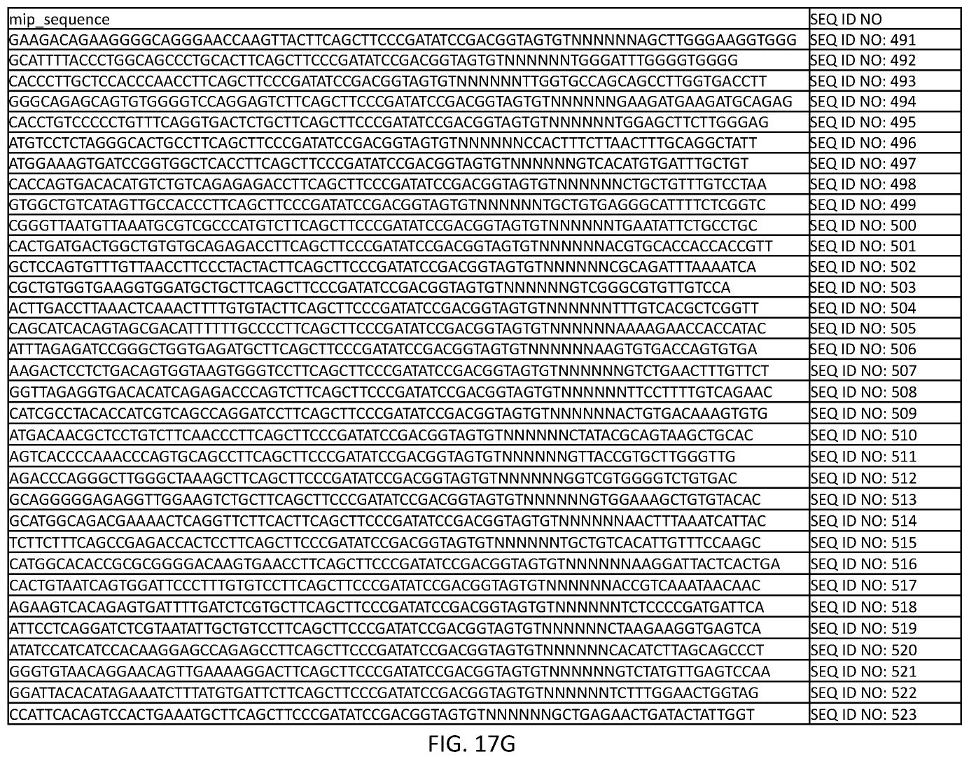

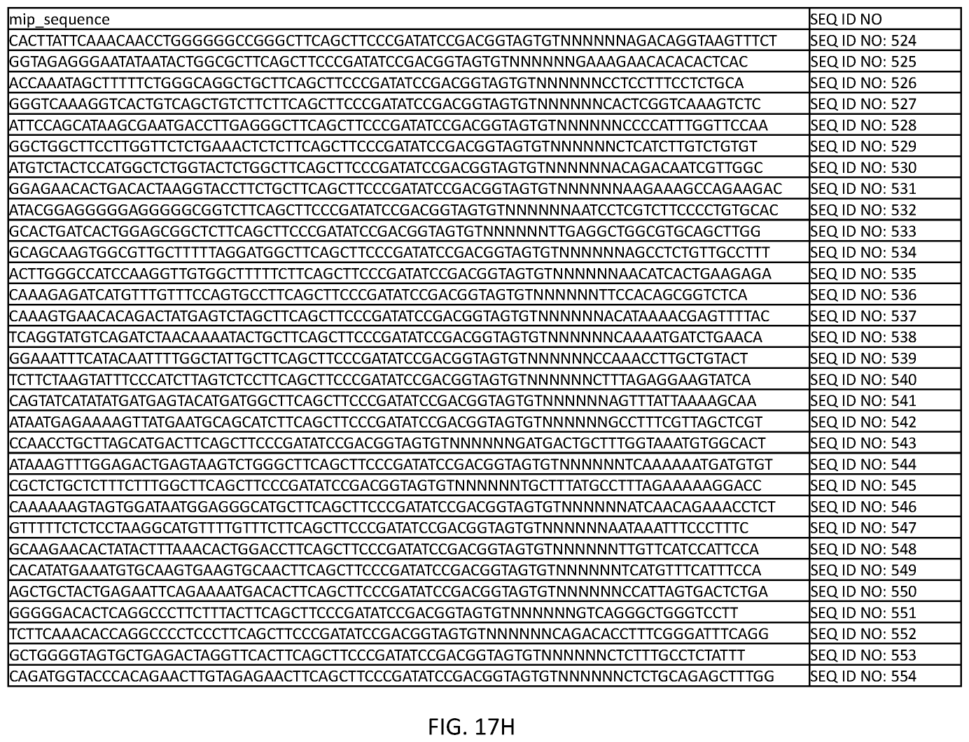

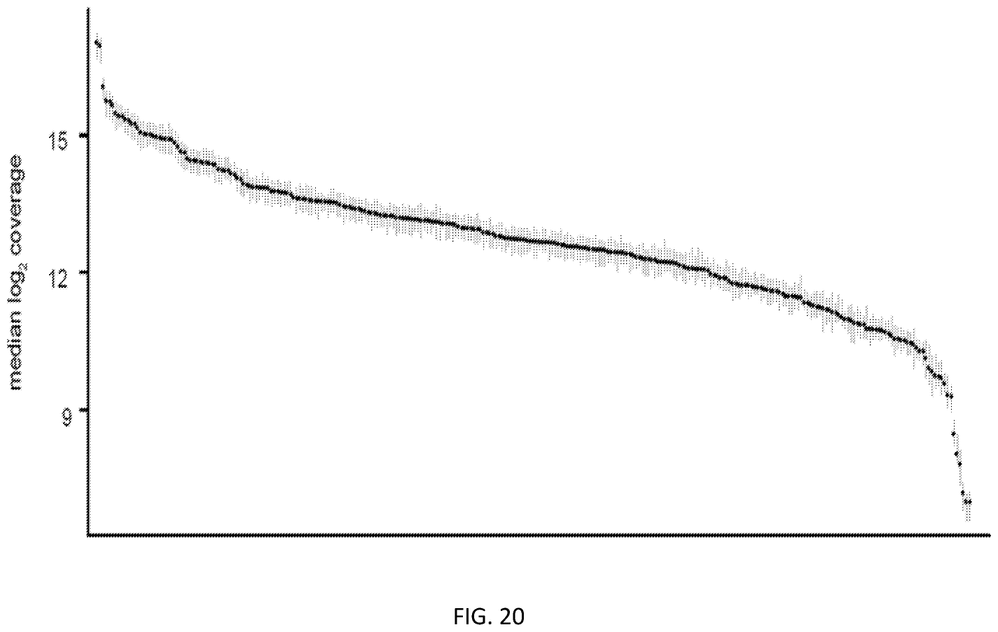

To understand the molecular alterations driving the development of tumors in mTSG-treated mice, Molecular Inversion Probes (MIPs) were designed to enable capture sequencing of the ±70 basepair (bp) regions surrounding the predicted cut site of each sgRNA in the mTSG library (namely, the +17 position of each 20 bp spacer sequence). As opposed to simply sequencing the sgRNA cassettes to find the relative enrichment of each sgRNA within the cell population, MIP capture sequencing enables a direct quantitative analysis of the mutations induced by the Cas9-sgRNA complex. To generate this pool of MIPs (termed mTSG-MIPs) (FIGS. 17A-17H; SEQ ID NOs 289-554), a total of 266 extension and ligation probes were synthesized targeting 266 genomic loci with an average size of 158±8 (SEM) bp, covering 278 unique sgRNA sites. Liver genomic DNA was extracted from PBS-treated (n=8 mice), vector-treated (n=8 mice), and mTSG-treated animals (n=27 mice; 37 liver lobes in total). In order to assess the potential for AAV-CRISPR mediated mutagenesis of other organs, DNA was also collected from all observed non-liver tumors (n=23), as well as a wide variety of tissues (such as brain, lung, colon, spleen and kidney) without detectable tumors under a fluorescent dissecting scope (n=57 samples) from all three groups. MIP capture sequencing was performed on all genomic DNA samples (total n=133). Sequencing depth of the sgRNA target regions was sufficiently powerful to detect variants at <0.01% frequency, with a mean read depth of 13,482±1049 (SEM) across all MIPs after mapping to the mouse genome. Median read depth across all MIPs approximated a lognormal distribution, indicating relatively even capture of the target loci (FIG. 1H and FIG. 20). Insertions and deletions (indels) were then called across all samples to reveal detectable indel variants at each sgRNA cut site. Single nucleotide variants (SNVs) were excluded from the analysis, as indels are the dominant variants generated by non-homologous end-joining (NHEJ) following Cas9 mediated double-strand breaks (DSBs) in vivo. For downstream analysis, only indels that overlapped the ±3 bp flanks around each of the predicted sgRNA cut sites were considered, as Cas9 tends to create DSBs in a tight window around the predicted sgRNA cut site in mammalian cells. A representative example of the genotypes observed by MIPs capture sequencing is shown at the Setd2 sgRNA 1 cut site for PBS, vector, or mTSG-treated samples (FIG. 2A), illustrating the diversity of Cas9-induced indels in mTSG-treated mice.

After collapsing each of the filtered indel calls to the closest sgRNA by summing their constituent variant frequencies, the overall spectrum of variant frequencies across all sequenced samples was plotted (FIG. 2C). The mean variant frequency was calculated for each sgRNA (FIG. 2C, right panel) and for each sample (FIG. 2C, bottom panel). The mTSG-treated organs without visible tumors (0.148±0.037 SEM) had significantly lower mean variant frequencies compared to mTSG-treated tumors and livers (BATs, 3.098±0.600; unpaired t-test, p<0.0001), non-liver tumors (1.919±0.338; p<0.0001), and livers (1.451±0.203; p<0.0001). Livers and other organs from vector-treated animals (0.398±0.179 and 0.054±0.004, respectively) and PBS-treated animals (0.140±0.067 and 0.063±0.021, respectively) all had significantly lower variant frequencies than mTSG-treated livers (p<0.0001 for all comparisons). The low background variant frequencies observed in vector and PBS treated samples may be due to noise generated during sequencing, as well as stochastic or germline mutations. The vector contains a Trp53 sgRNA, potentially contributing to higher variant frequencies in vector-treated livers due to genome instability of Trp53-deficient cells.

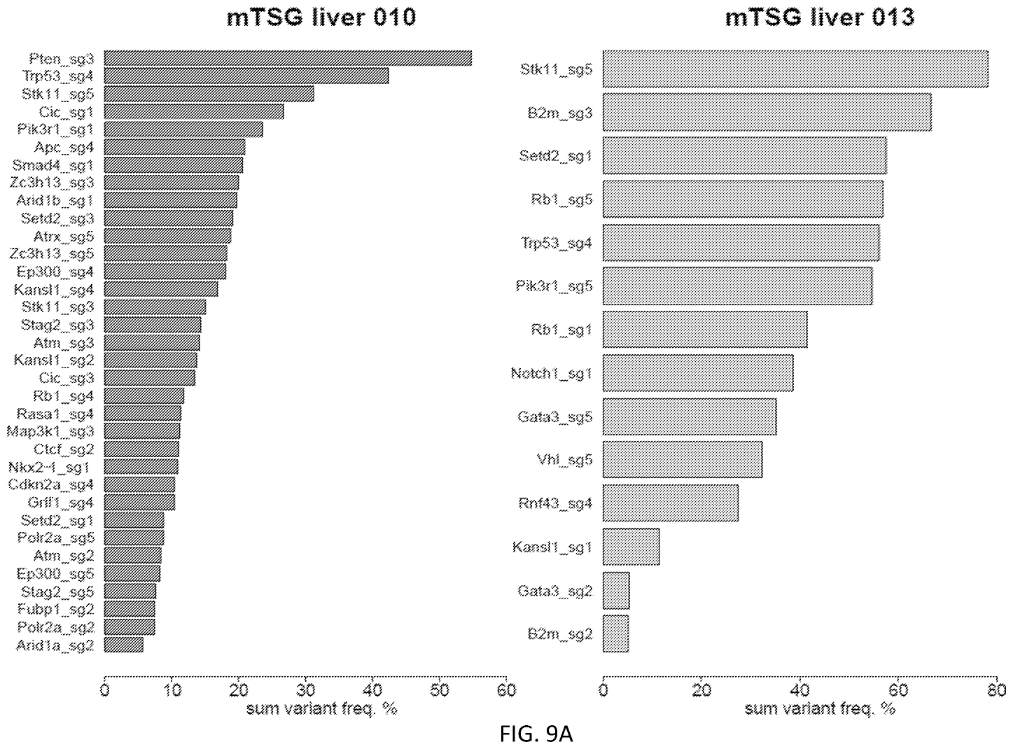

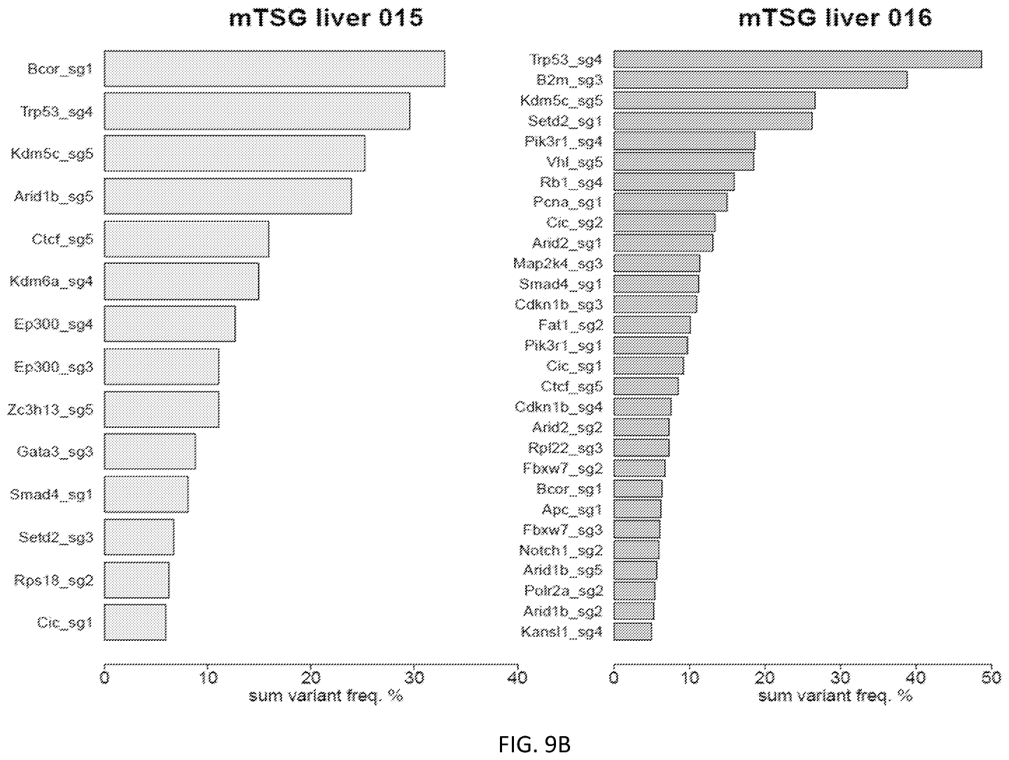

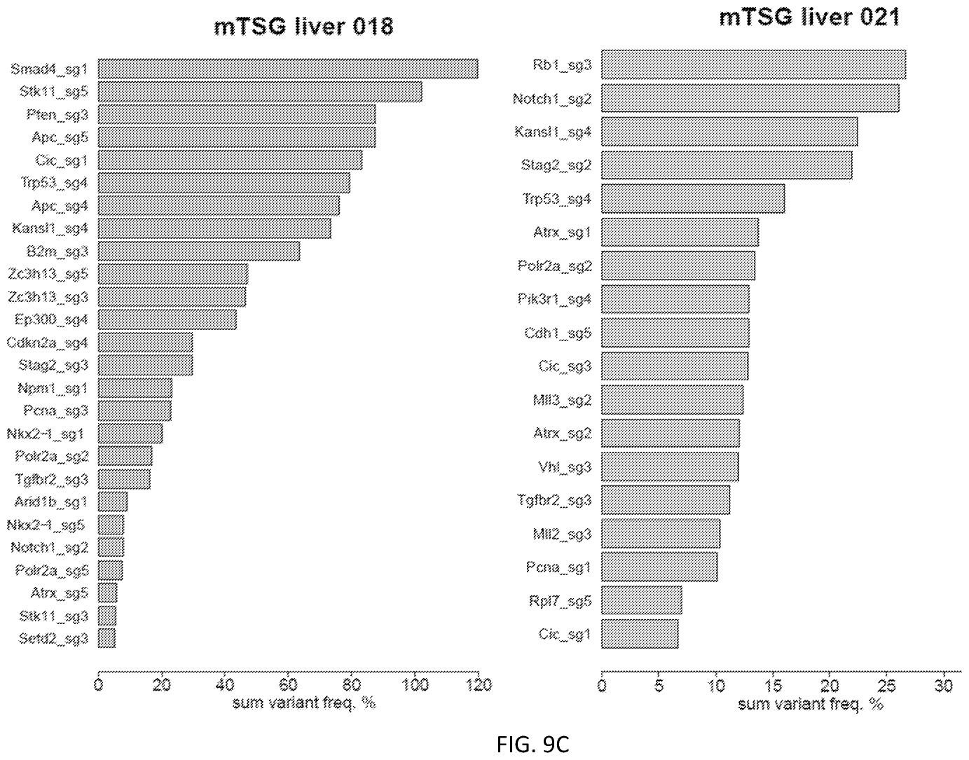

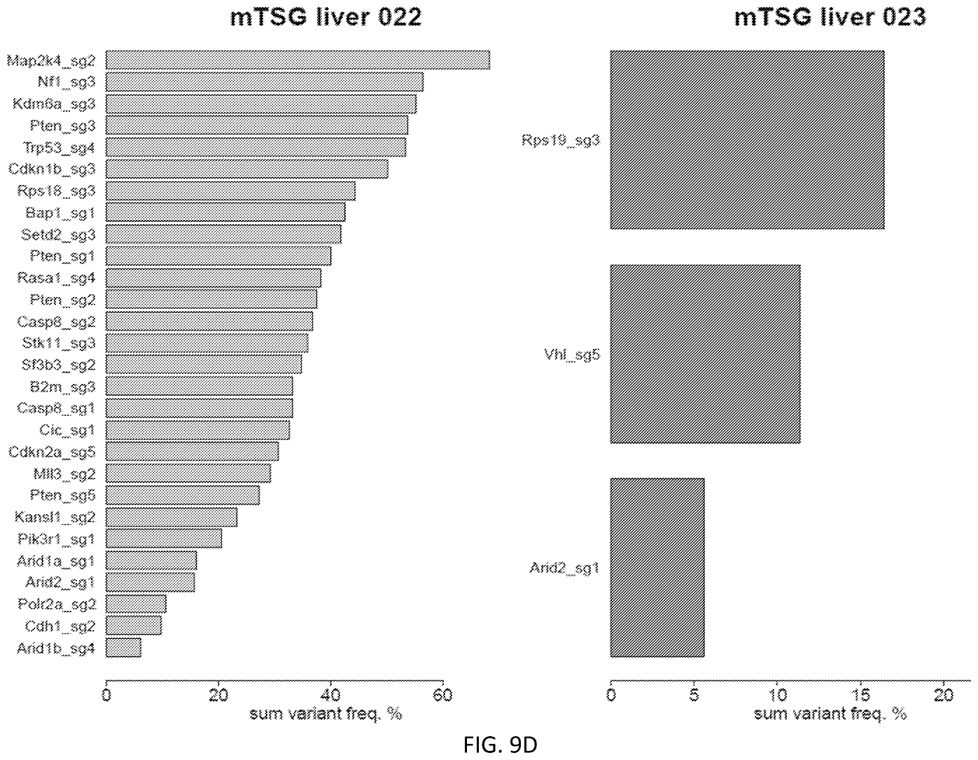

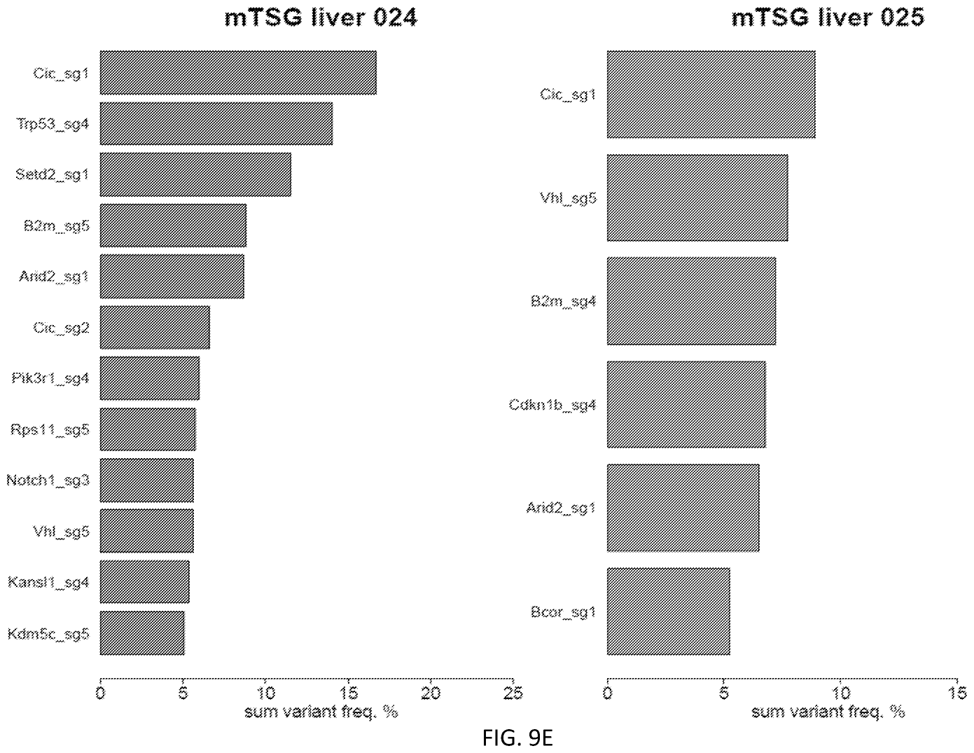

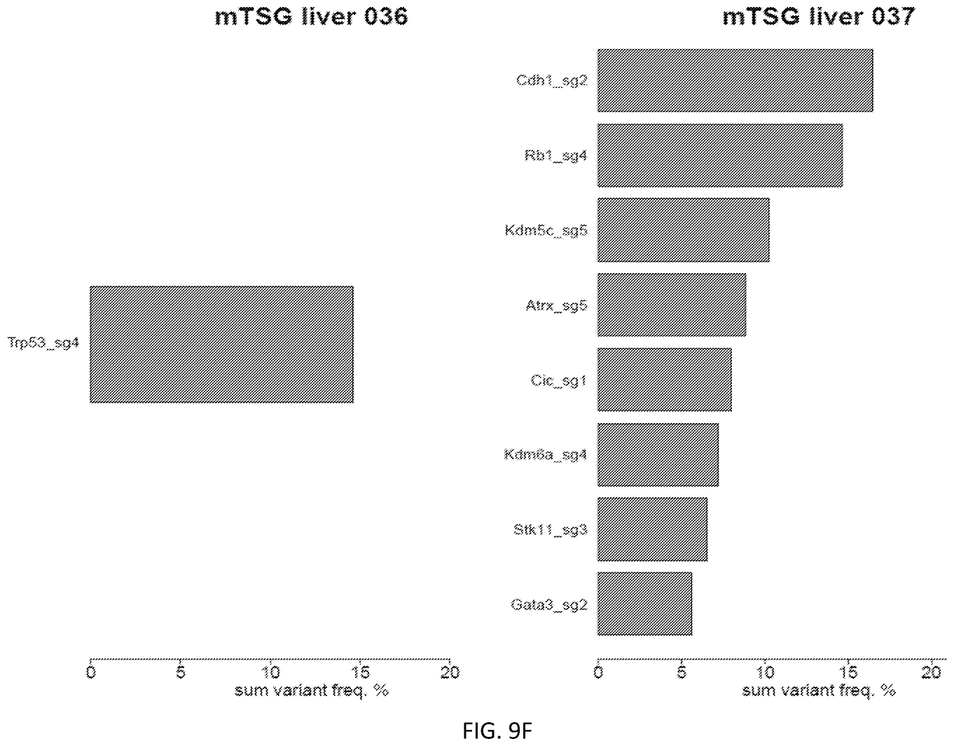

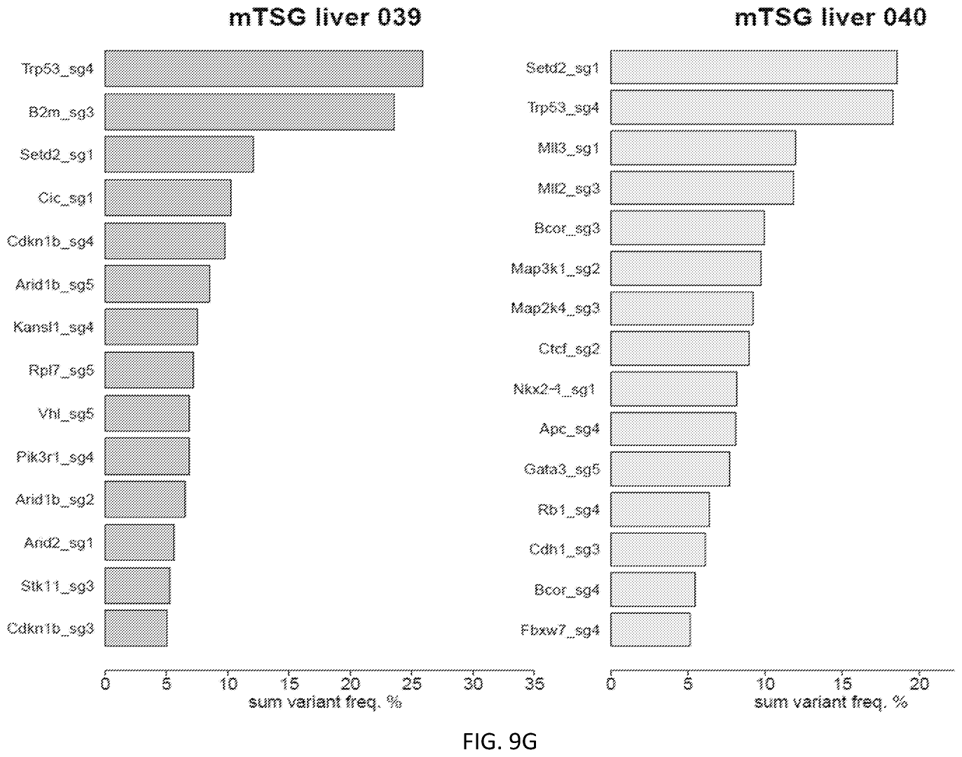

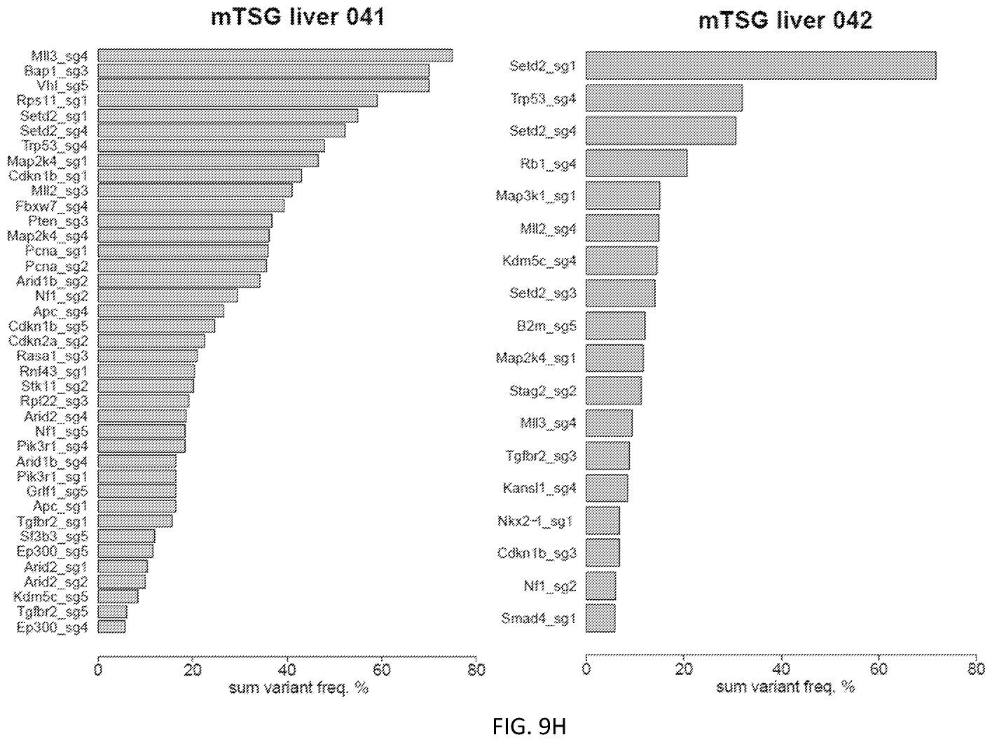

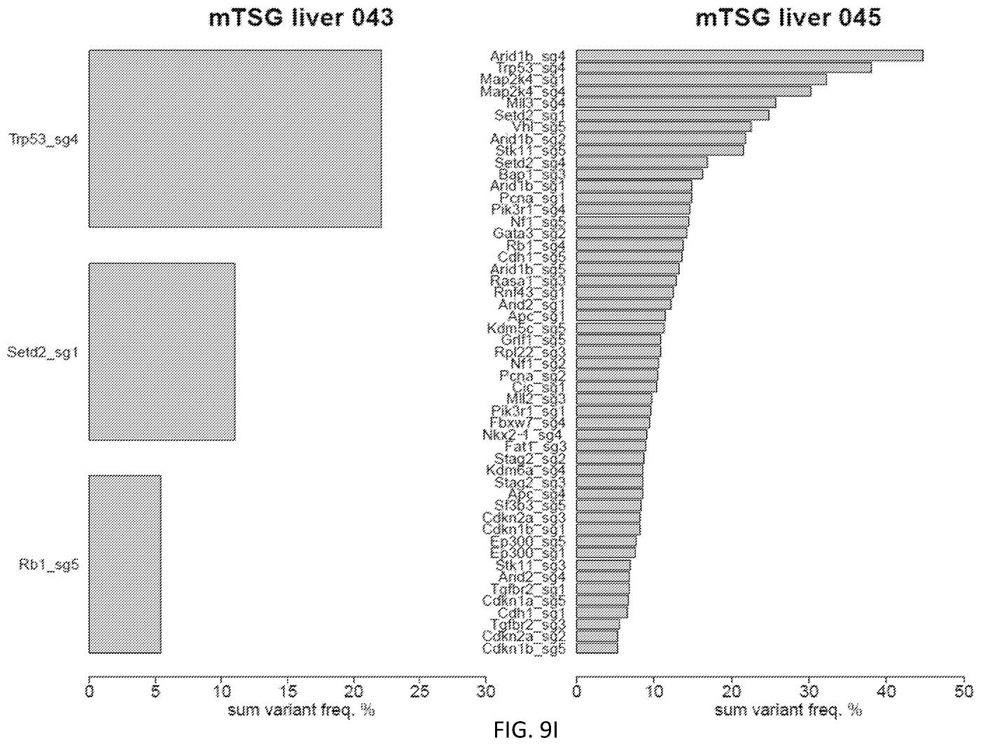

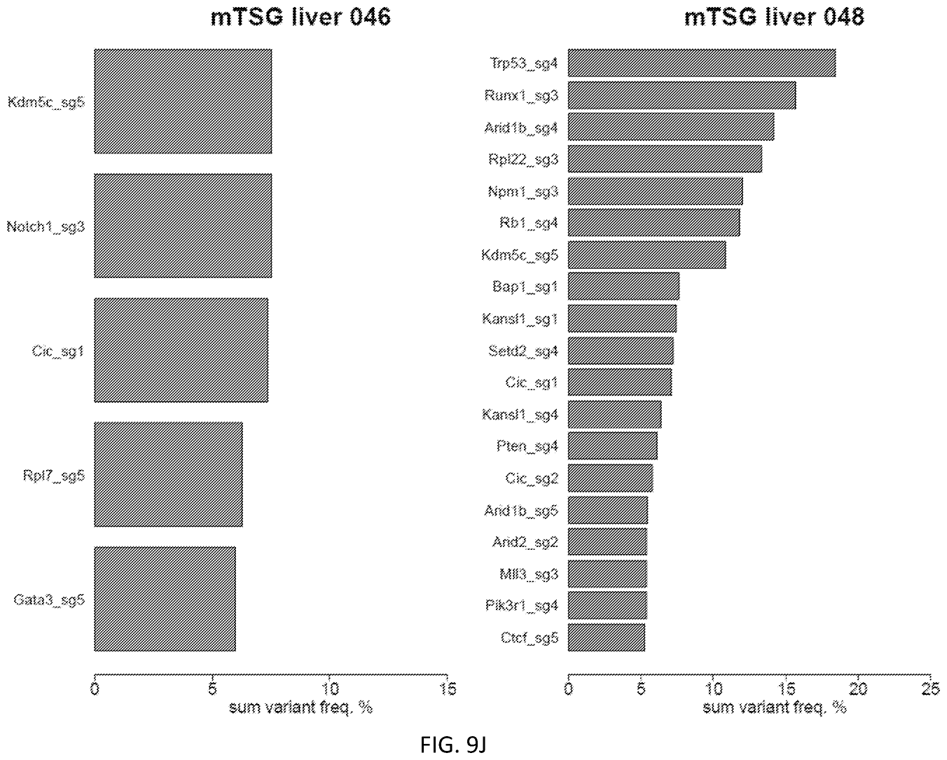

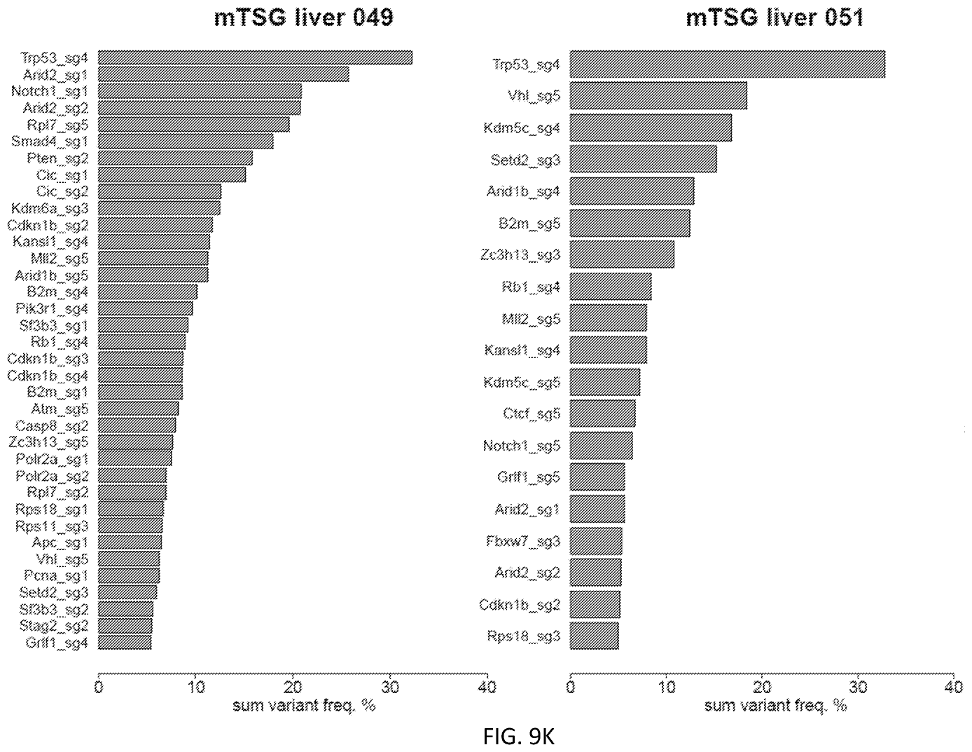

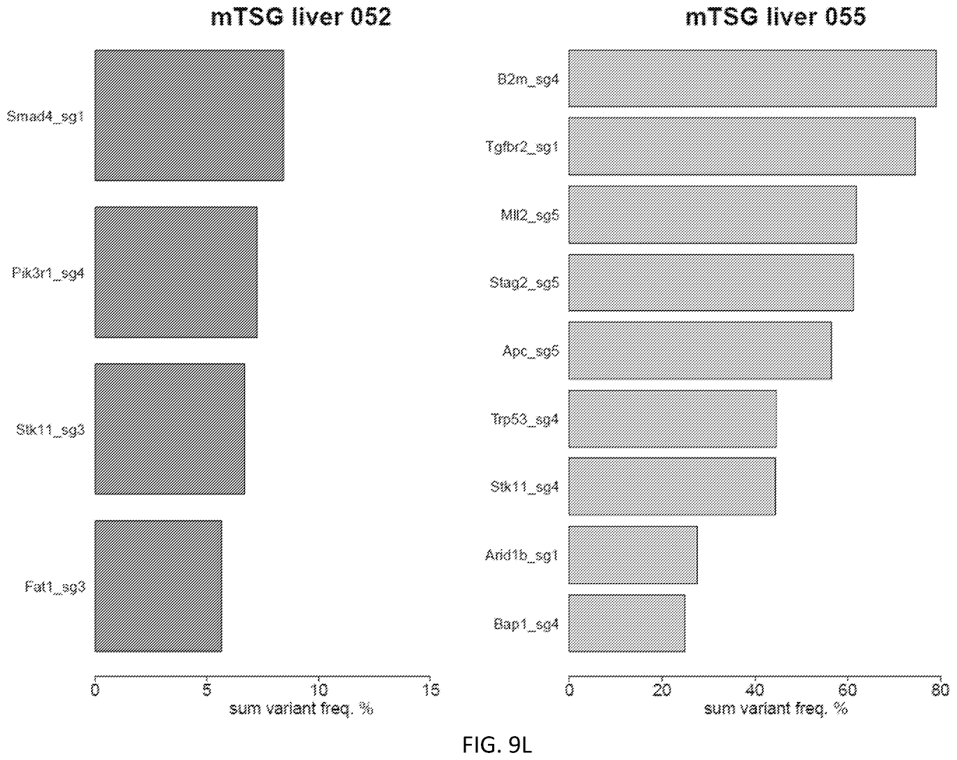

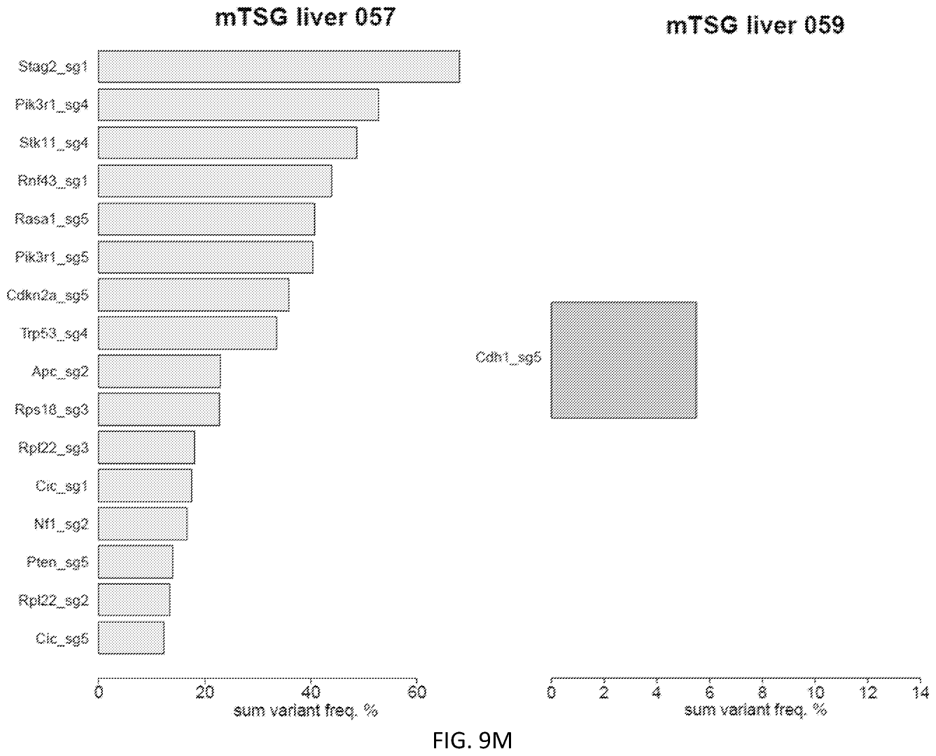

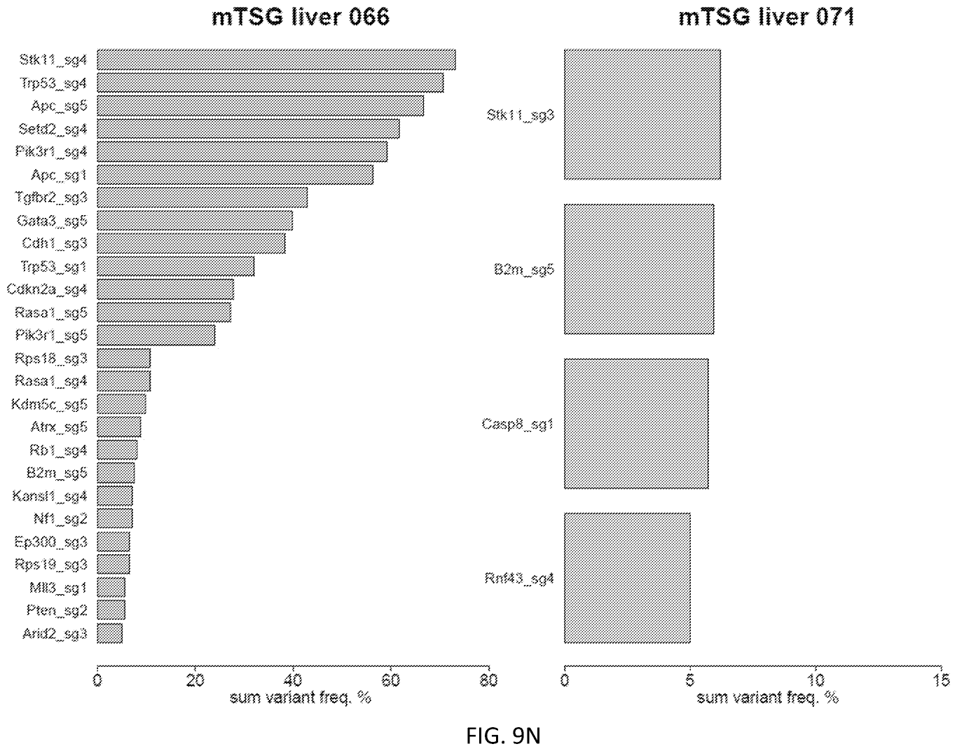

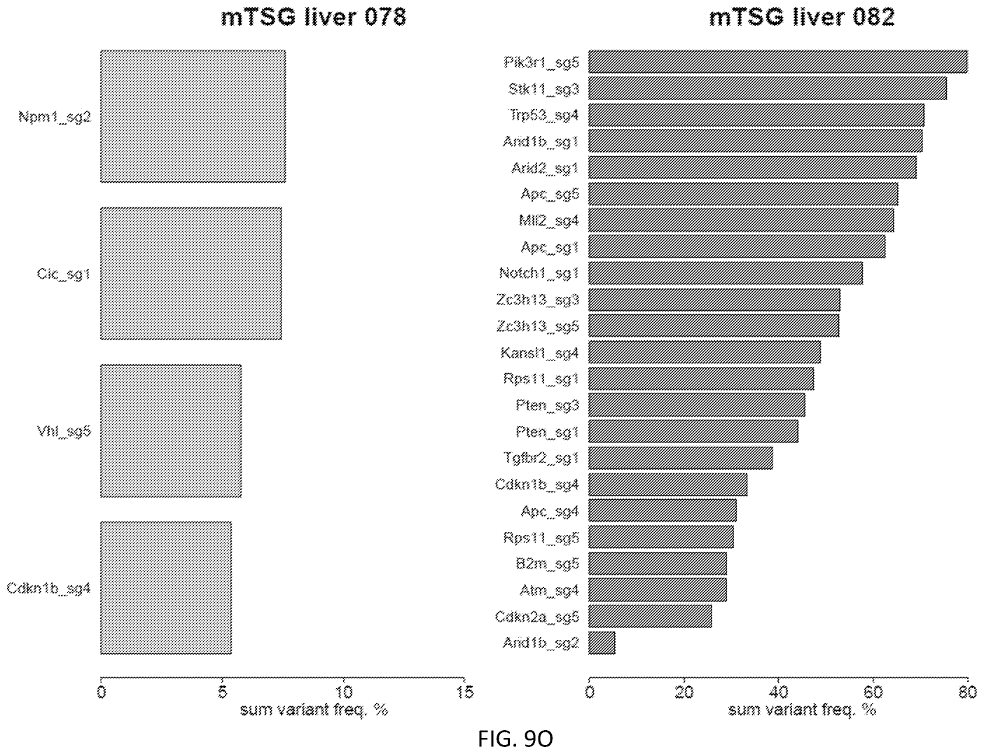

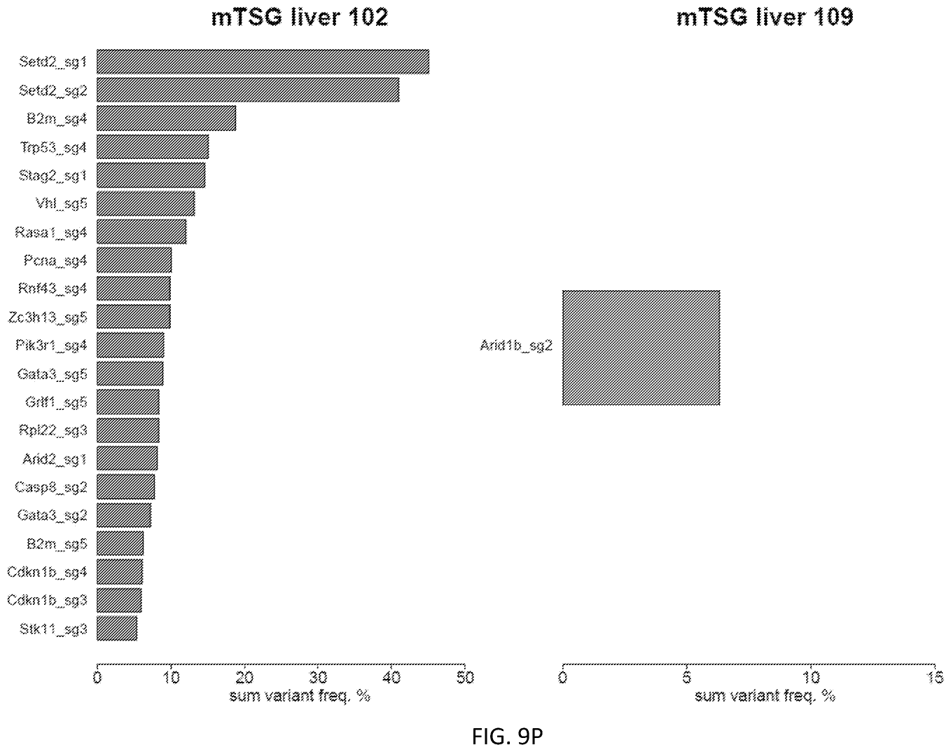

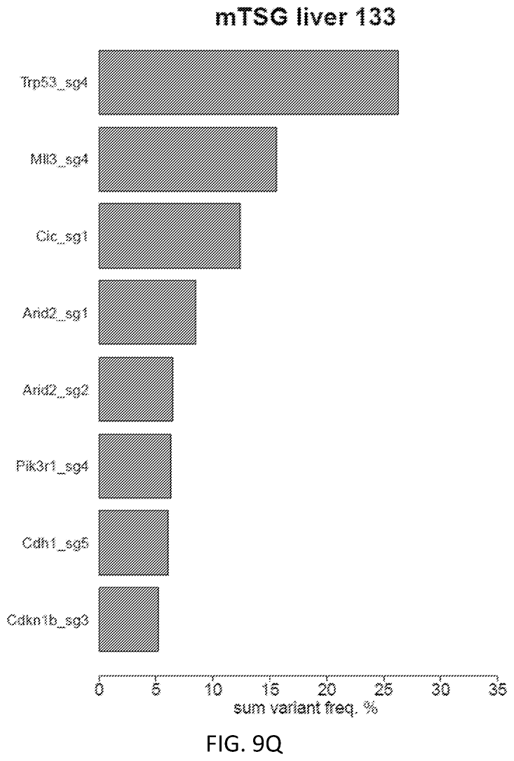

Significantly mutated sgRNA sites (SMSs) were identified in the mTSG-treated liver samples using a false-discovery rate method as compared to PBS and vector-treated liver samples, such that no control sample would have any called SMSs. Of most interest were the dominant clones that had undergone strong positive selection in the tumor, thus it was further required that at least 5% of the reads have an indel in that region in order to call an SMS. Different mTSG-treated liver samples presented with highly heterogeneous mutational signatures, indicating that a diverse array of mutations had undergone positive selection in different samples (FIG. 2B; FIGS. 9A-9Q).

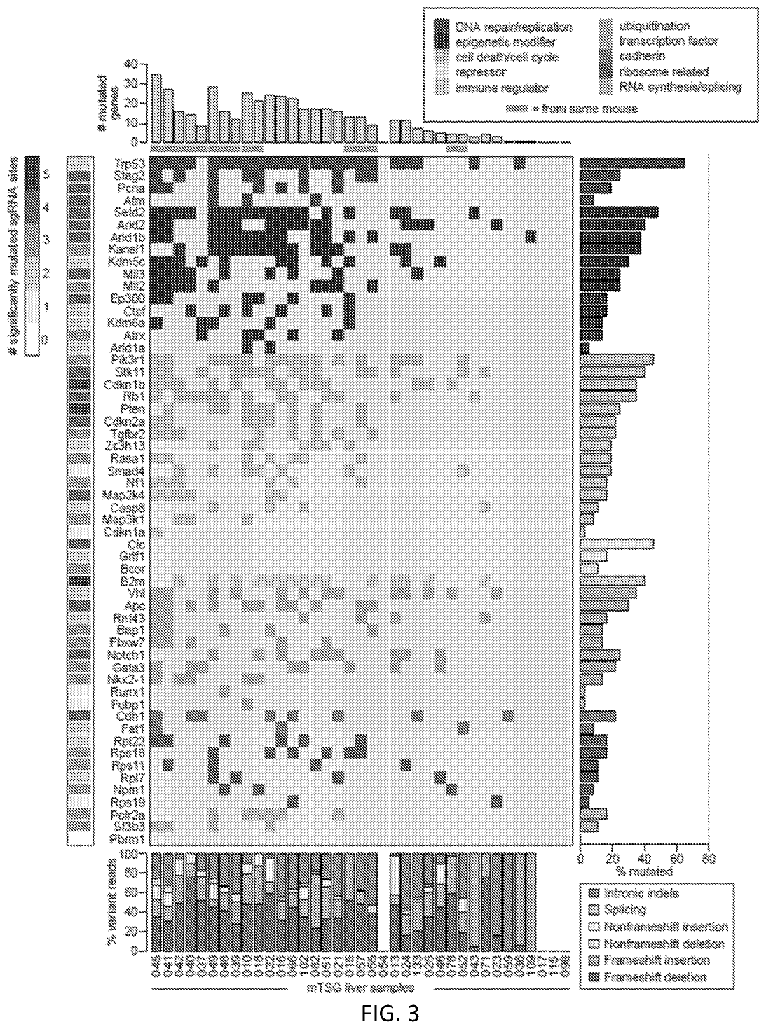

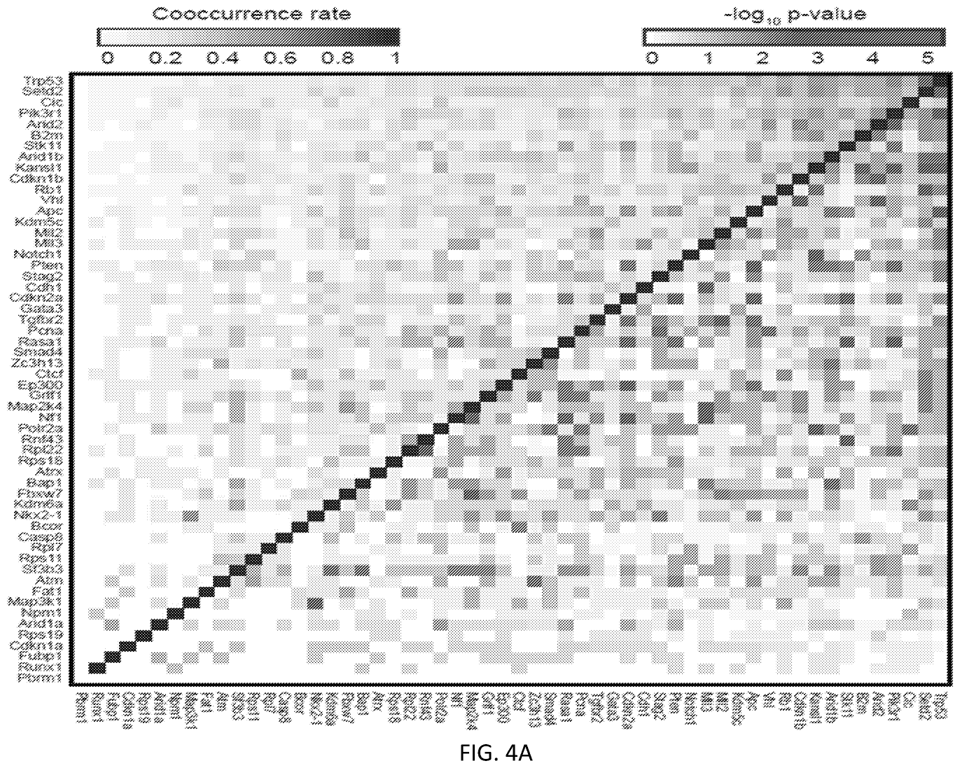

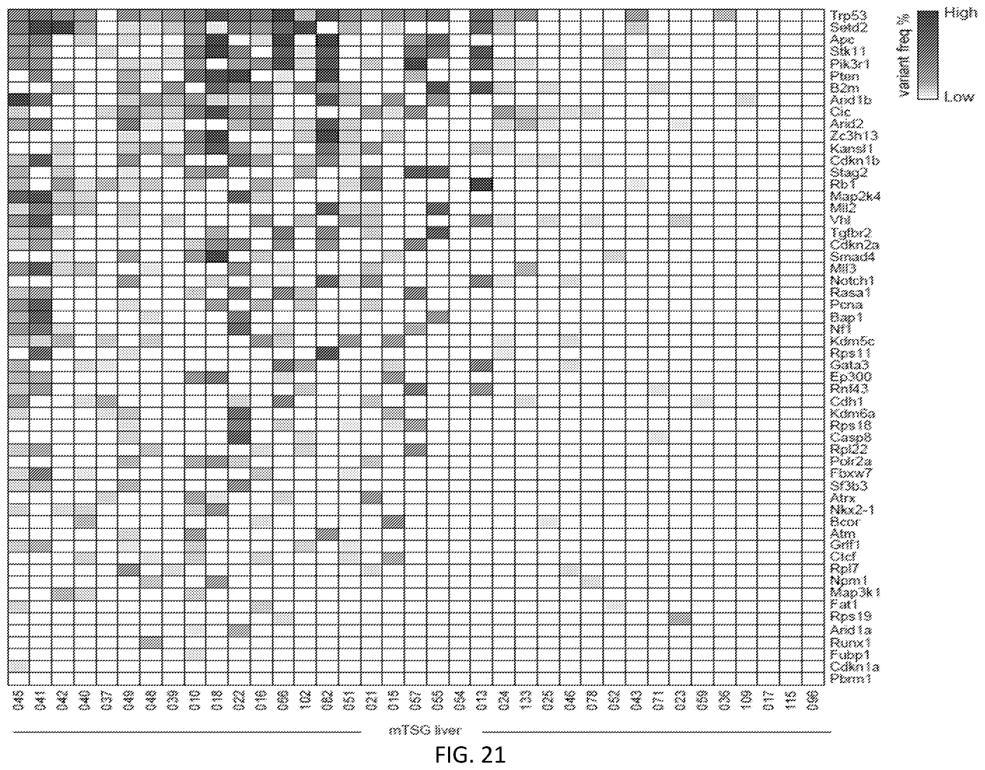

SMSs in each sample were collapsed to the gene level to find significantly mutated genes (SMGs). Analysis of all mTSG liver samples revealed a full mutational landscape of the entire cohort, unfolded as a binary mutation spectrum (FIG. 3) and a quantitative spectrum with sum allele frequencies of each gene in a tumor (FIG. 21). Out of 37 mTSG-treated liver samples, 33 (89%) were found to have major indels (≥5% sum variant frequency and FDR <0.0625) in one or more of the 56 genes in the mTSG library (average number of SMGs per sample=11.7±1.53). Trp53, Setd2, Cic, and Pik3r1 were the top mutated genes in the cohort (mutated in 24/37, 18/37, 17/37 and 17/37 samples, respectively). Trp53 is a well-known tumor suppressor that directly induces liver tumors upon loss-of-function in hepatocytes; Setd2 is an epigenetic modifier that has been implicated in clear cell renal carcinoma, but not yet functionally characterized in liver cancer; Cic is a transcriptional repressor that is a negative regulator of EGFR signaling; Pik3r1 is a modulator of PI3K signaling and loss-of-function mutations in this gene induce liver tumorigenesis in mice. In terms of cellular pathways, epigenetic modifiers and cell death/cell cycle regulators were frequently mutated, with multiple genes that were significantly mutated in more than 20% of samples (FIG. 3). While the importance of epigenetic modifiers in cancer is now widely accepted, direct functional validation of this family of genes in tumorigenesis has not yet been shown in an unbiased systems manner.

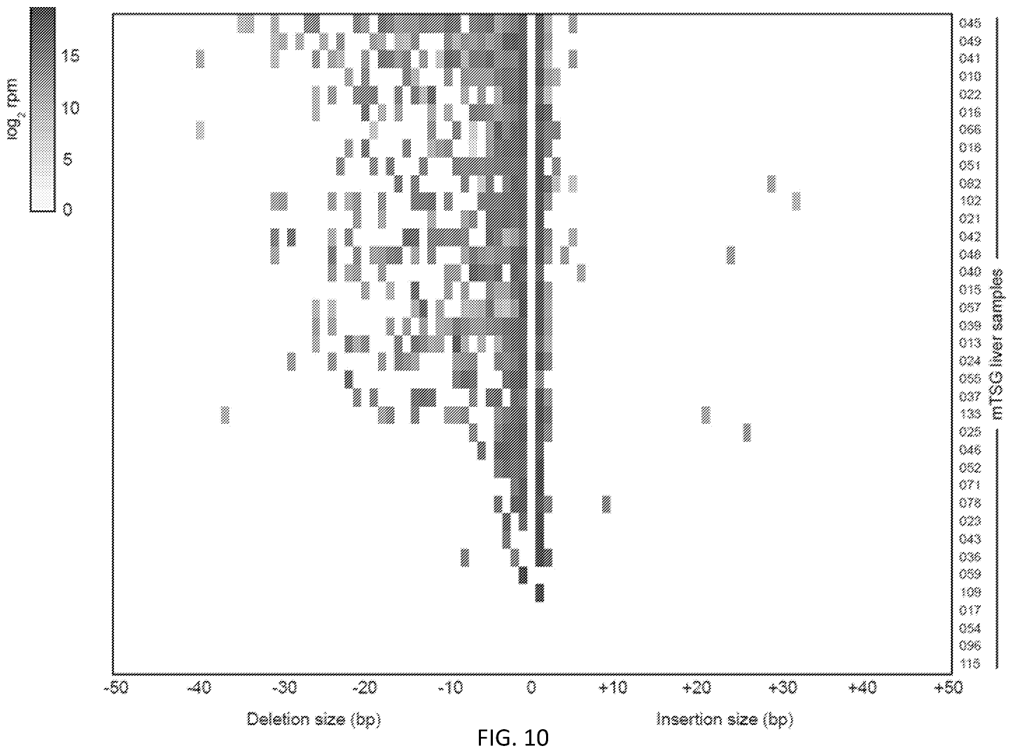

Of the genes that were significantly mutated in at least one sample, the vast majority (91%, or 50/55) had multiple SMSs (median=3 SMSs out of 5 total sgRNAs per gene), suggesting that these genes are indeed functional tumor suppressors (FIG. 3). ANNOVAR analysis of the indels present in the mTSG liver cohort revealed that frameshift insertions and frameshift deletions comprised the majority of total variant reads (median=59.2% across all samples) (FIG. 3; FIG. 10), consistent with the notion that frameshift mutations are expected to cause loss-of-function in genes. Intronic, splice site and non-frameshift mutations nevertheless comprised a sizeable proportion of total variant reads (FIG. 3).

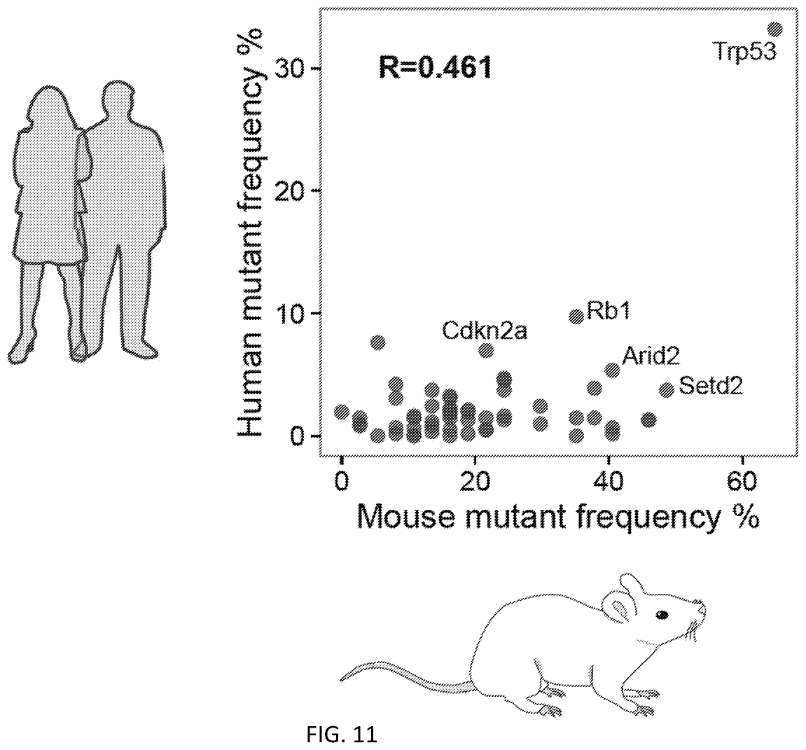

As the study was geared to assess the selective advantage granted upon deletion of each of the genes in the mTSG library, it was reasoned that the population-wide mutation frequency across all mTSG treated liver samples could be interpreted as a proxy for the degree to which each gene normally functions as a tumor suppressor. It was thus tested whether the population-wide mutation frequency in the mTSG treated mice was correlated with the population-wide mutational frequency in humans. Using LIHC data from public datasets (Fujimoto et al., Nat. Genet. 44, 760-764 (2012): Anh et al., Hepatol. Baltim. Md. 60, 1972-1982 (2014)), mouse and human mutation frequencies were significantly correlated (R=0.461, t test for correlation, p=4.78*10⁻⁴) (FIG. 11). These data demonstrated that the functional map of liver cancer tumor suppressors was significantly correlated with human LIHC data in the clinic.

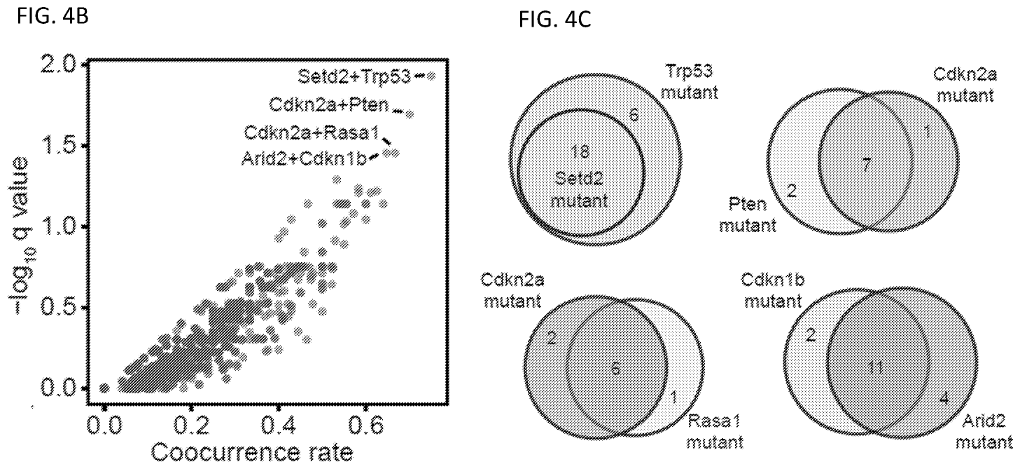

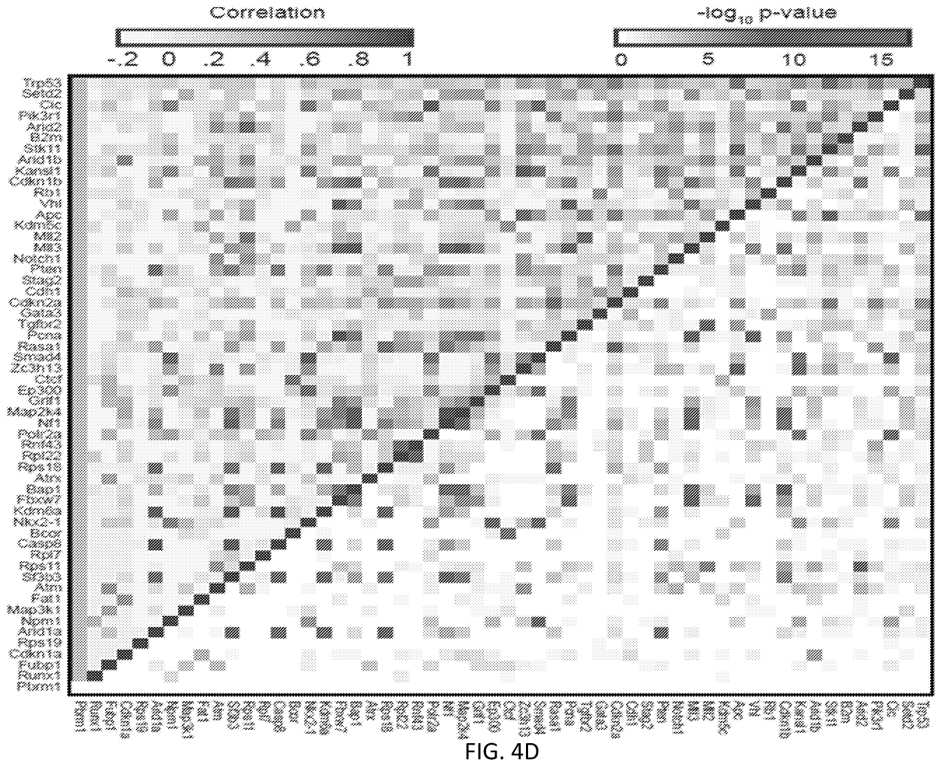

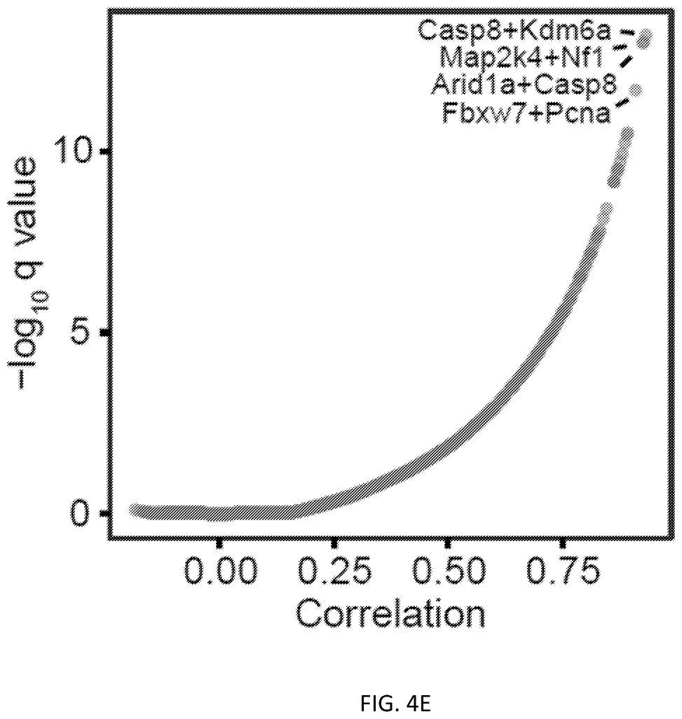

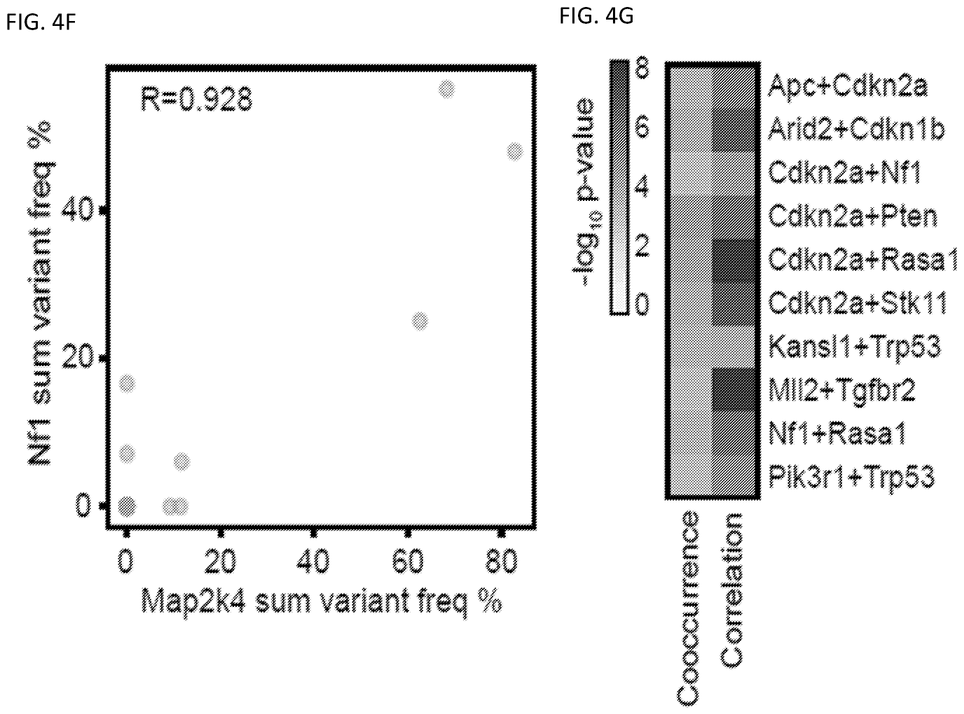

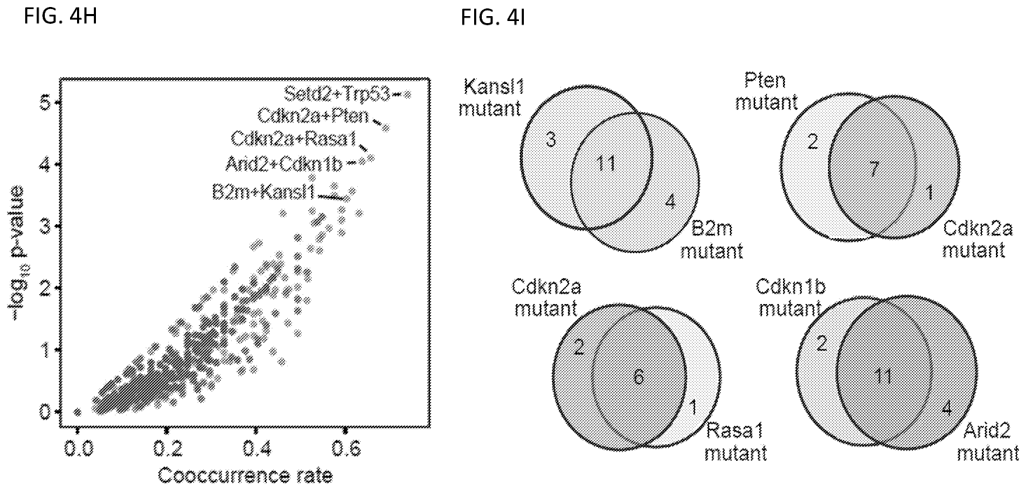

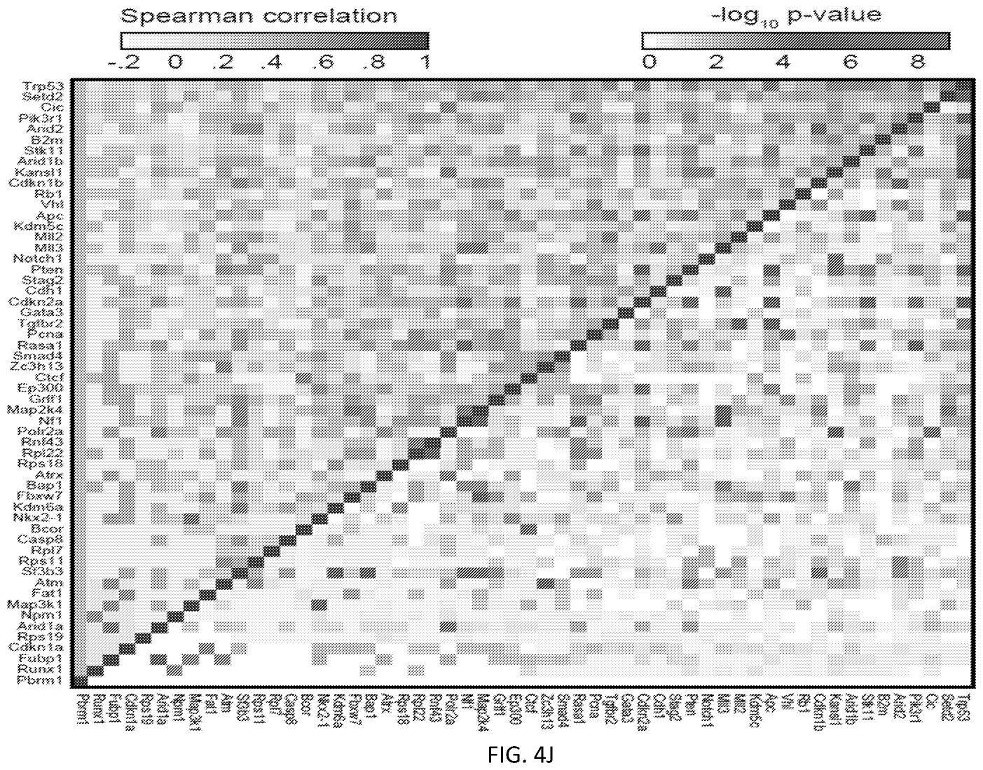

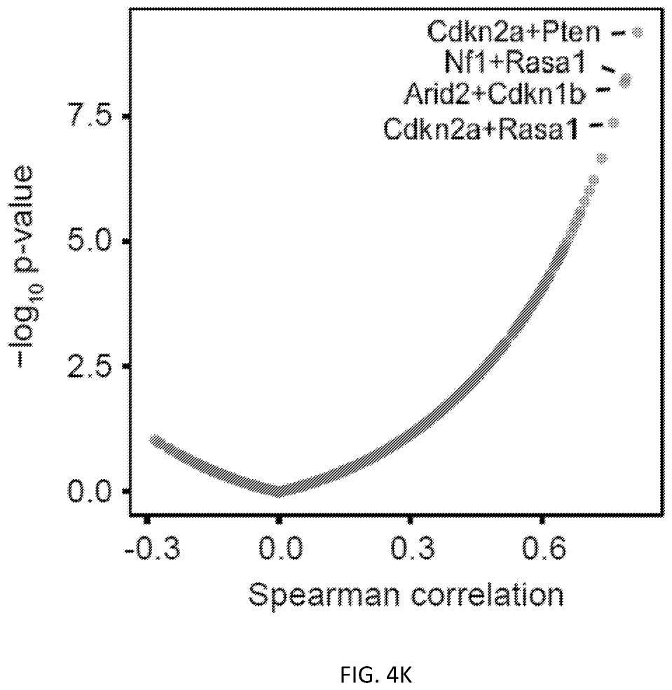

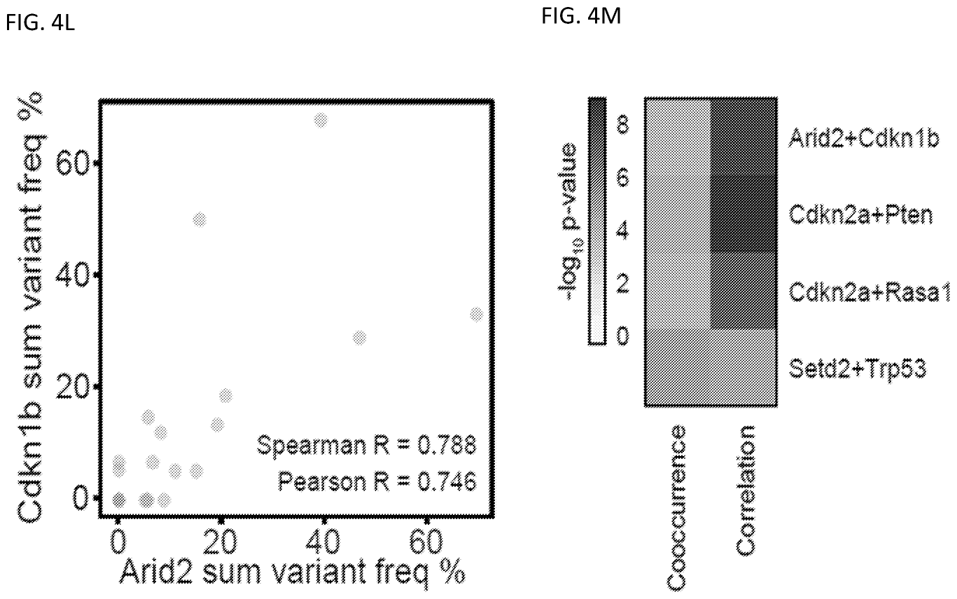

To explore synergistic effects between different genes in the mTSG library, co-mutation analysis was performed. For each pair of genes, the strength of mutational co-occurrence was determined by tabulating the number of samples that were double mutant, single mutant, or double wildtype (FIG. 4A). Out of all 1540 possible gene pairs, a total of 226 pairs were significantly enriched beyond what would be expected by chance (hypergeometric test, Benjamini-Hochberg adjusted p<0.05), with highly significant pairs such as Cdkn2a+Pten (co-occurrence rate=7/10=70%; hypergeometric test, p=2.63*10⁻⁵), Cdkn2a+Rasa1 (co-occurrence rate=6/9=67%; p=7.96*10⁻⁵), Arid2+Cdkn1b (co-occurrence rate=11/17=65%; p=9.13*10⁻⁵), and B2m+Kansl1 (co-occurrence rate=11/18=61%; p=3.6*10⁻⁴) (FIG. 4H-4I). Without wishing to be bound by any specific theory, loss-of-function mutations in both genes of these combinations might synergistically enhance tumor progression.

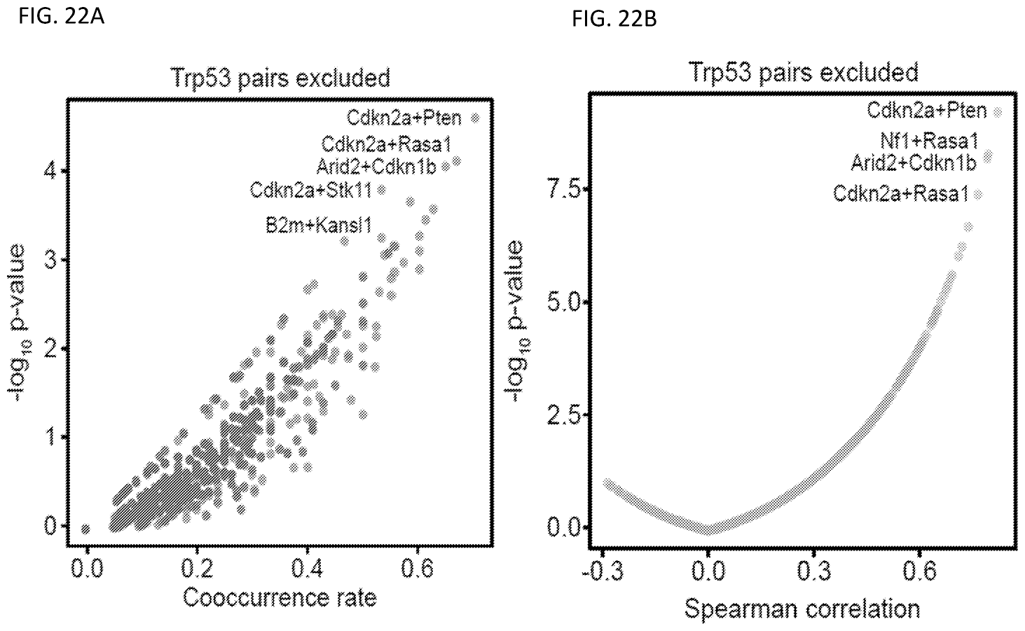

The correlation of gene mutation frequencies within individual tumors was investigated. Since the variant frequency is essentially a metric for the positive selection that acts on a given mutation, genes whose variant frequencies are highly correlated across samples could also be synergistic in driving tumorigenesis. A caveat is that some passenger mutations could be hitchhiking on strong drivers within a given tumor; however, the probability of finding a co-occurring passenger-driver mutation pair is vanishingly small across increasing numbers of mice. The total variant frequency was calculated for each gene by summing all the values from all five sgRNAs, using the summed gene level variant frequencies across each sample to calculate the Spearman correlation between all 1540 possible gene pairs, and assessed whether the correlations were statistically significant (FIG. 4J). A total of 128 gene pairs were significantly correlated (Spearman correlation, Benjamini-Hochberg adjusted p<0.05). The top four correlated pairs were Cdkn2a+Pten (Spearman R=0.817, p=6.97*10⁻¹⁰), Nf1+Rasa1 (R=0.791, p=5.86*10⁻⁹), Arid2+Cdkn1b (R=0.788, p=7.16*10⁻⁹), and Cdkn2a+Rasa1 (R=0.761, p=4.45*10⁻⁸) (FIGS. 4K-4M). The same analysis was performed using Pearson correlation, finding extensive similarities in the identified pairs (FIGS. 4D-4E). As the base vector contained a Trp53 sgRNA, we also performed the co-mutation analyses excluding all pairs involving Trp53 (FIG. 22A-22B). The correlation analysis thus revealed a number of highly significant associations in specific pairs of genes. Four gene pairs were statistically significant at Benjamini-Hochberg adjusted p<0.05 in both the co-occurrence and correlation analyses (FIG. 4M). Interestingly, one of the top gene pairs was Arid2+Cdkn1b, representing a previously unreported synergistic interaction between an epigenetic regulator and a cell cycle regulator.

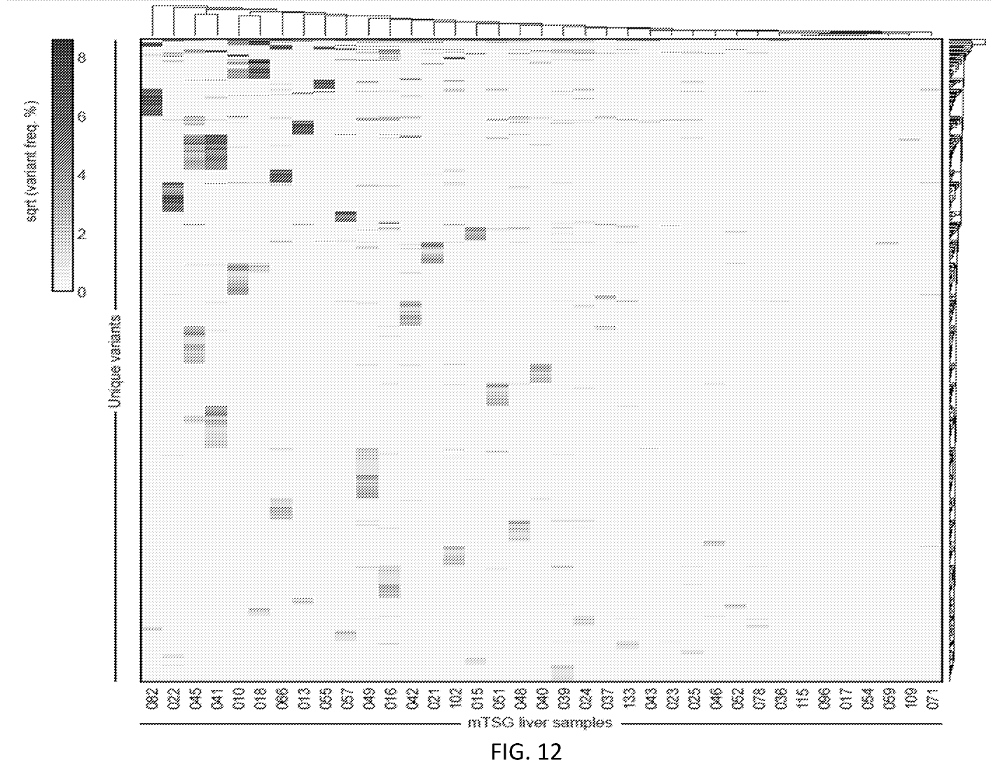

To examine the mutational landscape of the liver tumors induced by the AAV-CRISPR mTSG library at finer resolution, the analysis was reframed to the level of specific indel variants. Across all 37 mTSG-treated liver samples, 593 unique variants were identified that had a variant frequency ≥1% in at least one sample. The majority of these variants (80.94%) were deletions rather than insertions (FIG. 10). Hierarchical clustering of the variant-level data across all mTSG-treated liver samples revealed the existence of sample-specific variants. 70.25% (418/595) of the variants were sample-specific (private variants), while 29.75% (177/595) variants were found across multiple samples (shared variants) (FIG. 12). Shared variants could originate from convergent processes of NHEJ following Cas9/sgRNA mediated DSBs, leading to the same indel pattern. Alternatively, shared variants in different liver lobes from the same mouse could also arise from clonal expansion or metastasis.

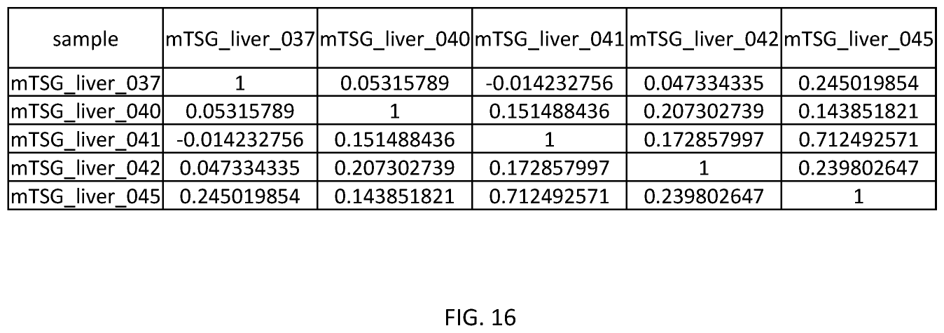

To further understand the clonal architecture in this genetically complex, highly heterogeneous yet fully gene-targeted, autochthonous tumor model, analysis was focused on a single mTSG-treated mouse that had presented with multiple visible tumors in several liver lobes, 5 of which had been harvested for MIPs capture sequencing (FIG. 5A). Analysis of the sgRNA-level variant frequencies in the 5 lobes revealed strong pairwise correlations between multiple lobes (FIG. 5B; FIG. 16). For instance, lobes 3 and 5 were significantly correlated (Spearman rank correlation (R)=0.700, p<2.2*10⁻¹⁶). Lobe 2 and lobe 4 were also significantly correlated though to a lesser extent (R=0.207, p=5.08*10⁻⁴). Furthermore, lobes 1, 2, and 4 were also significantly correlated with lobe 5 (R=0.248, p=2.99*10⁻⁵; R=0.146, p=0.146; R=0.243, p=4.31*10⁻⁵). The inter-lobe correlations are suggestive of similar variant compositions within these liver lobes.

To clearly delineate any potential clonal mixtures among the 5 liver lobes, the unique variant patterns across these samples were examined. 178 unique variants were identified (≥1% variant frequency threshold) represented within the 5 liver lobes. Using binary variant calls (i.e., whether a given variant is present or absent in a sample), the 178 variants were clustered into 8 groups (FIG. 5C). Variants in clusters 1, 2, 3, 5, and 6 were specific to a single lobe (private variant clusters), whereas variants in clusters 4, 7, and 8 were present across multiple lobes (shared variant clusters) (FIG. 5E). By averaging the variant frequencies within each cluster for a given sample, the relative contribution of each cluster to the overall composition of the 5 liver lobes was assessed (FIGS. 5D-5E). The degree of correlation between lobes (FIG. 5B) was echoed by their degree of variant cluster sharing (lobe 1 shares cluster 4 with lobe 5, lobes 2 and 4 share variant cluster 8 with lobe 5, lobe 3 share clusters 7 and 8 with lobe 5) (FIGS. 5D-5E). The presence of cluster 8 in 4 out of 5 lobes was especially notable, as it comprised a large percentage of the mutational burden in these 4 lobes (FIGS. 5D-5E). Cluster 8 was defined by mutations in MI13 (also known as Kmt2c), Setd2 and Trp53 (FIG. 5E). Variant-level analyses therefore recaptured the pairwise correlations identified on the sgRNA level, suggesting clonal mixture between individual liver lobes within a single mouse.

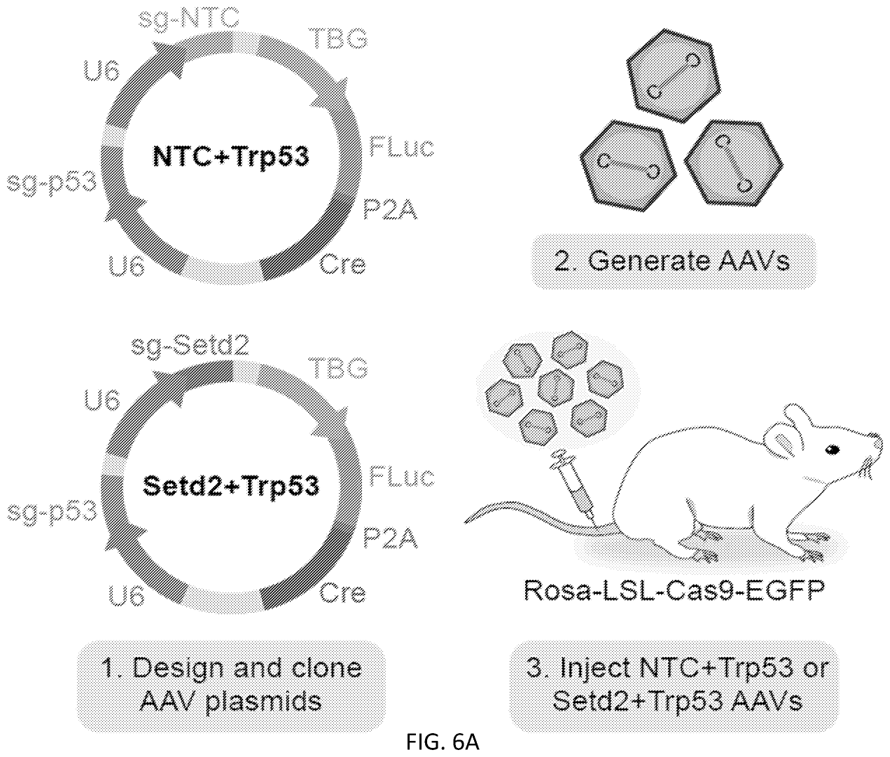

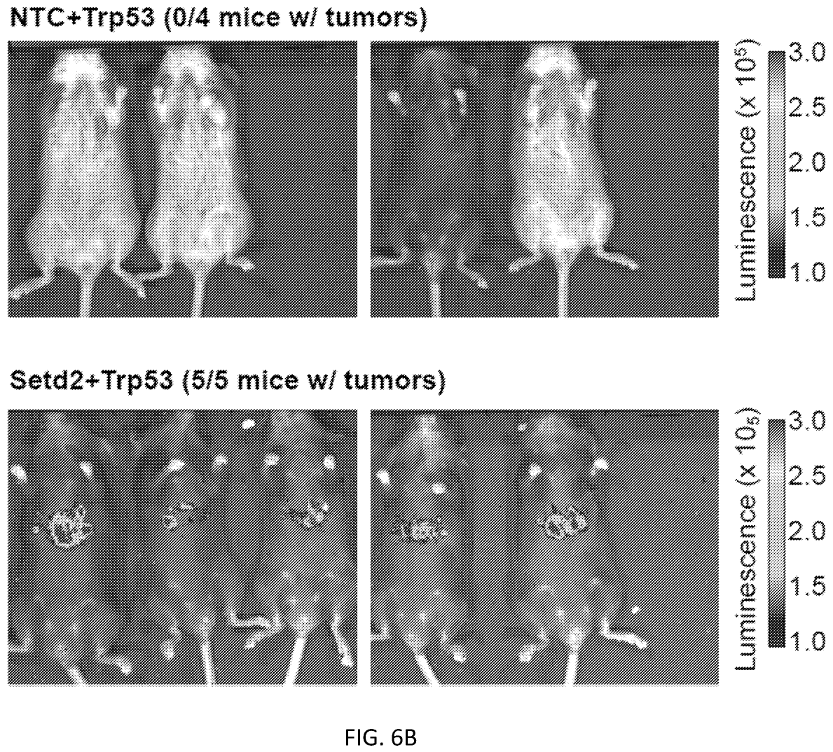

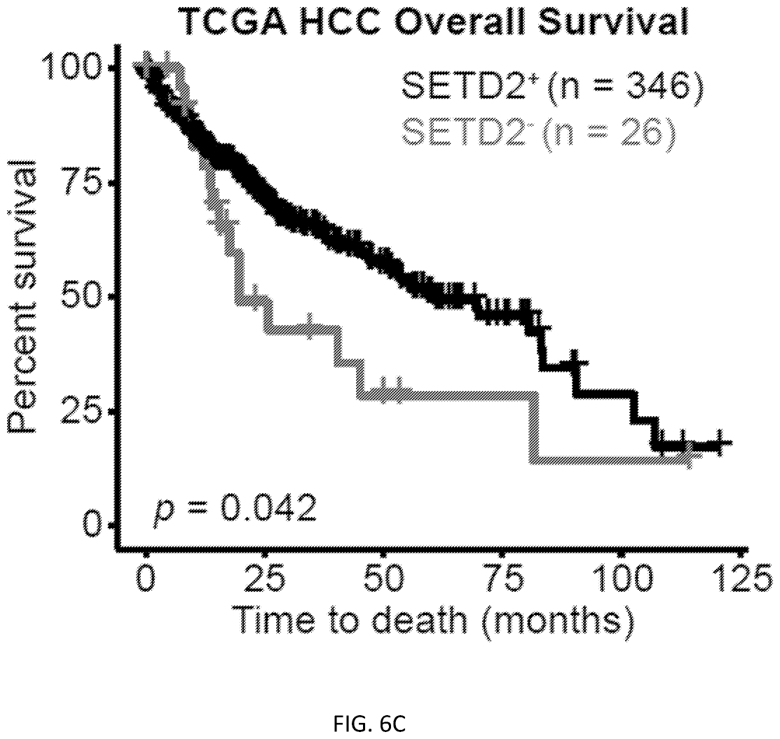

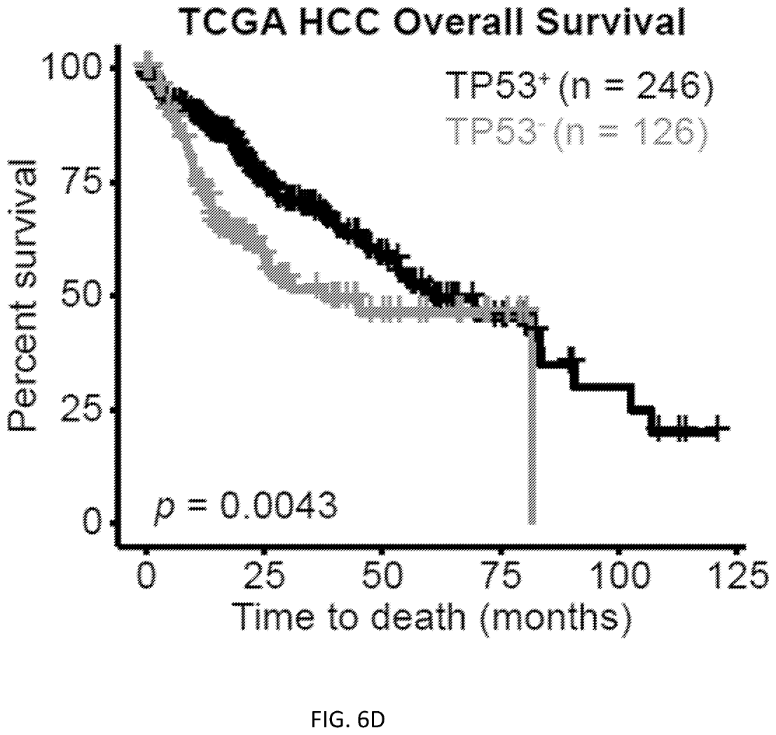

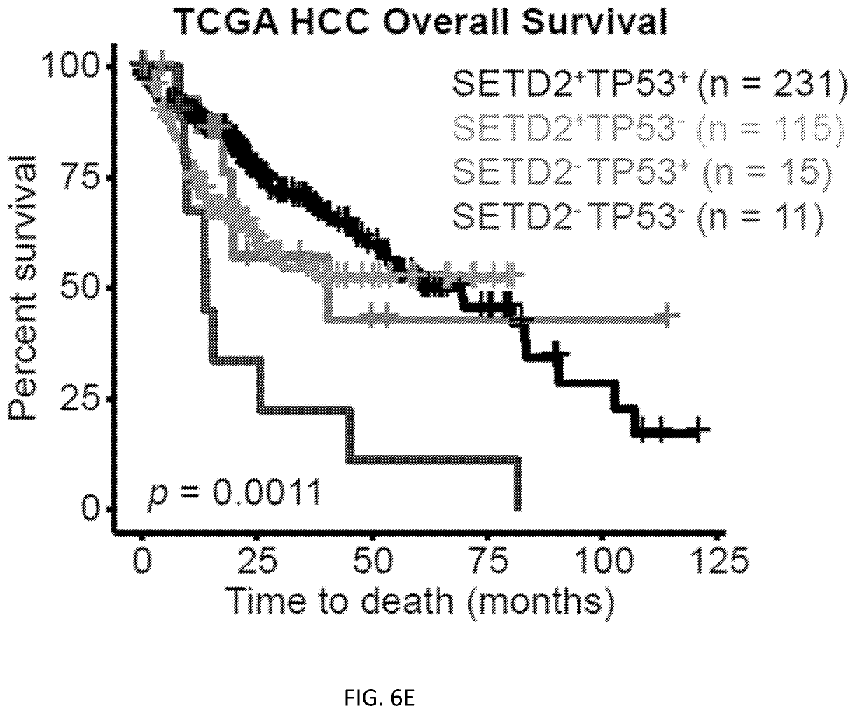

Given the repeated emergence of Setd2 and Trp53 in each arm of the analysis (i.e., population-level mutation frequencies, co-mutation analysis, and clonal mixture analysis), the Setd2+Trp53 gene pair was further investigated. An AAV vector for liver-specific CRISPR knockout that expressed Cre recombinase under a TBG promoter, together with a Trp53-targeting sgRNA cassette and an empty sgRNA cassette was generated (FIG. 6A). The vector also contained a firefly luciferase gene (FLuc) co-cistronic with Cre under the TBG promoter for live imaging of tumorigenesis in mice. Either a non-targeting control (NTC) sgRNA (making a NTC+Trp53 AAV vector), or an sgRNA targeting Setd2 (Setd2+Trp53 vector) was cloned into the empty sgRNA cassette of this vector (FIG. 6A). After AAV packaging, NTC+Trp53 AAVs or Setd2+Trp53 AAVs was injected into LSL-Cas9 mice (FIG. 6A). One month after injection, tumor growth was assessed using a bioluminescent imaging system (IVIS) for live imaging. Of the mice treated with NTC+Trp53 AAVs (n=4), none developed detectable tumors at this time point (FIG. 6B). In sharp contrast, all mice treated with Setd2+Trp53 AAVs (n=5) developed liver tumors (Setd2+Trp53 vs NTC+Trp53, one tailed Chi-square test, p=0.0013) (FIG. 6B). These data confirm the synergistic effect of mutations in Setd2 and Trp53 to drive liver tumorigenesis in healthy, immunocompetent mice.

To assess whether loss-of-function mutations in Setd2 and Trp53 are clinically relevant for human LIHC, patient data from the TCGA LIHC dataset was subsequently analyzed. All patients (n=372) were classified into “negative” or “positive” groups based on the integration of somatic mutations, copy number variations, and gene expression profiles. Specifically, a tumor was classified as negative for SETD2 or TP53 if it exhibited one or more of the following: 1) a non-silent mutation, 2) homozygous deletion, or 3) a gene expression z-score <−2, indicating an expression level at least two standard deviations below the mean. Using these criteria, 6.99% (26/372) of LIHC patients were classified as SETD2 negative (SETD2⁻), and 33.87% (126/372) as TP53 negative (TP53⁻). Kaplan-Meier survival analysis revealed statistically significant associations between SETD2 status and overall survival, with SETD2⁻ patients having worse survival times compared to SETD2⁺ patients (log-rank test, p=0.042) (FIG. 6C). A similar association was found with regards to TP53 status, with TP53⁻ patients having a worse prognosis compared to TP53+ patients (log-rank test, p=0.0043) (FIG. 6D).

After classifying all TCGA LIHC patients into 4 groups in terms of both SETD2 and TP53 status, Kaplan-Meier survival analysis was again performed. The SETD2TP53″ double-negative group (n=11) had the worst survival among all four groups (log-rank test, p=0.0011 by comparing all 4 survival curves; pairwise comparisons for SETD2TP53″ group: p<0.0001 vs. SETD2+TP53+, p=0.039 vs. SETD2TP53+, p=0.039 vs. SETD2 TP53⁻) (FIG. 6E). Taken together, these results collectively demonstrate that SETD2 and TP53 mutations, alone or in combination, are indicative biomarkers for LIHC prognosis, with the identification of SETD2⁻ TP53⁻ patients as being associated with particularly poor survival.

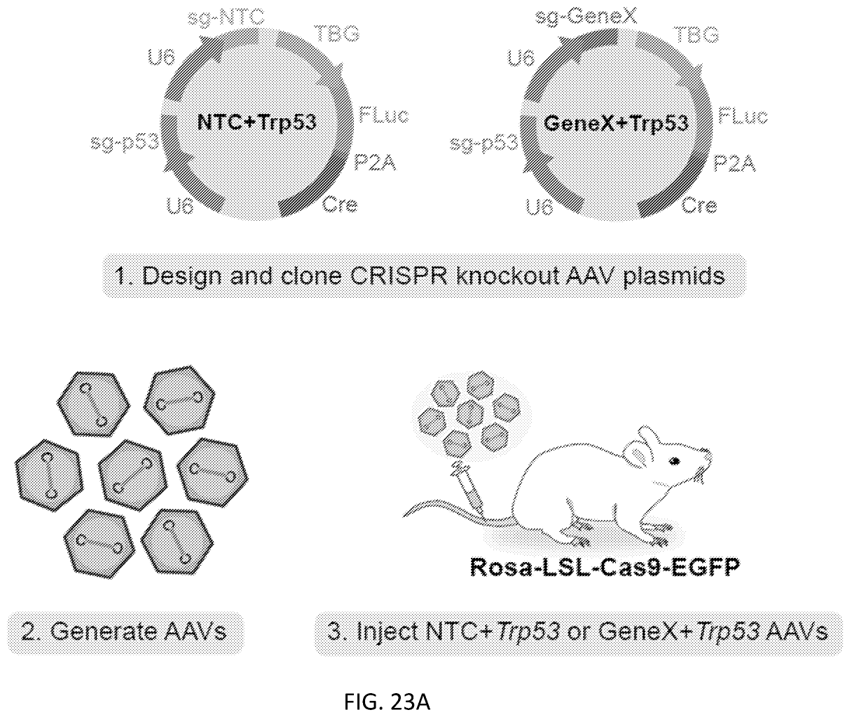

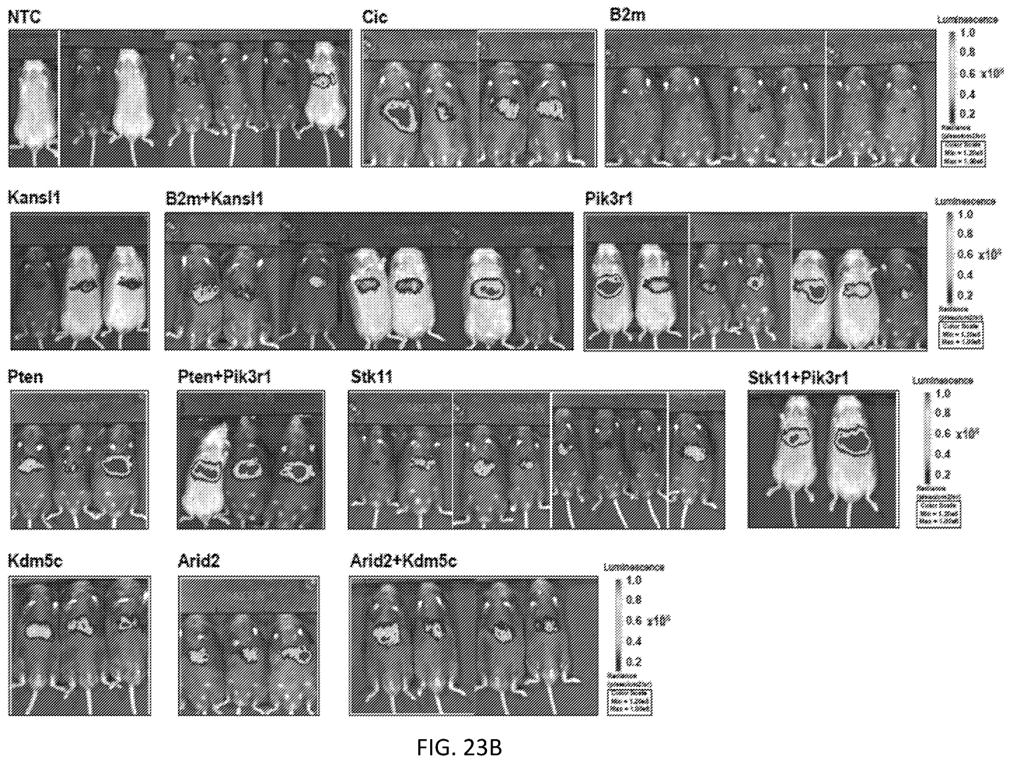

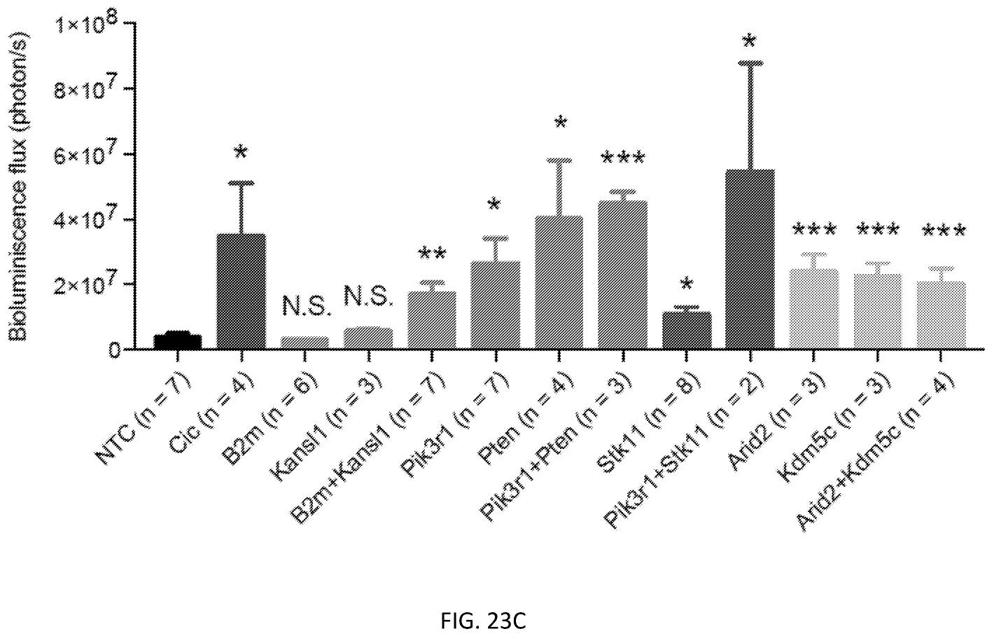

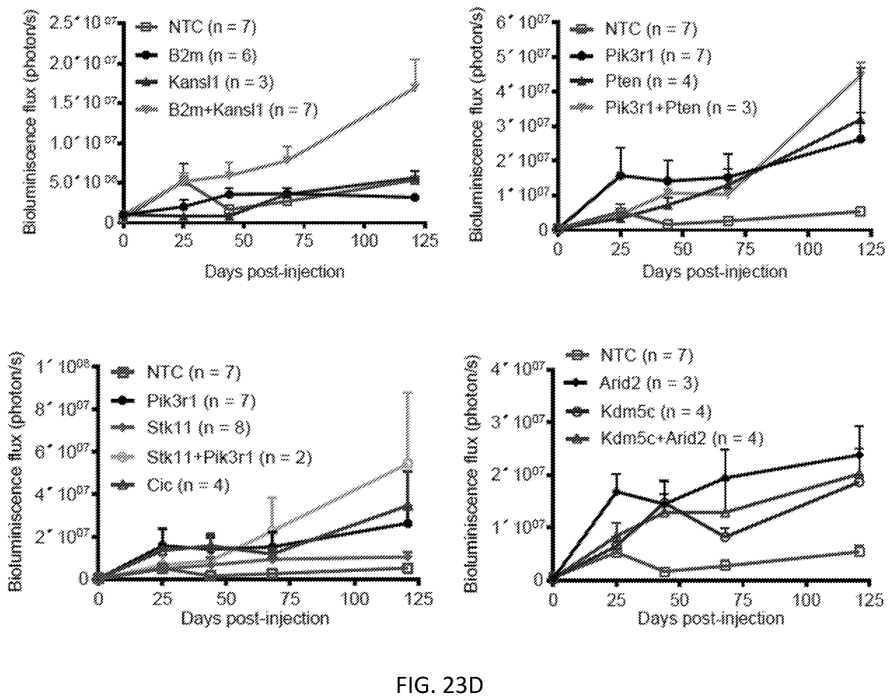

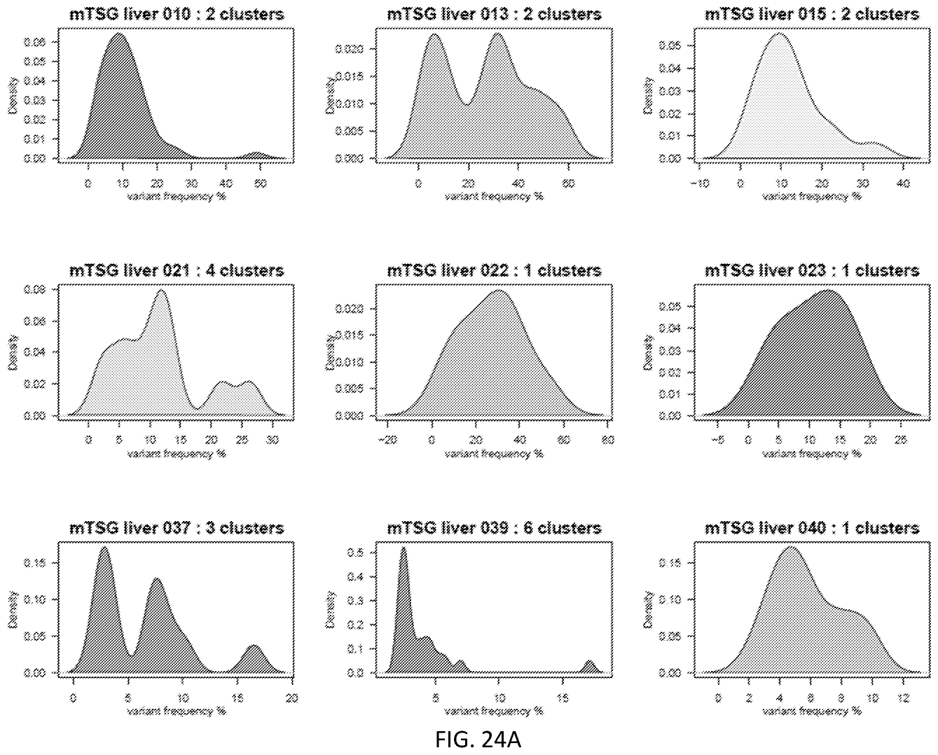

The functional roles of mutations in several of the top genes were individually tested, in a Trp53-sensitized background. Gene pairs were chosen based on their ranking in the screen, potential biological function, and literature. An AAV vector for liver-specific CRISPR knockout was generated that expressed Cre recombinase under a TBG promoter, together with a Trp53-targeting sgRNA cassette and an open (GeneX-targeting) sgRNA cassette (FIG. 23A). The vector also contained a firefly luciferase gene (FLuc) co-cistronic with Cre under the TBG promoter for live imaging of tumorigenesis in mice. Either a non-targeting control (NTC) sgRNA (thus only mutating Trp53), or a top candidate geneX-targeting sgRNA (GTS, thus mutating both GeneX and Trp53) was cloned into the 2^{nd} sgRNA expression cassette. After AAV packaging, NTC+Trp53 or GTS+Trp53 AAVs were injected into LSL-Cas9 mice (FIG. 23A). Growth of potential liver tumors was assessed the by monitoring their luciferase activities using a bioluminescent in vivo imaging system (IVIS) (FIG. 23B). Compared to mice treated with NTC AAVs (n=8), sgRNAs targeting multiple candidates identified in the screen, including Cic (n=4), Pik3r1 (n=7), Pten (n=4), Stk11 (n=8), Arid2 (n=3), and Kdm5c (n=3) had significantly stronger luciferase activity (two-sided unpaired t test, p<0.05 for all groups, (FIGS. 23B-23D), suggesting that knocking out these genes accelerated liver tumorigenesis at high penetrance in a Trp53-sensitized background. Double knockouts such as Pik3r1+Pten (n=3) and Arid2+Kdm5c (n=4) also had significantly stronger luciferase activity compared to NTC (two-sided unpaired t test, p<0.001), but not significant compared to respective single knockouts (FIG. 23C-23D), suggesting that these genes are strong drivers alone but do not have synergistic effect with each other. B2m+Kansl1 is one of the top co-occurring gene pairs identified in the screen (co-occurrence rate=11/18=61%, p=3.6*10⁻⁴). While LSL-Cas9 mice injected with AAVs for individual knockout of B2m or Kansl1 alone did not show significantly stronger luminescence intensities compared to NTC group, AAVs targeting the B2m+Kansl1 combination showed significantly higher luminescence intensities as compared to NTC (two-sided unpaired t test, p<0.01), B2m alone (p<0.01) and Kansl1 alone (p<0.05) (FIG. 23C-23D). These results suggested that combinatorial knockout of B2m and Kansl1 had a synergistic effect in accelerating liver tumor development, whereas the single knockouts of B2m or Kansl1 were not sufficient to induce liver tumorigenesis in a Trp53-sensitized background. In summary, the single and combinatorial AAV-CRISPR knockout experiments further confirmed the phenotypes of several top ranked genes and co-occurring gene pairs in liver tumorigenesis. The study demonstrates a powerful strategy for quantitatively mapping functional suppressors in the cancer genome and their synergistic relationships directly in vivo in a full immunocompetent setting.

Herein, an approach was developed for direct in vivo CRISPR screens to map a provisional functional cancer genome atlas (FCGA) of tumor suppressors in the mouse liver in an autochthonous manner. The genes selected for this study were clinically-relevant, significantly mutated genes in human cancers. As many of the genes have not been specifically studied in the context of cancer in vivo, these candidate tumor suppressors were functionally interrogated in a controlled, quantitative, and high-throughput manner in mice. Using an AAV library carrying 280 different CRISPR sgRNAs, 56 genes were tested for their ability to promote tumorigenesis in the mouse liver upon loss-of-function by Cas9 mutagenesis. Capture sequencing of the resultant liver tumors revealed a heterogeneous mutational landscape, indicating that several of the genes in the mTSG library indeed function as tumor suppressors. The importance of epigenetic control in cancer is now widely appreciated, in part due to tumor profiling studies that have identified recurrent mutations in epigenetic regulators across multiple cancer types. However, the direct contribution of most epigenetic factors to tumor suppression has not yet been rigorously demonstrated. It is thus noteworthy that several of the top drivers identified in our screen were epigenetic modifiers, functionally demonstrating the importance of this gene family in tumor suppression. The population-wide mutation frequency in mTSG treated mice was significantly correlated with population-wide mutation frequency in human LIHC.

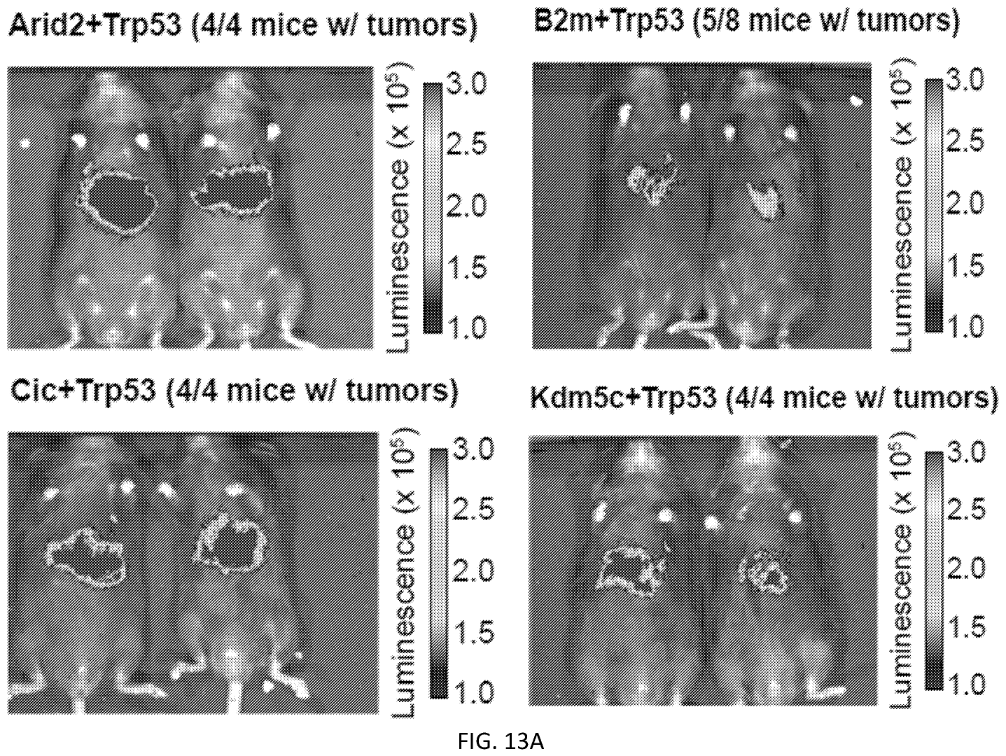

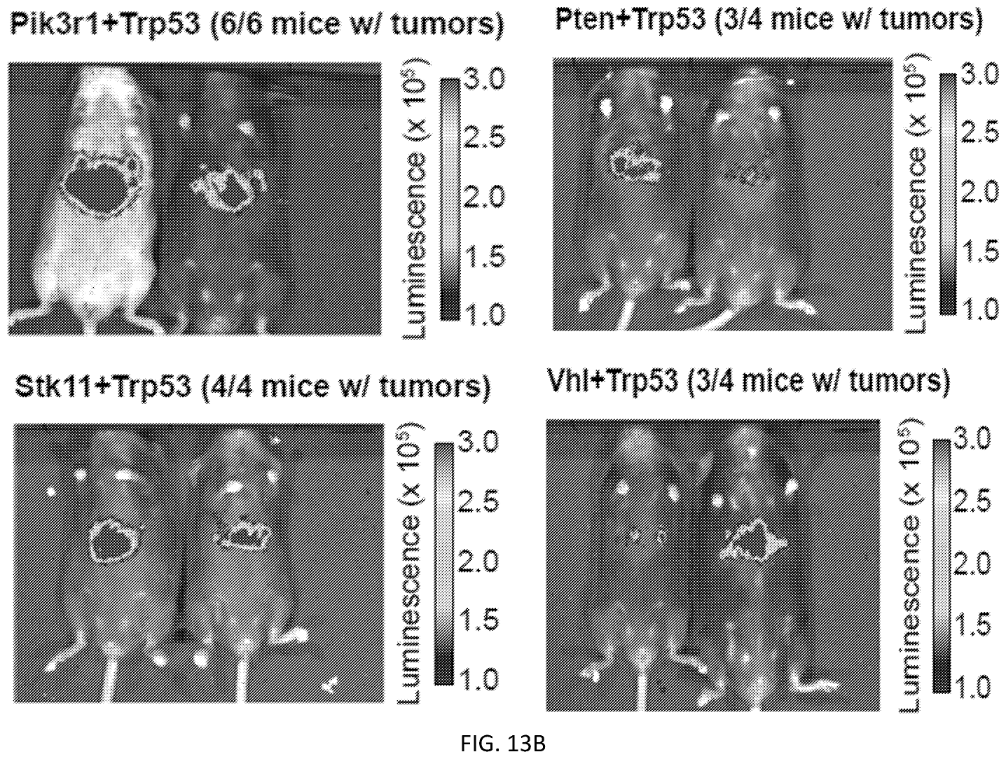

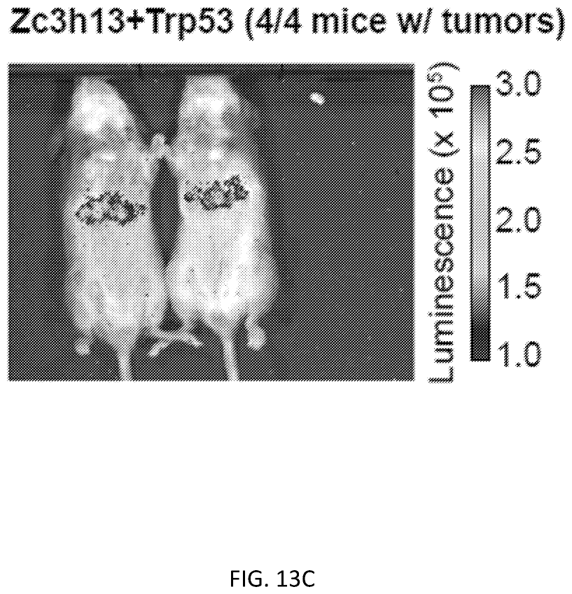

Co-mutation analysis identified several pairs of significantly co-occurring mutations, with Setd2+Trp53 as the top-ranked pair. MIP capture sequencing instead of conventional sgRNA sequencing enabled direct, multiplexed analysis of the indels induced by Cas9 mutagenesis. Variant compositions were systematically dissected across multiple liver lobes from a single mouse, uncovering evidence of clonal mixture between lobes. One variant cluster in particular was found in 4 out of 5 liver lobes, and this cluster was defined by mutations in Setd2 and Trp53. A dual-sgRNA approach was leveraged to simultaneously knockout Setd2 and Trp53 in the mouse liver, leading to rapid liver tumor growth within one month. Several other functional drivers identified in the screen also proved to be sufficient for driving liver tumorigenesis at high efficiency when paired with Trp53, including Arid2, B2m, Cic, Kdm5c, Pik3r1, Pten, Stk11, Vh1, and Zc3h13 (FIGS. 13A-13C). The clinical relevance of the Setd2+Trp53 pair in human LIHC was explored, and patients with SETD2 and TP53 double-mutant tumors had significantly worse survival than patients with single-mutant or wildtype tumors. It was thus demonstrated that massively parallel autochthonous in vivo CRISPR screens can be achieved through the use of pooled AAVs in conjunction with MIPS. To date, library-scale CRISPR screens have largely been limited to in vitro or cellular transplant studies. As AAV most often does not integrate into the genome thus direct sgRNA cassette readout is not feasible, a high-throughput in vivo CRISPR experiment was readout by targeted capture sequencing, demonstrating new approaches of doing in vivo CRISPR screens. Whereas traditional sgRNA sequencing can provide information about only the relative abundances of each sgRNA, capture sequencing enables high-resolution analysis of individual indel variants for clonal analysis of tumor heterogeneity.

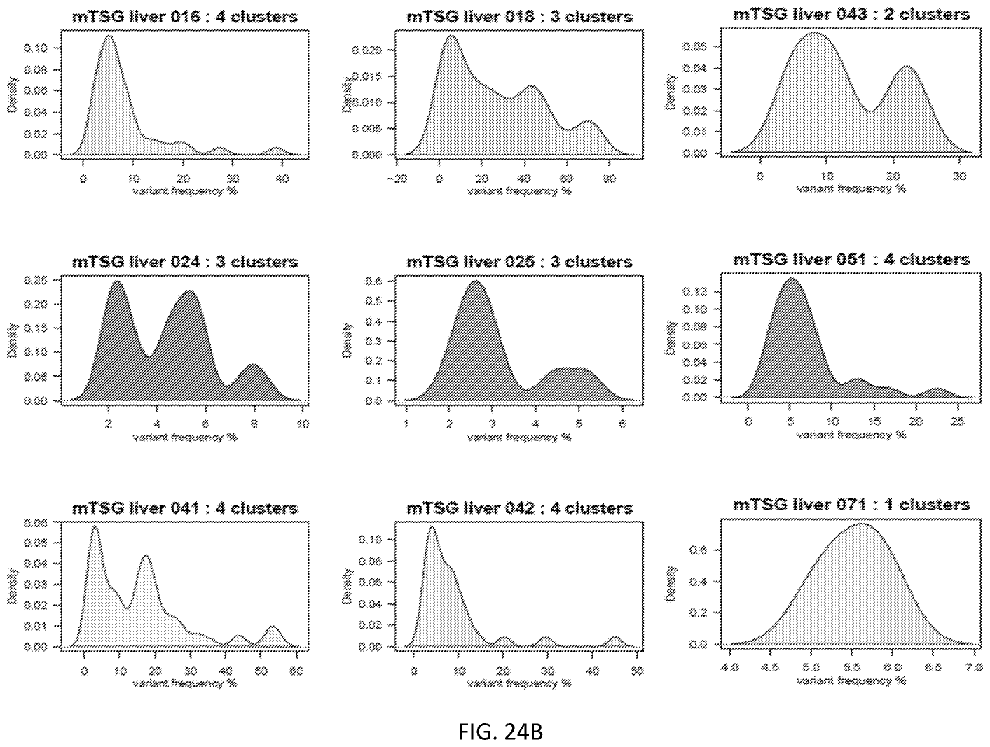

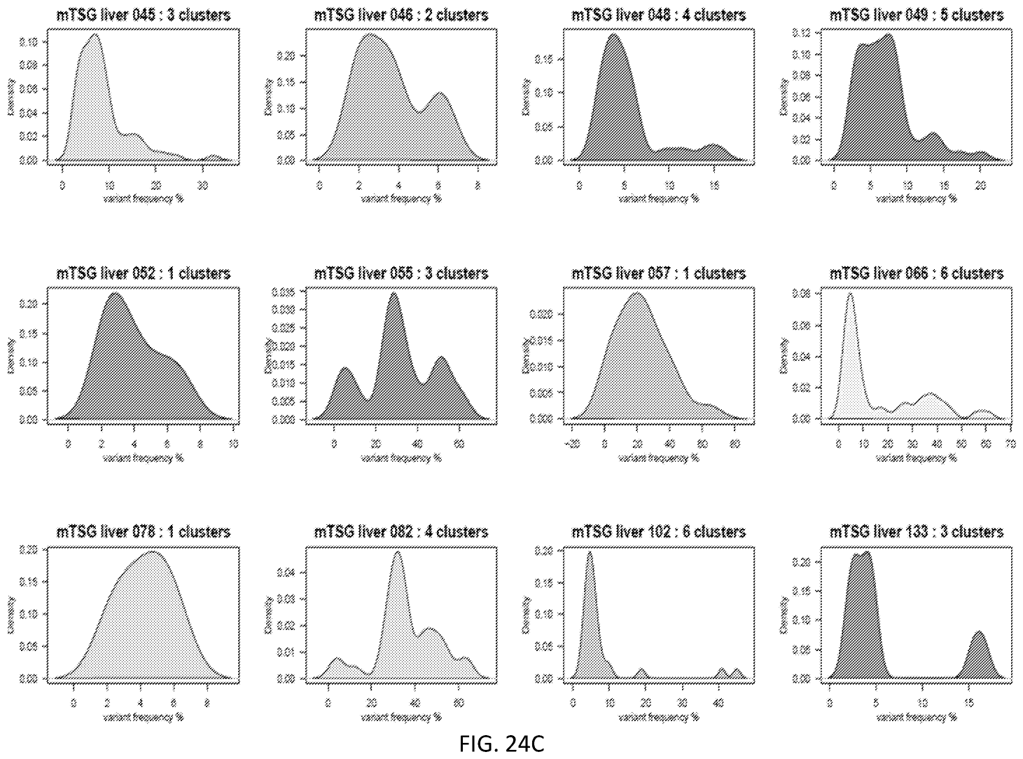

As an approximation to the clonality of the tumors, the number of major clusters was also calculated (FIGS. 24A-24C), in which each major cluster has one or more mutations at similar frequencies as compared to other mutants. From this analysis, it was discovered that 6/30 mTSG livers had single-cluster tumors, with the majority (24/30) being comprised of multiple clusters (FIGS. 24A-24C). Given the nature of pooled mutagenesis, the detected mutations comprising co-occurring gene pairs can either be in the same clone or in different clones within the same tumor. On the basis of allele frequency analysis, one would expect that most of significantly correlated gene pairs had co-evolved in the same clone.

This approach can be extended to identify genetic factors with a significant impact on various cancer types and other human diseases. The present strategy for selecting genes to target in the mTSG library was based on pan-cancer TCGA datasets, rather than being specific to LIHC. This was to identify genes that are more likely to function as tumor suppressors in a wide variety of tissues, with the overarching goal that the same AAV-CRISPR mTSG library could be used in other organs. This approach (AAV-CRISPR mutagenesis followed by MIPS) can be readily expanded to other organ systems, enabling the construction of a multi-organ FCGA of tumor suppressors.

Though the focus was on liver tumor suppressors in this study, given the immense programmability of CRISPR mediated genome editing, it is feasible to apply this AAV-CRISPR screen approach for targeting different gene sets of interest, coding and non-coding elements, and at genome-scale, to functionally assess phenotypes in an unbiased fashion for tackling a wide array of biological problems. The AAV-CRISPR genetically engineered mouse tumor models (GEMMs), developed in fully immunocompetent mice, preserved the native tumor microenvironment, and therefore can be used in high-throughput screening of immunotherapy responses in vivo.

### Other Embodiments

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A method of determining at least one cancer driver mutation in vivo in a cancer-affected subject, the method comprising:
administering to the subject a plurality of AAV-CRISPR vectors, wherein the AAV-CRISPR vectors comprise Cas9 and a plurality of short guide RNAs (sgRNAs) homologous to a plurality of tumor suppressor genes (TSGs); and
sequencing a plurality of nucleic acids isolated from the subject's cancer; whereby analysis of the sequencing data indicates whether any cancer driver mutation is present in the subject's cancer.

2. The method of claim 1, wherein the sgRNA sequences comprise at least one selected from the group consisting of SEQ ID NOs. 1-280.

3. The method of claim 1, wherein the sgRNA sequences comprise SEQ ID NOs. 1-280.

4. The method of claim 1, wherein the sequencing comprises targeted capture sequencing.

5. The method of claim 4, wherein the targeted capture sequencing is performed using a plurality of Molecular Inversion Probes (MIPs).

6. The method of claim 5, wherein the plurality of MIPs comprises at least one selected from the group consisting of SEQ ID NOs. 289-554.

7. The method of claim 5, wherein the plurality of MIPs comprises SEQ ID NOs. 289-554.

8. The method of claim 1, wherein the mutation is a nucleotide insertion.

9. The method of claim 8, wherein the insertion comprises more than one nucleotide base.

10. The method of claim 1, wherein the mutation is a nucleotide deletion.

11. The method of claim 10, wherein the deletion comprises more than one nucleotide base.

12. The method of claim 1, wherein the subject is a mammal.

13. The method of claim 1, wherein the animal is a mouse or a human.

14. A method of identifying a plurality of cancer driver mutations in a sample, the method comprising:
hybridizing a plurality of Molecular Inversion Probes (MIPs) to a plurality of nucleic acids from the sample, and
performing targeted capture sequencing on the plurality of nucleic acids, wherein analyzing the data from the targeted capture sequencing indicates the presence and/or nature of any plurality of cancer driver mutations in the sample.

15. The method of claim 14, wherein the MIPs comprise at least one selected from the group consisting of SEQ ID NOs. 289-554.

16. The method of claim 14, wherein the MIPs comprise SEQ ID NOs. 289-554.

17. A composition comprising a set of Molecular Inversion Probes (MIPs) comprising at least one selected from the group consisting of SEQ ID NOs. 289-554.

18. The composition of claim 17, which comprises SEQ ID NOs. 289-554.

19. A kit comprising the composition of claim 18, and instructional material for use thereof.

20. A kit for determining at least one cancer driver mutation in a sample, the kit comprising the composition of claim 18, reagents for measuring the at least one cancer driver mutation, and instructional material for use thereof.

21. A method of determining at least one cancer driver mutation in a sample, the method comprising:
contacting a plurality of Adeno-Associated Virus-Clustered Regularly Interspaced Short Palidromic Repeats (AAV-CRISPR) vectors with the sample, wherein the vectors comprise Cas9 and a plurality of nucleotide sequences homologous to a plurality of tumor suppressor genes (TSGs), thus generating a reaction mixture;
sequencing a plurality of nucleic acids isolated from the reaction mixture; and
analyzing the sequencing data as to identify any cancer driver mutation therein.

22. A method of determining treatment for a subject suffering from cancer, the method comprising:
contacting a plurality of AAV-CRISPR vectors with a sample from the subject, wherein the vectors comprise Cas9 and a plurality of nucleotide sequences homologous to a plurality of tumor suppressor genes (TSGs), thus generating a reaction mixture;
sequencing a plurality of nucleic acids isolated from the reaction mixture; and
analyzing the data from the sequencing as to identify any mutation in the plurality of nucleic acids,
whereby treatment for the subject suffering from cancer is determined based on the presence and/or nature of any mutation in the plurality of nucleic acids.

23. The method of claim 22, wherein the plurality of nucleotide sequences homologous to a plurality of TSGs comprises at least one selected from the group consisting of SEQ ID NOs. 1-280.

24. The method of claim 22, wherein the plurality of nucleotide sequences homologous to a plurality of TSGs comprises SEQ ID NOs. 1-280.

25. The method of claim 22, wherein the sequencing comprises targeted capture sequencing.

26. The method of claim 22, wherein the mutation is a nucleotide insertion.

27. The method of claim 26, wherein the insertion comprises more than one nucleotide base.

28. The method of claim 22, wherein the mutation is a nucleotide deletion.

29. The method of claim 28, wherein the deletion comprises more than one nucleotide base.

30. The method of claim 22, wherein the sample is a plurality of cancer cells from the subject.

31. The method of claim 22, wherein the sample is a tumor from the subject.

32. An AAV-CRISPR mTSG library comprising a plurality of AAV vectors comprising Cas9 and a plurality of nucleic acids homologous to a plurality of Tumor Suppressor Gene (TSGs).

33. The library of claim 32, wherein the plurality of nucleic acids comprises at least one selected from SEQ ID NOs. 1-280.

34. The library of claim 32, wherein the plurality of nucleic acids comprises SEQ ID NOs. 1-280.

35. A vector comprising an adeno-associated virus (AAV) genome, a U6 promoter gene, an sgRNA sequence, an EFS promoter gene, and a Cre recombinase gene.

36. A vector comprising an adeno-associated virus (AAV) genome, a U6 promoter gene, an sgRNA sequence, a TBG promoter gene, and a Cre recombinase gene.

37. The vector of claim 36, wherein the TBG promoter gene comprises the nucleic acid sequence of SEQ ID NO: 557.

38. A vector comprising the nucleic acid sequence of SEQ ID NO: 555.

39. A vector comprising the nucleic acid sequence of SEQ ID NO: 556.

40. A kit comprising a vector comprising the nucleic acid sequence of SEQ ID NO: 555, and instructional material for use thereof.

41. A kit comprising a vector comprising the nucleic acid sequence of SEQ ID NO: 556, and instructional material for use thereof.

42. A kit comprising an adeno-associated virus (AAV) genome, a U6 promoter gene, an sgRNA sequence, an EFS promoter gene, a Cre recombinase gene, and instructional material for use thereof.

43. A kit comprising an adeno-associated virus (AAV) genome, a U6 promoter gene, an sgRNA sequence, an TBG promoter gene, a Cre recombinase gene, and instructional material for use thereof.

44. The kit of claim 43, wherein the TBG promoter gene comprises the nucleic acid sequence of SEQ ID NO: 557.


SEQUENCE LISTING (557 sequences)

SEQ ID NO: 1 - DNA, 20 bp, Artificial sequence
  Artificially generated seqeunce
gggcagtgtttcaaaatcca

SEQ ID NO: 2 - DNA, 20 bp, Artificial sequence
  Artificially generated seqeunce
ggaacacgagccgtcagcgg

SEQ ID NO: 3 - DNA, 20 bp, Artificial sequence
  Artificially generated seqeunce
ggatttctgttctgcatcaa

SEQ ID NO: 4 - DNA, 20 bp, Artificial sequence
  Artificially generated seqeunce
ggtcccatctgttgcctcca

SEQ ID NO: 5 - DNA, 20 bp, Artificial sequence
  Artificially generated seqeunce
gtttggagatccactcgata

SEQ ID NO: 6 - DNA, 20 bp, Artificial sequence
  Artificially generated seqeunce
gtacccagtgcaagctacag

SEQ ID NO: 7 - DNA, 20 bp, Artificial sequence
  Artificially generated seqeunce
gggtaccccgctatatgttg

SEQ ID NO: 8 - DNA, 20 bp, Artificial sequence
  Artificially generated seqeunce
gcatctggcccccaggagat

SEQ ID NO: 9 - DNA, 20 bp, Artificial sequence
  Artificially generated seqeunce
ggtccgtactgagacatctg

SEQ ID NO: 10 - DNA, 20 bp, Artificial sequence
  Artificially generated seqeunce
gattctgactggcttccagg

SEQ ID NO: 11 - DNA, 20 bp, Artificial sequence
  Artificially generated seqeunce
gtgaaatactctccatcaag

SEQ ID NO: 12 - DNA, 20 bp, Artificial sequence
  Artificially generated seqeunce
gggagaggcgcttgtgcagg

SEQ ID NO: 13 - DNA, 20 bp, Artificial sequence
  Artificially generated seqeunce
gcaaagagaggtacgcaggc

SEQ ID NO: 14 - DNA, 20 bp, Artificial sequence
  Artificially generated seqeunce
gcggttcatgccccccatgc

SEQ ID NO: 15 - DNA, 20 bp, Artificial sequence
  Artificially generated seqeunce
ggtatactcagagccggcct

SEQ ID NO: 16 - DNA, 20 bp, Artificial sequence
  Artificially generated seqeunce
gccatggctgaaggggagcc

SEQ ID NO: 17 - DNA, 20 bp, Artificial sequence
  Artificially generated seqeunce
gactctgtaggtgacagcaa

SEQ ID NO: 18 - DNA, 20 bp, Artificial sequence
  Artificially generated seqeunce
gctcctaaacctacttgttc

SEQ ID NO: 19 - DNA, 20 bp, Artificial sequence
  Artificially generated seqeunce
gcatgctgtatggtaccagg

SEQ ID NO: 20 - DNA, 20 bp, Artificial sequence
  Artificially generated seqeunce
gaaggcatctaccgggtcag

SEQ ID NO: 21 - DNA, 20 bp, Artificial sequence
  Artificially generated seqeunce
ggacgatgatgaggacgatg

SEQ ID NO: 22 - DNA, 20 bp, Artificial sequence
  Artificially generated seqeunce
gtgtaatttcaaagccccct

SEQ ID NO: 23 - DNA, 20 bp, Artificial sequence
  Artificially generated seqeunce
gtggacagcatctagtaggt

SEQ ID NO: 24 - DNA, 20 bp, Artificial sequence
  Artificially generated seqeunce
gggcggttctcttcccaaag

SEQ ID NO: 25 - DNA, 20 bp, Artificial sequence
  Artificially generated seqeunce
ggaagagaaccgccctattg

SEQ ID NO: 26 - DNA, 20 bp, Artificial sequence
  Artificially generated seqeunce
gcatatgctcgtaaagtgga

SEQ ID NO: 27 - DNA, 20 bp, Artificial sequence
  Artificially generated seqeunce
gatgagtgaaaatgctggtg

SEQ ID NO: 28 - DNA, 20 bp, Artificial sequence
  Artificially generated seqeunce
ggctgagctgctgttggcaa

SEQ ID NO: 29 - DNA, 20 bp, Artificial sequence
  Artificially generated seqeunce
gccctaggtgctagtcctat

SEQ ID NO: 30 - DNA, 20 bp, Artificial sequence
  Artificially generated seqeunce
gctagtcctatgggtgtaaa

SEQ ID NO: 31 - DNA, 20 bp, Artificial sequence
  Artificially generated seqeunce
gccggctatgtcgggcctgt

SEQ ID NO: 32 - DNA, 20 bp, Artificial sequence
  Artificially generated seqeunce
gggattcagttctgctgagc

SEQ ID NO: 33 - DNA, 20 bp, Artificial sequence
  Artificially generated seqeunce
gtgtgtgagacatgtgacaa

SEQ ID NO: 34 - DNA, 20 bp, Artificial sequence
  Artificially generated seqeunce
gcaggcaggtcctccatagg

SEQ ID NO: 35 - DNA, 20 bp, Artificial sequence
  Artificially generated seqeunce
gcctcctctgccggagaaag

SEQ ID NO: 36 - DNA, 20 bp, Artificial sequence
  Artificially generated seqeunce
gactgtgaatggacagctga

SEQ ID NO: 37 - DNA, 20 bp, Artificial sequence
  Artificially generated seqeunce
gtcttctcaaaacatttcag

SEQ ID NO: 38 - DNA, 20 bp, Artificial sequence
  Artificially generated seqeunce
gcactgatggaaaatggtga

SEQ ID NO: 39 - DNA, 20 bp, Artificial sequence
  Artificially generated seqeunce
gactaccagttccaaagata

SEQ ID NO: 40 - DNA, 20 bp, Artificial sequence
  Artificially generated seqeunce
gaagcttctggttactttcc

SEQ ID NO: 41 - DNA, 20 bp, Artificial sequence
  Artificially generated seqeunce
gggattggccgcgaagttcc

SEQ ID NO: 42 - DNA, 20 bp, Artificial sequence
  Artificially generated seqeunce
ggggtacgaccgaaagagtt

SEQ ID NO: 43 - DNA, 20 bp, Artificial sequence
  Artificially generated seqeunce
gggtcgcctgccgctcgact

SEQ ID NO: 44 - DNA, 20 bp, Artificial sequence
  Artificially generated seqeunce
gggaacgtcgcccagaccga

SEQ ID NO: 45 - DNA, 20 bp, Artificial sequence
  Artificially generated seqeunce
ggggtacgaccgaaagagtt

SEQ ID NO: 46 - DNA, 20 bp, Artificial sequence
  Artificially generated seqeunce
gtacctgcaccaggaaaacc

SEQ ID NO: 47 - DNA, 20 bp, Artificial sequence
  Artificially generated seqeunce
gtggagccatacattgcatg

SEQ ID NO: 48 - DNA, 20 bp, Artificial sequence
  Artificially generated seqeunce
gggtgagttttctgtctagt

SEQ ID NO: 49 - DNA, 20 bp, Artificial sequence
  Artificially generated seqeunce
gcctttgtcatcagaattcg

SEQ ID NO: 50 - DNA, 20 bp, Artificial sequence
  Artificially generated seqeunce
gaaggcaaagcactatcaca

SEQ ID NO: 51 - DNA, 20 bp, Artificial sequence
  Artificially generated seqeunce
gcaatggtcttgagatctat

SEQ ID NO: 52 - DNA, 20 bp, Artificial sequence
  Artificially generated seqeunce
gaccattgctcagaggatac

SEQ ID NO: 53 - DNA, 20 bp, Artificial sequence
  Artificially generated seqeunce
gcctgggtctcaagtattca

SEQ ID NO: 54 - DNA, 20 bp, Artificial sequence
  Artificially generated seqeunce
gccaaaacatacaatgagcc

SEQ ID NO: 55 - DNA, 20 bp, Artificial sequence
  Artificially generated seqeunce
gtgcgaaggacctgtcagcc

SEQ ID NO: 56 - DNA, 20 bp, Artificial sequence
  Artificially generated seqeunce
gactgcatgggcagaaggga

SEQ ID NO: 57 - DNA, 20 bp, Artificial sequence
  Artificially generated seqeunce
gagacggcactttccttgtc

SEQ ID NO: 58 - DNA, 20 bp, Artificial sequence
  Artificially generated seqeunce
gttggctacagtagtgggct

SEQ ID NO: 59 - DNA, 20 bp, Artificial sequence
  Artificially generated seqeunce
ggcagtgctgcaggcaaaag

SEQ ID NO: 60 - DNA, 20 bp, Artificial sequence
  Artificially generated seqeunce
ggctgacgcagaaaggtgtg

SEQ ID NO: 61 - DNA, 20 bp, Artificial sequence
  Artificially generated seqeunce
ggccgcaagctgacgcctca

SEQ ID NO: 62 - DNA, 20 bp, Artificial sequence
  Artificially generated seqeunce
gcctcagggacagagagacc

SEQ ID NO: 63 - DNA, 20 bp, Artificial sequence
  Artificially generated seqeunce
gtccctgaggcgtcagcttg

SEQ ID NO: 64 - DNA, 20 bp, Artificial sequence
  Artificially generated seqeunce
gggccgcaagctgacgcctc

SEQ ID NO: 65 - DNA, 20 bp, Artificial sequence
  Artificially generated seqeunce
gttgaaacagagcggggggg

SEQ ID NO: 66 - DNA, 20 bp, Artificial sequence
  Artificially generated seqeunce
gtggatgaaaggctcttcat

SEQ ID NO: 67 - DNA, 20 bp, Artificial sequence
  Artificially generated seqeunce
ggttttgcacagtctcttcc

SEQ ID NO: 68 - DNA, 20 bp, Artificial sequence
  Artificially generated seqeunce
gacctcaggctgaacagcct

SEQ ID NO: 69 - DNA, 20 bp, Artificial sequence
  Artificially generated seqeunce
ggcccaggctgttcagcctg

SEQ ID NO: 70 - DNA, 20 bp, Artificial sequence
  Artificially generated seqeunce
gtccaccaccccctggtcac

SEQ ID NO: 71 - DNA, 20 bp, Artificial sequence
  Artificially generated seqeunce
gttggcactgatttcataac

SEQ ID NO: 72 - DNA, 20 bp, Artificial sequence
  Artificially generated seqeunce
gggagaagatagcaagatgc

SEQ ID NO: 73 - DNA, 20 bp, Artificial sequence
  Artificially generated seqeunce
gtggctactgaccaaaccca

SEQ ID NO: 74 - DNA, 20 bp, Artificial sequence
  Artificially generated seqeunce
gagaattcctaacagctatg

SEQ ID NO: 75 - DNA, 20 bp, Artificial sequence
  Artificially generated seqeunce
gctgccgatactccaaactt

SEQ ID NO: 76 - DNA, 20 bp, Artificial sequence
  Artificially generated seqeunce
gcaactattttacaacaatt

SEQ ID NO: 77 - DNA, 20 bp, Artificial sequence
  Artificially generated seqeunce
ggtaaattaaaacactcacc

SEQ ID NO: 78 - DNA, 20 bp, Artificial sequence
  Artificially generated seqeunce
gtaaattaaaacactcacct

SEQ ID NO: 79 - DNA, 20 bp, Artificial sequence
  Artificially generated seqeunce
gcagcattttcagttagctt

SEQ ID NO: 80 - DNA, 20 bp, Artificial sequence
  Artificially generated seqeunce
ggctattaaagcatttcagg

SEQ ID NO: 81 - DNA, 20 bp, Artificial sequence
  Artificially generated seqeunce
gtgattttgatctcgtgcct

SEQ ID NO: 82 - DNA, 20 bp, Artificial sequence
  Artificially generated seqeunce
gcaaggtacactgtaatcag

SEQ ID NO: 83 - DNA, 20 bp, Artificial sequence
  Artificially generated seqeunce
gtgcttatgaatccatgaaa

SEQ ID NO: 84 - DNA, 20 bp, Artificial sequence
  Artificially generated seqeunce
gtccaaatatatagtaaggt

SEQ ID NO: 85 - DNA, 20 bp, Artificial sequence
  Artificially generated seqeunce
gagacttgaggaaaatgtta

SEQ ID NO: 86 - DNA, 20 bp, Artificial sequence
  Artificially generated seqeunce
ggggccaagggtatgccaga

SEQ ID NO: 87 - DNA, 20 bp, Artificial sequence
  Artificially generated seqeunce
gactgtgggatcccagtttc

SEQ ID NO: 88 - DNA, 20 bp, Artificial sequence
  Artificially generated seqeunce
gtaggtaggaggtgaactca

SEQ ID NO: 89 - DNA, 20 bp, Artificial sequence
  Artificially generated seqeunce
gcatgttcaacatcgtaggt

SEQ ID NO: 90 - DNA, 20 bp, Artificial sequence
  Artificially generated seqeunce
ggagtcttctgcctggttcc

SEQ ID NO: 91 - DNA, 20 bp, Artificial sequence
  Artificially generated seqeunce
ggactacccaagtgtgcgga

SEQ ID NO: 92 - DNA, 20 bp, Artificial sequence
  Artificially generated seqeunce
gcaccttgagagtcagcacc

SEQ ID NO: 93 - DNA, 20 bp, Artificial sequence
  Artificially generated seqeunce
ggttaaccagaagtccatca

SEQ ID NO: 94 - DNA, 20 bp, Artificial sequence
  Artificially generated seqeunce
gtgccatccctcaatgtcga

SEQ ID NO: 95 - DNA, 20 bp, Artificial sequence
  Artificially generated seqeunce
gtcctgaggagatggaggct

SEQ ID NO: 96 - DNA, 20 bp, Artificial sequence
  Artificially generated seqeunce
gtctccttcttctagaggca

SEQ ID NO: 97 - DNA, 20 bp, Artificial sequence
  Artificially generated seqeunce
ggcaaaacagaataggagag

SEQ ID NO: 98 - DNA, 20 bp, Artificial sequence
  Artificially generated seqeunce
ggcaatttgatacaagtaac

SEQ ID NO: 99 - DNA, 20 bp, Artificial sequence
  Artificially generated seqeunce
gcaatttgatacaagtaact

SEQ ID NO: 100 - DNA, 20 bp, Artificial sequence
  Artificially generated seqeunce
gcacagtatcaaaatacttg

SEQ ID NO: 101 - DNA, 20 bp, Artificial sequence
  Artificially generated seqeunce
gacaaagttgatgaaactgg

SEQ ID NO: 102 - DNA, 20 bp, Artificial sequence
  Artificially generated seqeunce
gccgatttccttatccaaag

SEQ ID NO: 103 - DNA, 20 bp, Artificial sequence
  Artificially generated seqeunce
gacccaagtgcatcaagaca

SEQ ID NO: 104 - DNA, 20 bp, Artificial sequence
  Artificially generated seqeunce
gcacttgggtctattctttc

SEQ ID NO: 105 - DNA, 20 bp, Artificial sequence
  Artificially generated seqeunce
gggcgactgttggatctgta

SEQ ID NO: 106 - DNA, 20 bp, Artificial sequence
  Artificially generated seqeunce
gtccagtaaaagctggagga

SEQ ID NO: 107 - DNA, 20 bp, Artificial sequence
  Artificially generated seqeunce
gagtggttctgaaatccaca

SEQ ID NO: 108 - DNA, 20 bp, Artificial sequence
  Artificially generated seqeunce
ggagagcaatgttaagctct

SEQ ID NO: 109 - DNA, 20 bp, Artificial sequence
  Artificially generated seqeunce
gactgtgtgcagagagcaac

SEQ ID NO: 110 - DNA, 20 bp, Artificial sequence
  Artificially generated seqeunce
gtcacttctcattacagttt

SEQ ID NO: 111 - DNA, 20 bp, Artificial sequence
  Artificially generated seqeunce
gatgccctgcagaggaaagg

SEQ ID NO: 112 - DNA, 20 bp, Artificial sequence
  Artificially generated seqeunce
ggcagagcgcttcagtgagc

SEQ ID NO: 113 - DNA, 20 bp, Artificial sequence
  Artificially generated seqeunce
gacagtgtgctgagagaccg

SEQ ID NO: 114 - DNA, 20 bp, Artificial sequence
  Artificially generated seqeunce
ggccggaaattcccagcttc

SEQ ID NO: 115 - DNA, 20 bp, Artificial sequence
  Artificially generated seqeunce
gtgtttcttttggtcttagg

SEQ ID NO: 116 - DNA, 20 bp, Artificial sequence
  Artificially generated seqeunce
gtgtttctccctttaagtct

SEQ ID NO: 117 - DNA, 20 bp, Artificial sequence
  Artificially generated seqeunce
ggcagccccaattctgctca

SEQ ID NO: 118 - DNA, 20 bp, Artificial sequence
  Artificially generated seqeunce
gatattagccgtgactcaga

SEQ ID NO: 119 - DNA, 20 bp, Artificial sequence
  Artificially generated seqeunce
gaagacaaagatgattttaa

SEQ ID NO: 120 - DNA, 20 bp, Artificial sequence
  Artificially generated seqeunce
ggtttcctacaaaagagtta

SEQ ID NO: 121 - DNA, 20 bp, Artificial sequence
  Artificially generated seqeunce
gctcattggttggtttctgc

SEQ ID NO: 122 - DNA, 20 bp, Artificial sequence
  Artificially generated seqeunce
gcctttctccaaaaggtatg

SEQ ID NO: 123 - DNA, 20 bp, Artificial sequence
  Artificially generated seqeunce
ggttagtatggctccgcgtg

SEQ ID NO: 124 - DNA, 20 bp, Artificial sequence
  Artificially generated seqeunce
gcgttacttccagattaacc

SEQ ID NO: 125 - DNA, 20 bp, Artificial sequence
  Artificially generated seqeunce
gcgttacttccagattaacc

SEQ ID NO: 126 - DNA, 20 bp, Artificial sequence
  Artificially generated seqeunce
gataaacctcttaggattac

SEQ ID NO: 127 - DNA, 20 bp, Artificial sequence
  Artificially generated seqeunce
ggaacgggctggtgttaaaa

SEQ ID NO: 128 - DNA, 20 bp, Artificial sequence
  Artificially generated seqeunce
gtcttcccttttcaacaatc

SEQ ID NO: 129 - DNA, 20 bp, Artificial sequence
  Artificially generated seqeunce
gaaaagggaagaccagcccc

SEQ ID NO: 130 - DNA, 20 bp, Artificial sequence
  Artificially generated seqeunce
gttagcatacaagacctttc

SEQ ID NO: 131 - DNA, 20 bp, Artificial sequence
  Artificially generated seqeunce
ggaggcggtgagtagtaagg

SEQ ID NO: 132 - DNA, 20 bp, Artificial sequence
  Artificially generated seqeunce
gaggaggcggtgagtagtaa

SEQ ID NO: 133 - DNA, 20 bp, Artificial sequence
  Artificially generated seqeunce
gaattttggacatgctaggg

SEQ ID NO: 134 - DNA, 20 bp, Artificial sequence
  Artificially generated seqeunce
gtgagcctgttttctcctct

SEQ ID NO: 135 - DNA, 20 bp, Artificial sequence
  Artificially generated seqeunce
ggttacctagaggagaaaac

SEQ ID NO: 136 - DNA, 20 bp, Artificial sequence
  Artificially generated seqeunce
gtatacaccttcataacctg

SEQ ID NO: 137 - DNA, 20 bp, Artificial sequence
  Artificially generated seqeunce
gcagtggccattgtgcagac

SEQ ID NO: 138 - DNA, 20 bp, Artificial sequence
  Artificially generated seqeunce
ggcacctggtgaaagaggca

SEQ ID NO: 139 - DNA, 20 bp, Artificial sequence
  Artificially generated seqeunce
gccaacccttgtgagcacgc

SEQ ID NO: 140 - DNA, 20 bp, Artificial sequence
  Artificially generated seqeunce
gagcacactcatccacgtcc

SEQ ID NO: 141 - DNA, 20 bp, Artificial sequence
  Artificially generated seqeunce
ggtgacaagggtgtcgtcta

SEQ ID NO: 142 - DNA, 20 bp, Artificial sequence
  Artificially generated seqeunce
gtgtcgtctatggaacggat

SEQ ID NO: 143 - DNA, 20 bp, Artificial sequence
  Artificially generated seqeunce
ggtaaactttgtctgaagtc

SEQ ID NO: 144 - DNA, 20 bp, Artificial sequence
  Artificially generated seqeunce
ggagttgggttatgtcttcc

SEQ ID NO: 145 - DNA, 20 bp, Artificial sequence
  Artificially generated seqeunce
gactcactgtcttgttctct

SEQ ID NO: 146 - DNA, 20 bp, Artificial sequence
  Artificially generated seqeunce
gagtgtttgtacatagatac

SEQ ID NO: 147 - DNA, 20 bp, Artificial sequence
  Artificially generated seqeunce
gcagaacggaataaaatgat

SEQ ID NO: 148 - DNA, 20 bp, Artificial sequence
  Artificially generated seqeunce
gatgactgctttggtaaatg

SEQ ID NO: 149 - DNA, 20 bp, Artificial sequence
  Artificially generated seqeunce
gattacccacttaccatggc

SEQ ID NO: 150 - DNA, 20 bp, Artificial sequence
  Artificially generated seqeunce
gaggaccagccatggtaagt

SEQ ID NO: 151 - DNA, 20 bp, Artificial sequence
  Artificially generated seqeunce
gctctgcagagtatattccc

SEQ ID NO: 152 - DNA, 20 bp, Artificial sequence
  Artificially generated seqeunce
gcatgtaggtgatgcagggc

SEQ ID NO: 153 - DNA, 20 bp, Artificial sequence
  Artificially generated seqeunce
gtttgtcatcttcatctcct

SEQ ID NO: 154 - DNA, 20 bp, Artificial sequence
  Artificially generated seqeunce
gtatgccgaatgtgttcccg

SEQ ID NO: 155 - DNA, 20 bp, Artificial sequence
  Artificially generated seqeunce
gaccttcctagaaggcaagg

SEQ ID NO: 156 - DNA, 20 bp, Artificial sequence
  Artificially generated seqeunce
gtccatttcaaagtaagcaa

SEQ ID NO: 157 - DNA, 20 bp, Artificial sequence
  Artificially generated seqeunce
gcaatggagcaccagtactc

SEQ ID NO: 158 - DNA, 20 bp, Artificial sequence
  Artificially generated seqeunce
gatgattggaaatgggaggc

SEQ ID NO: 159 - DNA, 20 bp, Artificial sequence
  Artificially generated seqeunce
gtcacaacagggcagcttga

SEQ ID NO: 160 - DNA, 20 bp, Artificial sequence
  Artificially generated seqeunce
gatggctatgtggatccttc

SEQ ID NO: 161 - DNA, 20 bp, Artificial sequence
  Artificially generated seqeunce
gggctcccgtgggcacttca

SEQ ID NO: 162 - DNA, 20 bp, Artificial sequence
  Artificially generated seqeunce
gaaaaccctgaagtgcccac

SEQ ID NO: 163 - DNA, 20 bp, Artificial sequence
  Artificially generated seqeunce
gaagattccccgggtgggcc

SEQ ID NO: 164 - DNA, 20 bp, Artificial sequence
  Artificially generated seqeunce
gggtgggcccggaacatctc

SEQ ID NO: 165 - DNA, 20 bp, Artificial sequence
  Artificially generated seqeunce
gattgcgatgcgctcatggc

SEQ ID NO: 166 - DNA, 20 bp, Artificial sequence
  Artificially generated seqeunce
gcgcgcggggggcatgttgg

SEQ ID NO: 167 - DNA, 20 bp, Artificial sequence
  Artificially generated seqeunce
gcctcctccaggcgcgcggg

SEQ ID NO: 168 - DNA, 20 bp, Artificial sequence
  Artificially generated seqeunce
gtcctagtgtagggaccggg

SEQ ID NO: 169 - DNA, 20 bp, Artificial sequence
  Artificially generated seqeunce
gagggttgggcgtgggggct

SEQ ID NO: 170 - DNA, 20 bp, Artificial sequence
  Artificially generated seqeunce
gtagaggtgcgtatctgtca

SEQ ID NO: 171 - DNA, 20 bp, Artificial sequence
  Artificially generated seqeunce
gttcgaggtgaaccattaat

SEQ ID NO: 172 - DNA, 20 bp, Artificial sequence
  Artificially generated seqeunce
gaggtcagaacaggagcgct

SEQ ID NO: 173 - DNA, 20 bp, Artificial sequence
  Artificially generated seqeunce
ggctctctgagtagtgcagg

SEQ ID NO: 174 - DNA, 20 bp, Artificial sequence
  Artificially generated seqeunce
gaatcatggaatcccttgca

SEQ ID NO: 175 - DNA, 20 bp, Artificial sequence
  Artificially generated seqeunce
gaacctttttattcctagga

SEQ ID NO: 176 - DNA, 20 bp, Artificial sequence
  Artificially generated seqeunce
gaggcagaacgtcgtaaaga

SEQ ID NO: 177 - DNA, 20 bp, Artificial sequence
  Artificially generated seqeunce
gttctcttccggcgaggaga

SEQ ID NO: 178 - DNA, 20 bp, Artificial sequence
  Artificially generated seqeunce
ggaggtggactcggagtgcg

SEQ ID NO: 179 - DNA, 20 bp, Artificial sequence
  Artificially generated seqeunce
gagatgggaaggacagaggc

SEQ ID NO: 180 - DNA, 20 bp, Artificial sequence
  Artificially generated seqeunce
gactttctcagagaaggtga

SEQ ID NO: 181 - DNA, 20 bp, Artificial sequence
  Artificially generated seqeunce
gaaccgacaaacagtcctgg

SEQ ID NO: 182 - DNA, 20 bp, Artificial sequence
  Artificially generated seqeunce
ggtcaggcaccactgccatc

SEQ ID NO: 183 - DNA, 20 bp, Artificial sequence
  Artificially generated seqeunce
gtcctctccccagggcccta

SEQ ID NO: 184 - DNA, 20 bp, Artificial sequence
  Artificially generated seqeunce
gtggacagataaagctcgaa

SEQ ID NO: 185 - DNA, 20 bp, Artificial sequence
  Artificially generated seqeunce
gctatgtgcctatcacaggg

SEQ ID NO: 186 - DNA, 20 bp, Artificial sequence
  Artificially generated seqeunce
gggatacctacctgaatcca

SEQ ID NO: 187 - DNA, 20 bp, Artificial sequence
  Artificially generated seqeunce
gggaggtgggggactccacg

SEQ ID NO: 188 - DNA, 20 bp, Artificial sequence
  Artificially generated seqeunce
gtcccctttgtagatctaag

SEQ ID NO: 189 - DNA, 20 bp, Artificial sequence
  Artificially generated seqeunce
ggagatcccatgacttctac

SEQ ID NO: 190 - DNA, 20 bp, Artificial sequence
  Artificially generated seqeunce
ggggaggggacacctacaga

SEQ ID NO: 191 - DNA, 20 bp, Artificial sequence
  Artificially generated seqeunce
gagattattctctgtatttt

SEQ ID NO: 192 - DNA, 20 bp, Artificial sequence
  Artificially generated seqeunce
gtcttaatgtctttccttta

SEQ ID NO: 193 - DNA, 20 bp, Artificial sequence
  Artificially generated seqeunce
gatcttcttctcggccctaa

SEQ ID NO: 194 - DNA, 20 bp, Artificial sequence
  Artificially generated seqeunce
gttcacaatgagttagaaga

SEQ ID NO: 195 - DNA, 20 bp, Artificial sequence
  Artificially generated seqeunce
ggacactgagatatatctat

SEQ ID NO: 196 - DNA, 20 bp, Artificial sequence
  Artificially generated seqeunce
gtccatggtagttgatctta

SEQ ID NO: 197 - DNA, 20 bp, Artificial sequence
  Artificially generated seqeunce
gctgcagccaagagctcttg

SEQ ID NO: 198 - DNA, 20 bp, Artificial sequence
  Artificially generated seqeunce
gattatccgaattcttagca

SEQ ID NO: 199 - DNA, 20 bp, Artificial sequence
  Artificially generated seqeunce
gacaatctgatgctatatct

SEQ ID NO: 200 - DNA, 20 bp, Artificial sequence
  Artificially generated seqeunce
ggtatattttccaagtcttg

SEQ ID NO: 201 - DNA, 20 bp, Artificial sequence
  Artificially generated seqeunce
gtggagagctgtctcaccag

SEQ ID NO: 202 - DNA, 20 bp, Artificial sequence
  Artificially generated seqeunce
gggtgtggaggtgtctgatg

SEQ ID NO: 203 - DNA, 20 bp, Artificial sequence
  Artificially generated seqeunce
ggtcatgcacaggtggcggc

SEQ ID NO: 204 - DNA, 20 bp, Artificial sequence
  Artificially generated seqeunce
gatggcacagctctgaagag

SEQ ID NO: 205 - DNA, 20 bp, Artificial sequence
  Artificially generated seqeunce
gctctggaagtgcaggcttg

SEQ ID NO: 206 - DNA, 20 bp, Artificial sequence
  Artificially generated seqeunce
ggtggtgaggtccgaaggag

SEQ ID NO: 207 - DNA, 20 bp, Artificial sequence
  Artificially generated seqeunce
ggaagggtggtgaggtccga

SEQ ID NO: 208 - DNA, 20 bp, Artificial sequence
  Artificially generated seqeunce
gcccacaggcattgcagacc

SEQ ID NO: 209 - DNA, 20 bp, Artificial sequence
  Artificially generated seqeunce
gaggaacgctaatggggacc

SEQ ID NO: 210 - DNA, 20 bp, Artificial sequence
  Artificially generated seqeunce
gtaccatctcgccgccacag

SEQ ID NO: 211 - DNA, 20 bp, Artificial sequence
  Artificially generated seqeunce
ggttcattgtcactaacatc

SEQ ID NO: 212 - DNA, 20 bp, Artificial sequence
  Artificially generated seqeunce
gaatgctgatcttcatcaaa

SEQ ID NO: 213 - DNA, 20 bp, Artificial sequence
  Artificially generated seqeunce
gaacttgtcctcccgccgcg

SEQ ID NO: 214 - DNA, 20 bp, Artificial sequence
  Artificially generated seqeunce
gttcttcataccaggaccag

SEQ ID NO: 215 - DNA, 20 bp, Artificial sequence
  Artificially generated seqeunce
gggaattgtgactccctgat

SEQ ID NO: 216 - DNA, 20 bp, Artificial sequence
  Artificially generated seqeunce
gctgcagaagaaaaagatac

SEQ ID NO: 217 - DNA, 20 bp, Artificial sequence
  Artificially generated seqeunce
gcgccacttttgggggtaag

SEQ ID NO: 218 - DNA, 20 bp, Artificial sequence
  Artificially generated seqeunce
gaacctagattttgagacag

SEQ ID NO: 219 - DNA, 20 bp, Artificial sequence
  Artificially generated seqeunce
gaattttcttcagcctctcc

SEQ ID NO: 220 - DNA, 20 bp, Artificial sequence
  Artificially generated seqeunce
gagggctgcgccacttttgg

SEQ ID NO: 221 - DNA, 20 bp, Artificial sequence
  Artificially generated seqeunce
ggctacccaaatatgaatca

SEQ ID NO: 222 - DNA, 20 bp, Artificial sequence
  Artificially generated seqeunce
ggacccccatatcctatggg

SEQ ID NO: 223 - DNA, 20 bp, Artificial sequence
  Artificially generated seqeunce
gctgcctaggatagcctcct

SEQ ID NO: 224 - DNA, 20 bp, Artificial sequence
  Artificially generated seqeunce
gacgcatgagccattctccc

SEQ ID NO: 225 - DNA, 20 bp, Artificial sequence
  Artificially generated seqeunce
gaagtgtactggggcatctg

SEQ ID NO: 226 - DNA, 20 bp, Artificial sequence
  Artificially generated seqeunce
ggagagagtttacttccgag

SEQ ID NO: 227 - DNA, 20 bp, Artificial sequence
  Artificially generated seqeunce
gtctttgtcctgaggcctta

SEQ ID NO: 228 - DNA, 20 bp, Artificial sequence
  Artificially generated seqeunce
gtggagtgctgcactggccc

SEQ ID NO: 229 - DNA, 20 bp, Artificial sequence
  Artificially generated seqeunce
gctgtgagtgaatgatgttg

SEQ ID NO: 230 - DNA, 20 bp, Artificial sequence
  Artificially generated seqeunce
gccagtgttttgagttctag

SEQ ID NO: 231 - DNA, 20 bp, Artificial sequence
  Artificially generated seqeunce
gtctacaagcataatcacac

SEQ ID NO: 232 - DNA, 20 bp, Artificial sequence
  Artificially generated seqeunce
gattatgcttgtagacaggt

SEQ ID NO: 233 - DNA, 20 bp, Artificial sequence
  Artificially generated seqeunce
gatggcgtagagggggaaaa

SEQ ID NO: 234 - DNA, 20 bp, Artificial sequence
  Artificially generated seqeunce
gataactgtgctggtccaga

SEQ ID NO: 235 - DNA, 20 bp, Artificial sequence
  Artificially generated seqeunce
gctatgacagtgtcacaatg

SEQ ID NO: 236 - DNA, 20 bp, Artificial sequence
  Artificially generated seqeunce
gtacaggcaggaggcaactg

SEQ ID NO: 237 - DNA, 20 bp, Artificial sequence
  Artificially generated seqeunce
gcaggaggcaactggggact

SEQ ID NO: 238 - DNA, 20 bp, Artificial sequence
  Artificially generated seqeunce
ggggtgcacagtcttgatgg

SEQ ID NO: 239 - DNA, 20 bp, Artificial sequence
  Artificially generated seqeunce
gtgtagccgttctgctccac

SEQ ID NO: 240 - DNA, 20 bp, Artificial sequence
  Artificially generated seqeunce
gtaccttggccactagtggg

SEQ ID NO: 241 - DNA, 20 bp, Artificial sequence
  Artificially generated seqeunce
gtggaacggcacatgtgtga

SEQ ID NO: 242 - DNA, 20 bp, Artificial sequence
  Artificially generated seqeunce
ggaacggcacatgtgtgatg

SEQ ID NO: 243 - DNA, 20 bp, Artificial sequence
  Artificially generated seqeunce
gacttcaggaattagtacgc

SEQ ID NO: 244 - DNA, 20 bp, Artificial sequence
  Artificially generated seqeunce
gaaggtcactgggcttagga

SEQ ID NO: 245 - DNA, 20 bp, Artificial sequence
  Artificially generated seqeunce
gtctgcagatgaaggtcact

SEQ ID NO: 246 - DNA, 20 bp, Artificial sequence
  Artificially generated seqeunce
gactccttgtctgaccccac

SEQ ID NO: 247 - DNA, 20 bp, Artificial sequence
  Artificially generated seqeunce
gaggaccattgtcatccgcc

SEQ ID NO: 248 - DNA, 20 bp, Artificial sequence
  Artificially generated seqeunce
ggatgtaatggagatagtcc

SEQ ID NO: 249 - DNA, 20 bp, Artificial sequence
  Artificially generated seqeunce
gtcacctgaaacagggggac

SEQ ID NO: 250 - DNA, 20 bp, Artificial sequence
  Artificially generated seqeunce
gtcggatcctgtctggtgag

SEQ ID NO: 251 - DNA, 20 bp, Artificial sequence
  Artificially generated seqeunce
ggagcccgaggaggggtttg

SEQ ID NO: 252 - DNA, 20 bp, Artificial sequence
  Artificially generated seqeunce
gggcgcaggccttcctggag

SEQ ID NO: 253 - DNA, 20 bp, Artificial sequence
  Artificially generated seqeunce
gaagaaacaccctctggctg

SEQ ID NO: 254 - DNA, 20 bp, Artificial sequence
  Artificially generated seqeunce
gtgtctgggcttggtgggat

SEQ ID NO: 255 - DNA, 20 bp, Artificial sequence
  Artificially generated seqeunce
gtgctgcctaatctgtcgga

SEQ ID NO: 256 - DNA, 20 bp, Artificial sequence
  Artificially generated seqeunce
gctccacagtgccagcgttc

SEQ ID NO: 257 - DNA, 20 bp, Artificial sequence
  Artificially generated seqeunce
gcgaagaagaatctaagagg

SEQ ID NO: 258 - DNA, 20 bp, Artificial sequence
  Artificially generated seqeunce
ggagaagcactgccgggata

SEQ ID NO: 259 - DNA, 20 bp, Artificial sequence
  Artificially generated seqeunce
ggttagcggagcagtgtcca

SEQ ID NO: 260 - DNA, 20 bp, Artificial sequence
  Artificially generated seqeunce
ggtgctggcgcaggagagcc

SEQ ID NO: 261 - DNA, 20 bp, Artificial sequence
  Artificially generated seqeunce
gaaaacagccaaggtttgta

SEQ ID NO: 262 - DNA, 20 bp, Artificial sequence
  Artificially generated seqeunce
gggtcaagtgcctgagaatg

SEQ ID NO: 263 - DNA, 20 bp, Artificial sequence
  Artificially generated seqeunce
gagttaccctacatacactc

SEQ ID NO: 264 - DNA, 20 bp, Artificial sequence
  Artificially generated seqeunce
gttcaggctgctgaccttca

SEQ ID NO: 265 - DNA, 20 bp, Artificial sequence
  Artificially generated seqeunce
ggaggttcctgtcaaaggag

SEQ ID NO: 266 - DNA, 20 bp, Artificial sequence
  Artificially generated seqeunce
ggtcttgggctcggccatac

SEQ ID NO: 267 - DNA, 20 bp, Artificial sequence
  Artificially generated seqeunce
gggtgaattcagtgtgagcc

SEQ ID NO: 268 - DNA, 20 bp, Artificial sequence
  Artificially generated seqeunce
gagcccaagaccgtctactg

SEQ ID NO: 269 - DNA, 20 bp, Artificial sequence
  Artificially generated seqeunce
gtatgtatcagtctcagtgg

SEQ ID NO: 270 - DNA, 20 bp, Artificial sequence
  Artificially generated seqeunce
ggtcgcttcagtcgtcagca

SEQ ID NO: 271 - DNA, 20 bp, Artificial sequence
  Artificially generated seqeunce
gccgcttgcagcaggtcttt

SEQ ID NO: 272 - DNA, 20 bp, Artificial sequence
  Artificially generated seqeunce
gcagcaggtctttgggttcc

SEQ ID NO: 273 - DNA, 20 bp, Artificial sequence
  Artificially generated seqeunce
gagtgtatacatactttata

SEQ ID NO: 274 - DNA, 20 bp, Artificial sequence
  Artificially generated seqeunce
gtatgcatctccatgaaaaa

SEQ ID NO: 275 - DNA, 20 bp, Artificial sequence
  Artificially generated seqeunce
gatctgtacacttttcttat

SEQ ID NO: 276 - DNA, 20 bp, Artificial sequence
  Artificially generated seqeunce
gcggggcgcactgggcagcg

SEQ ID NO: 277 - DNA, 20 bp, Artificial sequence
  Artificially generated seqeunce
gccaccgctgcccactgaga

SEQ ID NO: 278 - DNA, 20 bp, Artificial sequence
  Artificially generated seqeunce
gacggcaaaccctgccaggc

SEQ ID NO: 279 - DNA, 20 bp, Artificial sequence
  Artificially generated seqeunce
gccatgcagcagcacgccgt

SEQ ID NO: 280 - DNA, 20 bp, Artificial sequence
  Artificially generated seqeunce
gccgtggggggctactgcaa

SEQ ID NO: 281 - DNA, 20 bp, Artificial sequence
  Artificially generated seqeunce
acggaggctaagcgtcgcaa

SEQ ID NO: 282 - DNA, 20 bp, Artificial sequence
  Artificially generated seqeunce
cgcttccgcggcccgttcaa

SEQ ID NO: 283 - DNA, 20 bp, Artificial sequence
  Artificially generated seqeunce
atcgtttccgcttaacggcg

SEQ ID NO: 284 - DNA, 20 bp, Artificial sequence
  Artificially generated seqeunce
gtaggcgcgccgctctctac

SEQ ID NO: 285 - DNA, 20 bp, Artificial sequence
  Artificially generated seqeunce
ccatatcggggcgagacatg

SEQ ID NO: 286 - DNA, 20 bp, Artificial sequence
  Artificially generated seqeunce
tactaacgccgctcctacag

SEQ ID NO: 287 - DNA, 20 bp, Artificial sequence
  Artificially generated seqeunce
tgaggatcatgtcgagcgcc

SEQ ID NO: 288 - DNA, 20 bp, Artificial sequence
  Artificially generated seqeunce
gggcccgcataggatatcgc

SEQ ID NO: 289 - DNA, 81 bp, Artificial sequence
  Artificially generated seqeunce
  misc_feature (52)..(57): n is a, c, g, or t
atggctccacaatccgcagcacttcagcttcccgatatccgacggtagtgtnnnnnntctgtcaaggcaatccgcttcttg

SEQ ID NO: 290 - DNA, 81 bp, Artificial sequence
  Artificially generated seqeunce
  misc_feature (54)..(59): n is a, c, g, or t
ggttttcctggtgcaggtacccccttcagcttcccgatatccgacggtagtgtnnnnnnttctgcagctccttcgtcttcg

SEQ ID NO: 291 - DNA, 81 bp, Artificial sequence
  Artificially generated seqeunce
  misc_feature (59)..(64): n is a, c, g, or t
agaattacggggggcgggaagttgtttgcttcagcttcccgatatccgacggtagtgtnnnnnnggagtacaggcagatgt

SEQ ID NO: 292 - DNA, 80 bp, Artificial sequence
  Artificially generated seqeunce
  misc_feature (59)..(64): n is a, c, g, or t
atgtgcccgcagaaccaaagctggccttcttcagcttcccgatatccgacggtagtgtnnnnnntgctgtgctctgtttc

SEQ ID NO: 293 - DNA, 80 bp, Artificial sequence
  Artificially generated seqeunce
  misc_feature (53)..(58): n is a, c, g, or t
ggttggttccttttcagctgcccttcagcttcccgatatccgacggtagtgtnnnnnngcatcctgaaaatgttttaagg

SEQ ID NO: 294 - DNA, 79 bp, Artificial sequence
  Artificially generated seqeunce
  misc_feature (53)..(58): n is a, c, g, or t
ggattgctgctcagcccctcagcttcagcttcccgatatccgacggtagtgtnnnnnngttgggttatgtcttcccggg

SEQ ID NO: 295 - DNA, 81 bp, Artificial sequence
  Artificially generated seqeunce
  misc_feature (60)..(65): n is a, c, g, or t
agtctggaaaacacaagcccaatccagggcttcagcttcccgatatccgacggtagtgtnnnnnnttctcatgcttctcct

SEQ ID NO: 296 - DNA, 80 bp, Artificial sequence
  Artificially generated seqeunce
  misc_feature (59)..(64): n is a, c, g, or t
gggaaacccaagatctttttcattcaggcttcagcttcccgatatccgacggtagtgtnnnnnnccacgagattctagaa

SEQ ID NO: 297 - DNA, 78 bp, Artificial sequence
  Artificially generated seqeunce
  misc_feature (54)..(59): n is a, c, g, or t
ggagtgcctgatgaggcaggcttcttcagcttcccgatatccgacggtagtgtnnnnnntgcttcatctgctgtatcc

SEQ ID NO: 298 - DNA, 78 bp, Artificial sequence
  Artificially generated seqeunce
  misc_feature (54)..(59): n is a, c, g, or t
cagaggtgctaggtcatcacaggcttcagcttcccgatatccgacggtagtgtnnnnnntgtagtggtgctgtagcac

SEQ ID NO: 299 - DNA, 81 bp, Artificial sequence
  Artificially generated seqeunce
  misc_feature (60)..(65): n is a, c, g, or t
cggcattatctacacccaggacttcacagcttcagcttcccgatatccgacggtagtgtnnnnnncttctttagctggttc

SEQ ID NO: 300 - DNA, 79 bp, Artificial sequence
  Artificially generated seqeunce
  misc_feature (56)..(61): n is a, c, g, or t
gtgctccggctacaggcttgaggtacttcagcttcccgatatccgacggtagtgtnnnnnngtgtctgggcttggtggg

SEQ ID NO: 301 - DNA, 81 bp, Artificial sequence
  Artificially generated seqeunce
  misc_feature (60)..(65): n is a, c, g, or t
gctttgtccactgtgtctgcaggtggatgcttcagcttcccgatatccgacggtagtgtnnnnnnccttcttccccaggtg

SEQ ID NO: 302 - DNA, 78 bp, Artificial sequence
  Artificially generated seqeunce
  misc_feature (57)..(62): n is a, c, g, or t
gctaggtcctcagtgccagggtggcccttcagcttcccgatatccgacggtagtgtnnnnnngaggaggggtttgggg

SEQ ID NO: 303 - DNA, 81 bp, Artificial sequence
  Artificially generated seqeunce
  misc_feature (60)..(65): n is a, c, g, or t
aacttccaaagttttacatgccattacaccttcagcttcccgatatccgacggtagtgtnnnnnngatttggtcacgtcca

SEQ ID NO: 304 - DNA, 80 bp, Artificial sequence
  Artificially generated seqeunce
  misc_feature (59)..(64): n is a, c, g, or t
ggtaactgaattttctgtggacgtgacccttcagcttcccgatatccgacggtagtgtnnnnnnagtttgtgtaatggca

SEQ ID NO: 305 - DNA, 81 bp, Artificial sequence
  Artificially generated seqeunce
  misc_feature (60)..(65): n is a, c, g, or t
gtaagggtttaggaggattgacttgccagcttcagcttcccgatatccgacggtagtgtnnnnnnggcatttactttggct

SEQ ID NO: 306 - DNA, 78 bp, Artificial sequence
  Artificially generated seqeunce
  misc_feature (56)..(61): n is a, c, g, or t
ggtgattagccaaagtaaatgccagcttcagcttcccgatatccgacggtagtgtnnnnnnggcaagtcaatcctcct

SEQ ID NO: 307 - DNA, 81 bp, Artificial sequence
  Artificially generated seqeunce
  misc_feature (57)..(62): n is a, c, g, or t
catggaatgcttgtcctggtcagacacttcagcttcccgatatccgacggtagtgtnnnnnnggtttccctagggggcttt

SEQ ID NO: 308 - DNA, 81 bp, Artificial sequence
  Artificially generated seqeunce
  misc_feature (52)..(57): n is a, c, g, or t
gacactgaccccaaatgacgccttcagcttcccgatatccgacggtagtgtnnnnnngatgctgtccactaatgtgcacag

SEQ ID NO: 309 - DNA, 81 bp, Artificial sequence
  Artificially generated seqeunce
  misc_feature (54)..(59): n is a, c, g, or t
ggcctgtggccaactcgtactaccttcagcttcccgatatccgacggtagtgtnnnnnngcgtctatgtaattgagattaa

SEQ ID NO: 310 - DNA, 81 bp, Artificial sequence
  Artificially generated seqeunce
  misc_feature (58)..(63): n is a, c, g, or t
ggaagactgatgtcaatacaaagatcccttcagcttcccgatatccgacggtagtgtnnnnnntgggggagaactcccttt

SEQ ID NO: 311 - DNA, 81 bp, Artificial sequence
  Artificially generated seqeunce
  misc_feature (58)..(63): n is a, c, g, or t
gctggggaagagacaaaacctcagcaccttcagcttcccgatatccgacggtagtgtnnnnnnctgttacataactatgac

SEQ ID NO: 312 - DNA, 81 bp, Artificial sequence
  Artificially generated seqeunce
  misc_feature (57)..(62): n is a, c, g, or t
atgtttctgttggctaaactttgtcacttcagcttcccgatatccgacggtagtgtnnnnnngttcttctgggacggtgct

SEQ ID NO: 313 - DNA, 81 bp, Artificial sequence
  Artificially generated seqeunce
  misc_feature (60)..(65): n is a, c, g, or t
gttggatctgtatggagctcatgtctacccttcagcttcccgatatccgacggtagtgtnnnnnnaaaaaaatatcacaag

SEQ ID NO: 314 - DNA, 81 bp, Artificial sequence
  Artificially generated seqeunce
  misc_feature (60)..(65): n is a, c, g, or t
ggaagagatcttaggcaaaatcactttagcttcagcttcccgatatccgacggtagtgtnnnnnntctgtttctgtcctaa

SEQ ID NO: 315 - DNA, 80 bp, Artificial sequence
  Artificially generated seqeunce
  misc_feature (57)..(62): n is a, c, g, or t
gcagaggtaaccaaccacgtggcagacttcagcttcccgatatccgacggtagtgtnnnnnnttgtgtactgtatggatg

SEQ ID NO: 316 - DNA, 81 bp, Artificial sequence
  Artificially generated seqeunce
  misc_feature (56)..(61): n is a, c, g, or t
gcccttaagatacatcccgccataccttcagcttcccgatatccgacggtagtgtnnnnnnggtttgtttgtttgtatgtt

SEQ ID NO: 317 - DNA, 81 bp, Artificial sequence
  Artificially generated seqeunce
  misc_feature (60)..(65): n is a, c, g, or t
gcatgaaccgccgacctatccttaccatccttcagcttcccgatatccgacggtagtgtnnnnnncaactaaaactgaaac

SEQ ID NO: 318 - DNA, 76 bp, Artificial sequence
  Artificially generated seqeunce
  misc_feature (53)..(58): n is a, c, g, or t
ggcctgggggaagacacaggatcttcagcttcccgatatccgacggtagtgtnnnnnngtggtagggggcgggact

SEQ ID NO: 319 - DNA, 80 bp, Artificial sequence
  Artificially generated seqeunce
  misc_feature (49)..(54): n is a, c, g, or t
cccacctgcacaagcgcccttcagcttcccgatatccgacggtagtgtnnnnnnttgtgcctgccctgggagagaccgcc

SEQ ID NO: 320 - DNA, 80 bp, Artificial sequence
  Artificially generated seqeunce
  misc_feature (55)..(60): n is a, c, g, or t
ggtgaagctcaacaggctcctccgcttcagcttcccgatatccgacggtagtgtnnnnnnatacatgcgagagacagagg

SEQ ID NO: 321 - DNA, 80 bp, Artificial sequence
  Artificially generated seqeunce
  misc_feature (58)..(63): n is a, c, g, or t
cagtggacccagtctctcgtagcctggcttcagcttcccgatatccgacggtagtgtnnnnnnaaccgaaaagtagacca

SEQ ID NO: 322 - DNA, 81 bp, Artificial sequence
  Artificially generated seqeunce
  misc_feature (60)..(65): n is a, c, g, or t
gcgtactaattcctgaagtctaggaaggacttcagcttcccgatatccgacggtagtgtnnnnnnccccattctacaggag

SEQ ID NO: 323 - DNA, 79 bp, Artificial sequence
  Artificially generated seqeunce
  misc_feature (56)..(61): n is a, c, g, or t
ggaaacagccagtatccaggagccacttcagcttcccgatatccgacggtagtgtnnnnnngggggtggagcacttgtt

SEQ ID NO: 324 - DNA, 80 bp, Artificial sequence
  Artificially generated seqeunce
  misc_feature (57)..(62): n is a, c, g, or t
attctgctgaagataattgctaactccttcagcttcccgatatccgacggtagtgtnnnnnngtgctgccattgcttgtg

SEQ ID NO: 325 - DNA, 81 bp, Artificial sequence
  Artificially generated seqeunce
  misc_feature (58)..(63): n is a, c, g, or t
aaggagggggccttatgccctcagtgtcttcagcttcccgatatccgacggtagtgtnnnnnnaaagcagccgatgtaaag

SEQ ID NO: 326 - DNA, 79 bp, Artificial sequence
  Artificially generated seqeunce
  misc_feature (57)..(62): n is a, c, g, or t
ggaaagagaagcgcagattcacatgccttcagcttcccgatatccgacggtagtgtnnnnnncttcatcagcgatggca

SEQ ID NO: 327 - DNA, 81 bp, Artificial sequence
  Artificially generated seqeunce
  misc_feature (58)..(63): n is a, c, g, or t
ctcttttcccaaagatttgttccaactcttcagcttcccgatatccgacggtagtgtnnnnnnaatgtgcatacacacaaa

SEQ ID NO: 328 - DNA, 81 bp, Artificial sequence
  Artificially generated seqeunce
  misc_feature (60)..(65): n is a, c, g, or t
caccacaagagctcttggctgcagcgatgcttcagcttcccgatatccgacggtagtgtnnnnnnataattgcagtgccaa

SEQ ID NO: 329 - DNA, 81 bp, Artificial sequence
  Artificially generated seqeunce
  misc_feature (60)..(65): n is a, c, g, or t
ggaggcacgaaatgacccggagctcctgacttcagcttcccgatatccgacggtagtgtnnnnnntcagcctcacaagaga

SEQ ID NO: 330 - DNA, 80 bp, Artificial sequence
  Artificially generated seqeunce
  misc_feature (59)..(64): n is a, c, g, or t
atgcagagggggcaagtccgatgctggtcttcagcttcccgatatccgacggtagtgtnnnnnnagacaaggtgctggtg

SEQ ID NO: 331 - DNA, 81 bp, Artificial sequence
  Artificially generated seqeunce
  misc_feature (53)..(58): n is a, c, g, or t
gggccttccaggacttcagtcgcttcagcttcccgatatccgacggtagtgtnnnnnntgaagtctttacttgtgaaacta

SEQ ID NO: 332 - DNA, 81 bp, Artificial sequence
  Artificially generated seqeunce
  misc_feature (60)..(65): n is a, c, g, or t
ggaaacctgggtctctcccctgggtaccacttcagcttcccgatatccgacggtagtgtnnnnnntgttgggttgggtgtg

SEQ ID NO: 333 - DNA, 79 bp, Artificial sequence
  Artificially generated seqeunce
  misc_feature (50)..(55): n is a, c, g, or t
ggggttctgagggaccacccttcagcttcccgatatccgacggtagtgtnnnnnnggctgtgaacttgagtaacagggg

SEQ ID NO: 334 - DNA, 80 bp, Artificial sequence
  Artificially generated seqeunce
  misc_feature (59)..(64): n is a, c, g, or t
ggacattctccagattacagggcactcacttcagcttcccgatatccgacggtagtgtnnnnnngttgaagcatcacaca

SEQ ID NO: 335 - DNA, 80 bp, Artificial sequence
  Artificially generated seqeunce
  misc_feature (55)..(60): n is a, c, g, or t
catggctaaatggctagccagttccttcagcttcccgatatccgacggtagtgtnnnnnntgtgctggctgcaacttgtg

SEQ ID NO: 336 - DNA, 76 bp, Artificial sequence
  Artificially generated seqeunce
  misc_feature (55)..(60): n is a, c, g, or t
gctgatggcacagctctgaagaggcttcagcttcccgatatccgacggtagtgtnnnnnnaggtagaggtgccggg

SEQ ID NO: 337 - DNA, 79 bp, Artificial sequence
  Artificially generated seqeunce
  misc_feature (55)..(60): n is a, c, g, or t
ggctgtgcccatggtggagcgagtcttcagcttcccgatatccgacggtagtgtnnnnnnagaatatcgagtcccagac

SEQ ID NO: 338 - DNA, 81 bp, Artificial sequence
  Artificially generated seqeunce
  misc_feature (60)..(65): n is a, c, g, or t
gcacagagcgaccatgccatgcagtctgtcttcagcttcccgatatccgacggtagtgtnnnnnnaacacactttaaggac

SEQ ID NO: 339 - DNA, 78 bp, Artificial sequence
  Artificially generated seqeunce
  misc_feature (57)..(62): n is a, c, g, or t
gctgagcttgctgctgctgcgggcatcttcagcttcccgatatccgacggtagtgtnnnnnnggtggcctgggtgtgc

SEQ ID NO: 340 - DNA, 81 bp, Artificial sequence
  Artificially generated seqeunce
  misc_feature (60)..(65): n is a, c, g, or t
gctttatggtcggacctggtgagacgtgacttcagcttcccgatatccgacggtagtgtnnnnnncatctcagtgggcagc

SEQ ID NO: 341 - DNA, 81 bp, Artificial sequence
  Artificially generated seqeunce
  misc_feature (60)..(65): n is a, c, g, or t
cggtcctagtcaaagacggcaaaccctgccttcagcttcccgatatccgacggtagtgtnnnnnntcaagatctggttcca

SEQ ID NO: 342 - DNA, 81 bp, Artificial sequence
  Artificially generated seqeunce
  misc_feature (60)..(65): n is a, c, g, or t
gcgacgtttcaagcaacagaagtacctgtcttcagcttcccgatatccgacggtagtgtnnnnnntttgtcgcttacagtc

SEQ ID NO: 343 - DNA, 81 bp, Artificial sequence
  Artificially generated seqeunce
  misc_feature (60)..(65): n is a, c, g, or t
catgaagcgggagactgtaagcgacaaaccttcagcttcccgatatccgacggtagtgtnnnnnncaggtacttctgttgc

SEQ ID NO: 344 - DNA, 76 bp, Artificial sequence
  Artificially generated seqeunce
  misc_feature (49)..(54): n is a, c, g, or t
ggtgacggcgccgtggtgcttcagcttcccgatatccgacggtagtgtnnnnnnttactggcggggaagcgcgggt

SEQ ID NO: 345 - DNA, 80 bp, Artificial sequence
  Artificially generated seqeunce
  misc_feature (51)..(56): n is a, c, g, or t
ccgcctaccacatgacggcgcttcagcttcccgatatccgacggtagtgtnnnnnntccaaagcacacgactccgttctc

SEQ ID NO: 346 - DNA, 80 bp, Artificial sequence
  Artificially generated seqeunce
  misc_feature (59)..(64): n is a, c, g, or t
gtcgttaggtaagagtcactttttcattcttcagcttcccgatatccgacggtagtgtnnnnnntggtatacagtttggg

SEQ ID NO: 347 - DNA, 81 bp, Artificial sequence
  Artificially generated seqeunce
  misc_feature (60)..(65): n is a, c, g, or t
attttaatttacccaaactgtataccatacttcagcttcccgatatccgacggtagtgtnnnnnntgaaaaagtgactctt

SEQ ID NO: 348 - DNA, 80 bp, Artificial sequence
  Artificially generated seqeunce
  misc_feature (59)..(64): n is a, c, g, or t
caggtaaattaagcgacagcttgtggtgcttcagcttcccgatatccgacggtagtgtnnnnnnaaactaacagcttaat

SEQ ID NO: 349 - DNA, 81 bp, Artificial sequence
  Artificially generated seqeunce
  misc_feature (60)..(65): n is a, c, g, or t
caaatctttctccatagtacctttcatagcttcagcttcccgatatccgacggtagtgtnnnnnnatgctgactattttct

SEQ ID NO: 350 - DNA, 80 bp, Artificial sequence
  Artificially generated seqeunce
  misc_feature (59)..(64): n is a, c, g, or t
gatcccaagttctttataaaagtttagacttcagcttcccgatatccgacggtagtgtnnnnnntggatggatgtgggtt

SEQ ID NO: 351 - DNA, 80 bp, Artificial sequence
  Artificially generated seqeunce
  misc_feature (56)..(61): n is a, c, g, or t
ggccttattgttgaagacctagtggcttcagcttcccgatatccgacggtagtgtnnnnnngtgtttttgcatatttcag

SEQ ID NO: 352 - DNA, 80 bp, Artificial sequence
  Artificially generated seqeunce
  misc_feature (59)..(64): n is a, c, g, or t
accagggagtattgactgcatgggcagacttcagcttcccgatatccgacggtagtgtnnnnnngcggaggaataaacaa

SEQ ID NO: 353 - DNA, 81 bp, Artificial sequence
  Artificially generated seqeunce
  misc_feature (51)..(56): n is a, c, g, or t
ggccttctcaccaacccccacttcagcttcccgatatccgacggtagtgtnnnnnncattccacgtcttctcgtcatggtg

SEQ ID NO: 354 - DNA, 80 bp, Artificial sequence
  Artificially generated seqeunce
  misc_feature (58)..(63): n is a, c, g, or t
gcccaggagggttgggttttttgggttcttcagcttcccgatatccgacggtagtgtnnnnnngaaaataccgaagagta

SEQ ID NO: 355 - DNA, 81 bp, Artificial sequence
  Artificially generated seqeunce
  misc_feature (60)..(65): n is a, c, g, or t
ggcggggactgggcagtgggagttttgtccttcagcttcccgatatccgacggtagtgtnnnnnncgaagacttggcagtg

SEQ ID NO: 356 - DNA, 81 bp, Artificial sequence
  Artificially generated seqeunce
  misc_feature (60)..(65): n is a, c, g, or t
gtagggggtgagtggtataatcctattttcttcagcttcccgatatccgacggtagtgtnnnnnngttgttcatgctgttg

SEQ ID NO: 357 - DNA, 81 bp, Artificial sequence
  Artificially generated seqeunce
  misc_feature (54)..(59): n is a, c, g, or t
ggtaaggccacagagcctctcctcttcagcttcccgatatccgacggtagtgtnnnnnngatgcaagtctaattttaaatg

SEQ ID NO: 358 - DNA, 81 bp, Artificial sequence
  Artificially generated seqeunce
  misc_feature (60)..(65): n is a, c, g, or t
gctgtgatccggccacgggctgctgtgggcttcagcttcccgatatccgacggtagtgtnnnnnntaaatcagaaacagct

SEQ ID NO: 359 - DNA, 80 bp, Artificial sequence
  Artificially generated seqeunce
  misc_feature (59)..(64): n is a, c, g, or t
atttattgggaccaggtttgctgtagctcttcagcttcccgatatccgacggtagtgtnnnnnnctgctctcctgattcc

SEQ ID NO: 360 - DNA, 80 bp, Artificial sequence
  Artificially generated seqeunce
  misc_feature (59)..(64): n is a, c, g, or t
gtggagatcccatgacttctacaggtagcttcagcttcccgatatccgacggtagtgtnnnnnnactgcggtgtatgagg

SEQ ID NO: 361 - DNA, 81 bp, Artificial sequence
  Artificially generated seqeunce
  misc_feature (60)..(65): n is a, c, g, or t
agtacacacctgtaatcccagctcttggtcttcagcttcccgatatccgacggtagtgtnnnnnncctttatatttttcag

SEQ ID NO: 362 - DNA, 81 bp, Artificial sequence
  Artificially generated seqeunce
  misc_feature (60)..(65): n is a, c, g, or t
caagcagatgggacacatctgctcctcttcttcagcttcccgatatccgacggtagtgtnnnnnnccttctgctccagaca

SEQ ID NO: 363 - DNA, 81 bp, Artificial sequence
  Artificially generated seqeunce
  misc_feature (60)..(65): n is a, c, g, or t
gtgggttagaggagattgacttaggtttacttcagcttcccgatatccgacggtagtgtnnnnnntttgctggttgaagaa

SEQ ID NO: 364 - DNA, 81 bp, Artificial sequence
  Artificially generated seqeunce
  misc_feature (60)..(65): n is a, c, g, or t
cagatgtccaccatcatacctgtatcctccttcagcttcccgatatccgacggtagtgtnnnnnnagtcccacttgtgtag

SEQ ID NO: 365 - DNA, 81 bp, Artificial sequence
  Artificially generated seqeunce
  misc_feature (60)..(65): n is a, c, g, or t
atgtttgttataaccttaaattccatccacttcagcttcccgatatccgacggtagtgtnnnnnnctgttgtttactatag

SEQ ID NO: 366 - DNA, 80 bp, Artificial sequence
  Artificially generated seqeunce
  misc_feature (59)..(64): n is a, c, g, or t
catttccctaaaacttcccaaaccctagcttcagcttcccgatatccgacggtagtgtnnnnnntgagcctgggtctcaa

SEQ ID NO: 367 - DNA, 81 bp, Artificial sequence
  Artificially generated seqeunce
  misc_feature (54)..(59): n is a, c, g, or t
gggtgtagtctgaaagctgattccttcagcttcccgatatccgacggtagtgtnnnnnntctgatttttagttattcagtc

SEQ ID NO: 368 - DNA, 76 bp, Artificial sequence
  Artificially generated seqeunce
  misc_feature (55)..(60): n is a, c, g, or t
catgcttccttctgattttctgggcttcagcttcccgatatccgacggtagtgtnnnnnncagaaccacgtcacct

SEQ ID NO: 369 - DNA, 80 bp, Artificial sequence
  Artificially generated seqeunce
  misc_feature (59)..(64): n is a, c, g, or t
gggtaagaacccttggttgtgtccttctcttcagcttcccgatatccgacggtagtgtnnnnnnagcttgtagcttctca

SEQ ID NO: 370 - DNA, 76 bp, Artificial sequence
  Artificially generated seqeunce
  misc_feature (53)..(58): n is a, c, g, or t
ggttgcctgtggcctgctctggcttcagcttcccgatatccgacggtagtgtnnnnnngctcgaagggtcatcatg

SEQ ID NO: 371 - DNA, 80 bp, Artificial sequence
  Artificially generated seqeunce
  misc_feature (59)..(64): n is a, c, g, or t
gccctgcagcaggtaagttgcctggcttcttcagcttcccgatatccgacggtagtgtnnnnnncttaacagcaggtcag

SEQ ID NO: 372 - DNA, 78 bp, Artificial sequence
  Artificially generated seqeunce
  misc_feature (51)..(56): n is a, c, g, or t
cctgcggtcaggcaccactgcttcagcttcccgatatccgacggtagtgtnnnnnnaggaagcaaaggaaagaaatgg

SEQ ID NO: 373 - DNA, 81 bp, Artificial sequence
  Artificially generated seqeunce
  misc_feature (56)..(61): n is a, c, g, or t
atgaatcacctgtgttcagcgcaggcttcagcttcccgatatccgacggtagtgtnnnnnngtgattgaaagttttatcaa

SEQ ID NO: 374 - DNA, 81 bp, Artificial sequence
  Artificially generated seqeunce
  misc_feature (60)..(65): n is a, c, g, or t
attagaataaaaacaattgtgtattccaccttcagcttcccgatatccgacggtagtgtnnnnnntaagagaaattacctc

SEQ ID NO: 375 - DNA, 77 bp, Artificial sequence
  Artificially generated seqeunce
  misc_feature (54)..(59): n is a, c, g, or t
gcatgaaaccctgggttcattgccttcagcttcccgatatccgacggtagtgtnnnnnntccgagcgctcctgttct

SEQ ID NO: 376 - DNA, 81 bp, Artificial sequence
  Artificially generated seqeunce
  misc_feature (57)..(62): n is a, c, g, or t
ataggactggtgtggtccttaaactgcttcagcttcccgatatccgacggtagtgtnnnnnngagaagtattacagatgga

SEQ ID NO: 377 - DNA, 81 bp, Artificial sequence
  Artificially generated seqeunce
  misc_feature (60)..(65): n is a, c, g, or t
cacttttactgagctacagaaaagcatagcttcagcttcccgatatccgacggtagtgtnnnnnngtttaatgaaaaaatc

SEQ ID NO: 378 - DNA, 77 bp, Artificial sequence
  Artificially generated seqeunce
  misc_feature (51)..(56): n is a, c, g, or t
gcggaaggctgctgtggtggcttcagcttcccgatatccgacggtagtgtnnnnnngggtgcatgctttttttttgt

SEQ ID NO: 379 - DNA, 81 bp, Artificial sequence
  Artificially generated seqeunce
  misc_feature (49)..(54): n is a, c, g, or t
cttctggagccccacctgcttcagcttcccgatatccgacggtagtgtnnnnnnctctgtccttcccatctcttggttttt

SEQ ID NO: 380 - DNA, 80 bp, Artificial sequence
  Artificially generated seqeunce
  misc_feature (58)..(63): n is a, c, g, or t
atcttctagtataggaggaggtggactcttcagcttcccgatatccgacggtagtgtnnnnnngtcaggatcttgcgttc

SEQ ID NO: 381 - DNA, 81 bp, Artificial sequence
  Artificially generated seqeunce
  misc_feature (53)..(58): n is a, c, g, or t
cccactgtctgtcttctcctcacttcagcttcccgatatccgacggtagtgtnnnnnntcttcttctccgggacagcacca

SEQ ID NO: 382 - DNA, 81 bp, Artificial sequence
  Artificially generated seqeunce
  misc_feature (54)..(59): n is a, c, g, or t
gacatcgctctccaatgcgagagcttcagcttcccgatatccgacggtagtgtnnnnnnatcactcgtcatcctcacagtc

SEQ ID NO: 383 - DNA, 79 bp, Artificial sequence
  Artificially generated seqeunce
  misc_feature (55)..(60): n is a, c, g, or t
caaaagtaagttgtgccctgagtccttcagcttcccgatatccgacggtagtgtnnnnnngtgttattaattgcgttgt

SEQ ID NO: 384 - DNA, 81 bp, Artificial sequence
  Artificially generated seqeunce
  misc_feature (57)..(62): n is a, c, g, or t
attactcaggatcttcgaaaccacctcttcagcttcccgatatccgacggtagtgtnnnnnnctgttagctctaattaagt

SEQ ID NO: 385 - DNA, 81 bp, Artificial sequence
  Artificially generated seqeunce
  misc_feature (60)..(65): n is a, c, g, or t
attggagacagacttcacttaattagagccttcagcttcccgatatccgacggtagtgtnnnnnntgaacaaggtggtttc

SEQ ID NO: 386 - DNA, 79 bp, Artificial sequence
  Artificially generated seqeunce
  misc_feature (57)..(62): n is a, c, g, or t
acctacttgtcttgggtaaggcagggcttcagcttcccgatatccgacggtagtgtnnnnnngttctagtgttcaagcc

SEQ ID NO: 387 - DNA, 81 bp, Artificial sequence
  Artificially generated seqeunce
  misc_feature (60)..(65): n is a, c, g, or t
atttctatgctgtctttctcagttcctctcttcagcttcccgatatccgacggtagtgtnnnnnngtgactttcactgact

SEQ ID NO: 388 - DNA, 79 bp, Artificial sequence
  Artificially generated seqeunce
  misc_feature (58)..(63): n is a, c, g, or t
gcctggacaccttagggaatatcgcagcttcagcttcccgatatccgacggtagtgtnnnnnntcgaaagctgggcatt

SEQ ID NO: 389 - DNA, 81 bp, Artificial sequence
  Artificially generated seqeunce
  misc_feature (53)..(58): n is a, c, g, or t
tctgcagaacccgctggccttccttcagcttcccgatatccgacggtagtgtnnnnnnaggatctgccacgtgcttacctc

SEQ ID NO: 390 - DNA, 79 bp, Artificial sequence
  Artificially generated seqeunce
  misc_feature (56)..(61): n is a, c, g, or t
gggataggtttttaaagatgaggggcttcagcttcccgatatccgacggtagtgtnnnnnncaatgttgctcgtttttc

SEQ ID NO: 391 - DNA, 80 bp, Artificial sequence
  Artificially generated seqeunce
  misc_feature (59)..(64): n is a, c, g, or t
cagtgcaccatgcttttgcccaagctcccttcagcttcccgatatccgacggtagtgtnnnnnnttcacatgcagtaaca

SEQ ID NO: 392 - DNA, 79 bp, Artificial sequence
  Artificially generated seqeunce
  misc_feature (57)..(62): n is a, c, g, or t
ggagacagaggcatgcatgtgacccacttcagcttcccgatatccgacggtagtgtnnnnnnaacagacgtgacaggct

SEQ ID NO: 393 - DNA, 81 bp, Artificial sequence
  Artificially generated seqeunce
  misc_feature (60)..(65): n is a, c, g, or t
ggtttgactagagtctgtcttgctttctccttcagcttcccgatatccgacggtagtgtnnnnnnatgttggtgtgtgaga

SEQ ID NO: 394 - DNA, 80 bp, Artificial sequence
  Artificially generated seqeunce
  misc_feature (56)..(61): n is a, c, g, or t
ggacctgcctgctcactcgtggaaacttcagcttcccgatatccgacggtagtgtnnnnnngtgtgccaatcgtgcaggt

SEQ ID NO: 395 - DNA, 80 bp, Artificial sequence
  Artificially generated seqeunce
  misc_feature (59)..(64): n is a, c, g, or t
catcactatcatggagcctgtctggacacttcagcttcccgatatccgacggtagtgtnnnnnntgtgcctgtatgcaga

SEQ ID NO: 396 - DNA, 76 bp, Artificial sequence
  Artificially generated seqeunce
  misc_feature (55)..(60): n is a, c, g, or t
gcgcggcgccggtggatgcgttggcttcagcttcccgatatccgacggtagtgtnnnnnntggcggggtctgtgca

SEQ ID NO: 397 - DNA, 79 bp, Artificial sequence
  Artificially generated seqeunce
  misc_feature (51)..(56): n is a, c, g, or t
gccgatgtcctagtgtagggcttcagcttcccgatatccgacggtagtgtnnnnnngtgggctccgtggggtacttacc

SEQ ID NO: 398 - DNA, 76 bp, Artificial sequence
  Artificially generated seqeunce
  misc_feature (51)..(56): n is a, c, g, or t
ggagcagctgcggcgcacggcttcagcttcccgatatccgacggtagtgtnnnnnngggtgaagggagagggaggg

SEQ ID NO: 399 - DNA, 81 bp, Artificial sequence
  Artificially generated seqeunce
  misc_feature (60)..(65): n is a, c, g, or t
gccgcggtagcatttctcagttctgccgacttcagcttcccgatatccgacggtagtgtnnnnnncaaataaacattacct

SEQ ID NO: 400 - DNA, 81 bp, Artificial sequence
  Artificially generated seqeunce
  misc_feature (50)..(55): n is a, c, g, or t
ggacacacacatccctccccttcagcttcccgatatccgacggtagtgtnnnnnntgtaccttctgtaagcgcttggttct

SEQ ID NO: 401 - DNA, 81 bp, Artificial sequence
  Artificially generated seqeunce
  misc_feature (60)..(65): n is a, c, g, or t
atcacttctgtgtacacagtgcccctgtgcttcagcttcccgatatccgacggtagtgtnnnnnnctctgctgaaagttac

SEQ ID NO: 402 - DNA, 77 bp, Artificial sequence
  Artificially generated seqeunce
  misc_feature (54)..(59): n is a, c, g, or t
gggtgaggcgacatggatggtcccttcagcttcccgatatccgacggtagtgtnnnnnnaaaaccttggcgagcaca

SEQ ID NO: 403 - DNA, 81 bp, Artificial sequence
  Artificially generated seqeunce
  misc_feature (51)..(56): n is a, c, g, or t
ggacaagggccagcccagcccttcagcttcccgatatccgacggtagtgtnnnnnnggatgtgcagaatatatttgcaggg

SEQ ID NO: 404 - DNA, 79 bp, Artificial sequence
  Artificially generated seqeunce
  misc_feature (52)..(57): n is a, c, g, or t
ggccgctgtagcttgcactggcttcagcttcccgatatccgacggtagtgtnnnnnntggccctccctgttgagacatg

SEQ ID NO: 405 - DNA, 81 bp, Artificial sequence
  Artificially generated seqeunce
  misc_feature (60)..(65): n is a, c, g, or t
gcatcgcaatcacggcgcaactgctcactcttcagcttcccgatatccgacggtagtgtnnnnnnctcttgtcccctccca

SEQ ID NO: 406 - DNA, 79 bp, Artificial sequence
  Artificially generated seqeunce
  misc_feature (56)..(61): n is a, c, g, or t
ggtcggacatcaccaggattggacacttcagcttcccgatatccgacggtagtgtnnnnnnaacgcgctcccagacgaa

SEQ ID NO: 407 - DNA, 81 bp, Artificial sequence
  Artificially generated seqeunce
  misc_feature (60)..(65): n is a, c, g, or t
gtaaggacaggagcagagaaggagaaagacttcagcttcccgatatccgacggtagtgtnnnnnntgggaggggacaagag

SEQ ID NO: 408 - DNA, 80 bp, Artificial sequence
  Artificially generated seqeunce
  misc_feature (49)..(54): n is a, c, g, or t
cggccctgagatgttccgcttcagcttcccgatatccgacggtagtgtnnnnnnaactgttcctctgagccccacgaggt

SEQ ID NO: 409 - DNA, 81 bp, Artificial sequence
  Artificially generated seqeunce
  misc_feature (60)..(65): n is a, c, g, or t
atgccaaggggaaggacatcattccccagcttcagcttcccgatatccgacggtagtgtnnnnnntaattaagacacacag

SEQ ID NO: 410 - DNA, 81 bp, Artificial sequence
  Artificially generated seqeunce
  misc_feature (59)..(64): n is a, c, g, or t
cacttacccccaaaagtggcgcagccctcttcagcttcccgatatccgacggtagtgtnnnnnnctcatttcttcttggat

SEQ ID NO: 411 - DNA, 81 bp, Artificial sequence
  Artificially generated seqeunce
  misc_feature (51)..(56): n is a, c, g, or t
cggacaacccgtgtccaccacttcagcttcccgatatccgacggtagtgtnnnnnnttgggagtgggcagcttaagcctca

SEQ ID NO: 412 - DNA, 81 bp, Artificial sequence
  Artificially generated seqeunce
  misc_feature (59)..(64): n is a, c, g, or t
ggtctagacaacaagctgcgtgaggacccttcagcttcccgatatccgacggtagtgtnnnnnnaacccacgacagtacaa

SEQ ID NO: 413 - DNA, 80 bp, Artificial sequence
  Artificially generated seqeunce
  misc_feature (59)..(64): n is a, c, g, or t
tccaacacgttcagaaagtgtctctaaacttcagcttcccgatatccgacggtagtgtnnnnnnagcctttgtgacagtt

SEQ ID NO: 414 - DNA, 81 bp, Artificial sequence
  Artificially generated seqeunce
  misc_feature (56)..(61): n is a, c, g, or t
gcagcagttgaaggccacgcacatgcttcagcttcccgatatccgacggtagtgtnnnnnnctcaatttgtgtaaacttaa

SEQ ID NO: 415 - DNA, 81 bp, Artificial sequence
  Artificially generated seqeunce
  misc_feature (60)..(65): n is a, c, g, or t
attataaatataggccactctgaagctcccttcagcttcccgatatccgacggtagtgtnnnnnnctagcactagcaagag

SEQ ID NO: 416 - DNA, 80 bp, Artificial sequence
  Artificially generated seqeunce
  misc_feature (59)..(64): n is a, c, g, or t
ggaacagatacgagcttactgtgaaacccttcagcttcccgatatccgacggtagtgtnnnnnnttcctctcctcatcca

SEQ ID NO: 417 - DNA, 79 bp, Artificial sequence
  Artificially generated seqeunce
  misc_feature (53)..(58): n is a, c, g, or t
gggagtggcaggaagcccacgacttcagcttcccgatatccgacggtagtgtnnnnnnggcaatgacaaagactctgta

SEQ ID NO: 418 - DNA, 81 bp, Artificial sequence
  Artificially generated seqeunce
  misc_feature (60)..(65): n is a, c, g, or t
caatggagcaccagtactcaggagctacacttcagcttcccgatatccgacggtagtgtnnnnnnttctttactctcgaaa

SEQ ID NO: 419 - DNA, 81 bp, Artificial sequence
  Artificially generated seqeunce
  misc_feature (60)..(65): n is a, c, g, or t
cacctcaccaagaaaccaaaaccaagaggcttcagcttcccgatatccgacggtagtgtnnnnnntccacatagccatcca

SEQ ID NO: 420 - DNA, 81 bp, Artificial sequence
  Artificially generated seqeunce
  misc_feature (60)..(65): n is a, c, g, or t
atgccagtgcatattacagggtaactaaacttcagcttcccgatatccgacggtagtgtnnnnnnagttttagcactggaa

SEQ ID NO: 421 - DNA, 81 bp, Artificial sequence
  Artificially generated seqeunce
  misc_feature (60)..(65): n is a, c, g, or t
atacaaaattgctataactgggttatccccttcagcttcccgatatccgacggtagtgtnnnnnnagtgccacgggtctgt

SEQ ID NO: 422 - DNA, 79 bp, Artificial sequence
  Artificially generated seqeunce
  misc_feature (58)..(63): n is a, c, g, or t
ggatcatttcacaagaaatgtcttcaacttcagcttcccgatatccgacggtagtgtnnnnnngttctgtttgtggaag

SEQ ID NO: 423 - DNA, 81 bp, Artificial sequence
  Artificially generated seqeunce
  misc_feature (60)..(65): n is a, c, g, or t
atttaaaaatgtaccacaaatagtcaacacttcagcttcccgatatccgacggtagtgtnnnnnntgtcattatctgcacg

SEQ ID NO: 424 - DNA, 81 bp, Artificial sequence
  Artificially generated seqeunce
  misc_feature (60)..(65): n is a, c, g, or t
cagcattcacaaattacaaaagtctgattcttcagcttcccgatatccgacggtagtgtnnnnnnttctttctctaggtga

SEQ ID NO: 425 - DNA, 81 bp, Artificial sequence
  Artificially generated seqeunce
  misc_feature (57)..(62): n is a, c, g, or t
aaacttgaataaactgaaatggacctcttcagcttcccgatatccgacggtagtgtnnnnnngcagttcaacttctgtgac

SEQ ID NO: 426 - DNA, 79 bp, Artificial sequence
  Artificially generated seqeunce
  misc_feature (57)..(62): n is a, c, g, or t
gggatgacatgtgtctggagagagcgcttcagcttcccgatatccgacggtagtgtnnnnnnttcgcacacttggagac

SEQ ID NO: 427 - DNA, 80 bp, Artificial sequence
  Artificially generated seqeunce
  misc_feature (59)..(64): n is a, c, g, or t
gtggtctgacagttcgcgcaggatgtcccttcagcttcccgatatccgacggtagtgtnnnnnnaaacccatcagaaatc

SEQ ID NO: 428 - DNA, 80 bp, Artificial sequence
  Artificially generated seqeunce
  misc_feature (53)..(58): n is a, c, g, or t
ggagccatcctgaccatgacctcttcagcttcccgatatccgacggtagtgtnnnnnntctcttctcatccccagtcggc

SEQ ID NO: 429 - DNA, 77 bp, Artificial sequence
  Artificially generated seqeunce
  misc_feature (56)..(61): n is a, c, g, or t
gggaaggaaagcgtgtgaaaacgagcttcagcttcccgatatccgacggtagtgtnnnnnnagccttcgcttgggct

SEQ ID NO: 430 - DNA, 80 bp, Artificial sequence
  Artificially generated seqeunce
  misc_feature (54)..(59): n is a, c, g, or t
ggacggcttcggtaggtccttctcttcagcttcccgatatccgacggtagtgtnnnnnntgaggatacgggtcctgtggt

SEQ ID NO: 431 - DNA, 78 bp, Artificial sequence
  Artificially generated seqeunce
  misc_feature (52)..(57): n is a, c, g, or t
ggcctcggcttcccagatgctcttcagcttcccgatatccgacggtagtgtnnnnnngtggacgggagatggtgagac

SEQ ID NO: 432 - DNA, 81 bp, Artificial sequence
  Artificially generated seqeunce
  misc_feature (59)..(64): n is a, c, g, or t
cagcccaggccatcaaaatggtggttctcttcagcttcccgatatccgacggtagtgtnnnnnncactcattaacatcaat

SEQ ID NO: 433 - DNA, 76 bp, Artificial sequence
  Artificially generated seqeunce
  misc_feature (52)..(57): n is a, c, g, or t
gggtggtcgggaggctgcaagcttcagcttcccgatatccgacggtagtgtnnnnnnaaccctgtcaacggcaaag

SEQ ID NO: 434 - DNA, 81 bp, Artificial sequence
  Artificially generated seqeunce
  misc_feature (60)..(65): n is a, c, g, or t
ggcacaggcagcaggaacgggctggctgtcttcagcttcccgatatccgacggtagtgtnnnnnnttgtctaggtctgctg

SEQ ID NO: 435 - DNA, 81 bp, Artificial sequence
  Artificially generated seqeunce
  misc_feature (50)..(55): n is a, c, g, or t
cctctttcccctctcttggcttcagcttcccgatatccgacggtagtgtnnnnnncggattggctgtgagttcaggaacta

SEQ ID NO: 436 - DNA, 81 bp, Artificial sequence
  Artificially generated seqeunce
  misc_feature (60)..(65): n is a, c, g, or t
ggggtaagcctcaagttcttccttactttcttcagcttcccgatatccgacggtagtgtnnnnnnaatccaaatgctgaag

SEQ ID NO: 437 - DNA, 80 bp, Artificial sequence
  Artificially generated seqeunce
  misc_feature (59)..(64): n is a, c, g, or t
cagtctcagtgggggtgaattcagtgtgcttcagcttcccgatatccgacggtagtgtnnnnnntcagtcttttgggggt

SEQ ID NO: 438 - DNA, 81 bp, Artificial sequence
  Artificially generated seqeunce
  misc_feature (59)..(64): n is a, c, g, or t
agccactccactgtctcctacagtaacacttcagcttcccgatatccgacggtagtgtnnnnnncaacccttccaaaatga

SEQ ID NO: 439 - DNA, 79 bp, Artificial sequence
  Artificially generated seqeunce
  misc_feature (57)..(62): n is a, c, g, or t
ggagtcttgagtagaccaagctatctcttcagcttcccgatatccgacggtagtgtnnnnnnggggtgaagttttctgc

SEQ ID NO: 440 - DNA, 81 bp, Artificial sequence
  Artificially generated seqeunce
  misc_feature (60)..(65): n is a, c, g, or t
agcactagtattcgaagcaccaagtaagtcttcagcttcccgatatccgacggtagtgtnnnnnnccgtgagtgtgaaatg

SEQ ID NO: 441 - DNA, 81 bp, Artificial sequence
  Artificially generated seqeunce
  misc_feature (60)..(65): n is a, c, g, or t
gtgtttctactctggaaacaagttaacagcttcagcttcccgatatccgacggtagtgtnnnnnncttccctatttccttt

SEQ ID NO: 442 - DNA, 81 bp, Artificial sequence
  Artificially generated seqeunce
  misc_feature (60)..(65): n is a, c, g, or t
aaaaaaaatcacagaaaaaaaggttactccttcagcttcccgatatccgacggtagtgtnnnnnnctcttctttacatttc

SEQ ID NO: 443 - DNA, 81 bp, Artificial sequence
  Artificially generated seqeunce
  misc_feature (60)..(65): n is a, c, g, or t
ggacctagggccaaatctctccaactcatcttcagcttcccgatatccgacggtagtgtnnnnnngttgtcatcagaacca

SEQ ID NO: 444 - DNA, 81 bp, Artificial sequence
  Artificially generated seqeunce
  misc_feature (60)..(65): n is a, c, g, or t
attgtctctcatctgtgatgaccacactccttcagcttcccgatatccgacggtagtgtnnnnnntcacaaggaacagtaa

SEQ ID NO: 445 - DNA, 81 bp, Artificial sequence
  Artificially generated seqeunce
  misc_feature (56)..(61): n is a, c, g, or t
gctaatgagctcttggagtggacaccttcagcttcccgatatccgacggtagtgtnnnnnnatcgactgaccggactctca

SEQ ID NO: 446 - DNA, 81 bp, Artificial sequence
  Artificially generated seqeunce
  misc_feature (60)..(65): n is a, c, g, or t
ttaaaatacttgagtttcctagccactctcttcagcttcccgatatccgacggtagtgtnnnnnnatagtcttcagtctta

SEQ ID NO: 447 - DNA, 81 bp, Artificial sequence
  Artificially generated seqeunce
  misc_feature (60)..(65): n is a, c, g, or t
atcccaaaataataaaagtcagcagttttcttcagcttcccgatatccgacggtagtgtnnnnnntagtcctgctttgctg

SEQ ID NO: 448 - DNA, 81 bp, Artificial sequence
  Artificially generated seqeunce
  misc_feature (53)..(58): n is a, c, g, or t
gggcctcaaaacactggtgctgcttcagcttcccgatatccgacggtagtgtnnnnnngcagttcaggaaatcatgattcc

SEQ ID NO: 449 - DNA, 79 bp, Artificial sequence
  Artificially generated seqeunce
  misc_feature (58)..(63): n is a, c, g, or t
gcaagccaaggaaatggtattagagttcttcagcttcccgatatccgacggtagtgtnnnnnnggaacgggctggtgtt

SEQ ID NO: 450 - DNA, 77 bp, Artificial sequence
  Artificially generated seqeunce
  misc_feature (50)..(55): n is a, c, g, or t
cgtgtctagcatgtggcttcttcagcttcccgatatccgacggtagtgtnnnnnnacacgccgggagcagtgcagaa

SEQ ID NO: 451 - DNA, 80 bp, Artificial sequence
  Artificially generated seqeunce
  misc_feature (59)..(64): n is a, c, g, or t
gcccaagagcggggacatcaagacatcgcttcagcttcccgatatccgacggtagtgtnnnnnnggtgatgatgatgggc

SEQ ID NO: 452 - DNA, 81 bp, Artificial sequence
  Artificially generated seqeunce
  misc_feature (52)..(57): n is a, c, g, or t
cgcacgatgtcttgatgtccccttcagcttcccgatatccgacggtagtgtnnnnnntgttctcaggctcatttgggttgc

SEQ ID NO: 453 - DNA, 76 bp, Artificial sequence
  Artificially generated seqeunce
  misc_feature (52)..(57): n is a, c, g, or t
ggtagtggggtcctcgcagttcttcagcttcccgatatccgacggtagtgtnnnnnnaaagagcacccagcggaac

SEQ ID NO: 454 - DNA, 79 bp, Artificial sequence
  Artificially generated seqeunce
  misc_feature (55)..(60): n is a, c, g, or t
gcggagattcagtgcccgcccaatcttcagcttcccgatatccgacggtagtgtnnnnnnactccttgcctacctgaat

SEQ ID NO: 455 - DNA, 81 bp, Artificial sequence
  Artificially generated seqeunce
  misc_feature (60)..(65): n is a, c, g, or t
cagtctgtctgcagcggactccatgctgccttcagcttcccgatatccgacggtagtgtnnnnnntggtaattgaaatgtt

SEQ ID NO: 456 - DNA, 81 bp, Artificial sequence
  Artificially generated seqeunce
  misc_feature (49)..(54): n is a, c, g, or t
cccatcatgcccccttgacttcagcttcccgatatccgacggtagtgtnnnnnntcaggtccagtagattttactaccgct

SEQ ID NO: 457 - DNA, 81 bp, Artificial sequence
  Artificially generated seqeunce
  misc_feature (60)..(65): n is a, c, g, or t
gggacccccatatcctatgggtggaaccacttcagcttcccgatatccgacggtagtgtnnnnnnttttttcctcctcatt

SEQ ID NO: 458 - DNA, 81 bp, Artificial sequence
  Artificially generated seqeunce
  misc_feature (60)..(65): n is a, c, g, or t
gcgttgggcaaggcattatagttaggctgcttcagcttcccgatatccgacggtagtgtnnnnnntgaggtggcatattgg

SEQ ID NO: 459 - DNA, 77 bp, Artificial sequence
  Artificially generated seqeunce
  misc_feature (55)..(60): n is a, c, g, or t
gggatgtgtcctccaccagggggacttcagcttcccgatatccgacggtagtgtnnnnnnaacgccaactaccccaa

SEQ ID NO: 460 - DNA, 81 bp, Artificial sequence
  Artificially generated seqeunce
  misc_feature (60)..(65): n is a, c, g, or t
gggtcatgaaaatgaacaaatccatcattcttcagcttcccgatatccgacggtagtgtnnnnnnccagcacccatagggt

SEQ ID NO: 461 - DNA, 77 bp, Artificial sequence
  Artificially generated seqeunce
  misc_feature (52)..(57): n is a, c, g, or t
cagccctgagtggcctggcaacttcagcttcccgatatccgacggtagtgtnnnnnntgtgcgctgcgggctcttag

SEQ ID NO: 462 - DNA, 81 bp, Artificial sequence
  Artificially generated seqeunce
  misc_feature (60)..(65): n is a, c, g, or t
gcaagagcaagatcactgtcacttcagagcttcagcttcccgatatccgacggtagtgtnnnnnntaaagagagagagctg

SEQ ID NO: 463 - DNA, 76 bp, Artificial sequence
  Artificially generated seqeunce
  misc_feature (54)..(59): n is a, c, g, or t
gctggagctgtccagtgtaggtgcttcagcttcccgatatccgacggtagtgtnnnnnnggcggaggagtcgtgac

SEQ ID NO: 464 - DNA, 81 bp, Artificial sequence
  Artificially generated seqeunce
  misc_feature (60)..(65): n is a, c, g, or t
ataaaatttaggaacaaaaccggtggtttcttcagcttcccgatatccgacggtagtgtnnnnnnatctcaccaaaaaata

SEQ ID NO: 465 - DNA, 81 bp, Artificial sequence
  Artificially generated seqeunce
  misc_feature (58)..(63): n is a, c, g, or t
ggtcagaaaacgcagcaacaacgctgccttcagcttcccgatatccgacggtagtgtnnnnnnaggttgcttcaaccggta

SEQ ID NO: 466 - DNA, 81 bp, Artificial sequence
  Artificially generated seqeunce
  misc_feature (54)..(59): n is a, c, g, or t
ggcagcatttgcttcttcgtgggcttcagcttcccgatatccgacggtagtgtnnnnnntgcactcttcttctaggtgagt

SEQ ID NO: 467 - DNA, 81 bp, Artificial sequence
  Artificially generated seqeunce
  misc_feature (60)..(65): n is a, c, g, or t
attgcatagagaactgacaccatattcctcttcagcttcccgatatccgacggtagtgtnnnnnnatgctatcttattttt

SEQ ID NO: 468 - DNA, 78 bp, Artificial sequence
  Artificially generated seqeunce
  misc_feature (54)..(59): n is a, c, g, or t
ggctggaaatgcaaagtaagtgacttcagcttcccgatatccgacggtagtgtnnnnnnggattgtgcaagttttagg

SEQ ID NO: 469 - DNA, 78 bp, Artificial sequence
  Artificially generated seqeunce
  misc_feature (51)..(56): n is a, c, g, or t
gcgtcgtgctgcctttgtggcttcagcttcccgatatccgacggtagtgtnnnnnncacgtccagcttgcgaatccga

SEQ ID NO: 470 - DNA, 81 bp, Artificial sequence
  Artificially generated seqeunce
  misc_feature (54)..(59): n is a, c, g, or t
gggtctgggcatggcacctgtttcttcagcttcccgatatccgacggtagtgtnnnnnngtaagattgactattaacctgg

SEQ ID NO: 471 - DNA, 78 bp, Artificial sequence
  Artificially generated seqeunce
  misc_feature (51)..(56): n is a, c, g, or t
ggtccccgcatccctgaagacttcagcttcccgatatccgacggtagtgtnnnnnngtttgggctacatagctccagg

SEQ ID NO: 472 - DNA, 81 bp, Artificial sequence
  Artificially generated seqeunce
  misc_feature (54)..(59): n is a, c, g, or t
ccaggctccgcacaacctgaaggcttcagcttcccgatatccgacggtagtgtnnnnnngctgaaactcaggaacacttaa

SEQ ID NO: 473 - DNA, 79 bp, Artificial sequence
  Artificially generated seqeunce
  misc_feature (54)..(59): n is a, c, g, or t
gatctacacataggacaggtcaccttcagcttcccgatatccgacggtagtgtnnnnnnaggagactggacatcgtcag

SEQ ID NO: 474 - DNA, 81 bp, Artificial sequence
  Artificially generated seqeunce
  misc_feature (60)..(65): n is a, c, g, or t
gctgggttagcggagcagtgtccagggatcttcagcttcccgatatccgacggtagtgtnnnnnnaggtggagaggggcag

SEQ ID NO: 475 - DNA, 78 bp, Artificial sequence
  Artificially generated seqeunce
  misc_feature (56)..(61): n is a, c, g, or t
gccgaagagatttctgcaggcggaacttcagcttcccgatatccgacggtagtgtnnnnnnttgggggggcgcggggg

SEQ ID NO: 476 - DNA, 81 bp, Artificial sequence
  Artificially generated seqeunce
  misc_feature (60)..(65): n is a, c, g, or t
gcgagtcagcgcaagtggaatttcgacttcttcagcttcccgatatccgacggtagtgtnnnnnnaggaggaagatgtcaa

SEQ ID NO: 477 - DNA, 80 bp, Artificial sequence
  Artificially generated seqeunce
  misc_feature (59)..(64): n is a, c, g, or t
gcgtgttctgctttagctctgggaaatccttcagcttcccgatatccgacggtagtgtnnnnnnaccaaatgcctgactc

SEQ ID NO: 478 - DNA, 81 bp, Artificial sequence
  Artificially generated seqeunce
  misc_feature (59)..(64): n is a, c, g, or t
gaggaagaacacaaaactgacaccccatcttcagcttcccgatatccgacggtagtgtnnnnnngatggtttgtgtgtgtt

SEQ ID NO: 479 - DNA, 80 bp, Artificial sequence
  Artificially generated seqeunce
  misc_feature (57)..(62): n is a, c, g, or t
ggcccaagggatgaacaacccactcacttcagcttcccgatatccgacggtagtgtnnnnnngggcttcttgggcgtctg

SEQ ID NO: 480 - DNA, 78 bp, Artificial sequence
  Artificially generated seqeunce
  misc_feature (57)..(62): n is a, c, g, or t
gtctggtaggagcgcgtggctgtgagcttcagcttcccgatatccgacggtagtgtnnnnnnggggttggaggaggtg

SEQ ID NO: 481 - DNA, 81 bp, Artificial sequence
  Artificially generated seqeunce
  misc_feature (60)..(65): n is a, c, g, or t
gtaggtgacagcaaaggcaggtgagatctcttcagcttcccgatatccgacggtagtgtnnnnnngtagaagaagaagggg

SEQ ID NO: 482 - DNA, 81 bp, Artificial sequence
  Artificially generated seqeunce
  misc_feature (60)..(65): n is a, c, g, or t
gcttgggatgggcaggaagttagtgcatgcttcagcttcccgatatccgacggtagtgtnnnnnngaagcagatggacagg

SEQ ID NO: 483 - DNA, 81 bp, Artificial sequence
  Artificially generated seqeunce
  misc_feature (60)..(65): n is a, c, g, or t
cagagcatgcagcttctgttccctgtcgtcttcagcttcccgatatccgacggtagtgtnnnnnntgctacatgagggcag

SEQ ID NO: 484 - DNA, 81 bp, Artificial sequence
  Artificially generated seqeunce
  misc_feature (60)..(65): n is a, c, g, or t
cagattgtggtctgaaagaaagccaaccacttcagcttcccgatatccgacggtagtgtnnnnnnaaaaaaagggcaaggc

SEQ ID NO: 485 - DNA, 76 bp, Artificial sequence
  Artificially generated seqeunce
  misc_feature (55)..(60): n is a, c, g, or t
ggtctgtgtacggggacaggggctcttcagcttcccgatatccgacggtagtgtnnnnnncaggctcggctcaggt

SEQ ID NO: 486 - DNA, 79 bp, Artificial sequence
  Artificially generated seqeunce
  misc_feature (56)..(61): n is a, c, g, or t
gctggaggccacactgggaaggggtcttcagcttcccgatatccgacggtagtgtnnnnnncttggtactttgtttgtg

SEQ ID NO: 487 - DNA, 79 bp, Artificial sequence
  Artificially generated seqeunce
  misc_feature (56)..(61): n is a, c, g, or t
ggtctgcagagtgagtgccaggactcttcagcttcccgatatccgacggtagtgtnnnnnngacaggtgagaggtgcac

SEQ ID NO: 488 - DNA, 81 bp, Artificial sequence
  Artificially generated seqeunce
  misc_feature (60)..(65): n is a, c, g, or t
gtatgatgcggggccagaaaccatctgtgcttcagcttcccgatatccgacggtagtgtnnnnnntcatccccttcttcct

SEQ ID NO: 489 - DNA, 81 bp, Artificial sequence
  Artificially generated seqeunce
  misc_feature (60)..(65): n is a, c, g, or t
gcaccccactgtgggagaaggctctgggtcttcagcttcccgatatccgacggtagtgtnnnnnntggggggaaggctttg

SEQ ID NO: 490 - DNA, 81 bp, Artificial sequence
  Artificially generated seqeunce
  misc_feature (60)..(65): n is a, c, g, or t
acagccaagcacttcaagaactgacccagcttcagcttcccgatatccgacggtagtgtnnnnnncaccttctgctttaca

SEQ ID NO: 491 - DNA, 81 bp, Artificial sequence
  Artificially generated seqeunce
  misc_feature (60)..(65): n is a, c, g, or t
gaagacagaaggggcagggaaccaagttacttcagcttcccgatatccgacggtagtgtnnnnnnagcttgggaaggtggg

SEQ ID NO: 492 - DNA, 77 bp, Artificial sequence
  Artificially generated seqeunce
  misc_feature (55)..(60): n is a, c, g, or t
gcattttaccctggcagccctgcacttcagcttcccgatatccgacggtagtgtnnnnnntgggatttggggtgggg

SEQ ID NO: 493 - DNA, 81 bp, Artificial sequence
  Artificially generated seqeunce
  misc_feature (50)..(55): n is a, c, g, or t
cacccttgctccacccaaccttcagcttcccgatatccgacggtagtgtnnnnnnttggtgccagcagccttggtgacctt

SEQ ID NO: 494 - DNA, 81 bp, Artificial sequence
  Artificially generated seqeunce
  misc_feature (58)..(63): n is a, c, g, or t
gggcagagcagtgtggggtccaggagtcttcagcttcccgatatccgacggtagtgtnnnnnngaagatgaagatgcagag

SEQ ID NO: 495 - DNA, 81 bp, Artificial sequence
  Artificially generated seqeunce
  misc_feature (60)..(65): n is a, c, g, or t
cacctgtccccctgtttcaggtgactctgcttcagcttcccgatatccgacggtagtgtnnnnnntggagcttcttgggag

SEQ ID NO: 496 - DNA, 81 bp, Artificial sequence
  Artificially generated seqeunce
  misc_feature (50)..(55): n is a, c, g, or t
atgtcctctagggcactgccttcagcttcccgatatccgacggtagtgtnnnnnnccactttcttaactttgcaggctatt

SEQ ID NO: 497 - DNA, 79 bp, Artificial sequence
  Artificially generated seqeunce
  misc_feature (55)..(60): n is a, c, g, or t
atggaaagtgatccggtggctcaccttcagcttcccgatatccgacggtagtgtnnnnnngtcacatgtgatttgctgt

SEQ ID NO: 498 - DNA, 81 bp, Artificial sequence
  Artificially generated seqeunce
  misc_feature (60)..(65): n is a, c, g, or t
caccagtgacacatgtctgtcagagagaccttcagcttcccgatatccgacggtagtgtnnnnnnctgctgtttgtcctaa

SEQ ID NO: 499 - DNA, 81 bp, Artificial sequence
  Artificially generated seqeunce
  misc_feature (52)..(57): n is a, c, g, or t
gtggctgtcatagttgccacccttcagcttcccgatatccgacggtagtgtnnnnnntgctgtgagggcattttctcggtc

SEQ ID NO: 500 - DNA, 81 bp, Artificial sequence
  Artificially generated seqeunce
  misc_feature (60)..(65): n is a, c, g, or t
cgggttaatgttaaatgcgtcgcccatgtcttcagcttcccgatatccgacggtagtgtnnnnnntgaatattctgcctgc

SEQ ID NO: 501 - DNA, 81 bp, Artificial sequence
  Artificially generated seqeunce
  misc_feature (58)..(63): n is a, c, g, or t
cactgatgactggctgtgtgcagagaccttcagcttcccgatatccgacggtagtgtnnnnnnacgtgcaccaccaccgtt

SEQ ID NO: 502 - DNA, 81 bp, Artificial sequence
  Artificially generated seqeunce
  misc_feature (60)..(65): n is a, c, g, or t
gctccagtgtttgttaaccttccctactacttcagcttcccgatatccgacggtagtgtnnnnnncgcagatttaaaatca

SEQ ID NO: 503 - DNA, 76 bp, Artificial sequence
  Artificially generated seqeunce
  misc_feature (54)..(59): n is a, c, g, or t
cgctgtggtgaaggtggatgctgcttcagcttcccgatatccgacggtagtgtnnnnnngtcgggcgtgttgtcca

SEQ ID NO: 504 - DNA, 81 bp, Artificial sequence
  Artificially generated seqeunce
  misc_feature (60)..(65): n is a, c, g, or t
acttgaccttaaactcaaacttttgtgtacttcagcttcccgatatccgacggtagtgtnnnnnntttgtcacgctcggtt

SEQ ID NO: 505 - DNA, 81 bp, Artificial sequence
  Artificially generated seqeunce
  misc_feature (60)..(65): n is a, c, g, or t
cagcatcacagtagcgacattttttgccccttcagcttcccgatatccgacggtagtgtnnnnnnaaaagaaccaccatac

SEQ ID NO: 506 - DNA, 79 bp, Artificial sequence
  Artificially generated seqeunce
  misc_feature (57)..(62): n is a, c, g, or t
atttagagatccgggctggtgagatgcttcagcttcccgatatccgacggtagtgtnnnnnnaagtgtgaccagtgtga

SEQ ID NO: 507 - DNA, 81 bp, Artificial sequence
  Artificially generated seqeunce
  misc_feature (60)..(65): n is a, c, g, or t
aagactcctctgacagtggtaagtgggtccttcagcttcccgatatccgacggtagtgtnnnnnngtctgaactttgttct

SEQ ID NO: 508 - DNA, 81 bp, Artificial sequence
  Artificially generated seqeunce
  misc_feature (60)..(65): n is a, c, g, or t
ggttagaggtgacacatcagagacccagtcttcagcttcccgatatccgacggtagtgtnnnnnnttccttttgtcagaac

SEQ ID NO: 509 - DNA, 81 bp, Artificial sequence
  Artificially generated seqeunce
  misc_feature (60)..(65): n is a, c, g, or t
catcgcctacaccatcgtcagccaggatccttcagcttcccgatatccgacggtagtgtnnnnnnactgtgacaaagtgtg

SEQ ID NO: 510 - DNA, 80 bp, Artificial sequence
  Artificially generated seqeunce
  misc_feature (55)..(60): n is a, c, g, or t
atgacaacgctcctgtcttcaacccttcagcttcccgatatccgacggtagtgtnnnnnnctatacgcagtaagctgcac

SEQ ID NO: 511 - DNA, 77 bp, Artificial sequence
  Artificially generated seqeunce
  misc_feature (54)..(59): n is a, c, g, or t
agtcaccccaaacccagtgcagccttcagcttcccgatatccgacggtagtgtnnnnnngttaccgtgcttgggttg

SEQ ID NO: 512 - DNA, 76 bp, Artificial sequence
  Artificially generated seqeunce
  misc_feature (53)..(58): n is a, c, g, or t
agacccagggcttgggctaaagcttcagcttcccgatatccgacggtagtgtnnnnnnggtcgtggggtctgtgac

SEQ ID NO: 513 - DNA, 78 bp, Artificial sequence
  Artificially generated seqeunce
  misc_feature (55)..(60): n is a, c, g, or t
gcagggggagaggttggaagtctgcttcagcttcccgatatccgacggtagtgtnnnnnngtggaaagctgtgtacac

SEQ ID NO: 514 - DNA, 81 bp, Artificial sequence
  Artificially generated seqeunce
  misc_feature (60)..(65): n is a, c, g, or t
gcatggcagacgaaaactcaggttcttcacttcagcttcccgatatccgacggtagtgtnnnnnnaactttaaatcattac

SEQ ID NO: 515 - DNA, 81 bp, Artificial sequence
  Artificially generated seqeunce
  misc_feature (54)..(59): n is a, c, g, or t
tcttctttcagccgagaccactccttcagcttcccgatatccgacggtagtgtnnnnnntgctgtcacattgtttccaagc

SEQ ID NO: 516 - DNA, 81 bp, Artificial sequence
  Artificially generated seqeunce
  misc_feature (60)..(65): n is a, c, g, or t
catggcacaccgcgcggggacaagtgaaccttcagcttcccgatatccgacggtagtgtnnnnnnaaggattactcactga

SEQ ID NO: 517 - DNA, 81 bp, Artificial sequence
  Artificially generated seqeunce
  misc_feature (60)..(65): n is a, c, g, or t
cactgtaatcagtggattccctttgtgtccttcagcttcccgatatccgacggtagtgtnnnnnnaccgtcaaataacaac

SEQ ID NO: 518 - DNA, 81 bp, Artificial sequence
  Artificially generated seqeunce
  misc_feature (60)..(65): n is a, c, g, or t
agaagtcacagagtgattttgatctcgtgcttcagcttcccgatatccgacggtagtgtnnnnnntctccccgatgattca

SEQ ID NO: 519 - DNA, 81 bp, Artificial sequence
  Artificially generated seqeunce
  misc_feature (60)..(65): n is a, c, g, or t
attcctcaggatctcgtaatattgctgtccttcagcttcccgatatccgacggtagtgtnnnnnnctaagaaggtgagtca

SEQ ID NO: 520 - DNA, 81 bp, Artificial sequence
  Artificially generated seqeunce
  misc_feature (59)..(64): n is a, c, g, or t
atatccatcatccacaaggagccagagccttcagcttcccgatatccgacggtagtgtnnnnnncacatcttagcagccct

SEQ ID NO: 521 - DNA, 80 bp, Artificial sequence
  Artificially generated seqeunce
  misc_feature (58)..(63): n is a, c, g, or t
gggtgtaacaggaacagttgaaaaggacttcagcttcccgatatccgacggtagtgtnnnnnngtctatgttgagtccaa

SEQ ID NO: 522 - DNA, 81 bp, Artificial sequence
  Artificially generated seqeunce
  misc_feature (60)..(65): n is a, c, g, or t
ggattacacatagaaatctttatgtgattcttcagcttcccgatatccgacggtagtgtnnnnnntctttggaactggtag

SEQ ID NO: 523 - DNA, 80 bp, Artificial sequence
  Artificially generated seqeunce
  misc_feature (53)..(58): n is a, c, g, or t
ccattcacagtccactgaaatgcttcagcttcccgatatccgacggtagtgtnnnnnngctgagaactgatactattggt

SEQ ID NO: 524 - DNA, 81 bp, Artificial sequence
  Artificially generated seqeunce
  misc_feature (60)..(65): n is a, c, g, or t
cacttattcaaacaacctggggggccgggcttcagcttcccgatatccgacggtagtgtnnnnnnagacaggtaagtttct

SEQ ID NO: 525 - DNA, 78 bp, Artificial sequence
  Artificially generated seqeunce
  misc_feature (55)..(60): n is a, c, g, or t
ggtagagggaatataatactggcgcttcagcttcccgatatccgacggtagtgtnnnnnngaaagaacacacactcac

SEQ ID NO: 526 - DNA, 79 bp, Artificial sequence
  Artificially generated seqeunce
  misc_feature (58)..(63): n is a, c, g, or t
accaaatagctttttctgggcaggctgcttcagcttcccgatatccgacggtagtgtnnnnnncctcctttcctctgca

SEQ ID NO: 527 - DNA, 80 bp, Artificial sequence
  Artificially generated seqeunce
  misc_feature (58)..(63): n is a, c, g, or t
gggtcaaaggtcactgtcagctgtcttcttcagcttcccgatatccgacggtagtgtnnnnnncactcggtcaaagtctc

SEQ ID NO: 528 - DNA, 81 bp, Artificial sequence
  Artificially generated seqeunce
  misc_feature (60)..(65): n is a, c, g, or t
attccagcataagcgaatgaccttgagggcttcagcttcccgatatccgacggtagtgtnnnnnnccccatttggttccaa

SEQ ID NO: 529 - DNA, 81 bp, Artificial sequence
  Artificially generated seqeunce
  misc_feature (60)..(65): n is a, c, g, or t
ggctggcttccttggttctctgaaactctcttcagcttcccgatatccgacggtagtgtnnnnnnctcatcttgtctgtgt

SEQ ID NO: 530 - DNA, 81 bp, Artificial sequence
  Artificially generated seqeunce
  misc_feature (60)..(65): n is a, c, g, or t
atgtctactccatggctctggtactctggcttcagcttcccgatatccgacggtagtgtnnnnnnacagacaatcgttggc

SEQ ID NO: 531 - DNA, 81 bp, Artificial sequence
  Artificially generated seqeunce
  misc_feature (60)..(65): n is a, c, g, or t
ggagaacactgacactaaggtaccttctgcttcagcttcccgatatccgacggtagtgtnnnnnnaagaaagccagaagac

SEQ ID NO: 532 - DNA, 81 bp, Artificial sequence
  Artificially generated seqeunce
  misc_feature (53)..(58): n is a, c, g, or t
atacggagggggagggggcggtcttcagcttcccgatatccgacggtagtgtnnnnnnaatcctcgtcttcccctgtgcac

SEQ ID NO: 533 - DNA, 79 bp, Artificial sequence
  Artificially generated seqeunce
  misc_feature (52)..(57): n is a, c, g, or t
gcactgatcactggagcggctcttcagcttcccgatatccgacggtagtgtnnnnnnttgaggctggcgtgcagcttgg

SEQ ID NO: 534 - DNA, 81 bp, Artificial sequence
  Artificially generated seqeunce
  misc_feature (60)..(65): n is a, c, g, or t
gcagcaagtggcgttgctttttaggatggcttcagcttcccgatatccgacggtagtgtnnnnnnagcctctgttgccttt

SEQ ID NO: 535 - DNA, 81 bp, Artificial sequence
  Artificially generated seqeunce
  misc_feature (60)..(65): n is a, c, g, or t
acttgggccatccaaggttgtggctttttcttcagcttcccgatatccgacggtagtgtnnnnnnaacatcactgaagaga

SEQ ID NO: 536 - DNA, 79 bp, Artificial sequence
  Artificially generated seqeunce
  misc_feature (58)..(63): n is a, c, g, or t
caaagagatcatgtttgtttccagtgccttcagcttcccgatatccgacggtagtgtnnnnnnttccacagcggtctca

SEQ ID NO: 537 - DNA, 81 bp, Artificial sequence
  Artificially generated seqeunce
  misc_feature (58)..(63): n is a, c, g, or t
caaagtgaacacagactatgagtctagcttcagcttcccgatatccgacggtagtgtnnnnnnacataaaacgagttttac

SEQ ID NO: 538 - DNA, 81 bp, Artificial sequence
  Artificially generated seqeunce
  misc_feature (60)..(65): n is a, c, g, or t
tcaggtatgtcagatctaacaaaatactgcttcagcttcccgatatccgacggtagtgtnnnnnncaaaatgatctgaaca

SEQ ID NO: 539 - DNA, 80 bp, Artificial sequence
  Artificially generated seqeunce
  misc_feature (58)..(63): n is a, c, g, or t
ggaaatttcatacaattttggctattgcttcagcttcccgatatccgacggtagtgtnnnnnnccaaaccttgctgtact

SEQ ID NO: 540 - DNA, 81 bp, Artificial sequence
  Artificially generated seqeunce
  misc_feature (59)..(64): n is a, c, g, or t
tcttctaagtatttcccatcttagtctccttcagcttcccgatatccgacggtagtgtnnnnnnctttagaggaagtatca

SEQ ID NO: 541 - DNA, 81 bp, Artificial sequence
  Artificially generated seqeunce
  misc_feature (60)..(65): n is a, c, g, or t
cagtatcatatatgatgagtacatgatggcttcagcttcccgatatccgacggtagtgtnnnnnnagtttattaaaagcaa

SEQ ID NO: 542 - DNA, 81 bp, Artificial sequence
  Artificially generated seqeunce
  misc_feature (59)..(64): n is a, c, g, or t
ataatgagaaaagttatgaatgcagcatcttcagcttcccgatatccgacggtagtgtnnnnnngcctttcgttagctcgt

SEQ ID NO: 543 - DNA, 81 bp, Artificial sequence
  Artificially generated seqeunce
  misc_feature (49)..(54): n is a, c, g, or t
ccaacctgcttagcatgacttcagcttcccgatatccgacggtagtgtnnnnnngatgactgctttggtaaatgtggcact

SEQ ID NO: 544 - DNA, 81 bp, Artificial sequence
  Artificially generated seqeunce
  misc_feature (60)..(65): n is a, c, g, or t
ataaagtttggagactgagtaagtctgggcttcagcttcccgatatccgacggtagtgtnnnnnntcaaaaaatgatgtgt

SEQ ID NO: 545 - DNA, 81 bp, Artificial sequence
  Artificially generated seqeunce
  misc_feature (50)..(55): n is a, c, g, or t
cgctctgctctttctttggcttcagcttcccgatatccgacggtagtgtnnnnnntgctttatgcctttagaaaaaggacc

SEQ ID NO: 546 - DNA, 81 bp, Artificial sequence
  Artificially generated seqeunce
  misc_feature (60)..(65): n is a, c, g, or t
caaaaaagtagtggataatggagggcatgcttcagcttcccgatatccgacggtagtgtnnnnnnatcaacagaaacctct

SEQ ID NO: 547 - DNA, 81 bp, Artificial sequence
  Artificially generated seqeunce
  misc_feature (60)..(65): n is a, c, g, or t
gtttttctctcctaaggcatgttttgtttcttcagcttcccgatatccgacggtagtgtnnnnnnaataaatttccctttc

SEQ ID NO: 548 - DNA, 81 bp, Artificial sequence
  Artificially generated seqeunce
  misc_feature (60)..(65): n is a, c, g, or t
gcaagaacactatactttaaacactggaccttcagcttcccgatatccgacggtagtgtnnnnnnttgttcatccattcca

SEQ ID NO: 549 - DNA, 81 bp, Artificial sequence
  Artificially generated seqeunce
  misc_feature (60)..(65): n is a, c, g, or t
cacatatgaaatgtgcaagtgaagtgcaacttcagcttcccgatatccgacggtagtgtnnnnnntcatgtttcatttcca

SEQ ID NO: 550 - DNA, 81 bp, Artificial sequence
  Artificially generated seqeunce
  misc_feature (60)..(65): n is a, c, g, or t
agctgctactgagaattcagaaaatgacacttcagcttcccgatatccgacggtagtgtnnnnnnccattagtgactctga

SEQ ID NO: 551 - DNA, 77 bp, Artificial sequence
  Artificially generated seqeunce
  misc_feature (55)..(60): n is a, c, g, or t
gggggacactcaggcccttctttacttcagcttcccgatatccgacggtagtgtnnnnnngtcagggctgggtcctt

SEQ ID NO: 552 - DNA, 81 bp, Artificial sequence
  Artificially generated seqeunce
  misc_feature (53)..(58): n is a, c, g, or t
tcttcaaacaccaggcccctcccttcagcttcccgatatccgacggtagtgtnnnnnncagacacctttcgggatttcagg

SEQ ID NO: 553 - DNA, 79 bp, Artificial sequence
  Artificially generated seqeunce
  misc_feature (58)..(63): n is a, c, g, or t
gctggggtagtgctgagactaggttcacttcagcttcccgatatccgacggtagtgtnnnnnnctctttgcctctattt

SEQ ID NO: 554 - DNA, 81 bp, Artificial sequence
  Artificially generated seqeunce
  misc_feature (60)..(65): n is a, c, g, or t
cagatggtacccacagaacttgtagagaacttcagcttcccgatatccgacggtagtgtnnnnnnctctgcagagctttgg

SEQ ID NO: 555 - DNA, 6766 bp, Artificial sequence
  Artificially generated seqeunce
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtgtaatagctcctgcatgggttttagagctagaaatagcaagttaaaataaggctagtccgttatcaacttgaaaaagtggcaccgagtcggtgctttttttctagaagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggaagagcgagctcttctgttttagagctagaaatagcaagttaaaataaggctagtccgttatcaacttgaaaaagtggcaccgagtcggtgcttttttggtaccaggtcttgaaaggagtgggaattggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggcgtacggccaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaggagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtggctagcggaagcggagccactaacttctccctgttgaaacaagcaggggatgtcgaagagaatcccgggccacccaagaagaagaggaaggtgtccaatctcctgactgttcaccagaacctccctgcgctgccagtagatgccactagcgatgaggtcaggaaaaatctcatggatatgtttagggatagacaggcgttttctgaacacacctggaaaatgctgcttagcgtgtgccgatcctgggcagcctggtgtaagctgaacaatcgcaaatggttccccgccgagccggaggacgtgcgcgattacctgctgtatctccaggcaagagggctggctgtcaagactatccagcagcacttgggccaactgaatatgctgcatcgacgcagcgggctcccccggcctagcgattcaaacgcagtctcccttgttatgaggagaattagaaaggaaaacgtagatgcgggtgagagggctaagcaggctctcgcttttgagcggactgatttcgaccaggtcagatccctgatggagaacagcgatcggtgccaggacatcaggaacctcgcatttctgggaattgcatataacacacttctgcgcatagctgagatcgcccggatcagagtgaaagacatcagtcgaacggacggcggccggatgcttattcatattggacgcacaaagacattggtcagcaccgctggcgttgaaaaggccttgtccctgggcgtaacgaagctggtggaaagatggatctcagtgtccggcgtggctgacgaccctaataattacttgttctgtcgagtgagaaaaaacggagtcgccgcgccctctgccaccagccaattgagtacacgggcccttgaagggatctttgaggcaacccaccgactcatatacggagccaaggatgacagtggccagaggtatctcgcctggtcaggtcattctgctagggtgggggccgcacgagacatggcgcgggcaggagtctccataccagagattatgcaagctggaggttggacaaatgtgaacatcgttatgaactatatccgcaatcttgactctgaaaccggggccatggtgagactgctcgaagatggtgactacccatacgatgttccagattacgcttaagaattcgatatcaagcttaataaaagatctttattttcattagatctgtgtgttggttttttgtgtggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 556 - DNA, 7019 bp, Artificial seqeunce
  Artifically generated seqeunce
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtgtaatagctcctgcatgggttttagagctagaaatagcaagttaaaataaggctagtccgttatcaacttgaaaaagtggcaccgagtcggtgctttttttctagaagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggaagagcgagctcttctgttttagagctagaaatagcaagttaaaataaggctagtccgttatcaacttgaaaaagtggcaccgagtcggtgcttttttggtaccgcggcctctagactcgaggggctggaagctacctttgacatcatttcctctgcgaatgcatgtataatttctacagaacctattagaaaggatcacccagcctctgcttttgtacaactttcccttaaaaaactgccaattccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactaagagtgctctagttttgcaatacaggacatgctataaaaatggaaagataccggtgccaccatggccccaaaggttaaccgtacggccaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaggagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtggctagcggaagcggagccactaacttctccctgttgaaacaagcaggggatgtcgaagagaatcccgggccacccaagaagaagaggaaggtgtccaatctcctgactgttcaccagaacctccctgcgctgccagtagatgccactagcgatgaggtcaggaaaaatctcatggatatgtttagggatagacaggcgttttctgaacacacctggaaaatgctgcttagcgtgtgccgatcctgggcagcctggtgtaagctgaacaatcgcaaatggttccccgccgagccggaggacgtgcgcgattacctgctgtatctccaggcaagagggctggctgtcaagactatccagcagcacttgggccaactgaatatgctgcatcgacgcagcgggctcccccggcctagcgattcaaacgcagtctcccttgttatgaggagaattagaaaggaaaacgtagatgcgggtgagagggctaagcaggctctcgcttttgagcggactgatttcgaccaggtcagatccctgatggagaacagcgatcggtgccaggacatcaggaacctcgcatttctgggaattgcatataacacacttctgcgcatagctgagatcgcccggatcagagtgaaagacatcagtcgaacggacggcggccggatgcttattcatattggacgcacaaagacattggtcagcaccgctggcgttgaaaaggccttgtccctgggcgtaacgaagctggtggaaagatggatctcagtgtccggcgtggctgacgaccctaataattacttgttctgtcgagtgagaaaaaacggagtcgccgcgccctctgccaccagccaattgagtacacgggcccttgaagggatctttgaggcaacccaccgactcatatacggagccaaggatgacagtggccagaggtatctcgcctggtcaggtcattctgctagggtgggggccgcacgagacatggcgcgggcaggagtctccataccagagattatgcaagctggaggttggacaaatgtgaacatcgttatgaactatatccgcaatcttgactctgaaaccggggccatggtgagactgctcgaagatggtgactacccatacgatgttccagattacgcttaagaattcgatatcaagcttaataaaagatctttattttcattagatctgtgtgttggttttttgtgtggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 557 - DNA, 502 bp, Artificial sequence
  Artificially generated seqeunce
gcggcctctagactcgaggggctggaagctacctttgacatcatttcctctgcgaatgcatgtataatttctacagaacctattagaaaggatcacccagcctctgcttttgtacaactttcccttaaaaaactgccaattccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactaagagtgctctagttttgcaatacaggacatgctataaaaatggaaagataccggtgccaccatggccccaaag